(19) United States
(12) Patent Application Publication
(10) Pub. No.: US 2024/0208953 A1
(43) Pub. Date: Jun. 27, 2024

(54) "Cyclopenta[c]pyrrol Negative Allosteric Modulators of NR2B"

(71) Applicant: Novartis AG, Basel (CH)

(72) Inventors: Kevin Matthew GARDINIER, Cambridge, MA (US); Mark Patrick HEALY, Cambridge, MA (US); Keith JENDZA, Cambridge, MA (US); Yue PAN, Cambridge, MA (US); Kate Yaping WANG, Cambridge, MA (US); Fan YANG, Cambridge, MA (US)

(21) Appl. No.: 18/551,739

(22) PCT Filed: Mar. 24, 2022
(86) PCT No.: PCT/US2022/021624

§ 371 Date: Sep. 21, 2023

Related U.S. Application Data
(60) Provisional application No. 63/166,516, filed on Mar. 26, 2021.

(51) Int. Cl. C07D 413/06; A61K 31/404; A61K 31/416; A61K 31/423; A61K 31/428; A61K 31/437; A61K 31/4709; A61K 31/536; A61K 31/538; A61K 31/5415; A61K 31/553; C07D 401/06; C07D 403/06; C07D 405/14; C07D 417/06; C07D 471/04

(52) U.S. Cl. CPC C07D 413/06; A61K 31/404; A61K 31/416; A61K 31/423; A61K 31/428; A61K 31/437; A61K 31/4709; A61K 31/536; A61K 31/538; A61K 31/5415; A61K 31/553; C07D 401/06; C07D 403/06; C07D 405/14; C07D 417/06; C07D 471/04

(57) ABSTRACT

The present disclosure provides a compound of formula (I), or a pharmaceutically acceptable salt thereof; a method for manufacturing the compounds of the disclosure, and its therapeutic uses. The present disclosure further provides a combination of pharmacologically active agents and a pharmaceutical composition.

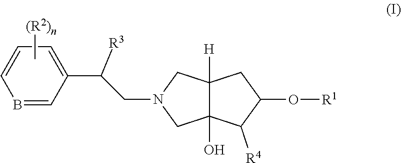

### FIELD OF THE DISCLOSURE

The present disclosure relates to compounds that selectively modulate the activity of NR1/NR2B receptors.

### BACKGROUND OF THE DISCLOSURE

The NMDA receptor is arguably an important signaling mechanism in the human brain. The brain processes a complex array of information to allow humans to function, storing information from the past and analyzing this information in the context of the present to respond and plan for the future. These incredibly complex computations are mediated at the molecular level by the continual adjustment of the strength of synapses, the nodes for communication between nerve cells (estimated at about 60 trillion in the human brain).

Glutamate is the major excitatory neurotransmitter in the brain, utilized at 80% of these synapses. NMDA receptors are one of three classes that mediate synaptic transmission using glutamate. NMDA receptors play a critical role in regulating the strength of synapses, that is, in regulating synaptic plasticity. Thus, the NMDA receptor is at the molecular core of brain function, and in particular the cognitive functions of learning and memory. These facts underlie the tremendous therapeutic utility of modulating NMDA receptor function with new drugs to treat a broad range of neuropsychiatric disease and cognitive dysfunction.

The molecular basis of NMDA receptor function is increasingly well understood. The NMDA receptor is composed of four protein subunits, two NR1 subunits and two NR2 subunits. An NR1 subunit derived from a single gene is ubiquitously expressed throughout the brain and is common to all NMDA receptors. However, the four different NR2 subunits, NR2A-D, are derived from separate genes that are differentially expressed in different brain regions and by distinct populations of neurons within a particular region. Furthermore, individual neurons may express more than one NR2 subunit and individual NMDA receptors expressed by such neurons may contain two of the same NR2 subunits (for example, 2 NR2B subunits) or two different subunits (one NR2A and one NR2B subunit). Therefore, a drug that selectively modulates the activity of one NR2 subunit may do so at receptors that express two of the targeted subunits, or only one of the targeted subunits. Thus there is a need for new treatments for diseases related to the NR1/NR2B receptor.

### SUMMARY OF THE DISCLOSURE

Various embodiments of the disclosure are described herein.

Within certain aspects, provided herein is a compound of formula (I) or a pharmaceutically acceptable salt thereof:

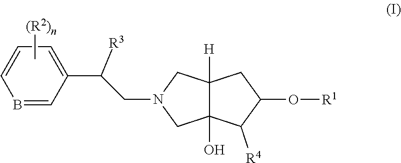

In another aspect, the disclosure provides a pharmaceutical composition comprising a compound of formula (I) or a pharmaceutically acceptable salt thereof.

The present disclosure further pertains to compounds that selectively modulate the activity of NMDA receptors that contain an NR2B subunit, which encompasses receptors containing two NR2B subunits or one NR2B subunit in combination with one other NR2 subunit (i.e., NR2A/NR2B, NR2B/NR2C, or NR2B/NR2D receptors). Such compounds can decrease the activity of NR2B-containing NMDA receptors. The present disclosure also pertains to the therapeutic uses of such compounds.

In a further aspect, the disclosure provides for a compound of formula (I), or a pharmaceutically acceptable salt thereof for use in therapy, in particular in the treatment of Parkinson's disease, Huntington's disease, Rett syndrome, amyotrophic lateral sclerosis, multiple sclerosis, seizure disorders, autism, autism spectrum disorders, Fragile X syndrome, tuberous sclerosis, Down's syndrome, pain, migraine, tinnitus, bipolar disorder, obsessive-compulsive disorder, anxiety disorder, post-traumatic stress disorder (PTSD), cocaine use disorder, major depressive disorder, refractory or treatment resistant depression, or suicidality, comprising administration of a therapeutically effective amount of a compound.

### DETAILED DESCRIPTION OF THE DISCLOSURE

The disclosure therefore provides a compound of Formula (I):

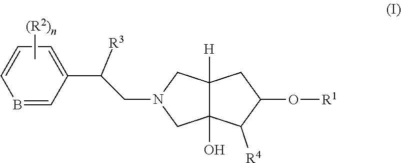

or a pharmaceutically acceptable salt thereof, wherein:
R¹ is a C₃₋₈cycloalkyl, three to seven membered heterocyclyl, phenyl, naphthyl, or heteroaryl, each of which is optionally substituted with one or more R⁵;
R² is OH, CN, halogen, OR⁶, SH, SR⁶, C₁₋₆ alkyl, haloC₁₋₆ alkyl, NH₂, NHR⁶, hydroxyC₁₋₆alkyl, N(R⁶)(R⁶′), NHS(O)₂R⁶, or NHCOR⁶, wherein R² is not OH when in the para position;
or two R² groups, together with the ring carbon atoms to which they are attached, combine to form a five- to seven-membered heterocyclic ring or a five- or six-membered heteroaryl ring;
R³ is H, O, or OH;
R⁴ is H or OH;
R⁵ is halogen, OH, C₁₋₆alkyl, OR⁶, CN, NH₂, NHR⁶, N(R⁶)(R⁶′), SH, SR⁶, SOR⁶, SO₂R⁶, SO₂NHR⁶, SO₂N(R⁶)(R⁶′), CONH₂, CONHR⁶,or CON(R⁶)(R⁶′);
each R⁶ and R⁶′ is independently selected from the group consisting of H, O—C₁₋₆alkyl, C₁₋₆alkyl, and haloC₁₋₆ alkyl;
B is N or CRx;
each Rx is independently H, C₁₋₃alkyl, or halogen; and
each n is independently 0, 1, 2, 3, or 4.

One embodiment is a compound of Formula II:

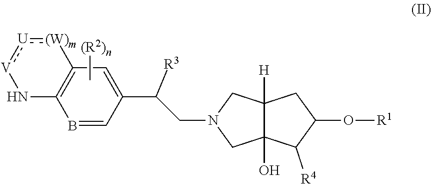

or a pharmaceutically acceptable sail, thereof wherein:
R¹ is a C₃₋₈cycloalkyl, three to seven membered heterocyclyl, phenyl, naphthyl, or heteroaryl, each of which is optionally substituted with one or more R⁵;
R² is OH, CN, halogen, OR⁶, SH, SR⁶, C₁₋₆alkyl, haloC₁₋₆ alkyl, NH₂, NHR⁶, hydroxyC₁₋₆ alkyl, N(R⁶)(R⁶′), NHS(O)₂R⁶, or NHCOR⁶;
R³ is H, O, or OH;
R⁴ is H or OH;
R⁵ is halogen, OH, C₁₋₆ alkyl, OR⁶, CN, NH₂, NHR⁶, N(R⁶)(R⁶′), SH, SR⁶, SOR⁶, SO₂R⁶, SO₂NHR⁶, SO₂N(R⁶)(R⁶′), CONH₂, CONHR⁶, or CON(R⁶)(R⁶′);
each R⁶ and R⁶′ is independently selected from the group consisting of H, O—C₁₋₆ alkyl, C₁₋₆ alkyl, and haloC₁₋₆ alkyl;
B is N or CRx;
V is carbonyl, CH, or N;
U is O, S, CRx, or CRxRx;
each Rx is independently H, C₁₋₃alkyl, or halogen;
each W is independently O, CH, or CH₂;
[symbol] is an optional double bond;
m is 0, 1, or 2; and
each n is independently 0, 1, 2, 3, or 4.

Another embodiment is a compound of Formula III:

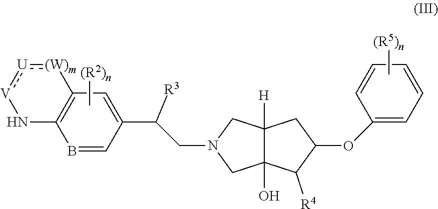

or a pharmaceutically acceptable salt, thereof wherein:
R² is OH, CN, halogen, OR⁶, SH, SR⁶, C₁₋₆alkyl, haloC₁₋₆ alkyl, NH₂, NHR⁶, hydroxyC₁₋₆alkyl, N(R⁶)(R⁶′), NHS(O)₂R⁶, NHCOR⁶;
R³ is H, O, or OH;
R⁴ is H or OH;
R⁵ is halogen, OH, C₁₋₆alkyl, OR⁶, CN, NH₂, NHR⁶, N(R⁶)(R⁶′), SH, SR⁶, SOR⁶, SO₂R⁶, SO₂NHR⁶, SO₂N(R⁶)(R⁶′), CONH₂, CONHR⁶, and CON(R⁶)(R⁶′);
each R⁶ and R⁶′ is independently selected from the group consisting of H, O—C₁₋₆alkyl,
C₁₋₆alkyl, and haloC₁₋₆ alkyl;
B is N or CRx;
V is carbonyl, CH, or N;
U is O, S, CRx, or CRxRx;
each Rx is independently H, C₁₋₃alkyl, or halogen;
each W is independently O, CH, or CH₂;
[symbol] is an optional double bond;
m is 0, 1, or 2; and
each n is independently 0, 1, 2, 3, or 4.

Another embodiment is a compound of Formula IV:

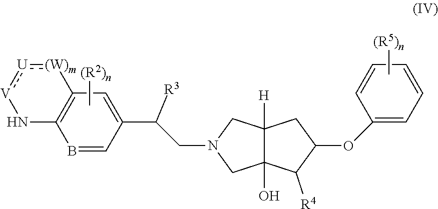

or a pharmaceutically acceptable salt, thereof wherein:
R² is halogen;
R³ is H or OH;
R⁴ is H or OH;
R⁵ is halogen;
B is N or CH;
V is carbonyl, CH, or N;
U is O, S, CRx, or CRxRx;
each Rx is independently H, C₁₋₃ alkyl, or halogen;
each W is independently O, CH, or CH₂;
[symbol] is an optional double bond;
m is 0, 1, or 2; and
each n is independently 0, 1, 2, 3, or 4.

Another embodiment is a compound of Formula IVa:

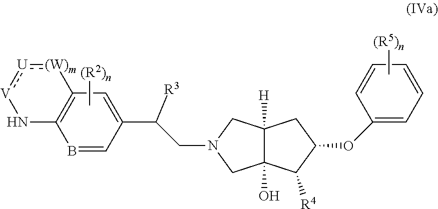

or a pharmaceutically acceptable salt, thereof.

Another embodiment is a compound of Formula IVb:

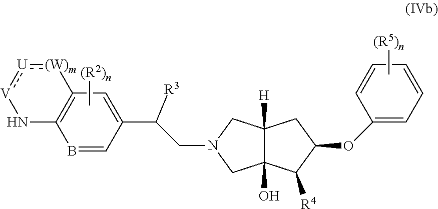

or a pharmaceutically acceptable salt, thereof.

Another embodiment is a compound of Formula IVc:

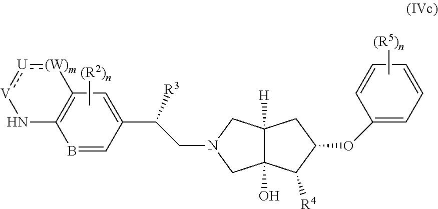

or a pharmaceutically acceptable salt, thereof.

Another embodiment is a compound of Formula IVd:

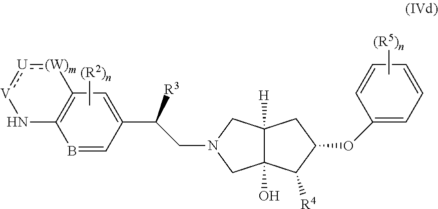

or a pharmaceutically acceptable salt, thereof.

Another embodiment is a compound of Formula IVe:

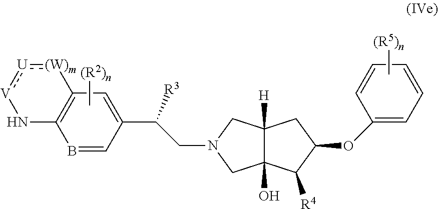

or a pharmaceutically acceptable salt, thereof.

Another embodiment is a compound of Formula IVf:

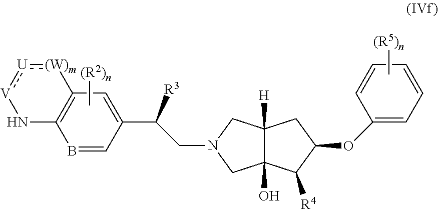

or a pharmaceutically acceptable sail, thereof.

Another embodiment is a compound of Formula V:

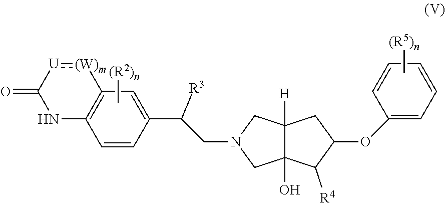

or a pharmaceutically acceptable salt, thereof.

Another embodiment is a compound of Formula Va:

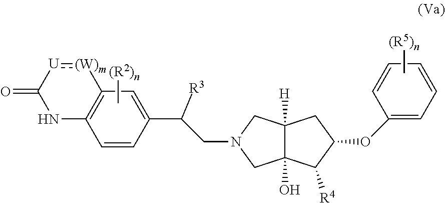

or a pharmaceutically acceptable salt, thereof.

Another embodiment is a compound of Formula Vb:

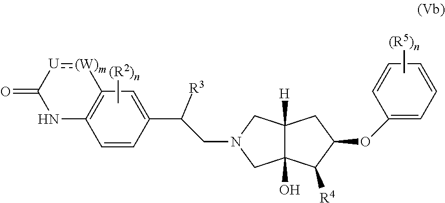

or a pharmaceutically acceptable salt, thereof.

Another embodiment is a compound of Formula Vc:

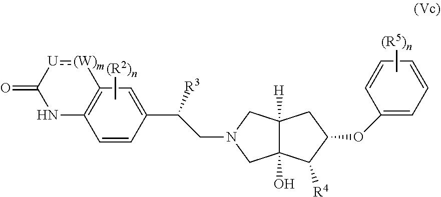

or a pharmaceutically acceptable salt, thereof.

Another embodiment is a compound of Formula Vd:

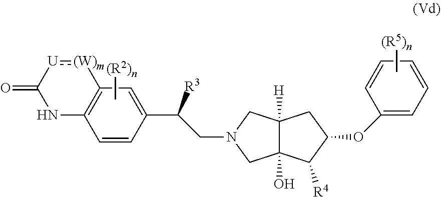

or a pharmaceutically acceptable sail, thereof.

Another embodiment is a compound of Formula Ve:

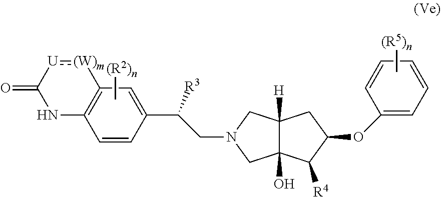

or a pharmaceutically acceptable salt, thereof.

Another embodiment is a compound of Formula Vf:

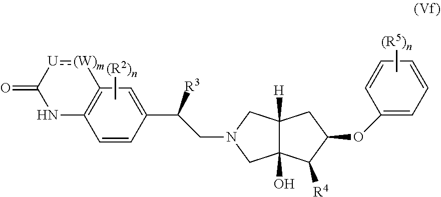

or a pharmaceutically acceptable salt, thereof.

In another embodiment, U is CRxRx, W is CH₂.

In another embodiment, U is CRxRx, W is CH₂, and m is 1.

In another embodiment, U is CRxRx, W is CH₂, and m is 2.

In another embodiment, U is CRx, W is CH, and m is 1.

In another embodiment, U is CRxRx, W is O and m is 1.

In another embodiment, U is CRxRx, one W is O, one W is CH₂, and m is 2.

In another embodiment, U is CRxRx, and m is 0.

In another embodiment, U is O, and W is CH₂.

In another embodiment, U is O, and W is CH₂, and m is 1.

In another embodiment, U is O, and W is CH₂, and m is 2.

In another embodiment, U is O, and m is 0.

In another embodiment, U is S, W is CH₂, and m is 1.

In another embodiment, U is S, and m is 0.

Another embodiment is the compound of Formula VI:

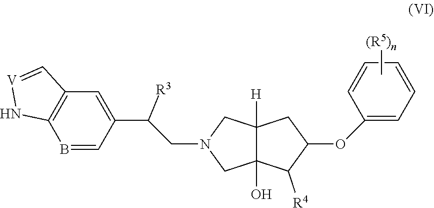

or a pharmaceutically acceptable sail, thereof, wherein:
R³ is H or OH;
R⁴ is H or OH;
R⁵ is halogen;
V is CH or N;
B is N or CH;
each n is independently 0, 1, 2, 3, or 4.

In another embodiment, the compound of Formula VIa:

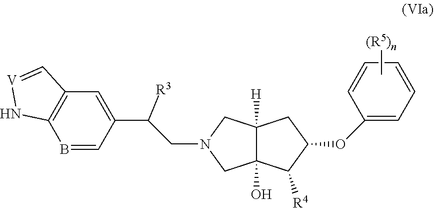

or a pharmaceutically acceptable salt, thereof.

In another embodiment, the compound of Formula VIb:

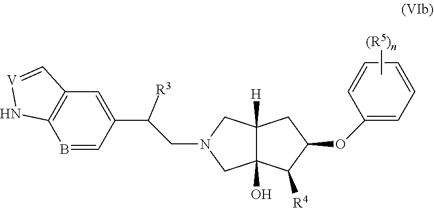

or a pharmaceutically acceptable salt, thereof.

In another embodiment, the compound of Formula VIc:

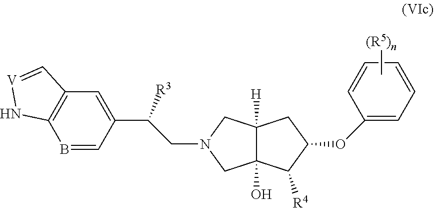

or a pharmaceutically acceptable salt, thereof.

In another embodiment, the compound of Formula VId:

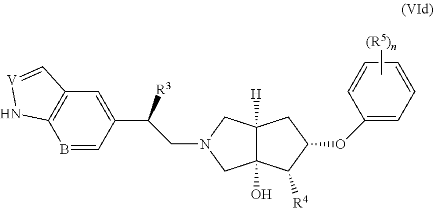

or a pharmaceutically acceptable salt, thereof.

In another embodiment, the compound of Formula Ille:

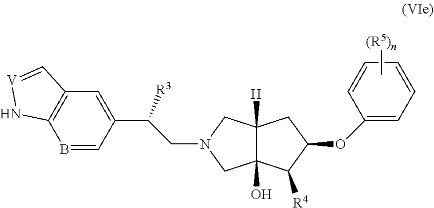

or a pharmaceutically acceptable sail, thereof.

In another embodiment, the compound of Formula VIf:

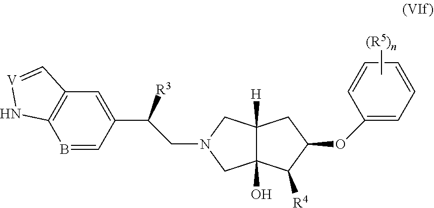

or a pharmaceutically acceptable salt, thereof.

In another embodiment, R² or R⁵ is F.

In another embodiment, R³ is H.

In another embodiment, R³ is OH.

In another embodiment, R⁴ is H.

In another embodiment, R⁴ is OH.

In another embodiment R² is CN, halogen, OR⁶, SH, SR⁶, C₁₋₆ alkyl, haloC₁₋₆ alkyl, or hydroxyC₁₋₆ alkyl.

In another embodiment R² is halogen, C₁₋₆ alkyl, haloC₁₋₆ alkyl, or hydroxyC₁₋₆ alkyl.

In another embodiment R² is halogen, C₁₋₆ alkyl, or haloC₁₋₆ alkyl.

In another embodiment R⁵ is halogen, OH, C₁₋₆ alkyl, OR⁶, CN, SH, or SR⁶.

In another embodiment R⁵ is halogen, OH, C₁₋₆ alkyl, or OR⁶.

In another embodiment R⁵ is halogen, OH, or C₁₋₆ alkyl.

Specific compounds include:
6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
5-((R)-1-hydroxy-2-((3aS,5S,6aR)-3a-hydroxy-5-phenoxyhexahydrocyclopenta[c]pyrrol-2(1H)-yl)ethyl)indolin-2-one;
5-((S)-1-hydroxy-2-((3aS,5S,6aR)-3a-hydroxy-5-phenoxyhexahydrocyclopenta[c]pyrrol-2(1H)-yl)ethyl)indolin-2-one;
5-((R)-2-((3aS,4S,5S,6aR)-3a,4-dihydroxy-5-phenoxyhexahydrocyclopenta[c]pyrrol-2(1H)-yl)-1-hydroxyethyl)indolin-2-one;
5-((S)-2-((3aS,4S,5S,6aR)-3a,4-dihydroxy-5-phenoxyhexahydrocyclopenta[c]pyrrol-2(1H)-yl)-1-hydroxyethyl)indolin-2-one;
5-((R)-2-((3aR,4R,5R,6aS)-3a,4-dihydroxy-5-phenoxyhexahydrocyclopenta[c]pyrrol-2(1H)-yl)-1-hydroxyethyl)indolin-2-one;
5-((S)-2-((3aR,4R,5R,6aS)-3a,4-dihydroxy-5-phenoxyhexahydrocyclopenta[c]pyrrol-2(1H)-yl)-1-hydroxyethyl)indolin-2-one;
6-((R)-2-((3aS,4S,5S,6aR)-3a,4-dihydroxy-5-phenoxyhexahydrocyclopenta[c]pyrrol-2(1H)-yl)-1-hydroxyethyl)-3,4-dihydroquinolin-2(1H)-one;
6-((S)-2-((3aS,4S,5S,6aR)-3a,4-dihydroxy-5-phenoxyhexahydrocyclopenta[c]pyrrol-2(1H)-yl)-1-hydroxyethyl)-3,4-dihydroquinolin-2(1H)-one;
6-((R)-2-((3aR,4R,5R,6aS)-3a,4-dihydroxy-5-phenoxyhexahydrocyclopenta[c]pyrrol-2(1H)-yl)-1-hydroxyethyl)-3,4-dihydroquinolin-2(1H)-one;
6-((S)-2-((3aR,4R,5R,6aS)-3a,4-dihydroxy-5-phenoxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one;
8-fluoro-6-((R)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one;
8-fluoro-6-((S)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one;
8-fluoro-6-((R)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one;
8-fluoro-6-((S)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one;
7-((R)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one;
7-((S)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one;
6-((R)-1-hydroxy-2-((3aS,5S,6aR)-3a-hydroxy-5-phenoxyhexahydrocyclopenta[c]pyrrol-2(1H)-yl)ethyl)quinolin-2(1H)-one;
6-((S)-1-hydroxy-2-((3aS,5S,6aR)-3a-hydroxy-5-phenoxyhexahydrocyclopenta[c]pyrrol-2(1H)-yl)ethyl)quinolin-2(1H)-one;
5-fluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
5-fluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
7-fluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
7-fluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
6-((R)-1-hydroxy-2-((3aS,5S,6aR)-3a-hydroxy-5-phenoxyhexahydrocyclopenta[c]pyrrol-2(1H)-yl)ethyl)-3,3-dimethyl-3,4-dihydroquinolin-2(1H)-one;
6-((S)-1-hydroxy-2-((3aS,5S,6aR)-3a-hydroxy-5-phenoxyhexahydrocyclopenta[c]pyrrol-2(1H)-yl)ethyl)-3,3-dimethyl-3,4-dihydroquinolin-2(1H)-one;
7-((R)-2-((3aS,5S,6aR)-5-(2-fluorophenoxy)-3a-hydroxyhexahydrocyclopenta[c]pyrrol-2(1H)-yl)-1-hydroxyethyl)-4,5-dihydrobenzo[d][1,3]oxazepin-2(1H)-one;
7-((S)-2-((3aS,5S,6aR)-5-(2-fluorophenoxy)-3a-hydroxyhexahydrocyclopenta[c]pyrrol-2(1H)-yl)-1-hydroxyethyl)-4,5-dihydrobenzo[d][1,3]oxazepin-2(1H)-one;
5-fluoro-7-((R)-1-hydroxy-2-((3aS,5S,6aR)-3a-hydroxy-5-phenoxyhexahydrocyclopenta[c]pyrrol-2(1H)-yl)ethyl)-2H-benzo[b][1,4]oxazin-3(4H)-one;
5-fluoro-7-((S)-1-hydroxy-2-((3aS,5S,6aR)-3a-hydroxy-5-phenoxyhexahydrocyclopenta[c]pyrrol-2(1H)-yl)ethyl)-2H-benzo[b][1,4]oxazin-3(4H)-one;
6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]thiazin-2-one;
6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]thiazin-2-one;
6-((R)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]thiazin-2-one;
6-((S)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]thiazin-2-one;
8-fluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]thiazin-2-one;
8-fluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]thiazin-2-one;
6-((R)-1-hydroxy-2-((3aS,5S,6aR)-3a-hydroxy-5-phenoxyhexahydrocyclopenta[c]pyrrol-2(1H)-yl)ethyl)benzo[d]thiazol-2(3H)-one;
6-((S)-1-hydroxy-2-((3aS,5S,6aR)-3a-hydroxy-5-phenoxyhexahydrocyclopenta[c]pyrrol-2(1H)-yl)ethyl)benzo[d]thiazol-2(3H)-one;
6-((R)-2-((3aS,5S,6aR)-5-(2-fluorophenoxy)-3a-hydroxyhexahydrocyclopenta[c]pyrrol-2(1H)-yl)-1-hydroxyethyl)benzo[d]thiazol-2(3H)-one;
6-((S)-2-((3aS,5S,6aR)-5-(2-fluorophenoxy)-3a-hydroxyhexahydrocyclopenta[c]pyrrol-2(1H)-yl)-1-hydroxyethyl)benzo[d]thiazol-2(3H)-one;
A mixture of:
(S)-3,8-difluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
(S)-3,8-difluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
(R)-3,8-difluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
(R)-3,8-difluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
3,8-difluoro-6-((R)-1-hydroxy-2-((3aS,5S,6aR)-3a-hydroxy-5-phenoxyhexahydrocyclopenta[c]pyrrol-2(1H)-yl)ethyl)quinolin-2(1H)-one;
3,8-difluoro-6-((S)-1-hydroxy-2-((3aS,5S,6aR)-3a-hydroxy-5-phenoxyhexahydrocyclopenta[c]pyrrol-2(1H)-yl)ethyl)quinolin-2(1H)-one;
(3aS,5S,6aR)-5-(2,4-difluorophenoxy)-2-((R)-2-hydroxy-2-(1H-indazol-5-yl)ethyl)hexahydrocyclopenta[c]pyrrol-3a(1H)-ol;
(3aS,5S,6aR)-5-(2,4-difluorophenoxy)-2-((S)-2-hydroxy-2-(1H-indazol-5-yl)ethyl)hexahydrocyclopenta[c]pyrrol-3a(1H)-ol;
(3aR,5R,6aS)-5-(2,4-difluorophenoxy)-2-((R)-2-hydroxy-2-(1H-indazol-5-yl)ethyl)hexahydrocyclopenta[c]pyrrol-3a(1H)-ol;
(3aR,5R,6aS)-5-(2,4-difluorophenoxy)-2-((R)-2-hydroxy-2-(1H-indazol-5-yl)ethyl)hexahydrocyclopenta[c]pyrrol-3a(1H)-ol;
6-((R)-2-((3aS,5S,6aR)-5-(4-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(4-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(4-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(4-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(3-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(3-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(3-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(3-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2,3-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2,3-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2,3-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2,3-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2,4-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2,4-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2,4-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2,4-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2,5-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2,5-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2,5-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2,5-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2,6-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2,6-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2,6-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2,6-difluorophenoxy)-3a-hydroxyhexahydrocyclopenta[c]pyrrol-2(1H)-yl)-1-hydroxyethyl)-3,4-dihydroquinolin-2(1H)-one;
6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
8-fluoro-6-((R)-2-((3aS,4S,5S,6aR)-5-(2-fluorophenoxy)-3a,4-dihydroxyhexahydrocyclopenta[c]pyrrol-2(1H)-yl)-1-hydroxyethyl)-3,4-dihydroquinolin-2(1H)-one;
8-fluoro-6-((S)-2-((3aS,4S,5S,6aR)-5-(2-fluorophenoxy)-3a,4-dihydroxyhexahydrocyclopenta[c]pyrrol-2(1H)-yl)-1-hydroxyethyl)-3,4-dihydroquinolin-2(1H)-one;
9-fluoro-7-((R)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one;
9-fluoro-7-((S)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one;
8-fluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
8-fluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
9-fluoro-7-((R)-1-hydroxy-2-((3aS,5S,6aR)-3a-hydroxy-5-phenoxyhexahydrocyclopenta[c]pyrrol-2(1H)-yl)ethyl)-1,5-dihydrobenzo[e][1,4]oxazepin-2(3H)-one;
9-fluoro-7-((S)-1-hydroxy-2-((3aS,5S,6aR)-3a-hydroxy-5-phenoxyhexahydrocyclopenta[c]pyrrol-2(1H)-yl)ethyl)-1,5-dihydrobenzo[e][1,4]oxazepin-2(3H)-one;
8-fluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
8-fluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
5-fluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
5-fluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
8-fluoro-6-((R)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]oxazin-2-one;
8-fluoro-6-((S)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]oxazin-2-one;
7-fluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
7-fluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
5,8-difluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
5,8-difluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
7,8-difluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
7,8-difluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
6-((R)-1-hydroxy-2-((3aS,5S,6aR)-3a-hydroxy-5-phenoxyhexahydrocyclopenta[c]pyrrol-2(1H)-yl)ethyl)benzo[d]oxazol-2(3H)-one;
6-((S)-1-hydroxy-2-((3aS,5S,6aR)-3a-hydroxy-5-phenoxyhexahydrocyclopenta[c]pyrrol-2(1H)-yl)ethyl)benzo[d]oxazol-2(3H)-one;
6-((R)-2-((3aS,5S,6aR)-5-(2-fluorophenoxy)-3a-hydroxyhexahydrocyclopenta[c]pyrrol-2(1H)-yl)-1-hydroxyethyl)benzo[d]oxazol-2(3H)-one;
6-((S)-2-((3aS,5S,6aR)-5-(2-fluorophenoxy)-3a-hydroxyhexahydrocyclopenta[c]pyrrol-2(1H)-yl)-1-hydroxyethyl)benzo[d]oxazol-2(3H)-one;
6-((R)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]oxazin-2-one;
6-((S)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]oxazin-2-one;
4-fluoro-6-((R)-1-hydroxy-2-((3aS,5S,6aR)-3a-hydroxy-5-phenoxyhexahydrocyclopenta[c]pyrrol-2(1H)-yl)ethyl)benzo[d]thiazol-2(3H)-one;
4-fluoro-6-((S)-1-hydroxy-2-((3aS,5S,6aR)-3a-hydroxy-5-phenoxyhexahydrocyclopenta[c]pyrrol-2(1H)-yl)ethyl)benzo[d]thiazol-2(3H)-one;
7-((R)-2-((3aS,5S,6aR)-5-(2-fluorophenoxy)-3a-hydroxyhexahydrocyclopenta[c]pyrrol-2(1H)-yl)-1-hydroxyethyl)-1,3,4,5-tetrahydro-2H-benzo[b]azepin-2-one;
7-((S)-2-((3aS,5S,6aR)-5-(2-fluorophenoxy)-3a-hydroxyhexahydrocyclopenta[c]pyrrol-2(1H)-yl)-1-hydroxyethyl)-1,3,4,5-tetrahydro-2H-benzo[b]azepin-2-one;
(3aS,4S,5S,6aR)-5-(2-fluorophenoxy)-2-((R)-2-hydroxy-2-(1H-pyrrolo[2,3-b]pyridin-5-yl)ethyl)hexahydrocyclopenta[c]pyrrole-3a,4(1H)-diol; and
(3aS,4S,5S,6aR)-5-(2-fluorophenoxy)-2-((S)-2-hydroxy-2-(1H-pyrrolo[2,3-b]pyridin-5-yl)ethyl)hexahydrocyclopenta[c]pyrrole-3a,4(1H)-diol, or a pharmaceutically acceptable salt thereof.

One embodiment is a pharmaceutical composition comprising a compound of Formula (1) or a pharmaceutically acceptable salt thereof.

Another embodiment is a method for the treatment of Parkinson's disease, Huntington's disease, amyotrophic lateral sclerosis, multiple sclerosis, seizure disorders, autism, autism spectrum disorders, Fragile X syndrome, tuberous sclerosis, Down's syndrome, bipolar disorder, obsessive-compulsive disorder, anxiety disorder, major depressive disorder, refractory or treatment resistant depression, or suicidality comprising administration of a therapeutically effective amount of a compound of Formula (1), or a pharmaceutically acceptable salt thereof to a patient in need of treatment thereof.

Another embodiment is a method for the treatment of post-traumatic stress disorder (PTSD).

Another embodiment is a method for the treatment of cocaine use disorder.

Another embodiment is a method for the treatment of pain and migraine.

Another embodiment is a method for the treatment of Rett Syndrome.

Another embodiment is a method for the treatment of tinnitus.

Unless specified otherwise, the term “compounds of the present disclosure” or “compound of the present disclosure” refers to compounds of formula (1) subformulae thereof, and exemplified compounds, and salts thereof, as well as all stereoisomers (including diastereoisomers and enantiomers), rotamers, tautomers and isotopically labeled compounds (including deuterium substitutions), as well as inherently formed moieties.

### Definitions

As used herein, the term “Halogen”, “halide”, or, alternatively, “halo” refers to bromo, chloro, fluoro or iodo.

As used herein, the term “C₁₋₆alkyl” refers to a straight or branched hydrocarbon chain radical consisting solely of carbon and hydrogen atoms, containing no unsaturation, having from one to six carbon atoms, and which is attached to the rest of the molecule by a single bond. The term “C₁₋₄alkyl” is to be construed accordingly. Examples of C₁₋₆alkyl include, but are not limited to, methyl, ethyl, n-propyl, 1-methylethyl (iso-propyl), n-butyl, n-pentyl and 1,1-dimethylethyl (t-butyl).

As used herein, the term “C₃₋₈cycloalkyl” refers to a monocyclic or polycyclic radical that contains only carbons and hydrogen, having from three to eight ring atoms, and can be saturated or partially unsaturated. Examples of C₃₋₈cycloalkyl include, but are not limited to cyclopropyl, cyclobutyl, cyclopentyl, cyclopentyenyl, cyclohexyl, cycloheptyl, and cyclooctyl.

As used herein, the term “hydroxyC₁₋₆alkyl” refers to a C₁₋₆alkyl radical as defined above, wherein one of the hydrogen atoms of the C₁₋₆alkyl radical is replaced by OH. Examples of hydroxyC₁₋₆alkyl include, but are not limited to, hydroxy-methyl, 2-hydroxy-ethyl, 2-hydroxy-propyl, 3-hydroxy-propyl and 5-hydroxy-pentyl.

As used herein, the term “haloC₁₋₆alkyl” refers to C₁₋₆alkyl radical, as defined above, substituted by one or more halo radicals, as defined above. Examples of haloC₁₋₆alkyl include, but are not limited to, trifluoromethyl, difluoromethyl, fluoromethyl, trichloromethyl, 2,2,2-trifluoroethyl, 1,3-dibromopropan-2-yl, 3-bromo-2-fluoropropyl and 1,4,4-trifluorobutan-2-yl.

As used herein, the term “Aryl” refers to an aromatic hydrocarbon ring system. Aryl groups are monocyclic ring systems or bicyclic ring systems. Monocyclic aryl ring refers to phenyl.

Bicyclic aryl rings refer to naphthyl. Aryl groups may be optionally substituted with one or more substituents as defined in formula (1).

As used herein, the term “Heterocyclic” or “heterocyclyl” refers to a 3 to 8 membered saturated or partially unsaturated monocyclic or bicyclic ring containing from 1 to 5 heteroatoms. Heterocyclic ring systems are not aromatic. Heterocyclic groups containing more than one heteroatom may contain different heteroatoms. Heterocyclic includes ring systems wherein a carbon atom is oxidized forming a cyclic ketone or lactam group. Heterocyclic also includes ring systems wherein a sulfur atom is oxidized to form SO or SO₂. Heterocyclic groups may be optionally substituted with one or more substituents as defined in formula (1). Heterocyclic groups are monocyclic, spiro, or fused or bridged bicyclic ring systems. Monocyclic heterocyclic have 3 to 7 ring atoms, unless otherwise defined. Examples of monocyclic heterocyclic groups include tetrahydrofuranyl, dihydrofuranyl, 1,4-dioxanyl, morpholinyl, 1,4-dithianyl, piperazinyl, piperidinyl, 1,3-dioxolanyl, imidazolidinyl, imidazolinyl, pyrrolinyl, pyrrolidinyl, tetrahydropyranyl, dihydropyranyl, oxathiolanyl, dithiolanyl, 1,3-dioxanyl, 1,3-dithianyl, oxathianyl, thiomorpholinyl and the like. Fused heterocyclic ring systems have from 8 to 11 ring atoms and include groups wherein a heterocyclic ring is fused to a phenyl or monocyclic heteroaryl ring. Examples of fused heterocyclic rings include 3,4-dihydroquinolin-2(1H)-onyl, indolin-2-onyl, quinolin-2(1H)-onyl, 1,3,4,5-tetrahydro-2H-benzo[b]azepin-2-onyl, 4,5-dihydrobenzo[d][1,3]oxazepin-2(1H)-onyl, 1,4-dihydro-2H-benzo[d][1,3]thiazin-2-onyl, benzo[d]thiazol-2(3H)-onyl, benzo[d]oxazol-2(3H)-onyl, 1H-indazolyl, 1H-indolyl, and the like.

As used herein, the term “Heteroaryl” refers to an aromatic ring system containing from 1 to 5 heteroatoms. Heteroaryl groups containing more than one heteroatom may contain different heteroatoms. Heteroaryl groups may be optionally substituted with one or more substituents as defined in formula (1). Heteroaryl groups are monocyclic ring systems or are fused bicyclic ring systems. Monocyclic heteroaryl rings have from 5 to 6 ring atoms. Bicyclic heteroaryl rings have from 8 to 10 member atoms. Heteroaryl includes, but is not limited to, pyrrolyl, pyrazolyl, imidazolyl, oxazolyl, isoxazolyl, oxadiazolyl, thiazolyl, isothiazolyl, thiadiazolyl, furanyl, furanzanyl, thienyl, triazolyl, pyridinyl, pyrimidinyl, pyridazinyl, trazinyl, tetrazinyl, tetrzolyl, indonyl, isoindolyl, indolizinyl, indazolyl, purinyl, quinolinyl, isoquinolinyl, quinoxalinyl, quinazolinyl, benzimidazolyl, benzopyranyl, benzopyranyl, benzoxazolyl, benzoisoxazolyl, benzofuranyl, benzothiazolyl, benzothienyl, and naphthyridinyl.

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present disclosure is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present disclosure. “Salts” include in particular “pharmaceutical acceptable salts”. The term “pharmaceutically acceptable salts” refers to salts that retain the biological effectiveness and properties of the compounds of this disclosure and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present disclosure provides compounds of the present disclosure in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the disclosure include, for example, isotopes of hydrogen.

For example, Formula (IV) is deuterated as shown in the compound of formula (IVg):

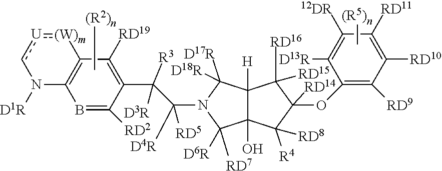

or a pharmaceutically acceptable salt thereof, wherein R⁵, R², and n are defined as in Formula (I), RD¹ through RD¹⁷ are independently H or D, and R³, R⁴ are independently H, D, or OH; V is carbonyl, CH, CD, or N; U is O, S, CRx, CRxRx; each Rx is independently H, D, C₁₋₃alkyl, or halogen; each W is independently O, CH, CD, CH₂ or CD₂; and B is N, CH, or CD.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present disclosure. The concentration of deuterium, may be defined by the isotopic enrichment factor. The term “isotopic enrichment factor” as used herein means the ratio between the isotopic abundance and the natural abundance of a specified isotope. If a substituent in a compound of this disclosure is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the disclosure include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. Accordingly, it should be understood that the disclosure includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present disclosure can generally be prepared by conventional techniques known to those skilled in the art or by processes analogous to those described in the accompanying Examples and Preparations using an appropriate isotopically-labeled reagent in place of the non-labeled reagent previously employed.

As used herein, the term “pharmaceutical composition” refers to a compound of the disclosure, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The term “a therapeutically effective amount” of a compound of the present disclosure refers to an amount of the compound of the present disclosure that will elicit the biological or medical response of a subject, for example, reduction or inhibition of an enzyme, receptor, ion channel, or a protein activity, or ameliorate symptoms, alleviate conditions, slow or delay disease progression, or prevent a disease, etc. In one embodiment, the term “a therapeutically effective amount” refers to the amount of the compound of the present disclosure that, when administered to a subject, is effective to (1) at least partially alleviate, prevent and/or ameliorate a condition, or a disorder or a disease (i) mediated by NR2B receptor, or (ii) associated with NR2B receptor activity, or (iii) characterized by activity (normal or abnormal) of NR2B receptor; or (2) reduce or inhibit the activity of NR2B receptor; or (3) reduce or inhibit the expression of NR2B receptor. In another embodiment, the term “a therapeutically effective amount” refers to the amount of the compound of the present disclosure that, when administered to a cell, or a tissue, or a non-cellular biological material, or a medium, is effective to at least partially reducing or inhibiting the activity of NR2B receptor; or at least partially reducing or inhibiting the expression of NR2B receptor. The meaning of the term “a therapeutically effective amount” as illustrated in the above embodiment for NR2B receptor also applies by the same means to any other relevant proteins/peptides/enzymes/receptors/ion channels, such as NMDA receptor, and the like.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder

As used herein, a subject is “in need of” a treatment if such subject would benefit biologically, medically or in quality of life from such treatment.

As used herein, the term “a,” “an,” “the” and similar terms used in the context of the present disclosure (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed.

Any asymmetric atom (e.g., carbon or the like) of the compound(s) of the present disclosure can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present disclosure can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present disclosure or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′p-toluoyl tartaric acid, mandelic acid, malic acid or camphor-10-sulfonic acid. Racemic compounds of the present disclosure or racemic intermediates can also be resolved by chiral chromatography, e.g., high pressure liquid chromatography (HPLC) using a chiral adsorbent.

The disclosure further includes any variant of the present processes, in which an intermediate obtainable at any stage thereof is used as starting material and the remaining steps are carried out, or in which the starting materials are formed in situ under the reaction conditions, or in which the reaction components are used in the form of their salts or optically pure material. Compounds of the present disclosure and intermediates can also be converted into each other according to methods generally known to those skilled in the art.

Pharmaceutical Compositions

In another aspect, the present disclosure provides a pharmaceutical composition comprising a compound of the present disclosure, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. The pharmaceutical compositions of the present disclosure can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Methods of Use

The compounds of the present disclosure in free form or in pharmaceutically acceptable salt form, exhibit valuable pharmacological properties, e.g. NR2B receptor modulating properties, for example as negative allosteric modulators of the NR2B receptor, e.g. as indicated in vitro and in vivo tests as provided in the next sections, and are therefore indicated for therapy or for use as research chemicals, e.g. as tool compounds.

Compounds of the present disclosure may be useful in the treatment of an indication selected from: Parkinson's disease, Huntington's disease, Rett syndrome, amyotrophic lateral sclerosis, multiple sclerosis, seizure disorders, autism, autism spectrum disorders, Fragile X syndrome, tuberous sclerosis, Down's syndrome, pain, migraine, tinnitus, bipolar disorder, obsessive-compulsive disorder, anxiety disorder, post-traumatic stress disorder (PTSD), cocaine use disorder, major depressive disorder, refractory or treatment resistant depression, or suicidality. Specifically compounds of the present disclosure may be useful in the treatment of an indication selected from: major depressive disorder, refractory or treatment resistant depression, and suicidality.

Thus, as a further aspect, the present disclosure provides the use of a compound of the present disclosure or a pharmaceutically acceptable salt thereof in therapy. In a further embodiment, the therapy is selected from a disease which may be treated by negative allosteric modulation of NR2B receptor. In another embodiment, the disease is selected from the afore-mentioned list.

Thus, as a further aspect, the present disclosure provides the use of a compound of the present disclosure or a pharmaceutically acceptable salt thereof, for the manufacture of a medicament. In a further embodiment, the medicament is for treatment of a disease which may be treated by negative allosteric modulation of NR2B receptor. In another embodiment, the disease is selected from the afore-mentioned list.

In one embodiment of the present disclosure, there is provided the compound of Formula (I) for use in the treatment of Parkinson's disease, Huntington's disease, Rett syndrome, amyotrophic lateral sclerosis, multiple sclerosis, seizure disorders, autism, autism spectrum disorders, Fragile X syndrome, tuberous sclerosis, Down's syndrome, pain, migraine, tinnitus, bipolar disorder, obsessive-compulsive disorder, anxiety disorder, post-traumatic stress disorder (PTSD), cocaine use disorder, major depressive disorder, refractory or treatment resistant depression, or suicidality. Specifically there is provided the compound of Formula (I) for use in the treatment of major depressive disorder, refractory or treatment resistant depression, or suicidality.

The pharmaceutical composition or combination of the present disclosure can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either internally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

Combinations

“Combination” refers to either a fixed combination in one dosage unit form, or a combined administration where a compound of the present disclosure and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present disclosure and a combination partner, are both administered to a patient simultaneously in the form of a single entity or dosage. The term “non-fixed combination” means that the therapeutic agents, e.g. a compound of the present disclosure and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The compound of the present disclosure may be administered either simultaneously with, or before or after, one or more other therapeutic agent. The compound of the present disclosure may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the present disclosure.

In one embodiment, the disclosure provides a product comprising a compound of the present disclosure and at least one other therapeutic agent as a combined preparation for simultaneous, separate or sequential use in therapy. In one embodiment, the therapy is the treatment of a disease or condition mediated by negative allosteric modulation of NR2B receptor. Products provided as a combined preparation include a composition comprising the compound of the present disclosure and the other therapeutic agent(s) together in the same pharmaceutical composition, or the compound of the present disclosure and the other therapeutic agent(s) in separate form, e.g. in the form of a kit.

In one embodiment, the disclosure provides a pharmaceutical composition comprising a compound of the present disclosure and another therapeutic agent(s). Optionally, the pharmaceutical composition may comprise a pharmaceutically acceptable carrier, as described above.

In one embodiment, the disclosure provides a kit comprising two or more separate pharmaceutical compositions, at least one of which contains a compound of the present disclosure. In one embodiment, the kit comprises means for separately retaining said compositions, such as a container, divided bottle, or divided foil packet. An example of such a kit is a blister pack, as typically used for the packaging of tablets, capsules and the like.

The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

In the combination therapies of the disclosure, the compound of the present disclosure and the other therapeutic agent may be manufactured and/or formulated by the same or different manufacturers. Moreover, the compound of the present disclosure and the other therapeutic may be brought together into a combination therapy: (i) prior to release of the combination product to physicians (e.g. in the case of a kit comprising the compound of the present disclosure and the other therapeutic agent); (ii) by the physician themselves (or under the guidance of the physician) shortly before administration; (iii) in the patient themselves, e.g. during sequential administration of the compound of the present disclosure and the other therapeutic agent.

Accordingly, the disclosure provides the use of a compound of the present disclosure for treating a disease or condition mediated by negative allosteric modulation of NR2B receptor, wherein the medicament is prepared for administration with another therapeutic agent. The disclosure also provides the use of another therapeutic agent for treating a disease or condition mediated by negative allosteric modulation of NR2B receptor, wherein the medicament is administered with a compound of the present disclosure.

The disclosure also provides a compound of the present disclosure for use in a method of treating a disease or condition mediated by negative allosteric modulation of NR2B receptor, wherein the compound of the present disclosure is prepared for administration with another therapeutic agent. The disclosure also provides another therapeutic agent for use in a method of treating a disease or condition mediated by negative allosteric modulation of NR2B receptor, wherein the other therapeutic agent is prepared for administration with a compound of the present disclosure. The disclosure also provides a compound of the present disclosure for use in a method of treating a disease or condition mediated by negative allosteric modulation of NR2B receptor, wherein the compound of the present disclosure is administered with another therapeutic agent. The disclosure also provides another therapeutic agent for use in a method of treating a disease or condition mediated by negative allosteric modulation of NR2B receptor, wherein the other therapeutic agent is administered with a compound of the present disclosure.

The disclosure also provides the use of a compound of the present disclosure for treating a disease or condition mediated by NR2B receptor, wherein the patient has previously (e.g. within 24 hours) been treated with another therapeutic agent. The disclosure also provides the use of another therapeutic agent for treating a disease or condition mediated by NR2B receptor, wherein the patient has previously (e.g. within 24 hours) been treated with compound of the present disclosure.

In one embodiment, the other therapeutic agent is selected from:
(a) lithium;
(b) stimulants, such as amphetamine and dextroamphetamine, (Adderall™) or methylphenidate Italin™);
(c) acetylcholinesterase inhibitors, such as donepezil (Aricept™), rivastigmine (Exelon™) and galantamine (Razadyne™);
(d) antidepressant medications for low mood and irritability, such as citalopram (Celexa™) fluoxetine (Prozac™), paroxeine (Paxil™), sertraline (Zoloft™), trazodone (Desyrel™), and tricyclic antidepressants such as amitriptyline (Elavil™);
(e) anxiolytics for anxiety, restlessness, verbally disruptive behavior and resistance, such as lorazepam (Ativan™) and oxazepam (Serax™);
(f) antipsychotic medications for hallucinations, delusions, aggression, agitation, hostility and uncooperativeness, such as aripiprazole (Abilify™), clozapine (Clozaril™), haloperidol (Haldol™), olanzapine (Zyprexa™), quetiapine (Seroquel™), risperidone (Risperdal™) and ziprasidone (Geodon™);
(g) mood stabilizers, such as carbamazepine (Tegretol™) and divalproex (Depakote™);
(h) pregabalin;
(i) gabapentin (Neurontin™);
(j) dopamine agonists such as L-DOPA, pramipexole (Mirapex™) and ropinerol (Requip™); (k) analgesics including opiates and non-opiates;
(k) carbidopa;
(l) triptans such as sumatriptan (Imitrex™) and zolmitriptan (Zomig™);
(m) nicotinic apha-7 agonists;
(n) mGluR5 antagonists;
(o) H3 agonists;
(p) amyloid therapy vaccines; and
(q) chemotherapy agents.

In one embodiment of the disclosure, there is provided a product comprising a NR2B modulator and aforementioned combination partners as a combined preparation for simultaneous, separate or sequential use in therapy.

In another embodiment of the disclosure, there is provided a product comprising a NR2B modulator and aforementioned combination partners as a combined preparation for simultaneous, separate or sequential use in therapy.

In one embodiment of the disclosure, there is provided a pharmaceutical composition comprising a NR2B modulator, aforementioned combination partners, and a pharmaceutically acceptable carrier.

In a further embodiment of the disclosure, there is provided a pharmaceutical composition comprising a NR2B modulator, aforementioned combination partners, and a pharmaceutically acceptable carrier.

Preparation of Compounds

Compounds of the present disclosure can be prepared as described in the following Examples.

Intermediates described herein can be prepared as shown in Scheme 1 below.

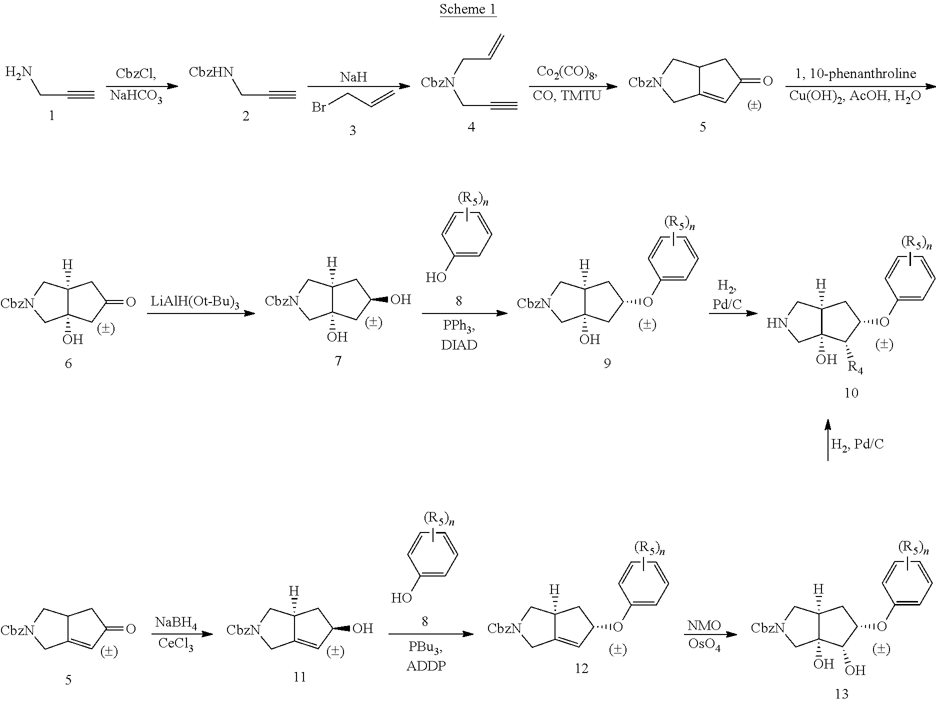

In Scheme 1, propargylamine 1 can be treated with benzyl chloroformate to give protected amine 2, which can then be allylated with allyl bromide to provide 4. This can undergo a Pauson-Khand cycloaddition to provide the bicyclic enone 5. This key intermediate can be oxidized at the bridgehead position to give cis-fused alcohol 6, which can be reduced to diol 7 with control of the relative stereochemistry. The Mitsunobu reaction with a phenol such as 8 (where R₅ and n are as defined in the claims) proceeds with inversion of stereochemistry, generating the desired all-cis configuration of an ether such as 9, which can be deprotected by hydrogenation to yield a free amine such as 10 (where R₄ is H). Alternatively, 5 can first be reduced under Luche conditions to allylic alcohol 11. The Mitsunobu-type reaction with a phenol such as 8 now gives an olefin such as 12, which can be subjected to dihydroxylation with osmium tetroxide, providing a diol such as 13. As before, hydrogenation of the protecting group can give a free amine such as 10 (where R₄ is OH). This can either be brought forward as a racemic mixture, or intermediates 7 or 13 can be chirally separated into their enantiomers, which can be brought separately through the rest of the sequence.

Compounds provided herein can be prepared as shown in Scheme 2 below.

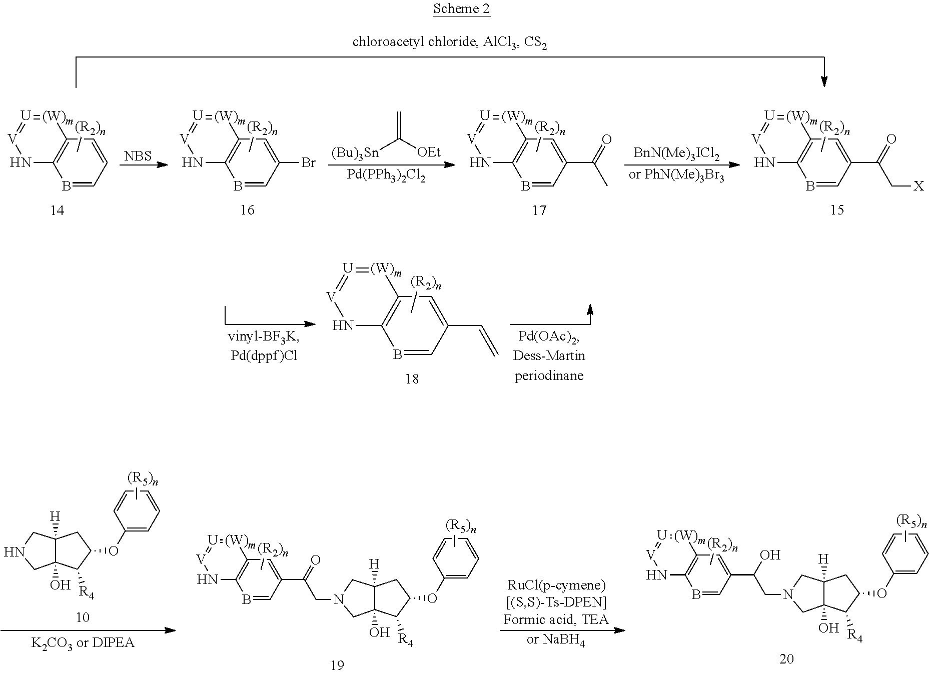

In Scheme 2, bicyclic compounds such as 14 (where R₂, B, U, V, W, m and n are as defined in the claims) are either commercially available or can be made through standard chemical transformations as described in the individual procedures. In many cases, they can be converted directly to an a-haloketone such as 15 through a Friedel-Crafts acylation with chloroacetyl chloride and a Lewis acid such as aluminum chloride. Alternatively, 14 can be treated with a brominating reagent such as N-bromosuccinimide to provide a bromide such as 16, which can either be converted directly to a ketone such as 17 by a Stille coupling with tributyl(1-ethoxyvinyl)stannane and a palladium catalyst, or through a two step process consisting of a Suzuki-Miyaura coupling with potassium vinyltrifluoroborate in the presence of a palladium catalyst and base to yield an olefin such as 18, followed by a Wacker-type oxidation to provide 17. This can be treated with a halogenating agent such as benzyltrimethylammonium dichloroiodate or phenyltrimethylammonium tribromide to form an a-haloketone such as 15. This can undergo a nucleophilic displacement with an amine such as 10 (where R₄, R₅, and n are as defined in the claims) in the presence of a base such as potassium carbonate or N,N-diisopropylethylamine to yield a ketone such as 19.

This can be reduced with formic acid and triethylamine in the presence of a chiral catalyst such as RuCl(p-cymene)[(S,S)-Ts-DPEN] to provide examples such as 20 with high levels of diastereoselectivity. Alternatively, a reducing agent such as sodium borohydride can be used to provide examples such as 20 as mixtures of diastereomers, which can be separated into single diastereomers by chiral chromatography.

Alternatively, compounds can be produced as shown in Scheme 3 below.

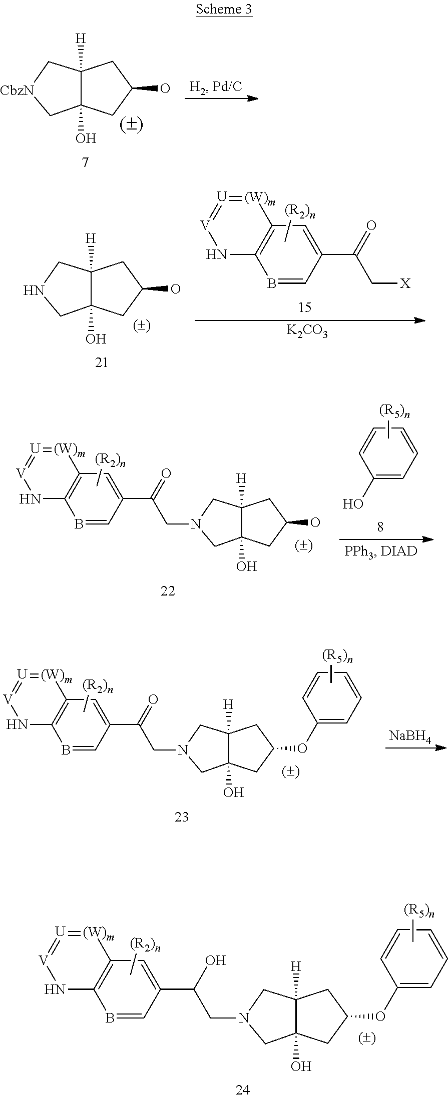

In Scheme 3, the Cbz protecting group of 7 can be removed by hydrogenation to yield free amine 21, which can react with an a-haloketone such as 15 (where R₂, B, U, V, W, m and n are as defined in the claims) to give a ketone such as 22. This can undergo a Mitsunobu reaction with a phenol such as 8 (where R₅ and n are as defined in the claims) to form a ketone such as 23. This can be reduced with a reducing agent such as sodium borohydride to provide examples such as 24 as mixtures of diastereomers, which can be separated into single diastereomers by chiral chromatography.

Alternatively, compounds can be produced as shown in Scheme 4 below.

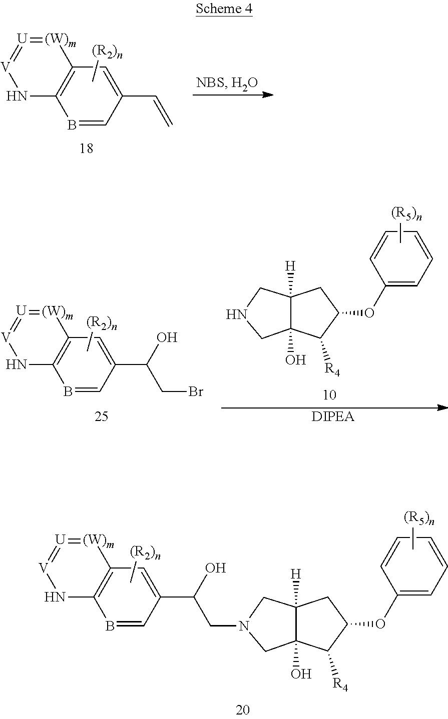

In Scheme 4, an olefin such as 18 (where R₂, B, U, V, W, m and n are as defined in the claims) can be treated with N-bromosuccinimide and water to provide a bromohydrin such as 25. This can undergo nucleophilic displacement with an amine such as 10 (where R₄, R₅ and n are as defined in the claims) in the presence of a base such as N,N-diisopropylethylamine to provide examples such as 20 as mixtures of diastereomers, which can be separated into single diastereomers by chiral chromatography.

Alternatively, compounds can be produced as shown in Scheme 5 below.

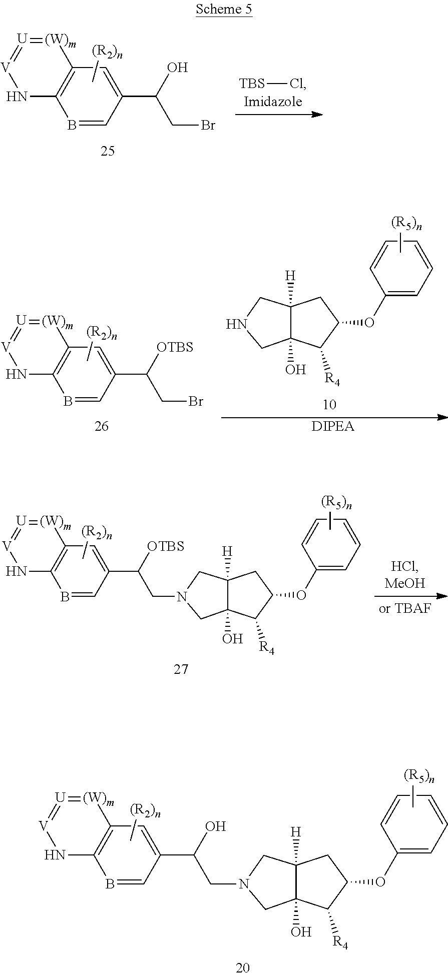

In scheme 5, an alcohol such as 25 (where R₂, B, U, V, W, m and n are as defined in the claims) can be protected using tert-butyldimethylsilyl chloride in the presence of a base such as imidazole to provide a silyl ether such as 26. This can undergo nucleophilic displacement with an amine such as 10 (where R₄, R₅ and n are as defined in the claims) in the presence of a base such as N,N-diisopropylethylamine to provide intermediates such as 27. This can be deprotected using an acid such as hydrochloric acid in an alcoholic solvent such as methanol, or with a fluoride source such as tetra-n-butylammonium fluoride, to provide examples such as 20 as mixtures of diastereomers, which can be separated into single diastereomers by chiral chromatography.

Alternatively, compounds can be produced as shown in Scheme 6 below.

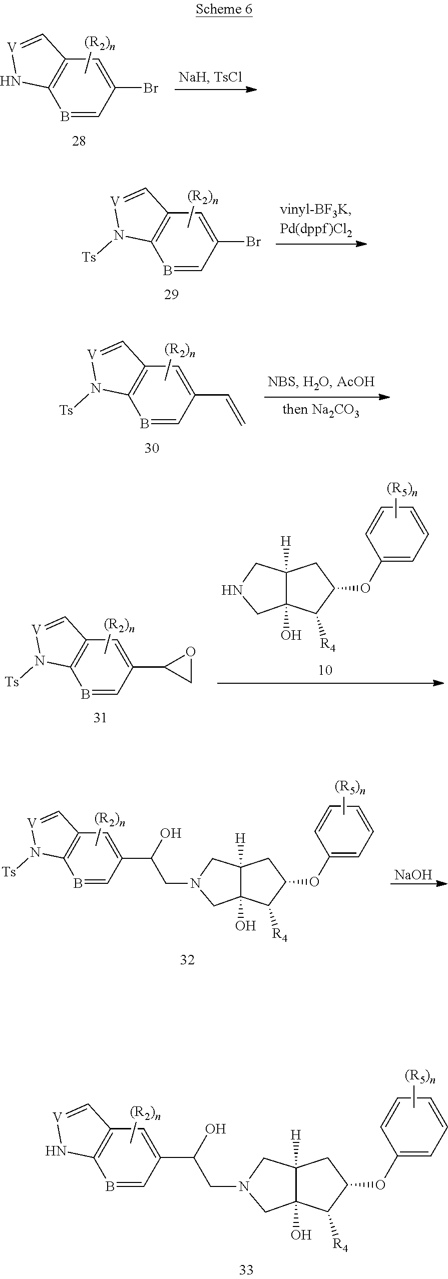

In Scheme 6, a heterocycle such as 28 (where R₂, B, V and n are as defined in the claims) can be treated with a base such as sodium hydride and tosyl chloride to provide the tosyl protected heterocycle 29. This can undergo a Suzuki-Miyaura coupling with potassium vinyltrifluoroborate in the presence of a palladium catalyst and base to yield an olefin such as 30, which can then be converted to an epoxide such as 31 with N-bromosuccinimide, water, and an acid such as acetic acid, followed by treatment with a base such as sodium carbonate. The epoxide of 31 can be opened through nucleophilic attack by an amine such as 10 (where R₄, R₅ and n are as defined in the claims) to provide an amino-alcohol such as 32. The tosyl group can then be removed using a base such as sodium hydroxide to provide examples such as 33 as mixtures of diastereomers, which can be separated into single diastereomers by chiral chromatography.

### INTERMEDIATES AND EXAMPLES

The following examples are intended to illustrate the disclosure and are not to be construed as being limitations thereon.

Many examples were made as mixtures of two or four stereoisomers, then separated into single isomers which were tested individually in the NR2B rat cortical neuron calcium influx assay described in the Biological Data section below. However, the stereochemistry of every enantiomer was not determined. The stereochemistry of Example 1A was determined by single crystal x-ray crystallography to be 6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H-one, as depicted below

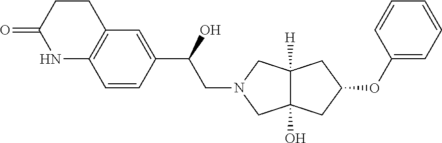 [SMILES: O=C1CCc2cc([C@@H](O)CN3C[C@H]4C[C@H](Oc5ccccc5)C[C@@]4(O)C3)ccc2N1]

### 6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one

From this crystal structure, structure-activity relationship analysis, chemical correlation, and knowledge of. WO 2016/049165 A1, it is assumed that the (3aS,5S,6aR) configuration of the hexahydrocyclopenta[c]pyrrole core [or the (3aS,4S,5S,6aR) configuration when R₄ is OH] is more active than the (3aR,5R,6aS) configuration [or the (3aR,4R,5R,6aS) configuration when R₄ is OH] in all of the Examples. Although there is strong evidence to suggest that the (3aS,5S,6aR) [or (3aS,4S,5S,6aR)] configuration is the more active configuration, there is still the chance that the (3aR,5R,6aS) [or (3aR,4R,5R,6aS)]configuration could be the more active configuration in some of the Examples.

Within sets of Examples where the stereochemistry of each Example has not been fully determined, the possible names and chemical structures have been listed according to their structural orientation. Generally, compounds containing the (3aS,5S,6aR) [or (3aS,4S,5S,6aR)] core have been listed before compounds containing the (3aR,5R,6aS) [or (3aR,4R,5R,6aS)] core, and compounds where the benzylic alcohol is in the R configuration (“up” orientation as drawn) have been listed before compounds where the benzylic alcohol is in the S configuration (“down” orientation as drawn). This order does not necessarily correspond to the A/B or A/B/C/D order within that set of Examples (the A/B or A/B/C/D order generally refers to the order that the compounds were obtained from chiral separation).

For illustration, within the set of Examples 5A/5B/5C/5D, the four possible names and chemical structures are listed as follows:
6-((R)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one
6-((R)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one

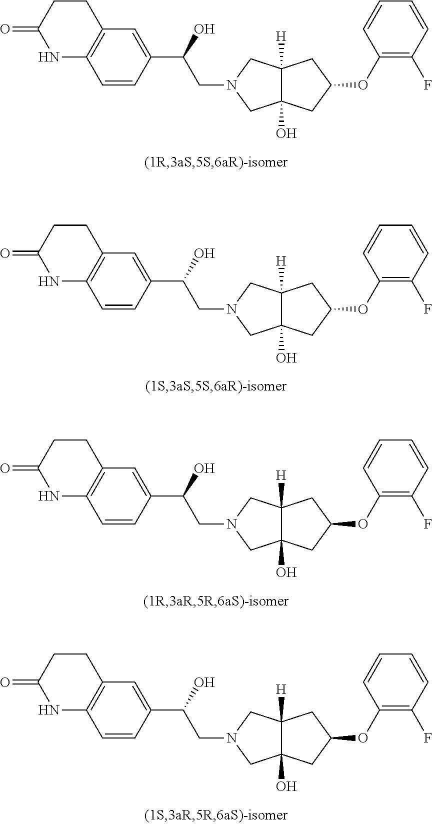 [SMILES: O=C1CCc2cc([C@@H](O)CN3C[C@H]4C[C@H](Oc5ccccc5F)C[C@@]4(O)C3)ccc2N1 ; O=C1CCc2cc([C@H](O)CN3C[C@H]4C[C@H](Oc5ccccc5F)C[C@@]4(O)C3)ccc2N1 ; O=C1CCc2cc([C@@H](O)CN3C[C@@H]4C[C@@H](Oc5ccccc5F)C[C@]4(O)C3)ccc2N1 ; O=C1CCc2cc([C@H](O)CN3C[C@@H]4C[C@@H](Oc5ccccc5F)C[C@]4(O)C3)ccc2N1]

In this case, although Examples 5C and 5D are more potent than Examples 5A and 5B in the NR2B rat cortical neuron calcium influx assay, and are therefore likely to contain the (3aS,5S,6aR) core and correspond to the top two structures drawn, the four possible names and structures are still listed in this order, in accordance with the ordering system used throughout the Examples.

### Abbreviations

Abbreviations used are those conventional in the art or the following:
Ac acetyl
ACN acetonitrile
AcOH acetic acid
ADDP 1,1′-(azodicarbonyl)dipiperidine
aq aqueous
atm atmosphere
BINAP 2,2′-bis(diphenylphosphino)-1,1′-binaphthyl
Bn benzyl
Boc tert-butyloxycarbonyl
Bu butyl
B₂(pin)₂ bis(pinacolato)diboron
C Celsius
Cbz carboxybenzyl
CDI carbonyldiimidazole
conc concentrated
DCM dichloromethane
DEA diethylamine
DIAD diisopropyl azodicarboxylate
DIPEA N,N-diisopropylethylanine
DMF N,N-dimethylformamide
DMSO dimethylsulfoxide
DMT dimercaptotriazine
DPEN 1,2-diphenylethylenediamine
dppf 1,1′-bis(diphenylphosphino)ferrocene
Et ethyl
EtOAc ethyl acetate
EtOH ethanol
Et₂O diethyl ether
FCC flash column chromatography
g gram(s)
h hour(s)
HBSS Hanks' balanced salt solution
HPLC high performance liquid chromatography
IACUC Institutional Animal Care and Use Committee
IC₅₀ half maximal inhibitory concentration
IPA isopropyl alcohol
L liter(s)
LAH lithium aluminum hydride
LCMS liquid chromatography and mass spectrometry
LiHMDS lithium hexamethyldisilazide
m-CPBA meta-chloroperoxybenzoic acid
Me methyl
MeOH methanol
mg milligram(s)
MHz megahertz
min minute(s)
mL milliliter(s)
mm millimeter(s)
mM millimolar
mmol millimole(s)
MS mass spectrometry
MTBE methyl tert-butyl ether
m/z mass to charge ratio
NADPH nicotinamide adenine dinucleotide phosphate
NBS N-bromosuccinimide
nm nanometer(s)
nM nanomolar
NMO N-methylmorpholine N-oxide
NMP N-methyl-2-pyrrolidone
NMR nuclear magnetic resonance
Pd/C palladium on carbon
PE petroleum ether
PG protecting group
Ph phenyl
PMB para-methoxybenzyl
ppm parts per million
rac racemic
Rf retention factor
Rt retention time
RT room temperature
SFC supercritical fluid chromatography
TBAF tetra-n-butylammonium fluoride
TBS tert-butyldimethylsilyl
t-Bu tert-butyl
t-BuOH tert-butanol
TEA triethylamine
TFA trifluoroacetic acid
THF tetrahydrofuran
TLC thin-layer chromatography
TMTU N,N,N,N-tetramethylthiourea
Ts tosyl
μL microliter(s)
μm micrometer(s); micron(s)
μM micromolar
UPLC ultra performance liquid chromatography
UV ultraviolet

General Procedures

Where no preparative route is described, the material is commercially available. Commercial reagents were used without additional purification unless otherwise stated. Room temperature (RT) is approximately 20-25° C. ¹H NMR were recorded on a 300 MHz Varian, a 400 MHz Varian or a 400 MHz Bruker NMR instrument. Chemical shifts are reported as parts per million (ppm) relative to tetramethylsilane and coupling constants (J) are reported in Hertz. Abbreviations for multiplicity are: s=singlet, d=doublet, t=triplet, q=quartet, dd=doublet of doublet, dt=doublet of triplet, br=broad.

LCMS Method A:

Instrument: Waters Acquity UPLC, photodiode array detector; Column: AcQuity UPLC BEH C₁₈, 1.7 μm, 2.1×30 mm; 2 min run time, 2% solvent B from 0 to 0.1 min, 2→98% solvent B from 0.1 to 1.8 min, 2% solvent B for 0.2 min. Solvents: Solvent A=0.1% formic acid in water (v/v), solvent B=0.1% formic acid in acetonitrile (v/v). Injection volume 2-5 uL; UV detection array 210-400 nm; mass detection 120-1250 (electrospray ionization); column at 50° C.; flow rate 1.0 mL/min.

LCMS Method B:

Instrument: Waters Acquity UPLC, photodiode array detector; Column AcQuity UPLC BEH C₁₈ 1.7 μm 21×30 mm; 5.2 min run time, 2→98% solvent B from 0 to 5.15 min, 98% solvent B from 5.15 to 5.20 min. Solvents: Solvent A=0.1% formic acid in water (v/v), solvent B=0.1% formic acid in acetonitrile (v/v). Injection volume 2-5 uL; UV detection array 210-400 nm; mass detection 120-1600; column at 50° C., flow rate 1.0 mL/min.

LCMS Method C:

Instrument: Waters Acquity UPLC, photodiode array detector; Column: AcQuity UPLC BEH C₁₈ 1.7 μm, 21×30 mm; 1.2 min run time, 2% solvent B from 0 to 0.1 min, 2→80% solvent B from 0.1 to 0.5 min, 80→95% solvent B from 0.5 to 0.6 min, 95% solvent B from 0.6 to 0.8 min, 95→2% solvent B from 0.8 to 0.9 min, 2% solvent B from 0.9 to 1.20 min. Solvents: Solvent A=0.05% formic acid in water (v/v), solvent B=0.04% formic acid in methanol (v/v). UV detection array 200-300 nm; mass detection 100-1600 (electrospray ionization); column at 55° C.; flow rate 1.0 mL/min.

LCMS Method D:

Instrument: API 2000, photodiode array detector; Column: Synergi 2.5 micron MAX-RP 100 A Mercury; 3.0 min run time, 30% solvent B from 0 to 0.5 min, 30→95% solvent B from 0.5 to 1.5 min, 95% solvent B from 1.5 to 2.4 min, 95→30% solvent B from 2.4 to 2.5 min, 30% solvent B from 2.5 to 3.0 min. Solvents: Solvent A=0.1% formic acid in water (v/v), solvent B=acetonitrile. UV detection array 190-400; Mass detection 100-1000 (electrospray ionization); Column at 30° C.; flow rate 2.0 mL/min.

LCMS Method E:

Instrument: API 2000, photodiode array detector; Column: Synergi 2.5 micron MAX-RP 100 A Mercury; 4.0 min run time, 20→50% solvent B from 0.0 to 0.2 min, 50→95% solvent B from 0.2 to 1.0 min, 95% solvent B from 1.0 to 2.5 min, 95→50% solvent B from 2.5 to 2.9 min, 50→20% solvent B from 2.9 to 3.2 min, 20% solvent B from 3.2 to 4.0 min. Solvents: Solvent A=0.1% formic acid in water (v/v), solvent B=acetonitrile. UV detection array 190-400; Mass detection 100-1000 (electrospray ionization); Column at 30° C.; flow rate 1.4 mL/min.

LCMS Method F:

Instrument: Shimadzu Nexera LCMS-2020, photodiode array detector; Column: Synergi 2.5 micron MAX-RP 100 A Mercury (20×4 mm); 3.0 min run time, 5% solvent B from 0 to 0.5 min, 5→95% solvent B from 0.5 to 1.0 min, 95% solvent B from 1.0 to 1.5 min, 95→5% solvent B from 1.5 to 2.0 min, 5% solvent B from 2.0 to 3.0 min. Solvents: Solvent A=0.1% formic acid in water (v/v), solvent B=0.1% formic acid in acetonitrile (v/v). UV detection array 200-400; Mass detection 100-1000 (electrospray ionization); Column at 40° C.; flow rate 2.0 mL/min.

LCMS Method G:

Instrument: API 3000, photodiode array detector; Column: Synergi 2.5 micron MAX-RP 100 A Mercury; 3.0 min run time, 10→20% solvent B from 0.0 to 0.5 min, 20→95% solvent B from 0.5 to 1.5 min, 95% solvent B from 1.5 to 2.0 min, 95→10% solvent B from 2.0 to 2.5 min, 10% solvent B from 2.5 to 3.0 min, 20% solvent B from 3.2 to 4.0 min. Solvents: Solvent A=0.1% formic acid in water (v/v), solvent B=acetonitrile. UV detection array 190-400; Mass detection 100-1000 (electrospray ionization); Column at 30° C.; flow rate 1.4 mL/min.

LCMS Method H:

Instrument: Waters Acquity UPLC, photodiode array detector; Column: SunFire C18 3.5 μm 3.0×30 mm; 2.2 min run time, 5→95% solvent B from 0.0 to 1.7 min, 95% solvent B from 1.7 to 2.0 min, 95→5% solvent B from 2.0 to 2.1 min, 5% solvent B from 2.1 to 2.2 min. Solvents: Solvent A=0.05% TFA in water (v/v), solvent B=acetonitrile. UV detection array 200-400 nm; mass detection 150-1600 (electrospray ionization); column at 40° C.; flow rate 2.0 mL/min.

LCMS Method I:

Column: Kinetex EVO C18 2.1×30 mm, 5 μm; 1.5 min run time, 5→95% solvent B from 0.0 to 0.8 min, 95% solvent B from 0.8 to 1.2 min, 95→5% solvent B from 1.2 to 1.21 min, 5% B from 1.21 to 1.5 min. Solvents: solvent A=0.05% NH₃·H₂O in water (v/v), solvent B=Acetonitrile. Mass detection 100-1000 (electrospray ionization); column at 40° C.; flow rate 1.5 mL/min.

LCMS Method J:

Column: Chromolith Flash RP-18e 25×2 mm; 1.5 min run time, 5% solvent B from 0.0 to 0.01 min, 5→95% solvent B from 0.01 to 0.80 min, 95% solvent B from 0.80 to 1.2 min, 95→5% solvent B from 1.2 to 1.21 min, 5% B from 1.21 to 1.5 min. Solvents: solvent A=0.0375% TFA in water (v/v), solvent B=0.01875% TFA in acetonitrile (v/v). Mass detection 100-1000 (electrospray ionization); column at 50° C.; flow rate 1.5 mL/min.

LCMS Method K:

Instrument: Waters Acquity UPLC, photodiode array detector; Column: AcQuity UPLC BEH C₁₈ 1.7 μm, 2.1×30 mm; 2 min run time, 2% solvent B from 0 to 0.1 min, 2→98% solvent B from 0.1 to 1.8 min, 2% solvent B for 0.2 min. Solvents: Solvent A=5 mM Ammonium Hydroxide in Water, solvent B=5 mM Ammonium Hydroxide in Acetonitrile. Injection volume 2-5 uL; UV detection array 210-400 nm; mass detection 120-1250 (electrospray ionization); column at 50° C.; flow rate 1.0 mL/min.

LCMS Method L:

Column: Chromolith Flash RP-18e 25×2 mm; 1.5 min run time, 0% solvent B from 0.0 to 0.01 min, 0→60% solvent B from 0.01 to 0.80 min, 60% solvent B from 0.80 to 1.2 min, 60→0% solvent B from 1.2 to 1.21 min, 0% B from 1.21 to 1.5 min. Solvents: solvent A=0.0375% TFA in water (v/v), solvent B=0.01875% TFA in acetonitrile (v/v). Mass detection 100-1000 (electrospray ionization); column at 50° C.; flow rate 1.5 mL/min.

### SYNTHESIS OF INTERMEDIATES AND EXAMPLES

### Intermediate 1

A racemic mixture of:
(3aS,5S,6aR)-5-phenoxyhexahydrocyclopenta[c]pyrrol-3a(1H)-ol
(3aR,5R,6aS)-5-phenoxyhexahydrocyclopenta[c]pyrrol-3a(1H)-ol

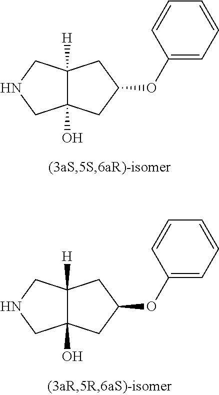 [SMILES: O[C@]12CNC[C@H]1C[C@H](Oc1ccccc1)C2 ; O[C@@]12CNC[C@@H]1C[C@@H](Oc1ccccc1)C2]

### Step 1: Benzyl prop-2-yn-1-ylcarbamate

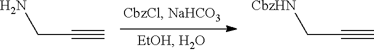

Benzyl chloroformate (273 g, 1.60 mol) was added dropwise to a stirred solution of propargylamine (80 g, 1.45 mol) and NaHCO₃ (243.6 g, 2.9 mol) in ethanol/water (2.4 L, 1:1, v/v) at 0° C. After stirring for 2 h at 0° C. and 12 h at 25° C., the mixture was diluted with water (1.0 L) and extracted with MTBE (1.0 L). The phases were separated and the aqueous layer was extracted with MTBE (500 mL×2). The combined organic layers were dried over anhydrous Na₂SO₄, filtered and evaporated to give the title intermediate (280 g, crude) as a yellow solid which was used without purification.

¹H NMR (400 MHz, CDCl₃) δ 7.38-7.32 (m, 5H), 5.24-5.08 (m, 3H), 4.05-3.93 (m, 2H), 2.26 (s, 1H).

### Step 2: Benzyl allyl(prop-2-yn-1-yl)carbamate

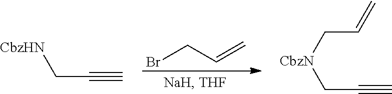

NaH (60% in mineral oil, 39 g, 0.98 mol) was added to a solution of benzyl prop-2-yn-1-ylcarbamate (155 g, 0.817 mol) and allyl bromide (149 g, 1.23 mol) in THF (2.0 L) at 0° C. and the reaction was stirred for 2 h at 25° C. The mixture was quenched with saturated aq.

NH₄Cl (500 mL) and the aqueous layer was extracted with EtOAc (3×500 mL). The combined organic layers were dried over anhydrous Na₂SO₄ and concentrated. The crude material was purified by FCC (10% EtOAc:PE) to give the title intermediate (135 g) as a colorless oil.

¹H NMR (400 MHz, CDCl₃) δ 7.44-7.31 (m, 5H), 5.87-5.74 (m, 1H), 5.29-5.15 (m, 4H), 4.17-3.96 (m, 4H), 2.23 (s, 1H).

### Step 3: (±)-Benzyl 5-oxo-3,3a,4,5-tetrahydrocyclopenta[c]pyrrole-2(1H)-carboxylate

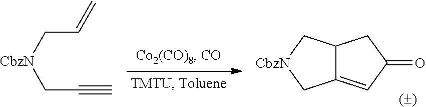 [SMILES: C#CCN(CC=C)C(=O)OCc1ccccc1 ; O=C1C=C2CN(C(=O)OCc3ccccc3)CC2C1]

To a solution of benzyl allyl(prop-2-yn-1-yl)carbamate (20 g, 89.6 mmol) and N,N,N,N-tetramethylthiourea (5.89 g, 44.5 mmol) in toluene (1.0 L) was added CO₂(CO)₈ (7.6 g, 22.4 mmol) at 25° C. under 1 atm CO pressure. The solution was heated to 80° C. and stirred for 3 h. The reaction mixture was cooled to RT, filtered through a pad of Celite and concentrated. The crude material was purified by FCC (15-50% EtOAc:PE) to provide the title intermediate (12 g) as a colorless oil.

H NMR (400 MHz, CDCl₃) δ 7.38-7.33 (m, 5H), 6.11-6.07 (m, 1H), 5.21-5.14 (m, 2H), 4.36-4.28 (m, 2H), 4.18-4.11 (m, 1H), 3.28-3.26 (m, 1H), 2.97-2.92 (m, 1H), 2.68-2.64 (m, 1H), 2.23-2.19 (m, 1H).

Step 4: A racemic mixture of:
Benzyl (3aS,6aR)-3a-hydroxy-5-oxohexahydrocyclopenta[c]pyrrole-2(1 h)-carboxylate
Benzyl (3aR,6aS)-3a-hydroxy-5-oxohexahydrocyclopenta[c]pyrrole-2(1 h)-carboxylate

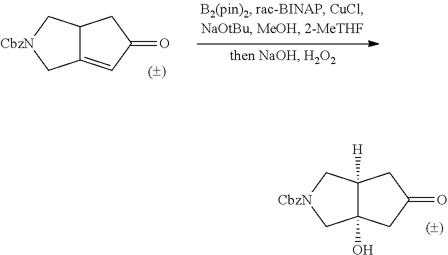 [SMILES: O=C1C=C2CN(C(=O)OCc3ccccc3)CC2C1 ; O=C1C[C@@H]2CN(C(=O)OCc3ccccc3)C[C@]2(O)C1]

2-methyltetrahydrofuran (125 mL) was purged with nitrogen for 10 minutes, then CuCl (485 mg, 4.9 mmol) and rac-BINAP (3.03 g, 4.9 mmol) were added. After 5 minutes NaOt-Bu (470 mg, 4.9 mmol) and bis(pinacolato)diboron (30 g, 117 mmol) were added and the reaction was purged with nitrogen for another 15 minutes. A solution of (±)-benzyl 5-oxo-3,3a,4,5-tetrahydrocyclopenta[c]pyrrole-2(1H)-carboxylate (25 g, 97 mmol) in 2-methyltetrahydrofuran (125 mL) was added and the reaction was stirred under nitrogen at RT for 2 h. The reaction was cooled to 10° C. and MeOH (6.25 g, 7.89 mL, 194 mmol) was added. This was stirred for 10 min, then warmed to RT for 30 min, then cooled again to 10° C. NaOH (4.66 g, 117 mmol) was added followed by 30% aq. H₂O₂(33 g, 99 mL, 292 mmol) dropwise, and this was stirred for 50 min. This was diluted with water (150 mL) and extracted with EtOAc (3×100 mL). The combined organic layers were washed with saturated aq. sodium thiosulfate (100 mL), dried with Na₂SO₄, filtered and concentrated. The crude material was purified by FCC (0-100% EtOAc:PE) to provide the title intermediate (20 g, 90% purity) as a light yellow oil.

¹H NMR (400 MHz, DMSO-d₆) δ 7.43-7.24 (m, 5H), 5.55 (s, 1H), 5.12-4.99 (m, 2H), 3.79-3.65 (m, 1H), 3.53-3.38 (m, 2H), 3.22-3.11 (m, 1H), 2.70-2.62 (m, 1H), 2.58-2.52 (m, 1H), 2.34-2.29 (m, 1H), 2.17-2.06 (m, 1H). 1H under solvent peak.

Step 5: A racemic mixture of:
Benzyl (3aS,5R,6aR)-3a,5-dihydroxyhexahydrocyclopenta[c]pyrrole-2(1H)-carboxylate
Benzyl (3aR,5S,6aS)-3a,5-dihydroxyhexahydrocyclopenta[c]pyrrole-2(1H-carboxylate

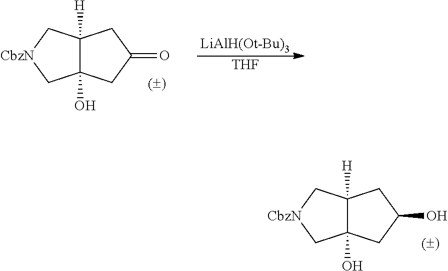 [SMILES: O=C1C[C@@H]2CN(C(=O)OCc3ccccc3)C[C@]2(O)C1 ; O=C(OCc1ccccc1)N1C[C@H]2C[C@@H](O)C[C@@]2(O)C1 ; "LiAlH(Ot-Bu)3" ; "THF"]

To a solution of a racemic mixture of benzyl (3aS,6aR)-3a-hydroxy-5-oxohexahydrocyclopenta[c]pyrrole-2(1H)-carboxylate and benzyl (3aR,6aS)-3a-hydroxy-5-oxohexahydrocyclopenta[c]pyrrole-2(1H)-carboxylate (20 g, 62.48 mmol, 90% purity) in THF (200 mL) was added dropwise a solution of LiAlH(Ot-Bu)₃ (124.9 mL, 124.9 mmol, 1.0 M in THF) at 0° C. The reaction was warmed to 25° C. and stirred for 2 h. The reaction mixture was added dropwise to a saturated solution of NH₄Cl (100 mL) at 0° C. The mixture was extracted with EtOAc (2×100 mL). The combined organic layers were washed with saturated brine (100 mL). The organic layer was dried over Na₂SO₄ and concentrated. The crude material was purified by FCC (0-15% MeOH:DCM) to provide the title intermediate (16 g) as a colorless oil.

LCMS: Rt 0.56 min; MS m/z 278.1 [M+H]+; Method J.

¹H NMR (400 MHz, DMSO-d₆) δ 7.39-7.29 (m, 5H), 5.06-5.01 (m, 3H), 4.67-4.65 (m, 1H), 4.28-4.19 (m, 1H), 3.65-3.52 (m, 2H), 3.38-3.34 (m, 1H), 3.27-3.17 (m, 1H), 2.32-2.13 (m, 2H), 2.05-1.92 (m, 1H), 1.73-1.64 (m, 1H), 1.29-1.16 (m, 1H).

Step 6: A racemic mixture of:
Benzyl (3aS,5S,6aR)-3a-hydroxy-5-phenoxyhexahydrocyclopenta[c]pyrrole-2(1H)-carboxylate
Benzyl (3aR,5R,6aS)-3a-hydroxy-5-phenoxyhexahydrocyclopenta[c]pyrrole-2(1H)-carboxylate

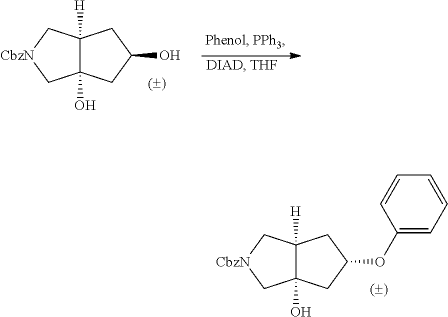

A dried reaction flask was charged with triphenylphosphine (12.58 g, 48.0 mmol), anhydrous THF (100 mL) and phenol (4.84 g, 51.4 mmol) with stirring under nitrogen at ambient temperature. A racemic mixture of benzyl (3aS,5R,6aR)-3a,5-dihydroxyhexahydrocyclopenta[c]pyrrole-2(1H)-carboxylate and benzyl (3aR,5S,6aS)-3a,5-dihydroxyhexahydrocyclopenta[c]pyrrole-2(1H)-carboxylate (9.5 g, 34.3 mmol) in anhydrous THF (10.5 mL) was added and the solution was cooled in an ice bath. A solution of DIAD (9.32 mL, 48.0 mmol) in anhydrous THF (50 mL) was added dropwise over 15-20 minutes with vigorous stirring, and a light yellow color persisted upon complete addition. The maximum internal temperature reached about 14° C. during the addition, and the reaction was aged in the bath for 45 minutes. The reaction was quenched with water (50 mL), and the mixture was stirred for about 30 minutes. The mixture was diluted with EtOAc (100 mL), and the organic layer was washed a second time with water (50 mL). The combined aqueous washes were back-extracted with EtOAc (100 mL), and the combined organic extracts were washed with saturated brine (2×100 mL), and then dried over Na₂SO₄, filtered and concentrated to a yellow oil. The residue was triturated with Et₂O (100 mL), resulting in an off-white precipitate, and the mixture was stirred in an ice/water bath while heptanes (50 mL) was added dropwise with vigorous stirring. The precipitate was collected and washed with 1:2 heptanes/Et₂O. The light yellow solid product was slurried again with Et₂O first by rotation on the rotovap at 35° C., and then with stirring at room temperature overnight. The slurry was filtered and all the filtrate was combined. The filtrate/wash was concentrated to dryness and the yellow oil was treated with Et₂O/heptane (2:1) and purified by FCC (10-60% EtOAc:Hexane) to give the title intermediate (11.46 g). LCMS: Rt 2.29 min; MS m/z 354.4 [M+H]+; Method B.

¹H NMR (400 MHz, CDCl₃) δ 7.38-7.28 (m, 7H), 7.01-6.96 (m, 1H), 6.88-6.85 (m, 2H), 5.14 (s, 2H), 4.95-4.92 (m, 1H), 3.81-3.78 (m, 2H), 3.50-3.46 (m, 1H), 3.30-3.25 (m, 1H), 2.76-2.72 (m, 2H), 2.47-2.41 (m, 1H), 2.32-2.27 (m, 1H), 2.18-2.10 (m, 1H), 1.75 (m, 1H).

Step 7: A racemic mixture of:
(3aS,5S,6aR)-5-phenoxyhexahydrocyclopenta[c]pyrrol-3a(1H)-ol
(3aR,5R,6aS)-5-phenoxyhexahydrocyclopenta[c]pyrrol-3a(1H)-ol

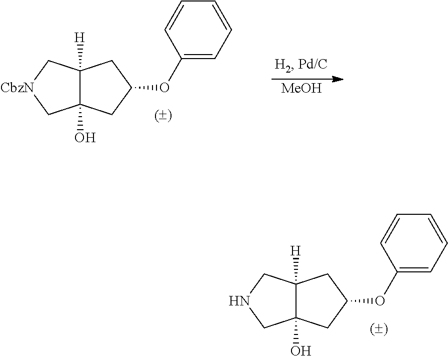 [SMILES: "MeOH" ; O=C(OCc1ccccc1)N1C[C@H]2C[C@H](Oc3ccccc3)C[C@@]2(O)C1 ; O[C@]12CNC[C@H]1C[C@H](Oc1ccccc1)C2]

The flask containing benzyl (3aS,5S,6aR)-3a-hydroxy-5-phenoxyhexahydrocyclopenta[c]pyrrole-2(1H)-carboxylate and benzyl (3aR,5R,6aS)-3a-hydroxy-5-phenoxyhexahydrocyclopenta[c]pyrrole-2(1H)-carboxylate (11.46 g, 32.4 mmol) was equipped with a magnetic stirbar and purged with nitrogen. To the flask was added anhydrous MeOH (200 mL) with stirring at ambient temperature. The flask was purged of oxygen by performing two vacuum-to-N₂ cycles on the manifold, and then Pd/C (10% Pd loading, Degussa wet-type, 0.724 g, 6.80 mmol) was charged with stirring. The flask was stoppered with a rubber septum and vacuum purged twice cycling from nitrogen to vacuum.

The H₂ balloon was affixed to a long syringe needle extending below the level of the liquid, and the vacuum was broken by opening the H₂ balloon to the evacuated flask using a plastic Luer stopcock. The reaction was vigorously stirred at room temperature for 2 h. A nitrogen inlet was placed into the flask and the flask was purged for 15 min. The reaction mixture was filtered through a pad of Celite, washing through with DCM. The filtrate was concentrated to yield the title intermediate as a white solid (6.3 g), which was used in the next step without purification.

LCMS: Rt 0.85 min; MS m/z 220.3 [M+H]⁺; Method B.

¹H NMR (400 MHz, Methanol-d₄) δ 7.31-7.19 (m, 2H), 6.97-6.82 (m, 3H), 3.24 (dd, J=11.6, 7.7 Hz, 1H), 2.94-2.81 (m, 2H), 2.66-2.48 (m, 2H), 2.31-2.15 (m, 2H), 2.09 (ddd, J=13.9, 4.7, 1.8 Hz, 1H), 1.81-1.69 (m, 1H). 1H under solvent peak.

### Intermediate 2

### (3aS,5S,6aR)-5-phenoxyhexahydrocyclopenta[c]pyrrol-3a(1H)-ol

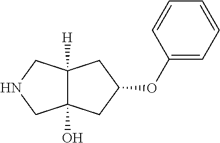 [SMILES: O[C@]12CNC[C@H]1C[C@H](Oc1ccccc1)C2]

### Step 1: Benzyl (3aS,5R,6aR)-3a,5-dihydroxyhexahydrocyclopenta[c]pyrrole-2(1H)-carboxylate

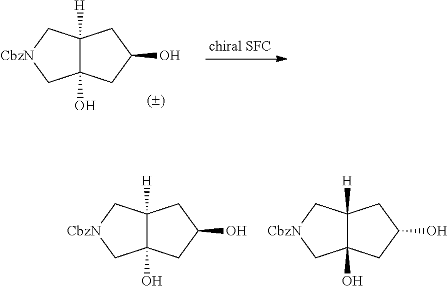 [SMILES: O=C(OCc1ccccc1)N1C[C@H]2C[C@@H](O)C[C@@]2(O)C1 ; O=C(OCc1ccccc1)N1C[C@H]2C[C@@H](O)C[C@@]2(O)C1 ; O=C(OCc1ccccc1)N1C[C@@H]2C[C@H](O)C[C@]2(O)C1]

The racemic mixture of benzyl (3aS,5R,6aR)-3a,5-dihydroxyhexahydrocyclopenta[c]pyrrole-2(1H)-carboxylate and benzyl (3aR,5S,6aS)-3a,5-dihydroxyhexahydrocyclopenta[c]pyrrole-2(1H)-carboxylate (from step 5 of Intermediate 1) (450 mg) was separated by chiral SFC using the condition below to provide benzyl (3aS,5R,6aR)-3a,5-dihydroxyhexahydrocyclopenta[c]pyrrole-2(1H)-carboxylate (190 mg, peak 1) as a colorless oil and benzyl (3aR,5S,6aS)-3a,5-dihydroxyhexahydrocyclopenta[c]pyrrole-2(1H)-carboxylate (220 mg, peak 2) as a colorless oil.

Column: Chiralpak AD (250 mm×30 mm, 10 μm), Flow rate: 70 g/min

Mobile phase: CO₂ (A), MeOH with 0.1% NH₄OH (B), Isocratic 60:40 (A:B)

Peak 1:

Chiral SFC: Rt 1.58 min (Column: Chiralpak AD-3 50×4.6 mm I.D., 3 μm, Flow rate: 3 mL/min, Mobile phase: CO₂ (A), MeOH with 0.05% DEA (B), Gradient elution: 5-40% B). ¹H NMR (400 MHz, CDC₃) δ 7.38-7.29 (m, 5H), 5.16 (s, 2H), 4.56-4.52 (m, 1H), 3.82-3.76 (m, 2H), 3.56-3.53 (m, 1H), 3.44-3.41 (m, 1H), 2.48-2.39 (m, 2H), 2.24-2.18 (m, 1H), 1.99-1.94 (m, 1H), 1.81 (br s, 1H), 1.65 (br s, 1H), 1.54-1.41 (m, 1H).

Peak 2:

Chiral SFC: Rt 2.04 min (Column: Chiralpak AD-3 50×4.6 mm I.D., 3 μm, Flow rate: 3 mL/min, Mobile phase: CO₂ (A), MeOH with 0.05% DEA (B), Gradient elution: 5-40% B). ¹H NMR (400 MHz, CDC₃) δ 7.38-7.31 (m, 5H), 5.14 (s, 2H), 4.56-4.51 (m, 1H), 3.82-3.76 (m, 2H), 3.56-3.52 (m, 1H), 3.44-3.41 (m, 1H), 2.47-2.39 (m, 2H), 2.24-2.18 (m, 1H), 1.99-1.94 (m, 1H), 1.82 (br s, 1H), 1.65 (br s, 1H), 1.51-1.41 (m, 1H).

### Step 2: Benzyl (3aS,5S,6aR)-3a-hydroxy-5-phenoxyhexahydrocyclopenta[c]pyrrole-2(1H)-carboxylate

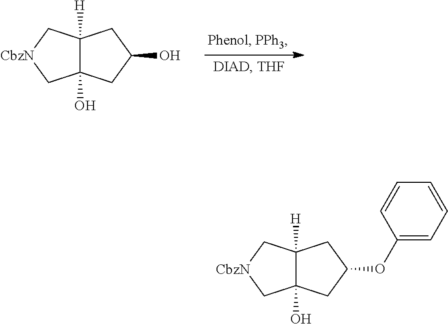 [SMILES: O=C(OCc1ccccc1)N1C[C@H]2C[C@@H](O)C[C@@]2(O)C1 ; O=C(OCc1ccccc1)N1C[C@H]2C[C@H](Oc3ccccc3)C[C@@]2(O)C1]

Starting with benzyl (3aS,5R,6aR)-3a,5-dihydroxyhexahydrocyclopenta[c]pyrrole-2(1H)-carboxylate (peak 1 from the previous step), and following the procedure used in step 6 of Intermediate 1, provided the title intermediate.

LCMS: Rt 0.84 min; MS m/z 354.2 [M+H]⁺; Method J.

¹H NMR (400 MHz, CDC₃) δ 7.38-7.28 (m, 7H), 7.01-6.96 (m, 1H), 6.88-6.85 (m, 2H), 5.14 (s, 2H), 4.95-4.92 (m, 1H), 3.81-3.78 (m, 2H), 3.50-3.46 (m, 1H), 3.30-3.25 (m, 1H), 2.76-2.72 (m, 2H), 2.47-2.41 (m, 1H), 2.32-2.27 (m, 1H), 2.18-2.10 (m, 1H), 1.75 (m, 1H).

### Step 3: (3aS,5S,6aR)-5-phenoxyhexahydrocyclopenta[c]pyrrol-3a(1H)-ol

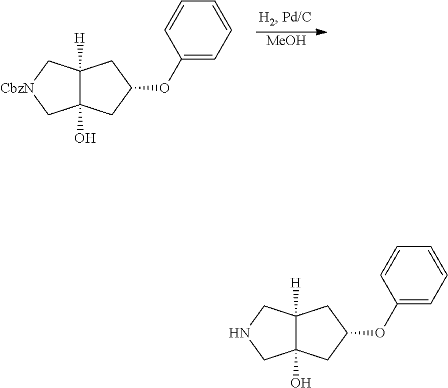 [SMILES: "MeOH" ; O=C(OCc1ccccc1)N1C[C@H]2C[C@H](Oc3ccccc3)C[C@@]2(O)C1 ; O[C@]12CNC[C@H]1C[C@H](Oc1ccccc1)C2]

Starting with benzyl (3aS,5S,6aR)-3a-hydroxy-5-phenoxyhexahydrocyclopenta[c]pyrrole-2(1H)-carboxylate, and following the procedure used in step 7 of Intermediate 1, provided the title intermediate.

LCMS: Rt 0.86 min; MS m/z 220.0 [M+H]⁺; Method I.

¹H NMR (400 MHz, DMSO-d₆) δ 7.33-7.21 (m, 2H), 6.94-6.84 (m, 3H), 4.88-4.66 (m, 2H), 3.06-3.01 (m, 1H), 2.72-2.65 (m, 2H), 2.53-2.51 (m, 1H), 2.46-2.42 (m, 1H), 2.30-2.14 (m, 2H), 2.04-1.94 (m, 1H), 1.92-1.86 (m, 1H), 1.80-1.71 (m, 1H).

### Intermediate 3

A racemic mixture of:
(3aS,5S,6aR)-5-(2-fluorophenoxy)hexahydrocyclopenta[c]pyrrol-3a(1H)-ol
(3aR,5R,6aS)-5-(2-fluorophenoxy)hexahydrocyclopenta[c]pyrrol-3a(1H)-ol

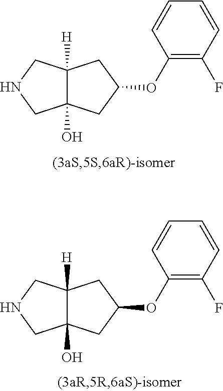 [SMILES: O[C@]12CNC[C@H]1C[C@H](Oc1ccccc1F)C2 ; O[C@@]12CNC[C@@H]1C[C@@H](Oc1ccccc1F)C2]

This was synthesized in a similar manner as Intermediate 1, using 2-fluorophenol in step 6.

LCMS: Rt 0.66 min; MS m/z 238.3 [M+H]⁺; Method B.

¹H NMR (400 MHz, DMSO-d₆) δ 7.26-7.14 (m, 2H), 7.14-7.02 (m, 1H), 6.96-6.87 (m, 1H), 4.88-4.79 (m, 1H), 4.73 (br s, 1H), 3.07-3.01 (m, 1H), 2.73-2.66 (m, 2H), 2.47-2.43 (m, 1H), 2.36-2.26 (m, 1H), 2.23-2.17 (m, 1H), 2.08-1.99 (m, 1H), 1.96-1.91 (m, 1H), 1.80-1.73 (m, 1H). 1H under solvent peak.

### Intermediate 4

### (3aS,5S,6aR)-5-(2-fluorophenoxy)hexahydrocyclopenta[c]pyrrol-3a(1H)-ol

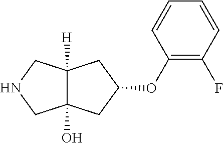 [SMILES: O[C@]12CNC[C@H]1C[C@H](Oc1ccccc1F)C2]

This was synthesized in a similar manner as Intermediate 2, using 2-fluorophenol in step 2.

LCMS: Rt 0.87 min; MS m/z 238.3 [M+H]⁺; Method I.

¹H NMR (400 MHz, CDC₃) δ 7.21-7.15 (m, 2H), 7.12-7.08 (m, 1H), 6.95-6.89 (m, 1H), 4.85-4.79 (m, 1H), 4.74 (br s, 1H), 3.07-3.01 (m, 1H), 2.73-2.66 (m, 2H), 2.47-2.43 (m, 1H), 2.36-2.25 (m, 1H), 2.23-2.17 (m, 1H), 2.08-1.99 (m, 1H), 1.97-1.91 (m, 1H), 1.79-1.73 (m, 1H). 1H under solvent peak.

### Intermediates 5 and 6

(3aS,4S,5S,6aR)-5-phenoxyhexahydrocyclopenta[c]pyrrole-3a,4(1H)-diol
(3aR,4R,5R,6aS)-5-phenoxyhexahydrocyclopenta[c]pyrrole-3a,4(1H)-diol

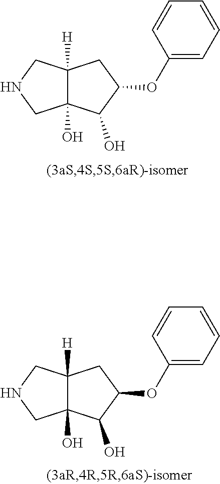 [SMILES: O[C@H]1[C@@H](Oc2ccccc2)C[C@@H]2CNC[C@@]21O ; O[C@@H]1[C@H](Oc2ccccc2)C[C@H]2CNC[C@]21O]

Step 1: A racemic mixture of:
Benzyl (3aS,5R)-5-hydroxy-3,3a,4,5-tetrahydrocyclopenta[c]pyrrole-2(1H)-carboxylate
Benzyl (3aR,5S)-5-hydroxy-3,3a,4,5-tetrahydrocyclopenta[c]pyrrole-2(1H)-carboxylate

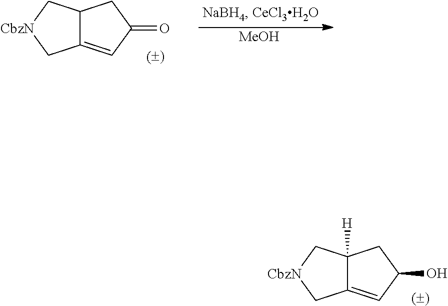 [SMILES: "MeOH" ; O=C1C=C2CN(C(=O)OCc3ccccc3)CC2C1 ; O=C(OCc1ccccc1)N1CC2=C[C@H](O)C[C@@H]2C1]

To a stirred solution of (±)-benzyl 5-oxo-3,3a,4,5-tetrahydrocyclopenta[c]pyrrole-2(1 h)-carboxylate (from step 3 of Intermediate 1) (2.0 g, 7.8 mmol) in methanol (500 mL) was added CeCl₃·H₂O (5.7 g, 23.3 mmol) followed by NaBH₄ (0.35 g, 9.36 mmol) at −70° C. The reaction mixture was stirred at RT for 4 h. The reaction mixture was concentrated, and the material was dissolved in EtOAc and washed with water. The organic layer was dried over Na₂SO₄, concentrated, and purified by FCC (60% EtOAc:Hexane) to provide the title intermediate (1.6 g).

LCMS: Rt 0.50 min; MS m/z 260.2 [M+H]⁺; Method D.

¹H NMR (400 MHz, CDC₃) δ 7.37-7.29 (m, 5H), 5.59 (d, J=16 Hz, 1H), 5.14 (m, 3H), 4.04 (dd, J=16.0, 6.0 Hz, 1H), 3.97-3.88 (m, 2H), 3.08-2.96 (m, 1H), 2.88 (t, J=9.6 Hz, 1H), 2.72-2.61 (m, 1H), 1.83 (t, J=10.0 Hz, 1H), 1.40-1.28 (m, 1H).

Step 2: A racemic mixture of:
Benzyl (3aS,5S)-5-phenoxy-3,3a,4,5-tetrahydrocyclopenta[c]pyrrole-2(1H)-carboxylate
Benzyl (3aR,5R)-5-phenoxy-3,3a 4,5-tetrahydrocyclopenta[c]pyrrole-2(1H-carboxylate

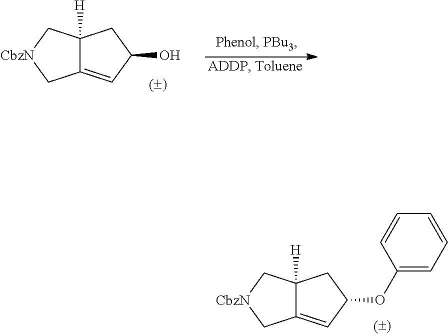 [SMILES: O=C(OCc1ccccc1)N1CC2=C[C@H](O)C[C@@H]2C1 ; "ADDP, Toluene" ; O=C(OCc1ccccc1)N1CC2=C[C@@H](Oc3ccccc3)C[C@@H]2C1]

To a solution of the racemate of benzyl (3aS,5R)-5-hydroxy-3,3a,4,5-tetrahydrocyclopenta[c]pyrrole-2(1H)-carboxylate and benzyl (3aR,5S)-5-hydroxy-3,3a,4,5-tetrahydrocyclopenta[c]pyrrole-2(1H)-carboxylate (6.0 g, 23.1 mmol), phenol (2.6 g, 27.7 mmol) and 1,1′-(azodicarbonyl)dipiperidine (11.6 g, 46.2 mmol) in toluene (500 mL) was added tributylphosphine (14 g, 69.3 mmol) at RT and the reaction mixture was stirred at 100° C. for 16 h. The reaction mixture was cooled to RT, filtered and the filtrate was concentrated. The crude material was purified by FCC (10% EtOAc:Hexane) to provide the title intermediate (3.5 g).

¹H NMR (400 MHz, CDC₃) δ 7.39-7.26 (m, 7H), 6.96-6.92 (m, 1H), 6.89 (d, J=8 Hz, 2H), 5.87 (d, J=14.8 Hz, 1H), 5.46 (dd, J=3.6, 2.4 Hz, 1H), 5.19-5.12 (m, 2H), 4.08-3.95 (m, 3H), 3.60-3.50 (m, 1H), 2.80 (dt, 10.4, 1.2 Hz, 1H), 2.39-2.30 (m, 1H), 1.90-1.83 (m, 1H).

Step 3: A racemic mixture of:
Benzyl (3aS,4S,5S,6aR)-3a,4-dihydroxy-5-phenoxyhexahydrocyclopenta[c]pyrrole-2(1H)-carboxylate
Benzyl (3aR,4R,5R,6aS)-3a,4-dihydroxy-5-phenoxyhexahydrocyclopenta[c]pyrrole-2(1H)-carboxylate

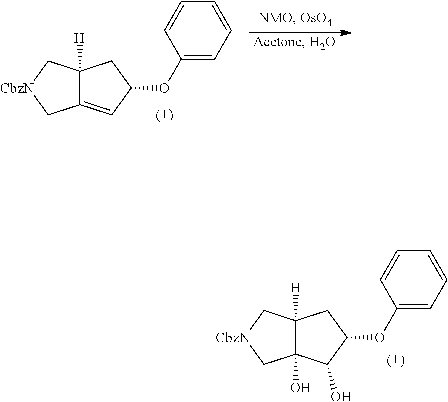

To a solution of the racemate of benzyl (3aS,5S)-5-phenoxy-3,3a,4,5-tetrahydrocyclopenta[c]pyrrole-2(1H)-carboxylate and benzyl (3aR,5R)-5-phenoxy-3,3a,4,5-tetrahydrocyclopenta[c]pyrrole-2(1H)-carboxylate (2.5 g, 7.4 mmol) and N-methyl morpholine N-oxide monohydrate (17 g, 126.5 mmol) in acetone (200 mL) and water (200 mL) was added a solution of OSO₄ (96 mg, 0.37 mmol) in t-BuOH (20 mL) at RT and the reaction mixture was stirred for 16 h. The reaction mixture was extracted with ethyl acetate, dried over Na₂SO₄, concentrated, and purified by FCC (50% EtOAc:Hexane) to provide the title intermediate (2.5 g).

LCMS: Rt 1.40 min; MS m/z 370.3 [M+H]⁺; Method D.

Step 4: Chiral separation of:
Benzyl (3aS,4S,5S,6aR)-3a,4-dihydroxy-5-phenoxyhexahydrocyclopenta[c]pyrrole-2(1H)-carboxylate
Benzyl (3aR,4R,5R,6aS)-3a,4-dihydroxy-5-phenoxyhexahydrocyclopenta[c]pyrrole-2(1H)-carboxylate

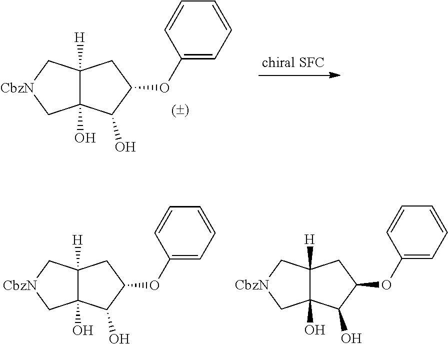 [SMILES: O=C(OCc1ccccc1)N1C[C@H]2C[C@H](Oc3ccccc3)[C@H](O)[C@@]2(O)C1 ; O=C(OCc1ccccc1)N1C[C@H]2C[C@H](Oc3ccccc3)[C@H](O)[C@@]2(O)C1 ; O=C(OCc1ccccc1)N1C[C@@H]2C[C@@H](Oc3ccccc3)[C@@H](O)[C@]2(O)C1]

The racemic mixture of benzyl (3aS,4S,5S,6aR)-3a,4-dihydroxy-5-phenoxyhexahydrocyclopenta[c]pyrrole-2(1H)-carboxylate and benzyl (3aR,4R,5R,6aS)-3a,4-dihydroxy-5-phenoxyhexahydrocyclopenta[c]pyrrole-2(1H)-carboxylate (2.5 g) was separated by chiral SFC using the method below to provide benzyl (3aS,4S,5S,6aR)-3a,4-dihydroxy-5-phenoxyhexahydrocyclopenta[c]pyrrole-2(1H)-carboxylate (chiral SFC Rt 7.23 min, 1.2 g) and benzyl (3aR,4R,5R,6aS)-3a,4-dihydroxy-5-phenoxyhexahydrocyclopenta[c]pyrrole-2(1H)-carboxylate (chiral SFC Rt 5.86 min, 1.2 g). Column: Chiralpak IG (10 mm×250 mm, 5 micron), Flow: 13 mL/min Mobile phase: CO₂ (A), EtOH:IPA, 1:1 (B), Isocratic 70:30 (A:B)

### Step 5: (3aS,4S,5S,6aR)-5-phenoxyhexahydrocyclopenta[c]pyrrole-3a,4(1H-diol

### Intermediate 5

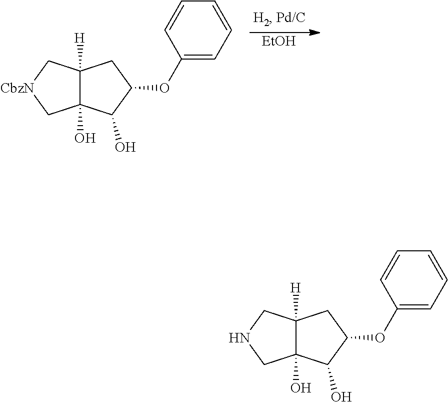 [SMILES: "EtOH" ; O=C(OCc1ccccc1)N1C[C@H]2C[C@H](Oc3ccccc3)[C@H](O)[C@@]2(O)C1 ; O[C@H]1[C@@H](Oc2ccccc2)C[C@@H]2CNC[C@@]21O]

A solution of benzyl (3aS,4S,5S,6aR)-3a,4-dihydroxy-5-phenoxyhexahydrocyclopenta[c]pyrrole-2(1H)-carboxylate (chiral SFC Rt 7.23 min from step 4) (1.2 g, 3.24 mmol) in EtOH (100 mL) was shaken with 10% Pd on carbon (120 mg) under H₂ (balloon pressure) for 6 h. The reaction mixture was filtered through Celite and concentrated to provide the title intermediate (750 mg) which was used without further purification.

LCMS: Rt 0.55 min; MS m/z 236.0 [M+H]⁺; Method E.

¹H NMR (400 MHz, Methanol-d₄) δ 7.27-7.23 (m, 2H), 7.01-6.99 (m, 2H), 6.92 (t, J=7.2 Hz, 1H), 4.78-4.73 (m, 1H), 3.94 (d, J=3.6 Hz, 1H), 3.23-3.19 (m, 1H), 2.97 (d, J=12.0 Hz, 1H), 2.86 (d, J=12.0 Hz, 1H), 2.70-2.65 (m, 1H), 2.54-2.49 (m, 1H), 2.30-2.23 (m, 1H), 1.60-1.55 (m, 1H).

### Step 6: (3aR,4R,5R,6aS)-5-phenoxyhexahydrocyclopenta[c]pyrrole-3a,4(1H)-diol (Intermediate 6)

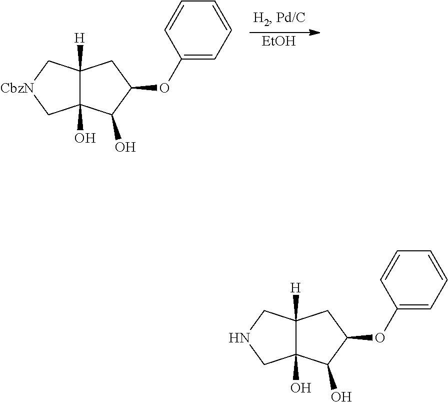 [SMILES: "EtOH" ; O=C(OCc1ccccc1)N1C[C@@H]2C[C@@H](Oc3ccccc3)[C@@H](O)[C@]2(O)C1 ; O[C@@H]1[C@H](Oc2ccccc2)C[C@H]2CNC[C@]21O]

Using the same method as step 5, starting from benzyl (3aR,4R,5R,6aS)-3a,4-dihydroxy-5-phenoxyhexahydrocyclopenta[c]pyrrole-2(1H)-carboxylate (chiral SFC Rt 5.86 min from step 4) (1.2 g, 3.24 mmol), provided the title intermediate (750 mg).

LCMS: Rt 0.55 min; MS m/z 236.0 [M+H]⁺; Method E.

¹H NMR (400 MHz, Methanol-d₄) δ 7.27-7.23 (m, 2H), 7.01-6.99 (m, 2H), 6.92 (t, J=7.2 Hz, 1H), 4.78-4.73 (m, 1H), 3.93 (d, J=4.0 Hz, 1H), 3.20-3.15 (m, 1H), 2.94 (d, J=12.4 Hz, 1H), 2.82 (d, J=12.0 Hz, 1H), 2.66-2.63 (m, 1H), 2.52-2.46 (m, 1H), 2.30-2.23 (m, 1H), 1.60-1.52 (m, 1H).

### Intermediate 7

A racemic mixture of:
(3aS,4S,5S,6aR)-5-(2-fluorophenoxy)hexahydrocyclopenta[c]pyrrole-3a,4(1H)-diol
(3aR,4R,5R,6aS)-5-(2-fluorophenoxy)hexahydrocyclopenta[c]pyrrole-3a,4(1H)-diol

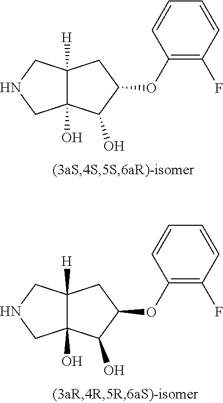 [SMILES: O[C@H]1[C@@H](Oc2ccccc2F)C[C@@H]2CNC[C@@]21O ; O[C@@H]1[C@H](Oc2ccccc2F)C[C@H]2CNC[C@]21O]

Steps 1-3: A racemic mixture of:
Benzyl (3aS,4S,5S,6aR)-5-(2-fluorophenoxy)-3a,4-dihydroxyhexahydrocyclopenta[c]pyrrole-2(1H)-carboxylate
Benzyl (3aR,4R,5R,6aS)-5-(2-fluorophenoxy)-3a,4-dihydroxyhexahydrocyclopenta[c]pyrrole-2(1H)-carboxylate

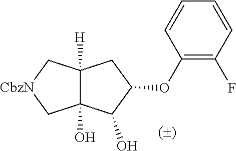 [SMILES: O=C(OCc1ccccc1)N1C[C@H]2C[C@H](Oc3ccccc3F)[C@H](O)[C@@]2(O)C1]

The title intermediate was synthesized using the same methods as steps 1-3 of Intermediates 5 and 6, using 2-fluorophenol in step 2 instead of phenol.

LCMS: Rt 1.44 min; MS m/z 388.0 [M+H]⁺; Method D.

Step 4: A racemic mixture of:
(3aS,4S,5S,6aR)-5-(2-fluorophenoxy)hexahydrocyclopenta[c]pyrrole-3a,4(1H)-diol
(3aR,4R,5R,6aS)-5-(2-fluorophenoxy)hexahydrocyclopenta[c]pyrrole-3a,4(1H)-diol

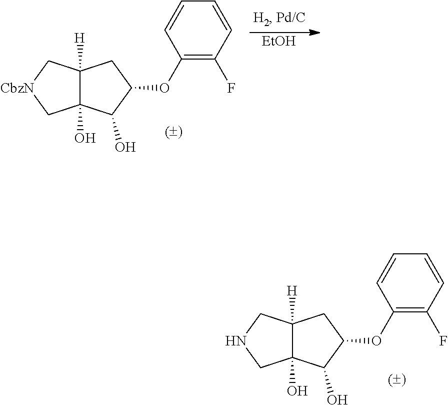 [SMILES: "EtOH" ; O=C(OCc1ccccc1)N1C[C@H]2C[C@H](Oc3ccccc3F)[C@H](O)[C@@]2(O)C1 ; O[C@H]1[C@@H](Oc2ccccc2F)C[C@@H]2CNC[C@@]21O]

Using the same method as step 5 of Intermediate 5, starting from a racemic mixture of benzyl (3aS,4S,5S,6aR)-5-(2-fluorophenoxy)-3a,4-dihydroxyhexahydrocyclopenta[c]pyrrole-2(1H)-carboxylate and benzyl (3aR,4R,5R,6aS)-5-(2-fluorophenoxy)-3a,4-dihydroxyhexahydrocyclopenta[c]pyrrole-2(1H)-carboxylate (200 mg), provided the title intermediate (130 mg).

LCMS: Rt 0.11 min; MS m/z 253.9 [M+H]⁺; Method D.

### Intermediate 8

### (3aS,4S,5S,6aR)-5-(2-fluorophenoxy)hexahydrocyclopenta[c]pyrrole-3a,4(1H)-diol

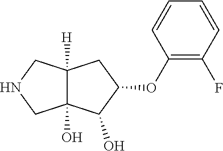 [SMILES: O[C@H]1[C@@H](Oc2ccccc2F)C[C@@H]2CNC[C@@]21O]

Step 1: Chiral separation of:
Benzyl (3aS,4S,5S,6aR)-5-(2-fluorophenoxy)-3a,4-dihydroxyhexahydrocyclopenta[c]pyrrole-2(1H)-carboxylate
Benzyl (3aR,4R,5R,6aR)-5-(2-fluorophenoxy)-3a,4-dihydroxyhexahydrocyclopenta[c]pyrrole-2(1H)-carboxylate

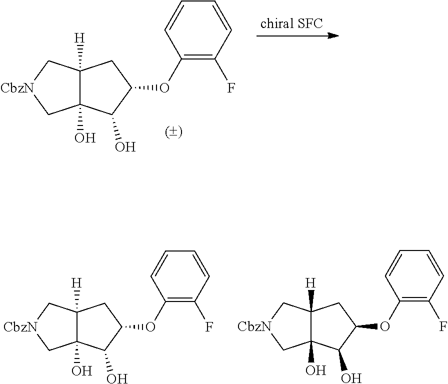 [SMILES: O=C(OCc1ccccc1)N1C[C@H]2C[C@H](Oc3ccccc3F)[C@H](O)[C@@]2(O)C1 ; O=C(OCc1ccccc1)N1C[C@H]2C[C@H](Oc3ccccc3F)[C@H](O)[C@@]2(O)C1 ; O=C(OCc1ccccc1)N1C[C@@H]2C[C@@H](Oc3ccccc3F)[C@@H](O)[C@]2(O)C1]

The racemic mixture of benzyl (3aS,4S,5S,6aR)-5-(2-fluorophenoxy)-3a,4-dihydroxyhexahydrocyclopenta[c]pyrrole-2(1H)-carboxylate and benzyl (3aR,4R,5R,6aS)-5-(2-fluorophenoxy)-3a,4-dihydroxyhexahydrocyclopenta[c]pyrrole-2(1H)-carboxylate (step 3 of Intermediate 7, 1.0 g) was separated by chiral SFC using the method below to provide benzyl (3aS,4S,5S,6aR)-5-(2-fluorophenoxy)-3a,4-dihydroxyhexahydrocyclopenta[c]pyrrole-2(1H)-carboxylate (chiral SFC Rt 13.24 min, 0.5 g) and benzyl (3aR,4R,5R,6aS)-5-(2-fluorophenoxy)-3a,4-dihydroxyhexahydrocyclopenta[c]pyrrole-2(1H)-carboxylate (chiral SFC Rt 19.13 min, 0.5 g).

Column: Chiralpak IG (10 mm×250 mm, 5 micron), Flow: 15 mL/min

Mobile phase: CO₂ (A), EtOH:IPA, 1:1 (B), Isocratic 70:30 (A:B)

### Step 2: (3aS,4S,5S,6aR)-5-(2-fluorophenoxy)hexahydrocyclopenta[c]pyrrole-3a,4(1H)-diol

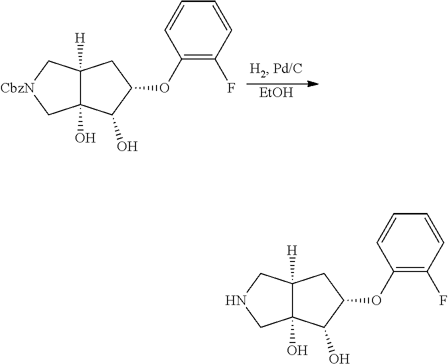 [SMILES: "EtOH" ; O=C(OCc1ccccc1)N1C[C@H]2C[C@H](Oc3ccccc3F)[C@H](O)[C@@]2(O)C1 ; O[C@H]1[C@@H](Oc2ccccc2F)C[C@@H]2CNC[C@@]21O]

Using the same method as step 5 of Intermediate 5, starting from benzyl (3aS,4S,5S,6aR)-5-(2-fluorophenoxy)-3a,4-dihydroxyhexahydrocyclopenta[c]pyrrole-2(1H)-carboxylate (chiral SFC Rt 13.24 min from step 1) (500 mg), provided the title intermediate (260 mg). LCMS: Rt 0.11 min; MS m/z 254.3 [M+H]⁺; Method D.

¹H NMR (400 MHz, Methanol-d₄) δ 7.19 (dt, J=8.4, 1.6 Hz, 1H), 7.11-7.06 (m, 2H), 6.97-6.91 (m, 1H), 4.78-4.73 (m, 1H), 3.92 (d, J=3.2 Hz, 1H), 3.16 (dd, J=12.0, 7.6 Hz, 1H), 2.93 (d, J=12.4 Hz, 1H), 2.78 (d, J=12.0 Hz, 1H), 2.62 (dd, J=11.2, 2.8 Hz, 1H), 2.55-2.49 (m, 1H), 2.32-2.24 (m, 1H), 1.55-1.49 (m, 1H).

### Intermediate 9

### (3aR,4R,5R,6aS)-5-(2-fluorophenoxy)hexahydrocyclopenta[c]pyrrole-3a,4(1H)-diol

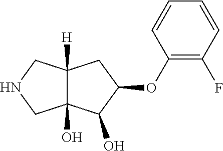 [SMILES: O[C@@H]1[C@H](Oc2ccccc2F)C[C@H]2CNC[C@]21O]

Using the same method as step 5 of Intermediate 5, starting from benzyl (3aR,4R,5R,6aS)-5-(2-fluorophenoxy)-3a,4-dihydroxyhexahydrocyclopenta[c]pyrrole-2(1H)-carboxylate (chiral SFC Rt 19.13 min from step 1 of Intermediate 8) (500 mg), provided the title intermediate (270 mg).

LCMS: Rt 0.10 min; MS m/z 254.0 [M+H]⁺; Method D.

¹H NMR (400 MHz, Methanol-d₄) δ 7.20 (dt, J=8.0, 1.6 Hz, 1H), 7.12-7.06 (m, 2H), 6.98-6.94 (m, 1H), 4.83-4.79 (m, 1H), 4.03 (d, J=4.0 Hz, 1H), 3.47-3.42 (m, 1H), 3.16-3.06 (m, 2H), 2.92-2.87 (m, 1H), 2.72-2.68 (m, 1H), 2.37-2.30 (m, 1H), 1.69-1.62 (m, 1H).

The following intermediate was made using similar procedures with the relevant starting materials:

| Intermediate | Name and structure | LCMS | ¹H NMR |
|---|---|---|---|
| 10 | A racemic mixture of: | Rt | (400 MHz, |
| | (3aS,5S,6aR)-5-(2,4- | 0.73 | Methanol- |
| | difluorophenoxy) | min; | d₄) δ 7.11 |
| | hexahydrocyclopenta[c]pyrrol- | MS | (td, J = |
| | 3a(1H)-ol | m/z | 9.2, 5.4 |
| | (3aR,5R,6aS)-5-(2,4- | 256.3 | Hz, 1H), |
| | difluorophenoxy) | [M + | 6.95 (ddd, |
| | hexahydrocyclopenta[c]pyrrol- | H]⁺; | J = 11.5, |
| | 3a(1H)-ol | Method | 8.6, 3.0 |
| | | H. | Hz, 1H), |
| | 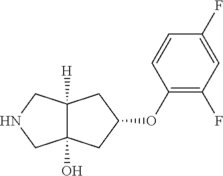 [SMILES: O[C@]12CNC[C@H]1C[C@H](Oc1ccc(F)cc1F)C2] (3aS,5S,6aR)-isomer 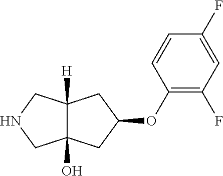 [SMILES: O[C@@]12CNC[C@@H]1C[C@@H](Oc1ccc(F)cc1F)C2] | | 6.91- 6.77 (m, 1H), 4.81- 4.74 (m, 1H), 3.28- 3.18 (m, 1H), 2.94- 2.80 (m, 2H), 2.68- 2.49 (m, 2H), 2.32- 2.17 (m, 2H), 2.17- 2.07 (m, 1H), 1.78- 1.66 (m, 1H). |
| | (3aS,5R,6aR)-isomer | | |

### Intermediate 11

### 6-(2-chloroacetyl)-3,4-dihydroquinolin-2(1H)-one

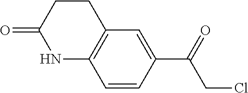 [SMILES: O=C1CCc2cc(C(=O)CCl)ccc2N1]

In a round bottom flask, to AlCl₃ (16.49 g, 124 mmol) under nitrogen was added CS₂ (88 mL) and this was cooled to 0° C. Chloroacetyl chloride (3.40 mL, 42.4 mmol) was added. After 10 minutes 3,4-dihydroquinolin-2(1H)-one (CAS #553-03-7) (5.20 g, 35.3 mmol) was added in two portions and the reaction was stirred at 45° C. for 20 min. The reaction was cooled to room temperature and the colorless solvent was decanted away, leaving behind a brown oily precipitate. This residue was placed in an ice bath and diluted slowly with ice and cold water. The tan precipitate was filtered and washed with water 3×, then dried to provide the title intermediate (7.46 g) as an offwhite solid which was used without further purification.

LCMS: Rt 0.67 min; MS m/z 224.2 [M+H]⁺; Method A.

¹H NMR (400 MHz, Methanol-d₄) δ 7.92-7.80 (m, 2H), 6.96 (d, J=8.3 Hz, 1H), 4.86 (s, 2H), 3.10-2.98 (m, 2H), 2.69-2.55 (m, 2H).

### Intermediate 12

### 6-(2-chloroacetyl)quinolin-2(1H)-one

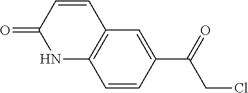 [SMILES: O=C(CCl)c1ccc2[nH]c(=O)ccc2c1]

To a suspension of 6-(2-chloroacetyl)-3,4-dihydroquinolin-2(1H)-one (Intermediate 11) (0.194 g, 0.867 mmol) in chloroform (17.35 mL) under nitrogen was added NBS (0.201 g, 1.13 mmol) and benzoyl peroxide (10.5 mg, 0.043 mmol) and the reaction was stirred at 60° C. for 2 h. The reaction was cooled and filtered, rinsing with chloroform 2×, and the solid was dried under vacuum to provide the title intermediate (114 mg) as a light brown solid which was used without further purification.

LCMS: Rt 0.66 min; MS m/z 222.1 [M+H]⁺; Method A.

¹H NMR (400 MHz, Methanol-d₄) δ 8.39 (d, J=2.0 Hz, 1H), 8.16 (dd, J=8.7, 2.0 Hz, 1H), 8.06 (d, J=9.5 Hz, 1H), 7.44 (d, J=8.7 Hz, 1H), 6.68 (d, J=9.6 Hz, 1H), 4.97 (s, 2H).

### Intermediate 13

### 6-(2-chloroacetyl)-5-fluoro-3,4-dihydroquinolin-2(1H)-one

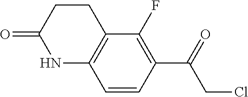 [SMILES: O=C1CCc2c(ccc(C(=O)CCl)c2F)N1]

### Step 1: (2-fluoro-6-nitrophenyl)methanol

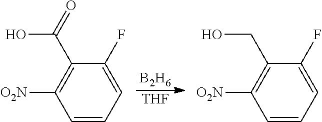

To a solution of 2-fluoro-6-nitrobenzoic acid (CAS #385-02-4) (5 g, 27 mmol) in THF (50 mL) under N₂ was added dropwise B₂H₆(10M in dimethyl sulfide, 10 mL, 108 mmol) and the reaction was stirred at RT for 30 min, then at 60° C. for 15.5 h. The reaction was quenched with MeOH (60 mL) very slowly, and the solution was stirred at RT for 2 h, and then concentrated to provide the title intermediate (4.2 g) as a yellow solid which was used without further purification.

LCMS: Rt 0.33 min; MS m/z 154.2 [M+H−H₂O]⁺; Method J.

¹H NMR (400 MHz, DMSO-d₆) δ 7.78-7.71 (m, 1H), 7.63-7.55 (m, 2H), 5.43 (br s, 1H), 4.70 (d, J=1.6 Hz, 2H).

### Step 2: 2-(bromomethyl)-1-fluoro-3-nitrobenzene

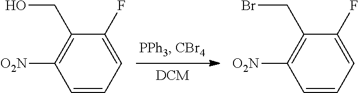 [SMILES: O=[N+]([O-])c1cccc(F)c1CO ; O=[N+]([O-])c1cccc(F)c1CBr]

To a solution of (2-fluoro-6-nitrophenyl)methanol (3.0 g, 17.5 mmol) in DCM (11.5 mL) was added CBr₄ (14.5 g, 43.8 mmol) and PPh₃ (11.5 g, 43.8 mmol) and the reaction was stirred at RT for 3 h. The reaction was quenched with saturated aqueous NH₄Cl (30 mL) and extracted with DCM (2×20 mL), dried with Na₂SO₄, filtered and concentrated. The crude material was purified by FCC (0-10% EtOAc:PE) to provide the title intermediate (2.1 g) as a light yellow oil.

¹H NMR (400 MHz, CDCl₃) δ 7.91-7.83 (m, 1H), 7.57-7.38 (m, 2H), 4.96 (d, J=1.6 Hz, 1H), 4.84 (d, J=1.6 Hz, 1H).

### Step 3: Diethyl 2-(2-fluoro-6-nitrobenzyl)malonate

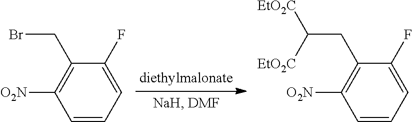 [SMILES: O=[N+]([O-])c1cccc(F)c1CBr ; CCOC(=O)C(Cc1c(F)cccc1[N+](=O)[O-])C(=O)OCC]

To a solution of diethyl malonate (1.72 g, 10.7 mmol) in DMF (12 mL) was added NaH (60% in mineral oil, 538 mg, 13.5 mmol) in portions at 0° C. The reaction was stirred at RT for 30 min, then a solution of 2-(bromomethyl)-1-fluoro-3-nitrobenzene (2.1 g, 8.97 mmol) in DMF (8 mL) was added dropwise and the reaction was stirred at RT for another 15.5 h. The reaction was poured into saturated aqueous NH₄Cl (20 mL), extracted with EtOAc (3×10 mL), dried with Na₂SO₄, filtered and concentrated. The crude material was purified by FCC (0-50% EtOAc:PE) to provide the title intermediate (2.3 g) as a light yellow oil.

LCMS: Rt 1.02 min; MS m/z 314.2 [M+H]⁺; Method I.

¹H NMR (400 MHz, CDCl₃) δ 7.80-7.74 (m, 1H), 7.44-7.31 (m, 2H), 4.21-4.15 (m, 4H), 3.79-3.73 (m, 1H), 3.57-3.53 (m, 2H), 1.25-1.21 (m, 6H).

### Step 4: Ethyl 5-fluoro-2-oxo-1,2,3,4-tetrahydroquinoline-3-carboxylate

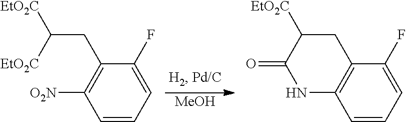 [SMILES: "MeOH" ; CCOC(=O)C(Cc1c(F)cccc1[N+](=O)[O-])C(=O)OCC ; CCOC(=O)C1Cc2c(F)cccc2NC1=O]

To a solution of diethyl 2-(2-fluoro-6-nitrobenzyl)malonate (2.3 g, 7.34 mmol) in MeOH (23 mL) was added 10% Pd/C (400 mg), and the reaction was stirred at RT for 16 h under H₂ (15 psi). The suspension was filtered through Celite, washing with EtOAc (3×5 mL). The combined filtrates were concentrated to provide the title intermediate (1.6 g) as a white solid which was used without further purification.

LCMS: Rt 0.81 min; MS m/z 238.1 [M+H]⁺; Method J.

¹H NMR (400 MHz, CDCl₃) δ 8.20 (br s, 1H), 7.20-7.12 (m, 1H), 6.82-6.73 (m, 1H), 6.60 (d, J=8.0 Hz, 1H), 4.28-4.18 (m, 2H), 3.70-3.56 (m, 1H), 3.49-3.35 (m, 1H), 3.25-3.15 (m, 1H), 1.28-1.24 (m, 3H).

### Step 5: 5-fluoro-3,4-dihydroquinolin-2(1H)-one

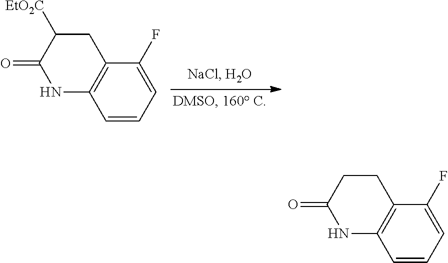 [SMILES: CCOC(=O)C1Cc2c(F)cccc2NC1=O ; O=C1CCc2c(F)cccc2N1]

To a solution of ethyl 5-fluoro-2-oxo-1,2,3,4-tetrahydroquinoline-3-carboxylate (1.6 g, 6.74 mmol) in DMSO (160 mL) and water (16 mL) was added NaCl (1.18 g, 20.2 mmol) and the reaction was stirred at 160° C. for 8 h. The reaction was cooled, diluted with water (100 mL) and extracted with EtOAc (3×30 mL). The combined organic phases were washed with saturated aqueous NaCl (3×40 mL), dried with Na₂SO₄, filtered and concentrated to provide the title intermediate (1 g) as a white solid which was used without further purification.

LCMS: Rt 0.52 min; MS m/z 166.0 [M+H]⁺; Method J.

¹H NMR (400 MHz, CDCl₃) δ 8.51 (br s, 1H), 7.18-7.11 (m, 1H), 6.81-6.69 (m, 1H), 6.60 (d, J=8.0 Hz, 1H), 3.03-2.99 (m, 2H), 2.71-2.59 (m, 2H).

### Step 6: 6-(2-chloroacetyl)-5-fluoro-3,4-dihydroquinolin-2(1H)-one

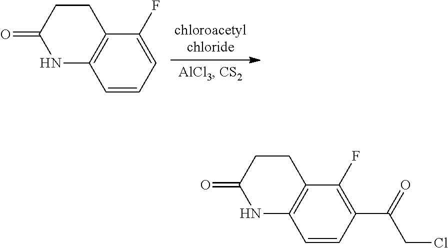 [SMILES: O=C1CCc2c(F)cccc2N1 ; O=C1CCc2c(ccc(C(=O)CCl)c2F)N1]

Using the same method as Intermediate 11, starting with 5-fluoro-3,4-dihydroquinolin-2(1H)-one (500 mg, 3.03 mmol), gave crude material which was purified by FCC (30-80% EtOAc:PE) to provide the title intermediate (300 mg) as a white solid.

¹H NMR (400 MHz, CDCl₃) δ 8.60 (br s, 1H), 7.89-7.85 (m, 1H), 6.72 (d, J=8.4 Hz, 1H), 4.70 (d, J=3.2 Hz, 2H), 3.10-3.06 (m, 2H), 2.74-2.69 (m, 2H).

### Intermediate 14

### 7-(2-chloroacetyl)-4,5-dihydrobenzo[d][1,3]oxazepin-2(1H)-one

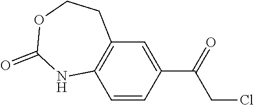 [SMILES: O=C1Nc2ccc(C(=O)CCl)cc2CCO1]

### Step 1: 4,5-dihydrobenzo[d][1,3]oxazepin-2(1H)-one

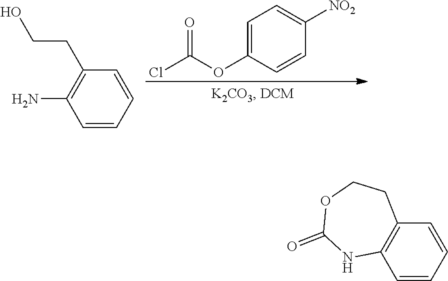

To a solution of 2-(2-aminophenyl)ethan-1-ol (CAS #5339-85-5) (4.8 g, 35.0 mmol) in DCM (96 mL) was added K₂CO₃ (9.67 g, 70.0 mmol) and 4-nitrophenyl carbonochloridate (10.6 g, 52.5 mmol) and this was stirred at RT for 16 h, then diluted with water (40 mL), extracted with DCM (3×30 mL), dried with Na₂SO₄, filtered and concentrated. The crude material was purified by FCC (0-90% EtOAc:PE) to provide the title intermediate (2.2 g) as a brown solid.

LCMS: Rt 0.57 min; MS m/z 164.0 [M+H]⁺; Method J.

¹H NMR (400 MHz, CDCl₃) δ 9.08-8.74 (m, 1H), 7.23-7.15 (m, 1H), 7.11-7.09 (m, 1H), 7.07-6.96 (m, 2H), 4.62-4.47 (m, 2H), 3.29-3.14 (m, 2H).

### Step 2: 7-(2-chloroacetyl)-4,5-dihydrobenzo[d][1,3]oxazepin-2(1H)-one

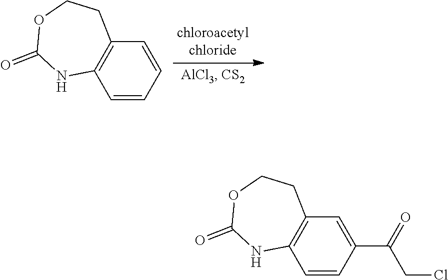 [SMILES: O=C1Nc2ccccc2CCO1 ; O=C1Nc2ccc(C(=O)CCl)cc2CCO1]

Using the same method as Intermediate 11, starting with 4,5-dihydrobenzo[d][1,3]oxazepin-2(1H)-one (500 mg, 3.06 mmol), provided the title intermediate (700 mg) as an offwhite solid which was used without further purification.

LCMS: Rt 0.64 min; MS m/z 240.0 [M+H]⁺; Method J.

¹H NMR (400 MHz, DMSO-d₆) δ 9.97 (s, 1H), 7.87-7.65 (m, 2H), 7.19 (d, J=8.4 Hz, 1H), 5.09 (s, 2H), 4.51-4.31 (m, 2H), 3.26-3.12 (m, 2H).

### Intermediate 15

### 6-(2-chloroacetyl)-1,4-dihydro-2H-benzo[d][1,3]thiazin-2-one

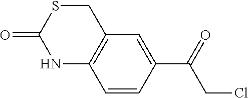 [SMILES: O=C1Nc2ccc(C(=O)CCl)cc2CS1]

### Step 1: 1,4-dihydro-2H-benzo[d][1,3]thiazine-2-thione

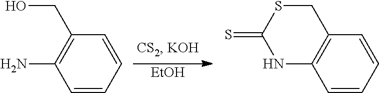 [SMILES: "EtOH" ; Nc1ccccc1CO ; S=C1Nc2ccccc2CS1]

To a solution of KOH (3.42 g, 60.9 mmol) in EtOH (30 mL) was added CS₂ (7.36 mL, 122 mmol) dropwise at 0° C. (2-aminophenyl)methanol (CAS #5344-90-1) (5 g, 41 mmol) was added and the reaction was heated to 80° C. for 20 h. The reaction was cooled and concentrated. KOH (10% aqueous, 80 mL) was added and the resulting precipitate was filtered away. The filtrate was made acidic with 1N HCl, and the solid was collected by filtration to provide the title intermediate (7 g) as a white solid which was used without further purification.

LCMS: Rt 0.60 min; MS m/z 181.9 [M+H]⁺; Method J.

¹H NMR (400 MHz, CDCl₃) δ 9.95 (br s, 1H), 7.35-7.28 (m, 1H), 7.23-7.16 (m, 2H), 6.98 (d, J=8.0 Hz, 1H), 4.03 (s, 2H).

### Step 2: 1,4-dihydro-2H-benzo[d][1,3]thiazin-2-one

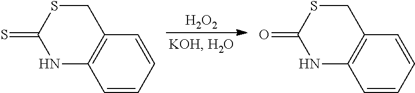

To a solution of 1,4-dihydro-2H-benzo[d][1,3]thiazine-2-thione (2 g, 11 mmol) in 1M aqueous KOH solution (120 mL) was added H₂O₂(3% aqueous, 120 mL). This was stirred at RT for 1 h, and the resulting precipitate was collected by filtration and washed with IPA (5 mL) to provide the title intermediate (1.48 g) as a white solid which was used without further purification.

LCMS: Rt 0.64 min; MS m/z 166.0 [M+H]⁺; Method J.

¹H NMR (400 MHz, CDCl₃) δ 8.55 (br s, 1H), 7.27 (s, 1H), 7.20 (d, J=7.2 Hz, 1H), 7.13-7.05 (m, 1H), 6.89 (d, J=8.0 Hz, 1H), 4.10 (s, 2H).

### Step 3: 6-(2-chloroacetyl)-1,4-dihydro-2H-benzo[d][1,3]thiazin-2-one

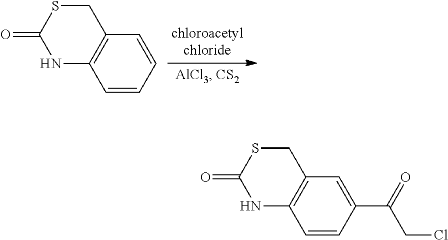 [SMILES: O=C1Nc2ccccc2CS1 ; O=C1Nc2ccc(C(=O)CCl)cc2CS1]

The method of Intermediate 11 was followed, starting with 1,4-dihydro-2H-benzo[d][1,3]thiazin-2-one (500 mg, 3.03 mmol). After diluting the reaction with ice, the mixture was extracted with EtOAc (3×20 mL), dried with Na₂SO₄, filtered and concentrated to provide the title intermediate (600 mg) as a white solid which was used without further purification.

LCMS: Rt 0.60 min; MS m/z 241.9 [M+H]⁺; Method J.

¹H NMR (400 MHz, DMSO-d₆) δ 11.13 (s, 1H), 7.99-7.81 (m, 2H), 7.11 (d, J=8.4 Hz, 1H), 5.11 (s, 2H), 4.30 (s, 2H).

### Intermediate 16

### 6-(2-chloroacetyl)-8-fluoro-1,4-dihydro-2H-benzo[d][1,3]thiazin-2-one

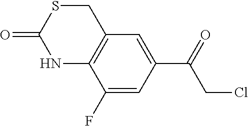 [SMILES: O=C1Nc2c(F)cc(C(=O)CCl)cc2CS1]

### Step 1: (2-amino-3-fluorophenyl)methanol

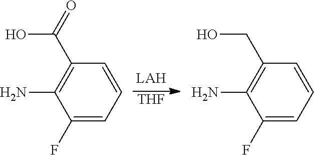 [SMILES: Nc1c(F)cccc1C(=O)O ; Nc1c(F)cccc1CO ; "THF"]

To a stirred suspension of LAH (13.7 g, 361 mmol) in THF (100 mL) under N₂ at 0° C. was added dropwise a solution of 2-amino-3-fluorobenzoic acid (CAS #825-22-9) (28 g, 180 mmol) in THF (200 mL), and the reaction was stirred at RT for 2 h. Water (13.7 mL) was added dropwise, then 15% aqueous NaOH (13.7 mL) was added dropwise. The reaction was diluted with THF (100 mL) and water (41.1 mL), then dried with Na₂SO₄ and filtered, washing through with EtOAc (2×100 mL). The combined organic phase was dried again with Na₂SO₄, filtered and concentrated. The crude material was purified by FCC (0-80% EtOAc:PE) to provide the title intermediate (20 g) as a yellow solid.

¹H NMR (400 MHz, CDCl₃) δ 7.01-6.95 (m, 1H), 6.87 (d, J=7.6 Hz, 1H), 6.68-6.62 (m, 1H), 4.71 (s, 2H), 4.25 (br s, 2H), 1.68 (br s, 1H).

### Step 2: 8-fluoro-1,4-dihydro-2H-benzo[d][1,3]thiazine-2-thione

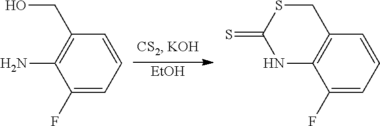 [SMILES: "EtOH" ; Nc1c(F)cccc1CO ; Fc1cccc2c1NC(=S)SC2]

Using the same method as step 1 of Intermediate 15, starting with (2-amino-3-fluorophenyl)methanol (5 g, 35 mmol), provided the title intermediate (9 g) as a white solid which was used without further purification.

LCMS: Rt 0.62 min; MS m/z 199.9 [M+H]⁺; Method J.

¹H NMR (400 MHz, CDCl₃) δ 9.47 (br s, 1H), 7.19-6.94 (m, 3H), 4.06 (s, 2H).

### Step 3: 8-fluoro-1,4-dihydro-2H-benzo[d][1,3]thiazin-2-one

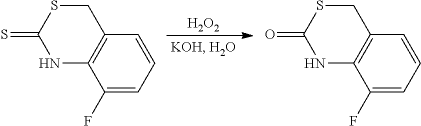

To a solution of 8-fluoro-1,4-dihydro-2H-benzo[d][1,3]thiazine-2-thione (2.0 g, 10.0 mmol) in 1 M aqueous KOH solution (20 mL) was added slowly H₂O₂(30% aqueous, 4.0 mL, 40.2 mmol). This was stirred at RT for 4 h. The pH was adjusted to ˜7 with 1 N HCl and diluted with saturated aqueous Na₂S₂O₃, then extracted with EtOAc (3×50 mL), dried with Na₂SO₄, filtered and concentrated. The crude material was purified by FCC (0-60% EtOAc:PE) to provide the title intermediate (1.0 g) as a white solid.

LCMS: Rt 0.49 m; MS m/z 183.9 [M+H]M; Method J.

¹H NMR (400 MHz, CDCl₃) δ 7.82 (br s, 1H), 7.13-6.98 (m, 3H), 4.14 (7, 2H).

### Step 4: 6-(2-chloroacetyl)-8-fluoro-1,4-dihydro-2H-benzo[d][1,3]thiazin-2-one

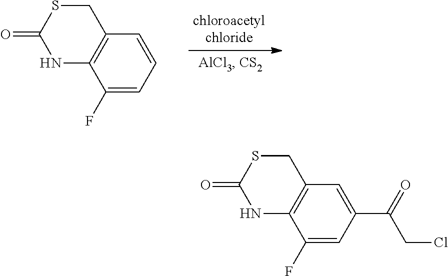 [SMILES: O=C1Nc2c(F)cccc2CS1 ; O=C1Nc2c(F)cc(C(=O)CCl)cc2CS1]

The method of Intermediate 11 was followed, starting with 8-fluoro-1,4-dihydro-2H-benzo[d][1,3]thiazin-2-one. After diluting the reaction with ice, the mixture was extracted with EtOAc 3×, dried with Na₂SO₄, filtered and concentrated to provide the title intermediate as a yellow solid which was used without further purification.

LCMS: Rt 0.70 m(-; MS m/z 259.9 [M+H]⁺; Method J.

H NMR (400 MHz, DMSO-d₆) δ 11.18 (s, 1H), 7.83-7.77 (m, 2H), 5.14 (s, 2H), 4.36 (s, 2H).

The following intermediates were made using similar procedures from the starting materials shown.

| Intermediate | Structure and name | Starting material | LCMS | ¹H NMR |
|---|---|---|---|---|
| 17 | 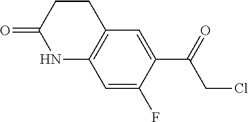 [SMILES: O=C1CCc2cc(C(=O)CCl)c(F)cc2N1] | 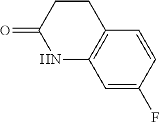 [SMILES: O=C1CCc2ccc(F)cc2N1] | Rt 0.58 min; MS m/z 242.0 [M + H]⁺; Method J. | (400 MHz, DMSO-d₆) δ 10.58 (s, 1H), 7.75 (d, J = 7.6 Hz, 1H), 6.72 (d, J = 12.0 Hz, 1H), 4.96 (d, J = 2.4 Hz, 2H), 2.93 (t, J = 7.6 Hz, 2H), 2.49-2.42 (m, 2H). |
| | 6-(2-chloroacetyl)-7-fluoro-3,4- | 7-fluoro-3,4- | | |
| | dihydroquinolin-2(1H)-one | dihydroquinolin- | | |
| | | 2(1H)-one | | |
| | | CAS# 4590-52-7 | | |
| 18 | 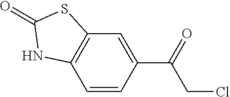 [SMILES: O=C(CCl)c1ccc2[nH]c(=O)sc2c1] | 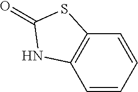 | Rt 0.59 min; MS m/z 227.9 [M + H]⁺; Method J. | (400 MHz, DMSO-d₆) δ 12.37 (s, 1H), 8.27 (d, J = 1.6 Hz, 1H), 7.92-7.90 (m, 1H), 7.23 (d, J = 8.4 Hz, 1H), 5.15 (s, 2H). |
| | 6-(2- | Benzo[d]thiazol- | | |
| | chloroacetyl)benzo[d] | 2(3H)-one | | |
| | thiazol-2(3H)-one | CAS# 934-34-9 | | |

### Intermediate 19

### 6-(2-chloroacetyl)-3,3-dimethyl-3,4-dihydroquinolin-2(1H-one

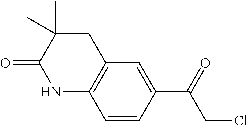 [SMILES: CC1(C)Cc2cc(C(=O)CCl)ccc2NC1=O]

### Step 1: 3-(2-bromophenyl)-2,2-dimethylpropanenitrile

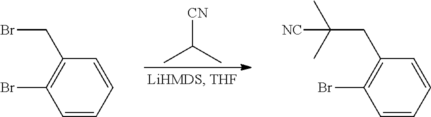

To a solution of isobutyronitrile (3.59 g, 52 mmol) in dry THF (30 mL) at 0° C. was added LiHMDS (1.OM in THF, 80 mL, 80 mmol) dropwise. The reaction was stirred for 30 min, then a solution of 1-bromo-2-(bromomethyl)benzene (CAS #3433-80-5) (10 g, 40 mmol) in dry THF (70 mL) was added and this was stirred at RT for 11.5 h. The reaction was diluted with saturated aqueous NH₄Cl (60 mL), extracted with EtOAc (3×100 mL), dried with Na₂SO₄, filtered and concentrated. The crude material was purified by FCC (0-20% EtOAc:PE) to provide the title intermediate (9.2 g) as a colorless oil.

LCMS: Rt 0.88 min; MS m/z 238.0 and 240.1 [M+H]⁺; Method J.

¹H NMR (400 MHz, CDCl₃) δ 7.61-7.58 (m, 1H), 7.53-7.50 (m, 1H), 7.35-7.30 (m, 1H), 7.18-7.13 (m, 1H), 3.09 (s, 2H), 1.44 (s, 6H).

### Step 2: 3,3-dimethyl-3,4-dihydroquinolin-2(1H-one

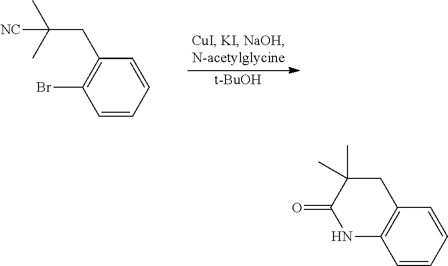

To a solution of 3-(2-bromophenyl)-2,2-dimethylpropanenitrile (5 g, 21 mmol) in t-BuOH (210 mL) was added CuI (600 mg, 3.15 mmol), KI (105 mg, 0.63 mmol), NaOH (3.36 g, 84.0 mmol) and N-acetylglycine (738 mg, 0.42 mmol), and the reaction was stirred at 100° C. for 72 h. The reaction was diluted with DCM, filtered, and the filtrate was concentrated. The crude material was purified by FCC (0-50% EtOAc:PE) to provide the title intermediate (2.2 g) as a white solid.

LCMS: Rt 0.65 min; MS m/z 176.1 [M+H]⁺; Method J.

¹H NMR (400 MHz, CDCl₃) δ 8.04 (br s, 1H), 7.22-7.11 (m, 2H), 7.03-6.95 (m, 1H), 6.77-6.74 (m, 1H), 2.81 (s, 2H), 1.22 (s, 6H).

### Step 3: 6-bromo-3,3-dimethyl-3,4-dihydroquinolin-2(1H)-one

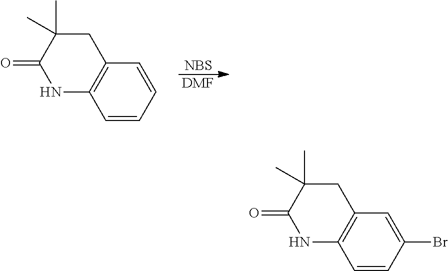

To a solution of 3,3-dimethyl-3,4-dihydroquinolin-2(1H)-one (1.1 g, 6.3 mmol) in DMF (11 mL) at 0° C. was added dropwise a solution of NBS (1.23 g, 6.91 mmol) in DMF (11 mL), and this was stirred at RT for 16 h. The reaction was diluted with water (30 mL), and the precipitated solid was collected by filtration and washed with water (10 mL) to provide the title intermediate (1.26 g) as a yellow solid which was used without further purification.

LCMS: Rt 0.75 min; MS m/z 254.0 and 256.0 [M+H]⁺; Method J.

¹H NMR (400 MHz, CDCl₃) δ 7.77 (br s, 1H), 7.32-7.27 (m, 2H), 6.68-6.58 (m, 1H), 2.78 (s, 2H), 1.21 (s, 6H).

### Step 4: 3,3-dimethyl-6-vinyl-3,4-dihydroquinolin-2(1H)-one

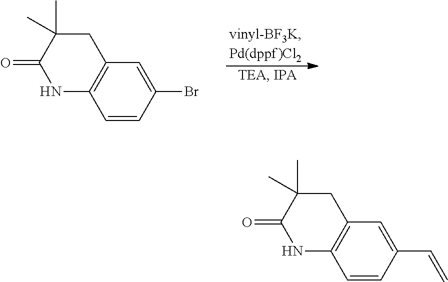

To a solution of 6-bromo-3,3-dimethyl-3,4-dihydroquinolin-2(1H)-one (1.26 g, 4.96 mmol) and potassium vinyltrifluoroborate (1.33 g, 9.92 mmol) in isopropanol (13 mL) was added triethylamine (2.07 mL, 14.9 mmol) and Pd(dppf)Cl₂ (363 mg, 0.50 mmol), and the reaction was stirred under N₂ at 90° C. for 16 h. The reaction was cooled and concentrated, then diluted with water (20 mL), extracted with EtOAc (3×40 mL), dried with Na₂SO₄, filtered and concentrated. The crude material was purified by FCC (30-70% EtOAc:PE) to provide the title intermediate (800 mg) as a yellow solid.

LCMS: Rt 0.83 min; MS m/z 202.1 [M+H]⁺; Method J.

¹H NMR (400 MHz, CDCl₃) δ 7.69 (br s, 1H), 7.26-7.19 (m, 2H), 6.76-6.58 (m, 2H), 5.69-5.64 (m, 1H), 5.20-5.17 (m, 1H), 2.81 (s, 2H), 1.22 (s, 6H).

### Step 5: 6-acetyl-3,3-dimethyl-3,4-dihydroquinolin-2(1H)-one

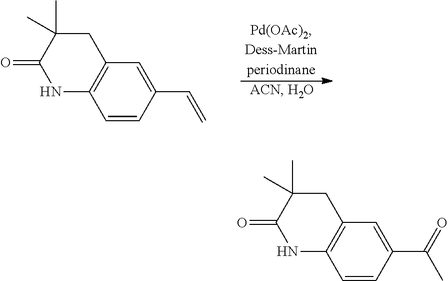

To a solution of 3,3-dimethyl-6-vinyl-3,4-dihydroquinolin-2(1H)-one (700 mg, 3.48 mmol) in acetonitrile (16.8 mL) and water (2.4 mL) was added Pd(OAc)₂ (78 mg, 0.35 mmol) and Dess-Martin periodinane (1.77 g, 4.17 mmol). This was stirred under N₂ at 60° C. for 2 h, then filtered through a small pad of silica gel, washing through with EtOAc (2×10 mL), and the filtrate was concentrated. The crude material was purified by FCC (50-80% EtOAc:PE) to provide the title intermediate (570 mg) as a yellow solid.

LCMS: Rt 0.80 min; MS m/z 218.2 [M+H]⁺; Method J.

¹H NMR (400 MHz, CDCl₃) δ 8.08 (br s, 1H), 7.84-7.80 (m, 2H), 6.82-6.80 (m, 1H), 2.87 (s, 2H), 2.58 (s, 3H), 1.23 (s, 6H).

### Step 6: 6-(2-chloroacetyl)-3,3-dimethyl-3,4-dihydroquinolin-2(1H-one

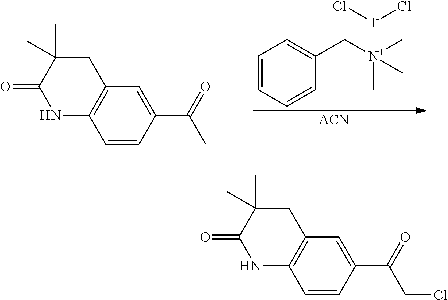

To a solution of 6-acetyl-3,3-dimethyl-3,4-dihydroquinolin-2(1H)-one (300 mg, 1.38 mmol) in acetonitrile (4.6 mL) was added benzyltrimethylammonium dichloroiodate (961 mg, 2.76 mmol), and the reaction was stirred under N₂ at 45° C. for 2 h. The reaction was concentrated, then diluted with water (10 mL) and extracted with EtOAc (3×20 mL). The combined organic layers were washed with saturated aqueous sodium thiosulfate, dried with Na₂SO₄, filtered and concentrated. The crude material was purified by FCC (60-100% EtOAc:PE) to provide the title intermediate (200 mg) as a yellow solid.

LCMS: Rt 0.78 min; MS m/z 252.1 [M+H]⁺; Method J.

¹H NMR (400 MHz, CDCl₃) δ 8.17 (br s, 1H), 7.84-7.81 (m, 2H), 6.86-6.83 (m, 1H), 4.66 (s, 2H), 2.88 (s, 2H), 1.24 (s, 6H).

### Intermediate 20

### 6-(2-chloroacetyl)-3,3-dimethyl-3,4-dihydroquinolin-2(1H)-one

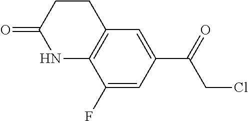 [SMILES: O=C1CCc2cc(C(=O)CCl)cc(F)c2N1]

### Steps 1 and 2: 8-fluoro-6-vinyl-3,4-dihydroquinolin-2(1H)-one

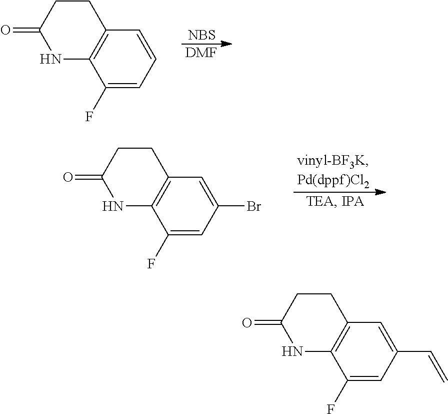

Using the same methods as steps 3 and 4 of Intermediate 19, starting with 8-fluoro-3,4-dihydroquinolin-2(1H)-one (CAS #143268-79-5) (700 mg, 4.24 mmol), provided the title intermediate (468 mg) as a yellow solid.

LCMS: Rt 0.76 min; MS m/z 192.1 [M+H]⁺; Method J.

¹H NMR (400 MHz, CDCl₃) δ 7.85 (br s, 1H), 7.08-6.97 (m, 2H), 6.65-6.51 (m, 1H), 5.68-5.64 (m, 1H), 5.25-5.22 (m, 1H), 3.02-2.98 (m, 2H), 2.70-2.63 (m, 2H).

### Steps 3 and 4: 6-(2-chloroacetyl)-3,3-dimethyl-3,4-dihydroquinolin-2(1H)-one

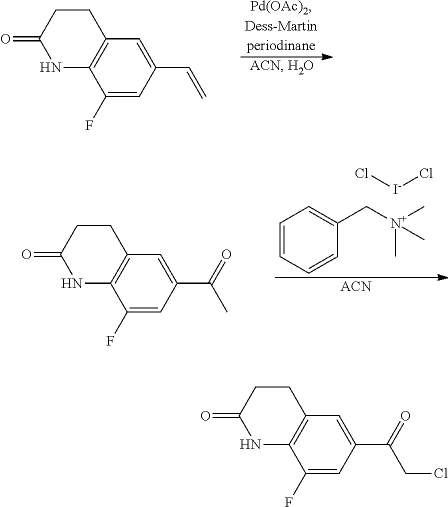

Using the same methods as steps 5 and 6 of Intermediate 19, starting with 8-fluoro-6-vinyl-3,4-dihydroquinolin-2(1H)-one (790 mg, 4.13 mmol), provided the title intermediate (500 mg) as a yellow solid.

LCMS: Rt 0.73 min; MS m/z 242.1 [M+H]⁺; Method J.

¹H NMR (400 MHz, DMSO-d₆) δ 10.52 (s, 1H), 7.74-7.66 (m, 2H), 5.11 (s, 2H), 3.03-2.99 (m, 2H), 2.56-2.50 (m, 2H).

### Intermediate 21

### 7-(2-chloroacetyl)-5-fluoro-2H-benzo[b][1,4]oxazin-3(4H)-one

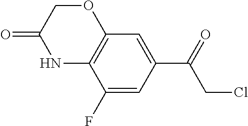 [SMILES: O=C1COc2cc(C(=O)CCl)cc(F)c2N1]

### Step 1: 4-fluorobenzo[d]oxazol-2(3H)-one

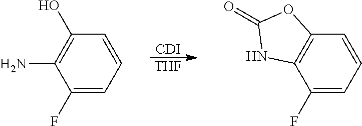

To a solution of 2-amino-3-fluorophenol (CAS #53981-23-0) (4.0 g, 31.5 mmol) in THF (60 mL) was added CDI (10.2 g, 62.9 mmol) in portions and the reaction was heated at 60° C. for 2 h. The reaction was diluted with EtOAc (100 mL), washed with 2N HCl (2×50 mL), washed with saturated brine (50 mL), dried with Na₂SO₄, filtered and concentrated. The crude material was purified by FCC (0-50% EtOAc:PE) to provide the title intermediate (3.7 g) as a light yellow solid.

LCMS: Rt 0.73 min; MS m/z 154.1 [M+H]⁺; Method L.

¹H NMR (400 MHz, DMSO-d₆) δ 12.25 (br s, 1H), 7.19-7.14 (m, 1H), 7.12-7.05 (m, 2H).

### Step 2: 6-bromo-4-fluorobenzo[d]oxazol-2(3H)-one

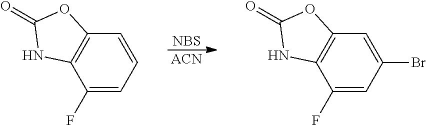

To a solution of 4-fluorobenzo[d]oxazol-2(3H)-one (3.7 g, 24.2 mmol) in acetonitrile (50 mL) was added NBS (5.16 g, 29.0 mmol). The reaction was stirred at RT for 16 h, then poured into water (50 mL) and partially concentrated to remove the acetonitrile. The aqueous layer was extracted with EtOAc (3×30 mL), dried with Na₂SO₄, filtered and concentrated. The crude material was purified by FCC (0-50% EtOAc:PE) to provide the title intermediate (5.3 g) as a light yellow solid.

LCMS: Rt 0.78 min; MS m/z 231.9 and 233.9 [M+H]⁺; Method L.

H NMR (400 MHz, DMSO-d₆) δ 12.45 (br s, 1H), 7.50 (s, 1H), 7.44-7.41 (m, 1H).

### Step 3: 2-amino-5-bromo-3-fluorophenol

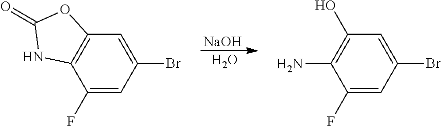

To a solution of 6-bromo-4-fluorobenzo[d]oxazol-2(3H)-one (5.3 g, 22.8 mmol) was added 3M aq. NaOH (50 mL), and this was stirred at 100° C. for 3 h. The reaction was cooled, acidified with 1 N aq. HCl until pH=6, extracted with EtOAc (3×50 mL), dried with Na₂SO₄, filtered and concentrated to provide the title intermediate (4.46 g) as a brown solid which was used without further purification.

LCMS: Rt 0.56 min; MS m/z 205.9 and 207.9 [M+H]⁺; Method L.

¹H NMR (400 MHz, DMSO-d₆) δ 9.96 (br s, 1H), 6.80-6.76 (m, 1H), 6.66 (s, 1H), 4.58 (br s, 2H).

### Step 4: 7-bromo-5-fluoro-2H-benzo[b][1,4]oxazin-3(4H)-one

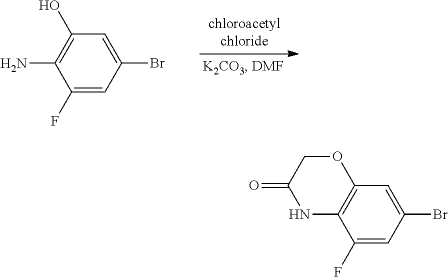 [SMILES: Nc1c(O)cc(Br)cc1F ; O=C1COc2cc(Br)cc(F)c2N1]

To a solution of 2-amino-5-bromo-3-fluorophenol (2 g, 9.7 mmol) in DMF (20 mL) was added chloroacetyl chloride (1.12 g, 9.71 mmol) and K₂CO₃ (2.68 g, 19.4 mmol), and this was stirred at 80° C. for 2 h. The reaction was cooled, poured into water (20 mL), extracted with DCM (5×20 mL), washed with saturated brine (20 mL), dried with Na₂SO₄, filtered and concentrated. The crude material was purified by FCC (0-100% EtOAc:PE) to provide the title intermediate (1.7 g) as an offwhite solid.

LCMS: Rt 0.64 min; MS m/z 246.0 and 247.9 [M+H]⁺; Method J.

¹H NMR (400 MHz, DMSO-d₆) δ 11.02 (s, 1H), 7.24-7.21 (m, 1H), 7.08-7.07 (m, 1H), 4.64 (s, 2H).

### Steps 5-7: 7-(2-chloroacetyl)-5-fluoro-2H-benzo[b][1,4]oxazin-3(4H)-one

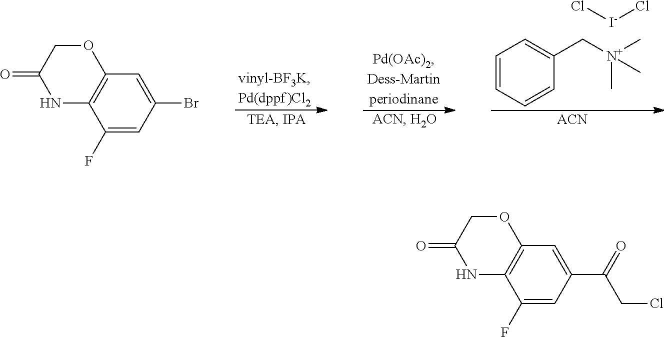

Using the same methods as steps 4-6 of Intermediate 19, starting with 7-bromo-5-fluoro-2H-benzo[b][1,4]oxazin-3(4H)-one, provided the title intermediate as a yellow solid.

LCMS: Rt 0.69 min; MS m/z 243.9 [M+H]⁺; Method L.

¹H NMR (400 MHz, DMSO-d₆) δ 11.32 (s, 1H), 7.55-7.52 (m, 1H), 7.44 (s, 1H), 5.14 (s, 2H), 4.72 (s, 2H).

### Intermediate 22

### (±)-6-(2-chloroacetyl)-3,8-difluoro-3,4-dihydroquinolin-2(1H)-one

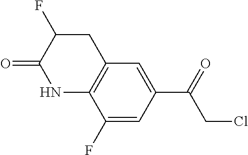 [SMILES: O=C(CCl)c1cc(F)c2c(c1)CC(F)C(=O)N2]

### Step 1: (3-fluoro-2-nitrophenyl)methanol

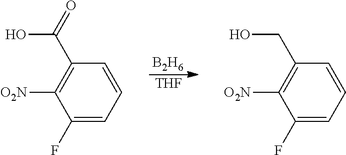

To 3-fluoro-2-nitrobenzoic acid (CAS #1000339-51-4) (5.0 g, 27 mmol) under N₂ at RT was added dropwise 1 M B₂H₆ in THF (100 mL, 100 mmol). The reaction was stirred at RT for 2 h, then at 70° C. for 6 h. The reaction was cooled to RT and MeOH (200 mL) was added dropwise, and this was stirred at RT for 2 h, then concentrated. The crude material was purified by FCC (0-50% EtOAc:PE) to provide the title intermediate (4.3 g) as a yellow solid. ¹H NMR (400 MHz, DMSO-d₆) δ 7.71-7.64 (m, 1H), 7.52-7.45 (m, 2H), 5.64 (br s, 1H), 4.63 (s, 2H).

### Step 2: 1-(bromomethyl)-3-fluoro-2-nitrobenzene

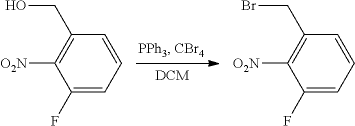 [SMILES: O=[N+]([O-])c1c(F)cccc1CO ; O=[N+]([O-])c1c(F)cccc1CBr]

Using the same method as step 2 of Intermediate 13, starting with (3-fluoro-2-nitrophenyl)methanol

(2.0 g, 11.7 mmol), provided the title intermediate (2.0 g) as a light yellow oil.

¹H NMR (400 MHz, DMSO-d₆) δ 7.75-7.71 (m, 1H), 7.65-7.58 (m, 2H), 4.80 (s, 2H).

### Step 3: Diethyl 2-fluoro-2-(3-fluoro-2-nitrobenzyl)malonate

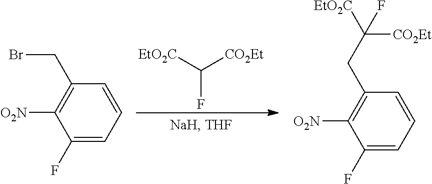 [SMILES: O=[N+]([O-])c1c(F)cccc1CBr ; CCOC(=O)C(F)C(=O)OCC ; CCOC(=O)C(F)(Cc1cccc(F)c1[N+](=O)[O-])C(=O)OCC]

To a solution of diethyl 2-fluoromalonate (CAS #685-88-1) (1.75 g, 9.83 mmol) in THF (40 mL) at 0° C. was added NaH (60% in mineral oil, 455 mg, 11.4 mmol) in portions, and this was stirred at RT for 30 min. 1-(bromomethyl)-3-fluoro-2-nitrobenzene (2.0 g, 8.6 mmol) was added and this was stirred at RT for 2 h. The reaction was poured into saturated aqueous NH₄Cl (40 mL), extracted with EtOAc (3×30 mL), dried with Na₂SO₄, filtered and concentrated. The crude material was purified by FCC (0-50% EtOAc:PE) to provide the title intermediate (2.3 g) as a light yellow oil.

¹H NMR (400 MHz, DMSO-d₆) δ 7.73-7.66 (m, 1H), 7.62-7.56 (m, 1H), 7.33-7.30 (m, 1H), 4.29-4.17 (m, 4H), 3.76 (s, 1H), 3.70 (s, 1H), 1.17 (t, J=7.2 Hz, 6H).

### Step 4: (+)-Ethyl 3,8-difluoro-2-oxo-1-,2,3,4-tetrahydroquinoline-3-carboxylate

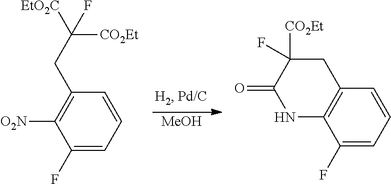 [SMILES: "MeOH" ; CCOC(=O)C(F)(Cc1cccc(F)c1[N+](=O)[O-])C(=O)OCC ; CCOC(=O)C1(F)Cc2cccc(F)c2NC1=O]

Using the same method as step 4 of Intermediate 13, starting with diethyl 2-fluoro-2-(3-fluoro-2-nitrobenzyl)malonate (2.3 g, 6.94 mmol), provided the title intermediate (1.5 g) as a light yellow solid which was used without further purification.

¹H NMR (400 MHz, DMSO-d₆) δ 11.08 (s, 1H), 7.22-7.14 (m, 1H), 7.13-7.08 (m, 1H), 7.07-7.00 (m, 1H), 4.29-4.17 (m, 2H), 3.66 (d, J=4.0 Hz, 1H), 3.60 (s, 1H), 1.14 (t, J=7.2 Hz, 3H).

### Step 5: (±)-3,8-difluoro-2-oxo-1,2,3,4-tetrahydroquinoline-3-carboxylic acid

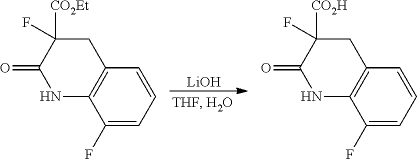

To a solution of (±)-ethyl 3,8-difluoro-2-oxo-1,2,3,4-tetrahydroquinoline-3-carboxylate (2.1 g, 8.23 mmol) in THF (20 mL) was added LiOH·H₂O (518 mg, 12.3 mmol) in water (20 mL) and this was stirred at RT for 2 h. The reaction was adjusted to pH 6 with saturated aqueous citric acid, extracted with EtOAc (3×20 mL), dried with Na₂SO₄, filtered and concentrated to provide the title intermediate (2.0 g) as a white solid which was used without further purification.

LCMS: Rt 0.43 min; MS m/z 228.0 [M+H]⁺; Method L.

### Step 6: (±)-3,8-difluoro-3,4-dihydroquinolin-2(1H)-one

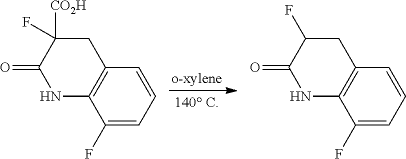

A solution of (±)-3,8-difluoro-2-oxo-1,2,3,4-tetrahydroquinoline-3-carboxylic acid (2.0 g) in o-xylene (40 mL) was stirred at 140° C. for 16 h, then cooled, concentrated, and purified by FCC (0-50% EtOAc:PE) to provide the title intermediate (1.5 g) as a light yellow solid.

¹H NMR (400 MHz, DMSO-d₆) δ 10.54 (br s, 1H), 7.18-7.06 (m, 2H), 7.03-6.96 (m, 1H), 5.37-5.17 (m, 1H), 3.44-3.34 (m, 1H), 3.29-3.23 (m, 1H).

### Step 7: (±)-6-bromo-3,8-difluoro-3,4-dihydroquinolin-2(1H)-one

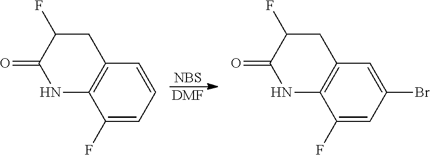

Using the same method as step 3 of Intermediate 19, starting with (±)-3,8-difluoro-3,4-dihydroquinolin-2(1H)-one (1.4 g, 7.64 mmol), provided the title intermediate (1.7 g) as a yellow solid which was used without further purification.

LCMS: Rt 0.64 min; MS m/z 262.0 and 264.0 [M+H]⁺; Method J.

¹H NMR (400 MHz, DMSO-d₆) δ 10.67 (s, 1H), 7.48-7.45 (m, 1H), 7.36 (s, 1H), 5.36-5.17 (m, 1H), 3.45-3.35 (m, 1H), 3.30 (br s, 1H).

### Step 8: (±)-6-acetyl-3,8-difluoro-3,4-dihydroquinolin-2(1H)-one

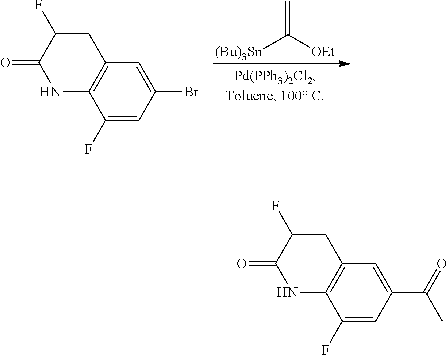

To a solution of (±)-6-bromo-3,8-difluoro-3,4-dihydroquinolin-2(1H)-one (500 mg, 1.91 mmol) in toluene (5 mL) was added tributyl(1-ethoxyvinyl)stannane (CAS #97674-02-7) (1.29 mL, 1.38 g, 3.82 mmol) and Pd(PPh₃)₂Cl₂ (134 mg, 0.19 mmol) and this was stirred at 100° C. for 16 h. The reaction was cooled, diluted with saturated aqueous KF (10 mL), extracted with EtOAc (3×20 mL), dried with Na₂SO₄, filtered and concentrated. The crude material was purified by FCC (0-60% EtOAc:PE) to provide the title intermediate (260 mg) as a yellow solid.

LCMS: Rt 0.32 min; MS m/z 226.1 [M+H]⁺; Method J.

¹H NMR (400 MHz, DMSO-d₆) δ 10.63 (s, 1H), 7.41-7.28 (m, 2H), 5.41-5.14 (m, 1H), 4.77-4.76 (m, 1H), 4.28-4.27 (m, 1H), 3.32 (s, 3H).

### Step 9: (±)-6-(2-chloroacetyl)-3,8-difluoro-3,4-dihydroquinolin-2(1H)-one

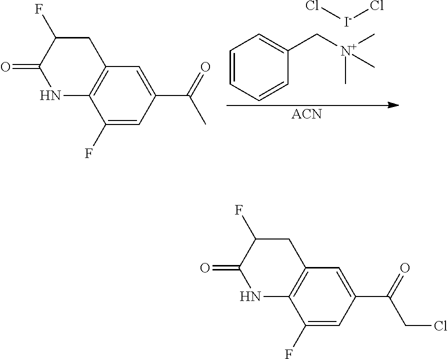

Using the same method as step 6 of Intermediate 19, starting with (±)-6-acetyl-3,8-difluoro-3,4-dihydroquinolin-2(1H)-one (160 mg, 0.710 mmol), provided the title intermediate (80 mg) as a yellow solid.

LCMS: Rt 0.55 min; MS m/z 260.0 [M+H]⁺; Method J.

¹H NMR (400 MHz, CDC₃) δ 7.77 (br s, 1H), 7.72-7.68 (m, 2H), 5.30-5.13 (m, 1H), 4.61 (s, 2H), 3.51-3.43 (m, 2H).

### Intermediate 23

### 6-(2-chloroacetyl)-3,3,8-trifluoro-3,4-dihydroquinolin-2(1H)-one

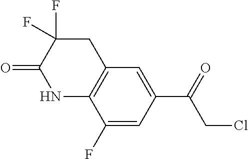 [SMILES: O=C(CCl)c1cc(F)c2c(c1)CC(F)(F)C(=O)N2]

### Step 1: Ethyl 2,2-difluoro-3-(3-fluoro-2-nitrophenyl)propanoate

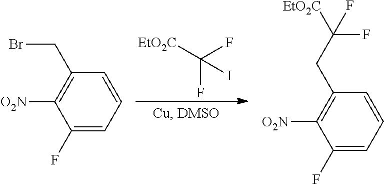 [SMILES: O=[N+]([O-])c1c(F)cccc1CBr ; CCOC(=O)C(F)(F)I ; CCOC(=O)C(F)(F)Cc1cccc(F)c1[N+](=O)[O-]]

To a solution of ethyl 2,2-difluoro-2-iodoacetate (CAS #7648-30-8) (6.4 g, 25.6 mmol) in DMSO (40 mL) was added Cu (3.58 g, 56.4 mmol) and 1-(bromomethyl)-3-fluoro-2-nitrobenzene (from step 2 of Intermediate 22, 4.0 g, 17.1 mmol) and this was stirred at RT for 16 h. The reaction was diluted with water (100 mL) and filtered, rinsing through with EtOAc (2×10 mL). The combined filtrate was extracted with EtOAc (3×20 mL), washed with saturated brine (50 mL), dried with Na₂SO₄, filtered and concentrated. The crude material was purified by FCC (0-15% EtOAc:PE) to provide the title intermediate (3.0 g) as a light yellow oil.

¹H NMR (400 MHz, DMSO-d₆) δ 7.77-7.71 (m, 1H), 7.67-7.61 (m, 1H), 7.45-7.42 (m, 1H), 4.31-4.25 (m, 2H), 3.81-3.71 (m, 2H), 1.25-1.20 (m, 3H).

### Step 2: 3,3,8-trifluoro-3,4-dihydroquinolin-2(1H)-one

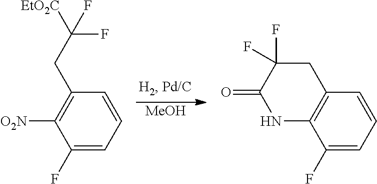 [SMILES: "MeOH" ; CCOC(=O)C(F)(F)Cc1cccc(F)c1[N+](=O)[O-] ; O=C1Nc2c(F)cccc2CC1(F)F]

Using the same method as step 4 of Intermediate 13, starting with ethyl 2,2-difluoro-3-(3-fluoro-2-nitrophenyl)propanoate (1.5 g, 5.41 mmol), provided the title intermediate (920 mg) as a light yellow solid which was used without further purification.

¹H NMR (400 MHz, DMSO-d₆) δ 11.18 (br s, 1H), 7.27-7.03 (m, 3H), 3.73 (t, J=17.2 Hz, 2H).

### Steps 3-5: 6-(2-chloroacetyl)-3,3,8-trifluoro-3,4-dihydroquinolin-2(1H)-one

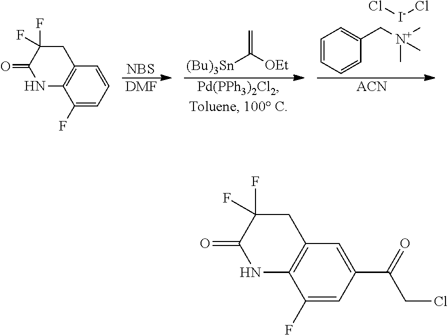

Using the same methods as steps 7-9 of Intermediate 22, starting with 3,3,8-trifluoro-3,4-dihydroquinolin-2(1H)-one, provided the title intermediate as a yellow solid.

LCMS: Rt 0.69 min; MS m/z 277.9 [M+H]⁺; Method J.

¹H NMR (400 MHz, CDC₃) δ 8.29 (br s, 1H), 7.80-7.62 (m, 2H), 4.61 (s, 2H), 3.66-3.58 (m, 2H).

### Intermediate 24

### 2-bromo-1-(1-(tetrahydro-2H-pyran-2-yl)-1H-indazol-5-yl)ethan-1-one

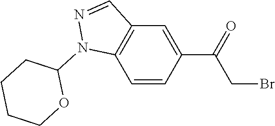 [SMILES: O=C(CBr)c1ccc2c(cnn2C2CCCCO2)c1]

### Step 1: 1-(tetrahydro-2H-pyran-2-yl)-1H-indazole-5-carbonitrile

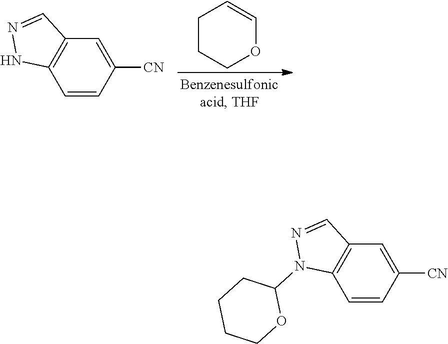 [SMILES: N#Cc1ccc2[nH]ncc2c1 ; C1=COCCC1 ; N#Cc1ccc2c(cnn2C2CCCCO2)c1]

To a solution of 1H-indazole-5-carbonitrile (CAS #74626-47-4) (2.0 g, 14.0 mmol) and benzenesulfonic acid (221 mg, 1.40 mmol) in THF (50 mL) was added 3,4-dihydro-2H-pyran (CAS #110-87-2) (4.70 g, 55.9 mmol), and this was stirred at RT for 3 h then at 50° C. overnight. The reaction was concentrated and purified by FCC (0-25% EtOAc:Heptane) to provide the title intermediate (3.2 g) as a light pink oil.

¹H NMR (400 MHz, DCM-d₂) δ 8.17 (t, J=1.1 Hz, 1H), 8.13 (d, J=1.0 Hz, 1H), 7.76 (dt, J=8.7, 1.0 Hz, 1H), 7.63 (dd, J=8.8, 1.5 Hz, 1H), 5.78 (dd, J=9.3, 2.7 Hz, 1H), 4.07-3.98 (m, 1H), 3.84-3.73 (m, 1H), 2.58-2.46 (m, 1H), 2.23-2.06 (m, 2H), 1.89-1.64 (m, 3H).

### Step 2: 1-(1-(tetrahydro-2H-pyran-2-yl)-1H-indazol-5-yl)ethan-1-one

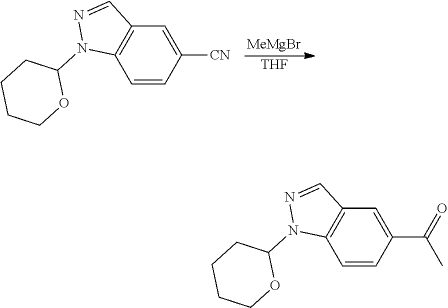 [SMILES: "MeMgBr" ; "THF" ; N#Cc1ccc2c(cnn2C2CCCCO2)c1 ; CC(=O)c1ccc2c(cnn2C2CCCCO2)c1]

To a solution of 1-(tetrahydro-2H-pyran-2-yl)-1H-indazole-5-carbonitrile (3.38 g, 14.9 mmol) in THF (50 mL) under N₂ at 0° C. was added methylmagnesium bromide (3.0 M in diethyl ether, 24.8 mL, 74.4 mmol) dropwise. The resulting suspension was heated at 60° C. for 3 h and 73° C. for 2 h, then diluted with water (100 mL) and 1N HCl until pH=7. This was extracted with EtOAc, washed with saturated brine, dried with Na₂SO₄, filtered and concentrated to provide the title intermediate (3.78 g) as an orange oil which was used without further purification.

LCMS: Rt 1.11 min; MS m/z 245.2 [M+H]⁺; Method K.

### Step 3: 2-bromo-1-(1-(tetrahydro-2H-pyran-2-yl)-1H-indazol-5-yl)ethan-1-one

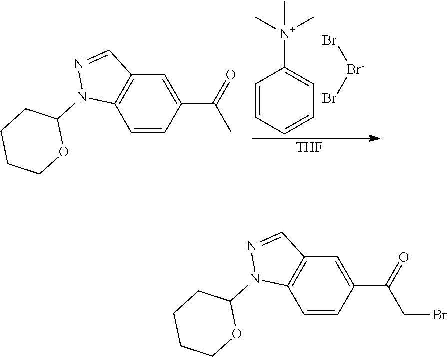

To a solution of 1-(1-(tetrahydro-2H-pyran-2-yl)-1H-indazol-5-yl)ethan-1-one (1.86 g, 7.6 mmol) in THF (25 mL) at 0° C. was added a solution of phenyltrimethylammonium tribromide (3.0 g, 8.0 mmol) in THF (25 mL). After 10 min, the reaction was filtered and the filtrate was concentrated and purified by FCC (0-20% EtOAc:Heptane) to provide the title intermediate (880 mg) as a pale yellow oil.

LCMS: Rt 1.26 min; MS m/z 323.2 and 325.2 [M+H]⁺; Method K.

### Intermediate 25

### 6-(2-bromo-1-hydroxyethyl)-1,4-dihydro-2H-benzo[d][1,3]oxazin-2-one

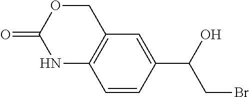 [SMILES: O=C1Nc2ccc(C(O)CBr)cc2CO1]

### Step 1: 6-bromo-1,4-dihydro-2H-benzo[d][1,3]oxazin-2-one

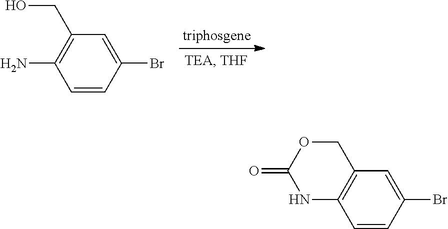 [SMILES: Nc1ccc(Br)cc1CO ; O=C1Nc2ccc(Br)cc2CO1]

To a solution of (2-amino-5-bromophenyl)methanol (CAS #20712-12-3) (1.2 g, 5.94 mmol) in dry THF (20 mL) at 0° C. was added slowly a solution of triphosgene (2.11 g, 7.13 mmol) in THF (5 mL). After 10 minutes, triethylamine (2.92 mL, 20.79 mmol) was added dropwise and the reaction was warmed to RT and stirred for 1 h. The reaction was poured onto crushed ice, extracted with ethyl acetate (3×30 mL), dried with Na₂SO₄, filtered and concentrated. The crude material was purified by FCC (0-50% EtOAc:PE) to provide the title intermediate (850 mg) as a white solid.

LCMS: Rt 0.60 min; MS m/z 228.0 and 230.0 [M+H]⁺; Method J.

¹H NMR (400 MHz, CDCl₃) δ 8.40 (br s, 1H), 7.41-7.38 (m, 1H), 7.27-7.26 (m, 1H), 6.76-6.73 (m, 1H), 5.30 (s, 2H).

### Step 2: 6-vinyl-1,4-dihydro-2H-benzo[d][1,3]oxazin-2-one

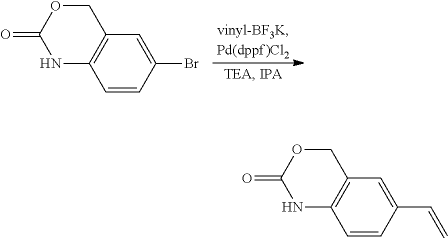

Using the same method as step 4 of Intermediate 19, starting with 6-bromo-1,4-dihydro-2H-benzo[d][1,3]oxazin-2-one, provided the title intermediate (400 mg) as a white solid.

¹H NMR (400 MHz, CDCl₃) δ 8.33-8.26 (m, 1H), 7.33-7.30 (m, 1H), 7.18 (s, 1H), 6.82-6.79 (m, 1H), 6.69-6.62 (m, 1H), 5.70-5.65 (m, 1H), 5.34 (s, 2H), 5.24-5.20 (m, 1H).

### Step 3: 6-(2-bromo-1-hydroxyethyl)-1,4-dihydro-2H-benzo[d][1,3]oxazin-2-one

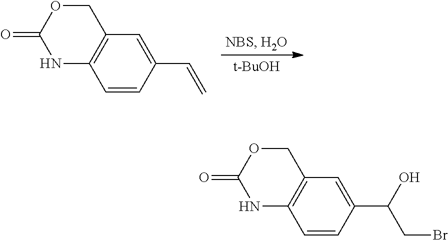

To a solution of 6-vinyl-1,4-dihydro-2H-benzo[d][1,3]oxazin-2-one (190 mg, 0.57 mmol) in H₂O (1.5 mL) and t-BuOH (0.75 mL) was added NBS (91 mg, 0.51 mmol) and this was stirred at 40° C. for 1 h.

The reaction was diluted with H₂O (10 mL), extracted with EtOAc (2×5 mL), dried with Na₂SO₄, filtered and concentrated. The crude material was purified by Prep-TLC (2:1 EtOAc:PE, R_{f}=0.5) to provide the title intermediate (210 mg) as a yellow solid.

LCMS: Rt 0.62 min; MS m/z 272.0 and 274.0 [M+H]⁺; Method J.

### Intermediate 26

### 6-(2-bromo-1-hydroxyethyl)-8-fluoro-3,4-dihydroquinolin-2(1H)-one

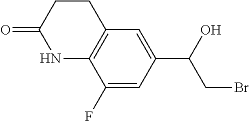 [SMILES: O=C1CCc2cc(C(O)CBr)cc(F)c2N1]

Using the same method as step 3 of Intermediate 25, starting with 8-fluoro-6-vinyl-3,4-dihydroquinolin-2(1H)-one (from step 2 of Intermediate 20, 400 mg, 2.09 mmol), provided the title intermediate (564 mg) as a white solid.

LCMS: Rt 0.66 min; MS m/z 288.0 and 290.0 [M+H]⁺; Method J.

¹H NMR (400 MHz, Methanol-d₄) δ 7.21-6.97 (m, 2H), 4.83-4.79 (m, 1H), 3.66-3.57 (m, 1H), 3.56-3.48 (m, 1H), 3.02-2.99 (m, 2H), 2.62-2.57 (m, 2H).

### Intermediate 27

### 7-(2-bromo-1-hydroxyethyl)-9-fluoro-1,3,4,5-tetrahydro-2H-benzo[b]azepin-2-one

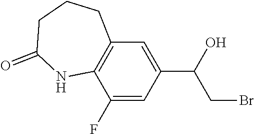 [SMILES: O=C1CCCc2cc(C(O)CBr)cc(F)c2N1]

### Step 1: 7-bromo-9-fluoro-1,3,4,5-tetrahydro-2H-benzo[b]azepin-2-one

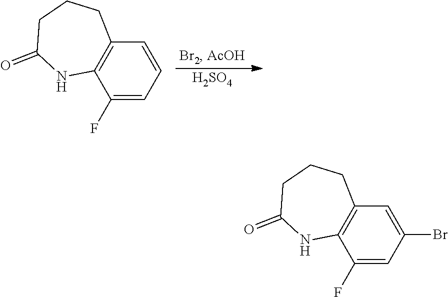

To a solution of 9-fluoro-1,3,4,5-tetrahydro-2H-benzo[b]azepin-2-one (CAS #1151397-80-6) (1 g, 5.6 mmol) in AcOH (10 mL) at RT was added H₂SO₄ (0.05 mL), followed by a solution of Br₂ (1.96 g, 0.63 mL, 12.3 mmol) in AcOH (8.6 mL) dropwise. The reaction vessel was sealed and stirred at RT for 12 h, and then poured into ice and neutralized with ammonium hydroxide until pH=7. This was extracted with EtOAc (3×10 mL), washed with saturated aqueous NaHCO₃ (20 mL), then with saturated brine (20 mL), dried with Na₂SO₄, filtered and concentrated. The crude material was purified by FCC (0-50% EtOAc:PE) to provide the title intermediate (850 mg) as an offwhite solid.

LCMS: Rt 0.68 min; MS m/z 258.0 and 260.0 [M+H]⁺; Method J.

¹H NMR (400 MHz, DMSO-d₆) δ 9.50 (s, 1H), 7.51-7.44 (m, 1H), 7.37 (br s, 1H), 2.72 (t, J=7.2 Hz, 2H), 2.19-2.07 (m, 4H).

### Steps 2 and 3: 7-(2-bromo-1-hydroxyethyl)-9-fluoro-1,3,4,5-tetrahydro-2H-benzo[b]azepin-2-one

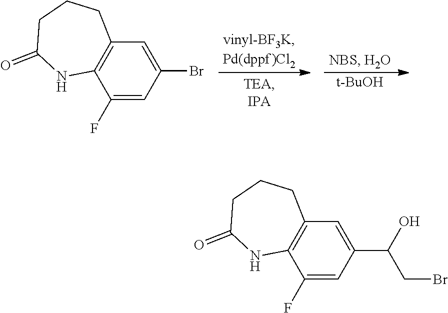

Using the same methods as steps 2 and 3 of Intermediate 25, starting with 7-bromo-9-fluoro-1,3,4,5-tetrahydro-2H-benzo[b]azepin-2-one, provided the title intermediate as a white solid.

LCMS: Rt 0.58 min; MS m/z 302.0 and 304.0 [M+H]⁺; Method J.

¹H NMR (400 MHz, DMSO-d₆) δ 9.44 (s, 1H), 7.19-7.13 (m, 2H), 5.91-5.90 (m, 1H), 4.80-4.75 (m, 1H), 3.70-3.67 (m, 1H), 3.59-3.55 (m, 1H), 2.73-2.69 (m, 2H), 2.16-2.09 (m, 4H).

### Intermediate 28

### 6-(2-bromo-1-((tert-butyldimethylsilyl)oxy)ethyl)-4-fluorobenzo[d]thiazol-2(3H)-one

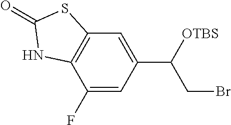 [SMILES: CC(C)(C)[Si](C)(C)OC(CBr)c1cc(F)c2[nH]c(=O)sc2c1]

### Step 1: 6-bromo-4-fluorobenzo[d]thiazole-2-thiol

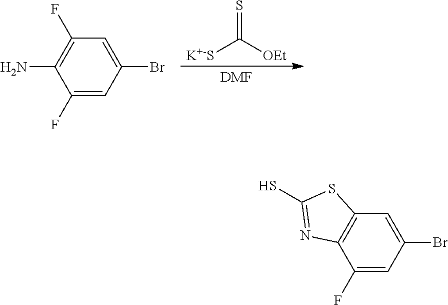

To a solution of 4-bromo-2,6-difluoroaniline (CAS #67567-26-4) (15.0 g, 72.1 mmol) in DMF (300 mL) was added potassium O-ethylcarbonodithioate (CAS #140-89-6) (25.43 g, 158.6 mmol) and this was stirred at 120° C. for 16 h. The reaction was cooled, then poured into water (200 mL) and acidified with 2N HCl until pH=4. The resulting precipitate was collected by filtration, washed with water (2×40 mL), and dried to provide the title intermediate (20 g, crude) as a yellow solid which was used without further purification.

LCMS: Rt 0.86 min; MS m/z 264.0 and 266.0 [M+H]⁺; Method J.

### Step 2: 6-bromo-4-fluoro-2-(methylthio)benzo[d]thiazole

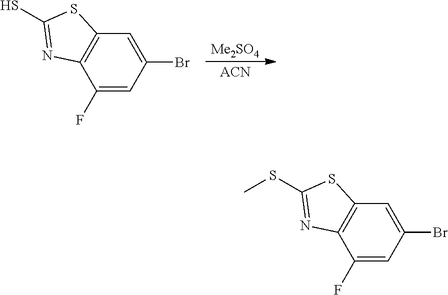

To a suspension of 6-bromo-4-fluorobenzo[d]thiazole-2-thiol (20 g, crude) in acetonitrile (400 mL) was added Me₂SO₄ (28.65 g, 21.5 mL, 227.2 mmol) and this was stirred at 80° C. for 2.5 h. The reaction was cooled to RT and the resulting precipitate was collected by filtration and dried to provide the title intermediate (20 g, crude) as a light yellow solid which was used without further purification.

LCMS: Rt 0.98 min; MS m/z 277.9 and 279.9 [M+H]⁺; Method J.

¹H NMR (400 MHz, DMSO-d₆) δ 8.18 (s, 1H), 7.66 (m, 1H), 2.85 (s, 3H).

### Step 3: 6-bromo-4-fluoro-2-(methylsulfonyl)benzo[d]thiazole

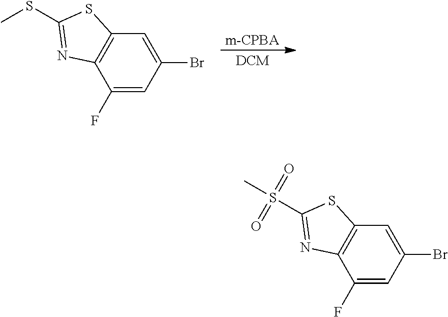

To a solution of 6-bromo-4-fluoro-2-(methylthio)benzo[d]thiazole (8 g, crude) in DCM (80 mL) was added m-CPBA (12.8 g, 85% purity, 63.3 mmol) and this was stirred at RT for 2 h. The reaction was diluted with sat. aq. NaHCO₃ (3×50 mL), extracted with DCM (2×40 mL), dried with Na₂SO₄, filtered and concentrated to provide the title intermediate (12 g, crude) as a white solid which was used without further purification.

LCMS: Rt 0.86 min; MS m/z 309.9 and 311.9 [M+H]⁺; Method J.

### Step 4: 6-bromo-4-fluorobenzo[d]thiazol-2(3H)-one

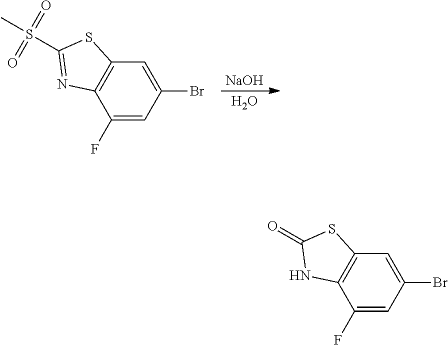

A solution of 6-bromo-4-fluoro-2-(methylsulfonyl)benzo[d]thiazole (12 g, crude) in 5N aq. NaOH (100 mL) was stirred at 100° C. for 2 h. The reaction was cooled, diluted with water (10 mL) and acidified with 2N HCl until pH=4. The resulting precipitate was collected by filtration, dissolved in EtOAc (100 mL), washed with sat. aq. NaHCO₃ (3×100 mL), dried with Na₂SO₄, filtered and concentrated. The crude material was triturated with 5:1 PE:EtOAc (50 mL) and filtered to provide the title intermediate (2.5 g) as a white solid which was used without further purification.

LCMS: Rt 0.76 min; MS m/z 247.8 and 249.8 [M+H]⁺; Method J.

### Steps 5 and 6: 6-(2-bromo-1-hydroxyethyl)-4-fluorobenzo[d]thiazol-2(3H)-one

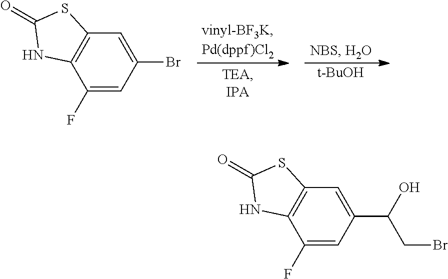

Using the same methods as steps 2 and 3 of Intermediate 25, starting with 6-bromo-4-fluorobenzo[d]thiazol-2(3H)-one, provided the title intermediate as a yellow solid.

LCMS: Rt 0.65 min; MS m/z 291.8 and 293.8 [M+H]⁺; Method J.

¹H NMR (400 MHz, DMSO-d₆) δ 12.40 (s, 1H), 7.46 (s, 1H), 7.25-7.22 (m, 1H), 5.97 (br s, 1H), 4.83-4.81 (m, 1H), 3.71-3.67 (m, 1H), 3.43-3.40 (m, 1H).

### Step 7: 6-(2-bromo-1-((tert-butyldimethylsilyl)oxy)ethyl)-4-fluorobenzo[ ]thiazol-2(3H)-one

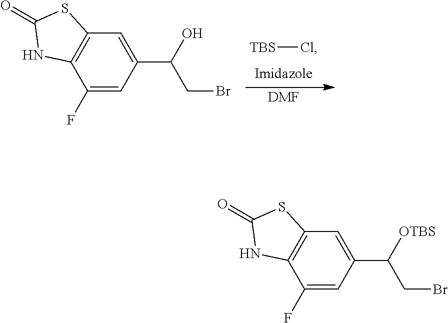

To a solution of 6-(2-bromo-1-hydroxyethyl)-4-fluorobenzo[d]thiazol-2(3H)-one (1.3 g, 4.45 mmol) in DMF (13 mL) was added TBS-Cl (2.0 g, 13.3 mmol) and imidazole (1.2 g, 17.8 mmol) and the reaction was stirred at 60° C. for 6 h. The reaction was cooled, diluted with water (20 mL), extracted with EtOAc (3×20 mL), washed with saturated brine (50 mL), dried with Na₂SO₄, filtered and concentrated. The crude material was purified by FCC (0-60% EtOAc:PE) to provide the title intermediate (1.8 g) as a yellow oil.

LCMS: Rt 1.02 min; MS m/z 405.8 and 407.8 [M+H]⁺; Method J.

¹H NMR (400 MHz, CDCl₃) δ 9.09 (br s, 1H), 7.20 (s, 1H), 7.09-7.06 (m, 1H), 4.85-4.82 (m, 1H), 3.47-3.38 (m, 2H), 0.91 (s, 9H), 0.13-0.11 (m, 6H).

### Intermediate 29

### 7-(2-bromo-1-((tert-butyldimethylsilyl)oxy)ethyl)-9-fluoro-1,5-dihydrobenzo[e][1,4]oxazepin-2(3H)-one

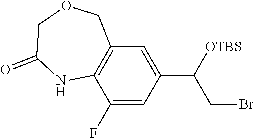 [SMILES: CC(C)(C)[Si](C)(C)OC(CBr)c1cc(F)c2c(c1)COCC(=O)N2]

### Step 1: 2-chloro-N-(2-fluoro-6-(hydroxymethyl)phenyl)acetamide

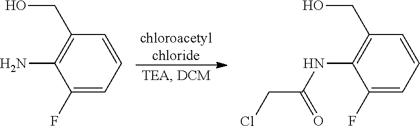 [SMILES: Nc1c(F)cccc1CO ; "TEA, DCM" ; "chloroacetylchloride" ; O=C(CCl)Nc1c(F)cccc1CO]

To a solution of (2-amino-3-fluorophenyl)methanol (CAS #906811-49-2) (2.5 g, 17.7 mmol) in DCM (50 mL) was added triethylamine (3.58 g, 25.4 mmol). This was cooled to 0° C. and chloroacetyl chloride (2.4 g, 21.2 mmol) was added, then this was stirred at RT for 16 h. The reaction was washed with sat. aq. NH₄Cl (40 mL) and the aqueous layer was extracted with DCM (2×20 mL). The combined organic layers were dried with Na₂SO₄, filtered and concentrated. The crude material was purified by FCC (0-60% EtOAc:PE) to provide the title intermediate (1.5 g) as a light yellow solid.

¹H NMR (400 MHz, DMSO-d₆) δ 9.75 (s, 1H), 7.43-7.27 (m, 2H), 7.23-7.10 (m, 1H), 5.28 (t, J=5.8 Hz, 1H), 4.46-4.42 (m, 2H), 4.32 (s, 2H).

### Step 2: 9-fluoro-1,5-dihydrobenzo[e][1,4]oxazepin-2(3H)-one

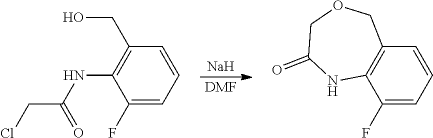

To a solution of 2-chloro-N-(2-fluoro-6-(hydroxymethyl)phenyl)acetamide in DMF (30 mL) at 0° C. was added NaH (60% in mineral oil, 827 mg, 20.7 mmol) in portions, and this was stirred at RT for 2 h. The reaction was diluted with sat. aq. NH₄Cl (20 mL), extracted with EtOAc (3×15 mL), washed with sat. brine (15 mL), dried with Na₂SO₄, filtered and concentrated. The crude material was purified by FCC (0-100% EtOAc:PE) to provide the title intermediate (1.0 g) as a yellow solid.

¹H NMR (400 MHz, DMSO-d₆) δ 9.77 (br s, 1H), 7.27-7.18 (m, 1H), 7.11-7.05 (m, 2H), 4.72 (s, 2H), 4.36 (s, 2H).

### Step 3: 7-bromo-9-fluoro-1,5-dihydrobenzo[e][1,4]oxazepin-2(3H)-one

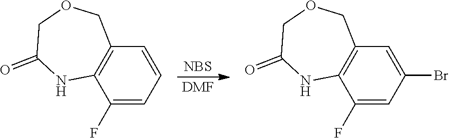

Using the same method as step 3 of Intermediate 19, starting with 9-fluoro-1,5-dihydrobenzo[e][1,4]oxazepin-2(3H)-one (1 g, 5.5 mmol), provided the title intermediate (1.4 g) as a white solid which was used without further purification.

LCMS: Rt 0.68 min; MS m/z 259.8 and 261.9 [M+H]⁺; Method J.

¹H NMR (400 MHz, DMSO-d₆) δ 9.86 (s, 1H), 7.57-7.54 (m, 1H), 7.37 (s, 1H), 4.72 (s, 2H), 4.39 (s, 2H).

### Steps 4 and 5: 7-(2-bromo-1-hydroxyethyl)-9-fluoro-1,5-dihydrobenzo[e][1,4]oxazepin-2(3H)-one

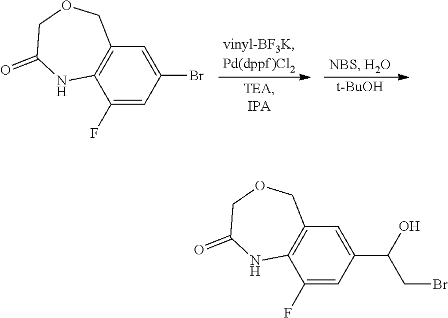

Using the same methods as steps 2 and 3 of Intermediate 25, starting with 7-bromo-9-fluoro-1,5-dihydrobenzo[e][1,4]oxazepin-2(3H)-one, provided the title intermediate as a colorless oil.

LCMS: Rt 0.41 min; MS m/z 304.0 and 306.0 [M+H]⁺; Method J.

### Step 6: 7-(2-bromo-1-((tert-butyldimethylsilyl)oxy)ethyl)-9-fluoro-1,5-dihydrobenzo[e][1,4]oxazepin-2(3H)-one

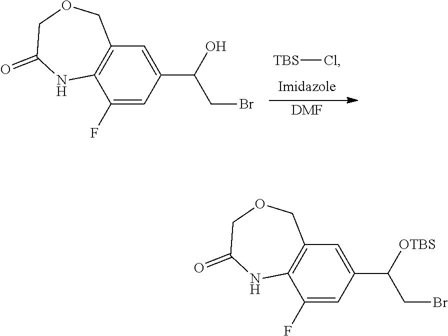

Using the same method as step 7 of Intermediate 28, starting with 7-(2-bromo-1-hydroxyethyl)-9-fluoro-1,5-dihydrobenzo[e][1,4]oxazepin-2(3H)-one, provided the title intermediate as a white solid.

LCMS: Rt 1.11 min; MS m/z 418.1 and 420.0 [M+H]⁺; Method J.

¹H NMR (400 MHz, CDC₃) δ 7.82 (br s, 1H), 7.12-7.10 (m, 1H), 6.88 (s, 1H), 4.77 (s, 3H), 4.63 (s, 2H), 3.58-3.31 (m, 2H), 0.91-0.89 (m, 9H), 0.12 (d, J=4.0 Hz, 3H), −0.03-−0.06 (m, 3H).

### Intermediate 30

### 6-(2-bromo-1-((tert-butyldimethylsilyl)oxy)ethyl)-5-fluoro-1,4-dihydro-2H-benzo[d][1,3]oxazin-2-one

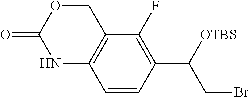 [SMILES: CC(C)(C)[Si](C)(C)OC(CBr)c1ccc2c(c1F)COC(=O)N2]

### Step 1: (2-amino-6-fluorophenyl)methanol

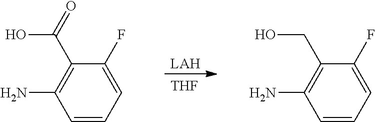 [SMILES: Nc1cccc(F)c1C(=O)O ; Nc1cccc(F)c1CO ; "THF"]

Using the same method as step 1 of Intermediate 16, starting with 2-amino-6-fluorobenzoic acid (CAS #434-76-4) (5 g, 32 mmol), provided the title intermediate (4 g) as a yellow solid. ¹H NMR (400 MHz, DMSO-d₆) δ 6.98-6.92 (m, 1H), 6.45 (d, J=8.4 Hz, 1H), 6.35-6.18 (m, 1H), 5.28 (br s, 2H), 4.94-4.92 (m, 1H), 4.44-4.43 (m, 2H).

### Step 2: 5-fluoro-1,4-dihydro-2H-benzo[d][1,3]oxazin-2-one

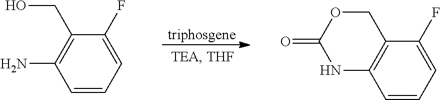 [SMILES: Nc1cccc(F)c1CO ; "TEA, THF" ; O=C1Nc2cccc(F)c2CO1]

Using the same method as step 1 of Intermediate 25, starting with (2-amino-6-fluorophenyl)methanol (4 g, 28 mmol), provided the title intermediate (3 g) as a white solid.

LCMS: Rt 0.30 min; MS m/z 168.0 [M+H]⁺; Method J.

¹H NMR (400 MHz, DMSO-d₆) δ 10.38 (br s, 1H), 7.31-7.27 (m, 1H), 6.93-6.78 (m, 1H), 6.71 (d, J=8.0 Hz, 1H), 5.37 (s, 2H).

### Step 3: 6-bromo-5-fluoro-1,4-dihydro-2H-benzo[d][1,3]oxazin-2-one

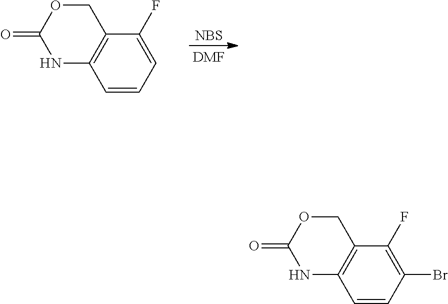

Using the same method as step 3 of Intermediate 19, starting with 5-fluoro-1,4-dihydro-2H-benzo[d][1,3]oxazin-2-one (1.5 g, 9.0 mmol), provided the title intermediate (1.6 g) as a white solid which was used without further purification.

LCMS: Rt 0.61 min; MS m/z 245.9 and 247.9 [M+H]⁺; Method J.

¹H NMR (400 MHz, DMSO-d₆) δ 10.51 (s, 1H), 7.56 (t, J=8.0 Hz, 1H), 6.68-6.66 (m, 1H), 5.40 (s, 2H).

### Steps 4-6: 6-(2-bromo-1-((tert-butyldimethylsilyl)oxy)ethyl)-5-fluoro-1,4-dihydro-2H-benzo[d][1,3]oxazin-2-one

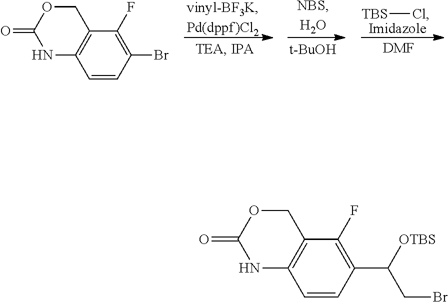

Using the same methods as steps 4-6 of Intermediate 29, starting with 6-bromo-5-fluoro-1,4-dihydro-2H-benzo[d][1,3]oxazin-2-one, provided the title intermediate as a white solid.

LCMS: Rt 1.10 min; MS m/z 403.9 and 405.9 [M+H]⁺; Method J.

¹H NMR (400 MHz, CDC₃) δ 7.94 (br s, 1H), 7.42 (t, J=8.0 Hz, 1H), 6.64 (d, J=8.4 Hz, 1H), 5.43 (s, 2H), 5.15-5.13 (m, 1H), 3.53-3.40 (m, 2H), 0.94-0.87 (m, 9H), 0.14 (s, 3H), −0.04 (s, 3H).

The following intermediates were made using similar procedures from the starting materials shown.

| Intermediate | Structure and name | Starting material | LCMS | ¹H NMR |
|---|---|---|---|---|
| 31 | 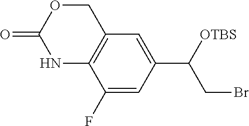 [SMILES: CC(C)(C)[Si](C)(C)OC(CBr)c1cc(F)c2c(c1)COC(=O)N2] 6-(2-bromo-1-((tert- butyldimethylsilyl)oxy)ethyl)- 8-fluoro-1,4-dihydro-2H- benzo[d][1,3]oxazin-2-one | 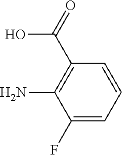 [SMILES: Nc1c(F)cccc1C(=O)O] 2-amino-3- fluorobenzoic acid (CAS# 825-22-9) | Rt 1.09 min; MS m/z 404.1 and 406.1 [M + H]⁺; Method J. | (400 MHz, DMSO-d₆) δ 10.41 (s, 1H), 7.21 (d, J = 11.2 Hz, 1H), 7.08 (s, 1H), 5.31 (s, 2H), 4.93-4.91 (m, 1H), 3.65-3.49 (m, 2H) 0.85 (s, 9H), 0.09 (s, 3H), −0.08 (s, 3H). |
| 32 | 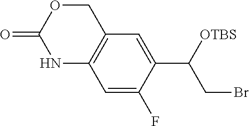 [SMILES: CC(C)(C)[Si](C)(C)OC(CBr)c1cc2c(cc1F)NC(=O)OC2] 6-(2-bromo-1-((tert- butyldimethylsilyl)oxy)ethyl)- 7-fluoro-1,4-dihydro-2H- benzo[d][1,3]oxazin-2-one | 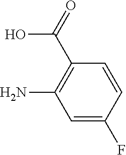 [SMILES: Nc1cc(F)ccc1C(=O)O] 2-amino-4-fluorobenzoic acid (CAS# 446-32-2) | Rt 1.09 min; MS m/z 403.9 and 405.9 [M + H^{]+}; Method J. | (400 MHz, CDCl₃) δ 8.23 (s, 1H), 7.27 (s, 1H), 6.58 (d, J = 10.0 Hz, 1H), 5.33 (s, 2H) 5.20-5.15 (m, 1H), 3.56-3.41 (m, 2H), 0.92 (s, 9H), 0.16 (s, 3H), −0.02 (s, 3H). |
| 33 | 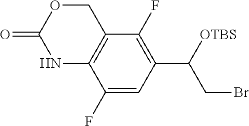 [SMILES: CC(C)(C)[Si](C)(C)OC(CBr)c1cc(F)c2c(c1F)COC(=O)N2] 6-(2-bromo-1-((tert- butyldimethylsilyl)oxy)ethyl)- 5,8-difluoro-1,4-dihydro- 2H-benzo[d][1,3]oxazin-2-one | 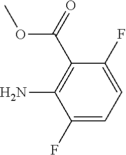 Methyl 2-amino-3,6- difluorobenzoate (CAS# 1184204-30-5) | Rt 1.14 min; MS m/z 422.1 and 424.1 [M + H]⁺; Method J. | (400 MHz, CDCl₃) δ 7.27-7.21 (m, 2H) 5.43 (s, 2H), 5.13 (m, 1H), 3.51-3.41 (m, 2H), 5.16-0.92 (s, 9H), 0.16 (s, 3H), −0.01 (s, 3H). |
| 34 | 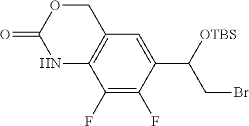 [SMILES: CC(C)(C)[Si](C)(C)OC(CBr)c1cc2c(c(F)c1F)NC(=O)OC2] 6-(2-bromo-1-((tert- butyldimethylsilyl)oxy)ethyl)- 7,8-difluoro-1,4-dihydro- 2H-benzo[d][1,3]oxazin-2-one | 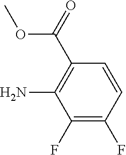 Methyl 2-amino-3,4- difluorobenzoate (CAS# 170108-07-3) | Rt 1.12 min; MS m/z 422.1 and 424.1 [M + H]⁺; Method J. | (400 MHz, CDCl₃) δ 7.51 (s, 1H), 7.06- 7.04 (m, 1H), 5.33 (s, 2H), 5.18-5.15 (m, 1H), 3.51-3.45 (m, 2H), 0.90 (s, 9H), 0.15 (s, 3H), − 0.02 (s, 3H). |
| 35 | 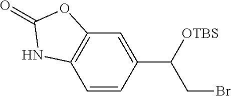 [SMILES: CC(C)(C)[Si](C)(C)OC(CBr)c1ccc2[nH]c(=O)oc2c1] 6-(2-bromo-1-((tert- butyldimethylsilyl)oxy)ethyl) benzo[d]oxazol-2(3H)-one | 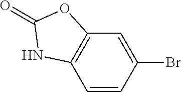 [SMILES: O=c1[nH]c2ccc(Br)cc2o1] 6-bromobenzo[d]oxazol- 2(3H)-one (CAS# 19932-85-5) | Rt 1.09 min; MS m/z 371.9 and 373.9 [M + H]⁺; Method J. | (400 MHz, CDCl₃) δ 8.84 (br s, 1H), 7.27- 7.25 (m, 1H), 7.18- 7.13 (m, 1H), 7.05 (d, J = 8.0 Hz, 1H), 4.90-4.81 (m, 1H), 3.53-3.35 (m, 2H), 0.90 (s, 9H), 0.13 (s, 3H), −0.07 (s, 3H). |
| 36 | 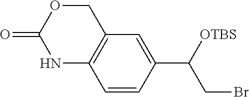 [SMILES: CC(C)(C)[Si](C)(C)OC(CBr)c1ccc2c(c1)COC(=O)N2] 6-(2-bromo-1-((tert- butyldimethylsilyl)oxy)ethyl)- 1,4-dihydro-2H- | 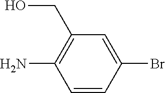 (2-amino-5- bromophenyl)methanol (CAS# 20712-12-3) | Rt 1.05 min; MS m/z 386.1 and 388.1 [M + H]⁺; Method J. | (400 MHz, CDCl₃) δ 8.86 (br s, 1H), 7.27-7.22 (m, 1H), 7.11 (s, 1H), 6.87-6.84 (m, 1H), 5.34 (s, 2H), 4.84- 4.76 (m, 1H), 3.52-3.31 (m, 2H), 0.89 (s, 9H), 0.11 (s, 3H), −0.08 (s, 3H). |
| | benzo[d][1,3]oxazin-2-one | | | |
| 37 | 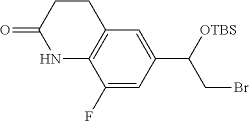 [SMILES: CC(C)(C)[Si](C)(C)OC(CBr)c1cc(F)c2c(c1)CCC(=O)N2] 6-(2-bromo-1-((tert- butyldimethylsilyl)oxy)ethyl)-8-fluoro-3,4- dihydroquinolin-2(1H)-one | 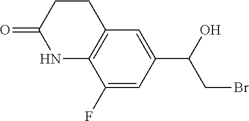 [SMILES: O=C1CCc2cc(C(O)CBr)cc(F)c2N1] 6-(2-bromo-1- hydroxyethyl)-8-fluoro- 3,4-dihydroquinolin-2(1H)-one (Intermediate 26) | Rt 1.10 min; MS m/z 402.1 and 404.1 [M + H]⁺; Method J. | (400 MHz, CDCl₃) δ 7.72 (br s, 1H), 7.03- 7.01 (m, 1H), 6.93 (s, 1H), 4.84-4.71 (m, 1H), 3.51-3.29 (m, 2H), 3.09-2.93 (m, 2H), 2.70-2.67 (m, 2H), 0.91 (s, 9H), 0.12 (s, 3H), −0.05 (s, 3H). |
| 38 | 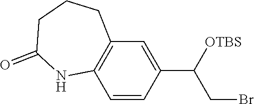 [SMILES: CC(C)(C)[Si](C)(C)OC(CBr)c1ccc2c(c1)CCCC(=O)N2] 7-(2-bromo-1-((tert- butyldimethylsilyl)oxy)ethyl)- 1,3,4,5-tetrahydro-2H- | 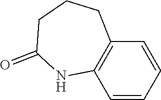 [SMILES: O=C1CCCc2ccccc2N1] 1,3,4,5-tetrahydro-2H- benzo[b]azepin-2-one (CAS# 4424-80-0) | Rt 1.12 min; MS m/z 398.1and 400.1 [M + H]⁺; Method J. | (400 MHz, CDCl₃) δ 7.88 (br s, 1H), 7.24-7.18 (m, 2H), 6.97-6.94 (m, 1H), 4.88-4.75 (m, 1H), 3.50- 3.43 (m, 2H), 2.83-2.79 (m, 2H), 2.41-2.30 (m, 2H), 2.29-2.18 (m, 2H), 0.89 (s, 9H), 0.12 (s, 3H), −0.07 (s, 3H). |
| | benzo[b]azepin-2-one | | | |

### Intermediate 39

### 5-(oxiran-2-yl)-1-tosyl-1H-pyrrolo[2,3-b]pyridine

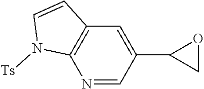 [SMILES: Cc1ccc(S(=O)(=O)n2ccc3cc(C4CO4)cnc32)cc1]

### Step 1: 5-bromo-1-tosyl-1H-pyrrolo[2,3-b]pyridine

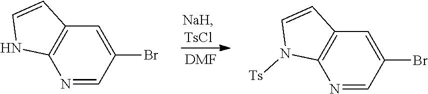

To a solution of 5-bromo-1H-pyrrolo[2,3-b]pyridine (CAS #183208-35-7) (2.5 g, 12.7 mmol) in DMF (50 mL) at 0° C. was added NaH (60% in mineral oil, 761 mg, 19.0 mmol) and this was stirred at RT for 20 minutes, then cooled again to 0° C. and 4-methylbenzenesulfonyl chloride (2.9 g, 15.2 mmol) was added. The reaction was stirred at RT for 2 h, then poured into ice water. The resulting solid was filtered to provide the title intermediate (3.0 g) which was used without further purification.

LCMS: Rt 1.87 min; MS m/z 351.1 and 353.1 [M+H]⁺; Method D.

### Step 2: 1-tosyl-5-vinyl-1H-pyrrolo[2,3-b]pyridine

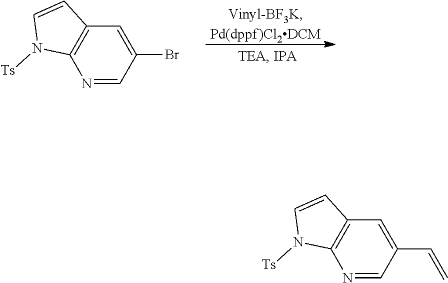 [SMILES: Cc1ccc(S(=O)(=O)n2ccc3cc(Br)cnc32)cc1 ; "Vinyl-BF3K,Pd(dppf)Cl2·DCM" ; "TEA, IPA" ; C=Cc1cnc2c(ccn2S(=O)(=O)c2ccc(C)cc2)c1]

To a solution of 5-bromo-1-tosyl-1H-pyrrolo[2,3-b]pyridine (3.0 g, 8.5 mmol) and potassium vinyltrifluoroborate (2.28 g, 17.1 mmol) in THF (90 mL) and water (20 mL) was added Cs₂CO₃ (8.35 g, 25.6 mmol) and the reaction was degassed with argon for 10 min. Pd(PPh₃)₄ was added and the reaction was stirred at 90° C. for 16 h. The reaction was extracted with EtOAc, dried with Na₂SO₄, filtered and concentrated. The crude material was purified by FCC (10% EtOAc:Hexane) to provide the title intermediate (2.0 g).

LCMS: Rt 1.80 min; MS m/z 299.2 [M+H]⁺; Method D.

### Step 3: 5-(oxiran-2-yl)-1-tosyl-1H-pyrrolo[2,3-b]pyridine

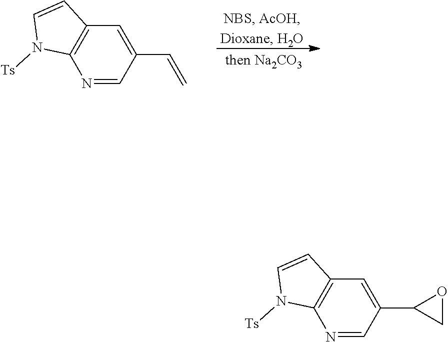 [SMILES: C=Cc1cnc2c(ccn2S(=O)(=O)c2ccc(C)cc2)c1 ; "NBS, AcOH,Dioxane, H2O" ; "then Na2CO3" ; Cc1ccc(S(=O)(=O)n2ccc3cc(C4CO4)cnc32)cc1]

To a solution of 1-tosyl-5-vinyl-1H-pyrrolo[2,3-b]pyridine (2.0 g, 6.7 mmol) in dioxane (30 mL) and water (150 mL) was added AcOH (403 mg, 6.7 mmol) and NBS (870 mg, 7.4 mmol) and the reaction was stirred at RT for 1 h. Na₂CO₃ (2.13 g, 20.1 mmol) was added and the reaction was stirred for 16 h, then extracted with EtOAc, dried with Na₂SO₄, filtered and concentrated. The crude material was purified by FCC (20% EtOAc:Hexane) to provide the title intermediate (1.5 g).

LCMS: Rt 1.58 min; MS m/z 315.2 [M+H]⁺; Method D.

### Intermediate 40

A racemic mixture of:
6-(2-((3aS,5R,6aR)-3a,5-dihydroxyhexahydrocyclopenta[c]pyrrol-2(1H)-yl)acetyl)-3,4-dihydroquinolin-2(1H)-one
6-(2-((3aR,5S,6aS)-3a,5-dihydroxyhexahydrocyclopenta[c]pyrrol-2(1H)-yl)acetyl)-3,4-dihydroquinolin-2(1H-one

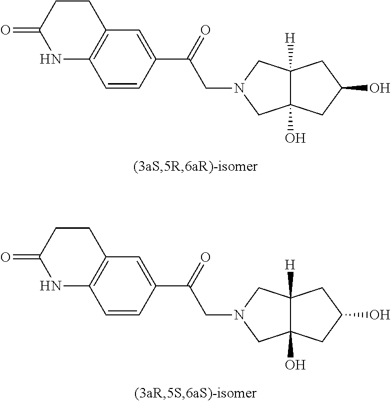 [SMILES: O=C1CCc2cc(C(=O)CN3C[C@H]4C[C@@H](O)C[C@@]4(O)C3)ccc2N1 ; O=C1CCc2cc(C(=O)CN3C[C@@H]4C[C@H](O)C[C@]4(O)C3)ccc2N1]

Step 1: A racemic mixture of:
(3aS,5R,6aR)-hexahydrocyclopenta[c]pyrrole-3a,5(1H)-diol
(3aR,5S,6aS)-hexahydrocyclopenta[c]pyrrole-3a,5(1H)-diol

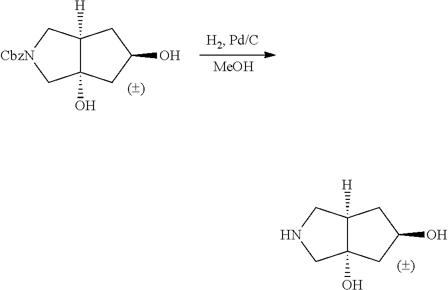 [SMILES: "MeOH" ; O=C(OCc1ccccc1)N1C[C@H]2C[C@@H](O)C[C@@]2(O)C1 ; "H2, Pd/C" ; O[C@@H]1C[C@@H]2CNC[C@]2(O)C1]

Using the same method as step 4 of Intermediate 13, starting with a racemic mixture of benzyl (3aS,5R,6aR)-3a,5-dihydroxyhexahydrocyclopenta[c]pyrrole-2(1H)-carboxylate and benzyl (3aR,5S,6aS)-3a,5-dihydroxyhexahydrocyclopenta[c]pyrrole-2(1 h)-carboxylate (from step 5 of Intermediate 1) (2.4 g, 8.65 mmol), provided the title intermediate (1.2 g) as a colorless gum which was used without further purification.

¹H NMR (400 MHz, DMSO-d₆) δ 4.69 (br s, 1H), 4.05-3.98 (m, 1H), 2.95-2.87 (m, 1H), 2.82-2.75 (m, 1H), 2.58-2.52 (m, 2H), 2.14-1.99 (m, 2H), 1.94-1.89 (m, 1H), 1.63-1.57 (m, 1H), 1.23-1.16 (m, 1H). 2H under solvent peak.

Step 2: A racemic mixture of
6-(2-((3aS,5R,6aR)-3a,5-dihydroxyhexahydrocyclopenta[c]pyrrol-2(1H)-yl)acetyl)-3,4-dihydroquinolin-2(1H)-one
6-(2-((3aR,5S,6aS)-3a,5-dihydroxyhexahydrocyclopenta[c]pyrrol-2(1H)-yl)acetyl)-3,4-dihydroquinolin-2(1H)-one

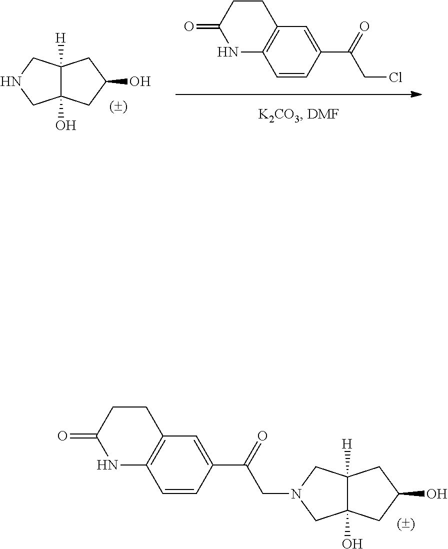

To a solution of a racemic mixture of (3aS,5R,6aR)-hexahydrocyclopenta[c]pyrrole-3a,5(1H)-diol and (3aR,5S,6aS)-hexahydrocyclopenta[c]pyrrole-3a,5(1H)-diol (900 mg, 6.29 mmol) in DMF (10 mL) was added 6-(2-chloroacetyl)-3,4-dihydroquinolin-2(1H)-one (Intermediate 11, 1.41 g, 6.29 mmol) and K₂CO₃ (1.74 g, 12.6 mmol) and this was stirred at RT for 4 h. The reaction was diluted with water (10 mL), extracted with EtOAc (3×10 mL), washed with sat. brine (15 mL), dried with Na₂SO₄, filtered and concentrated. The crude material was purified by preparative HPLC (Waters Xbridge C18, 150×50 mm, 10 micron, Mobile Phase A: Water with 10 mM NH₄HCO₃; B: Acetonitrile, Gradient 5-30% B) to provide the title intermediate (1.2 g) as a white solid.

LCMS: Rt 0.75 min; MS m/z 331.3 [M+H]⁺; Method I.

¹H NMR (400 MHz, CDC₃) δ 8.04-7.88 (m, 1H), 7.82-7.71 (m, 2H), 6.81-6.78 (m, 1H), 6.17-5.95 (br s, 1H), 4.21 (t, J=4.0 Hz, 1H), 3.93 (s, 2H), 3.37 (d, J=9.2 Hz, 1H), 3.08-2.93 (m, 3H), 2.77-2.65 (m, 3H), 2.52-2.33 (m, 3H), 2.25-2.19 (m, 1H), 2.14-2.05 (m, 1H), 1.82-1.68 (m, 2H).

### Example 1A

6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one

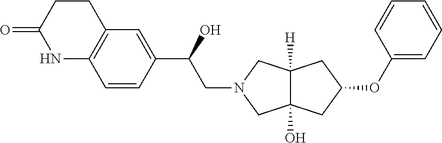 [SMILES: O=C1CCc2cc([C@@H](O)CN3C[C@H]4C[C@H](Oc5ccccc5)C[C@@]4(O)C3)ccc2N1]

### Step 1: 6-(2-((3aS,5S,6aR)-3a-hydroxy-5-phenoxyhexahydrocyclopenta[c]pyrrol-2(1H)-yl)acetyl)-3,4-dihydroquinolin-2(1H)-one

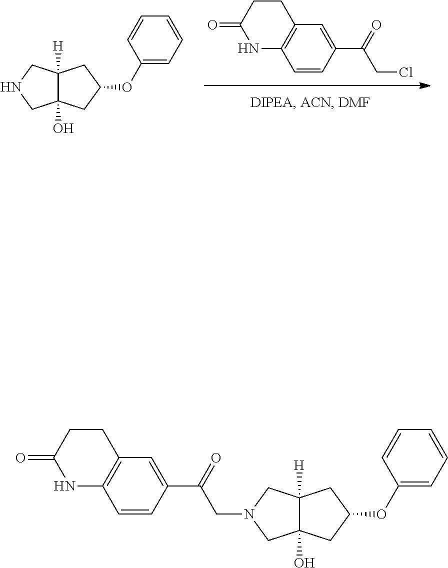

To a solution of Intermediate 11 (8.10 g, 32.6 mmol) and Intermediate 2 (6.5 g, 29.6 mmol) in CH₃CN (100 mL) and DMF (10 mL) was added DIPEA (10.35 mL, 59.3 mmol) and this was stirred at RT overnight. The reaction was concentrated, diluted with EtOAc and washed with water 3×. The aqueous layers were combined and extracted with EtOAc. The organic layers were combined, dried with Na₂SO₄, filtered and concentrated. The crude material was purified by FCC (100% EtOAc) to provide the title intermediate (6.0 g) as a light yellow foam.

LCMS: Rt 0.67 min; MS m/z 407.4 [M+H]⁺; Method A.

### Step 2: 6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one

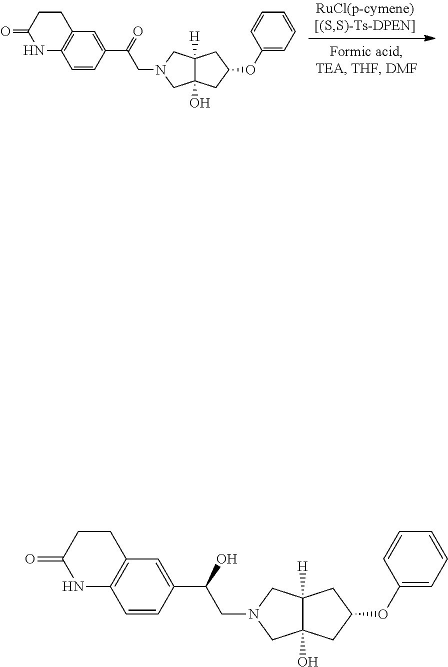 [SMILES: O=C1CCc2cc(C(=O)CN3C[C@H]4C[C@H](Oc5ccccc5)C[C@@]4(O)C3)ccc2N1 ; "Formic acid, TEA, THF, DMF" ; O=C1CCc2cc([C@@H](O)CN3C[C@H]4C[C@H](Oc5ccccc5)C[C@@]4(O)C3)ccc2N1]

To a solution of triethylamine (4.11 mL, 29.5 mmol) in THF (20 mL) at 0° C. was added formic acid (3.40 mL, 89 mmol), and this was added to a solution of 6-(2-((3aS,5S,6aR)-3a-hydroxy-5-phenoxyhexahydrocyclopenta[c]pyrrol-2(1H)-yl)acetyl)-3,4-dihydroquinolin-2(1H)-one (6.0 g, 14.8 mmol) in THF (50 mL) under nitrogen. A solution of RuCl(p-cymene)[(S,S)-Ts-DPEN] (CAS #192139-90-5) (0.240 g, 0.369 mmol) in DMF (5 mL) was added and the reaction was stirred at RT for 2 days. Another solution of triethylamine (4.11 mL) and formic acid (3.40 mL) in THF (10 mL) at 0° C. was added, followed by another solution of RuCl(p-cymene)[(S,S)-Ts-DPEN] (100 mg) in DMF (3 mL), and this was stirred at RT for 9 days. The reaction was partially concentrated to remove THF, diluted with EtOAc and washed with water 2×. The aqueous layers were combined and extracted with EtOAc. The organic layers were combined, dried with Na₂SO₄, filtered and concentrated. The crude material was purified by FCC (100% EtOAc, then 0-10% MeOH:DCM) to provide a brown oil. This was dissolved in DCM (40 mL) and MeOH (40 mL) and SiliaMetS DMT resin (Silicycle, 2 g, 0.64 mmol/g loading) was added and the slurry was stirred at RT for 5 h. The reaction was filtered, rinsing through with DCM, and the filtrate was treated with additional SiliaMetS DMT resin (2 g) and stirred overnight. The reaction was filtered, concentrated, and dissolved in EtOAc. This was concentrated to remove residual MeOH and DCM, then dissolved again in EtOAc. This was concentrated again until precipitation was observed, at which point the flask was cooled at 0° C. for 20 min. The solid was collected by filtration, washed with EtOAc 3×, and dried. The mother liquor was partially concentrated and sonicated until precipitation occurred. The solid was collected as before, and the process was repeated to obtain a third batch of solid. All three batches were combined and lyophilized to provide the title compound (1.59 g) as an offwhite solid.

LCMS: Rt 0.60 min; MS m/z 409.5 [M+H]⁺; Method A.

¹H NMR (400 MHz, Methanol-d₄) δ 7.27-7.15 (m, 4H), 6.92-6.79 (m, 4H), 4.77 (p, J=5.8 Hz, 1H), 4.69 (dd, J=8.3, 5.0 Hz, 1H), 2.91 (td, J=7.5, 2.0 Hz, 2H), 2.86-2.77 (m, 2H), 2.73 (dd, J=12.4, 8.3 Hz, 1H), 2.62 (d, J=9.3 Hz, 1H), 2.56 (dd, J=12.4, 5.0 Hz, 1H), 2.52-2.39 (m, 4H), 2.27 (dd, J=13.2, 5.4 Hz, 1H), 2.18-2.08 (m, 1H), 2.01 (dd, J=12.9, 6.6 Hz, 1H), 1.83 (dt, J=13.0, 5.0 Hz, 1H).

X-ray structure of Example 1A complexed with DCM:

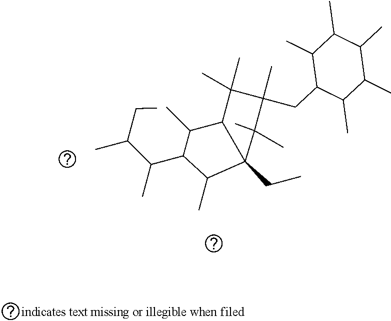

### Example 1B

6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one

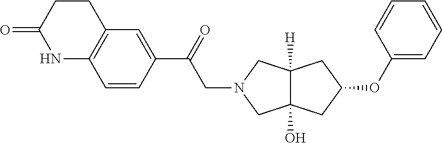 [SMILES: O=C1CCc2cc(C(=O)CN3C[C@H]4C[C@H](Oc5ccccc5)C[C@@]4(O)C3)ccc2N1]

Step 1: A mixture of:
6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one
6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one

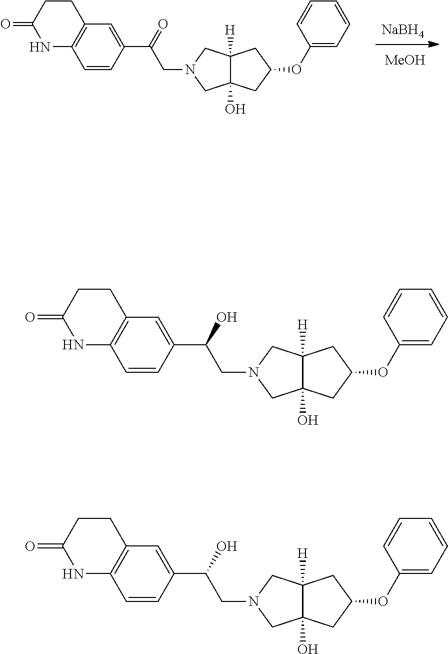 [SMILES: "MeOH" ; O=C1CCc2cc(C(=O)CN3C[C@H]4C[C@H](Oc5ccccc5)C[C@@]4(O)C3)ccc2N1 ; "NaBH4" ; O=C1CCc2cc([C@@H](O)CN3C[C@H]4C[C@H](Oc5ccccc5)C[C@@]4(O)C3)ccc2N1 ; O=C1CCc2cc([C@H](O)CN3C[C@H]4C[C@H](Oc5ccccc5)C[C@@]4(O)C3)ccc2N1]

To a suspension of 6-(2-((3aS,5S,6aR)-3a-hydroxy-5-phenoxyhexahydrocyclopenta[c]pyrrol-2(1H)-yl)acetyl)-3,4-dihydroquinolin-2(1H)-one (from step 1 of Example 1A) (300 mg, 0.73 mmol) in MeOH (15 mL) was added NaBH₄ (55 mg, 1.46 mmol) and this was stirred at RT for 1 h. The reaction was diluted with water, extracted with EtOAc, dried with Na₂SO₄, filtered and concentrated. The crude material was purified by FCC (5% MeOH:DCM), then by preparative HPLC using the method below to provide the title intermediates (75 mg).

Column: Kinetex (21.2 mm×150 mm), Flow: 20.0 mL/min

Mobile phase: 0.02% NH₄OH in water (A), Acetonitrile (B)

LCMS: Rt 0.11 min; MS m/z 409.2 [M+H]⁺; Method D.

### Step 2: 6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one

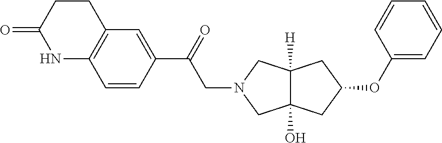 [SMILES: O=C1CCc2cc(C(=O)CN3C[C@H]4C[C@H](Oc5ccccc5)C[C@@]4(O)C3)ccc2N1]

The mixture from the previous step (75 mg) was separated using the following chiral HPLC method:

Column: C-4, Flow: 19 mL/min

Mobile phase: Hexane (A), EtOH:MeOH 80:20 with 0.1% DEA (B), Isocratic: 80:20 (A:B)

Example 1B (chiral HPLC Rt 7.08 min): 32 mg.

LCMS: Rt 0.43 min; MS m/z 409.2 [M+H]⁺; Method C.

¹H NMR (400 MHz, Methanol-d₄) δ 7.26-7.16 (m, 4H), 6.90-6.78 (m, 4H), 4.77 (p, J=5.8 Hz, 1H), 4.70 (dd, J=8.2, 5.1 Hz, 1H), 2.95-2.83 (m, 3H), 2.81 (d, J=9.3 Hz, 1H), 2.71 (dd, J=12.4, 8.2 Hz, 1H), 2.62-2.52 (m, 2H), 2.52-2.40 (m, 4H), 2.29-2.21 (m, 1H), 2.20-2.11 (m, 1H), 2.03-1.94 (m, 1H), 1.89-1.77 (m, 1H).

### Examples 2A and 2B

### 5-((R)-1-hydroxy-2-((3aS,5S,6aR)-3a-hydroxy-5-phenoxyhexahydrocyclopenta[c]pyrrol-2(1H)-yl)ethyl)indolin-2-one

### 5-((S)-1-hydroxy-2-((3aS,5S,6aR)-3a-hydroxy-5-phenoxyhexahydrocyclopenta[c]pyrrol-2(1H)-yl)ethyl)indolin-2-one

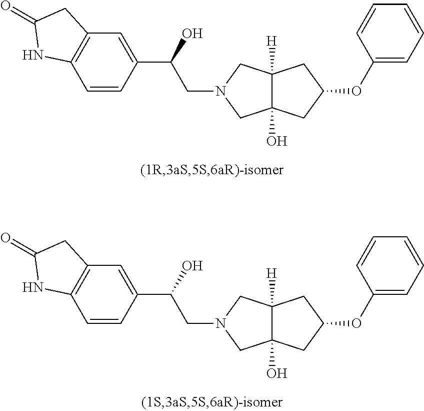 [SMILES: O=C1Cc2cc([C@@H](O)CN3C[C@H]4C[C@H](Oc5ccccc5)C[C@@]4(O)C3)ccc2N1 ; O=C1Cc2cc([C@H](O)CN3C[C@H]4C[C@H](Oc5ccccc5)C[C@@]4(O)C3)ccc2N1]

### Step 1: 5-(2-((3aS,5S,6aR)-3a-hydroxy-5-phenoxyhexahydrocyclopenta[c]pyrrol-2(1H)-yl)acetyl)indolin-2-one

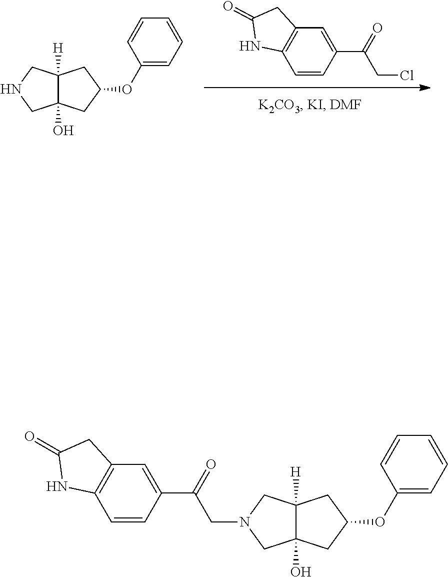

To a stirred suspension of 5-(2-chloroacetyl)indolin-2-one (CAS #65435-04-3) (150 mg, 0.71 mmol) and potassium carbonate (196 mg, 1.42 mmol) and potassium iodide (5.0 mg, 0.03 mmol) in DMF (1.0 mL) was added Intermediate 2 (156 mg, 0.71 mmol) and this was stirred at RT for 1 h. The reaction was poured into ice water, and the precipitate was filtered and dried to provide the title intermediate (250 mg) which was used without further purification.

LCMS: Rt 0.12 min; MS m/z 393.2 [M+H]⁺; Method D.

Step 2: A mixture of:
5-((R)-1-hydroxy-2-((3aS,5S,6aR)-3a-hydroxy-5-phenoxyhexahydrocyclopenta[c]pyrrol-2(1H)-yl)ethyl)indolin-2-one
5-((S)-1-hydroxy-2-((3aS,5S,6aR)-3a-hydroxy-5-phenoxyhexahydrocyclopenta[c]pyrrol-2(1H)-yl)ethyl)indolin-2-one

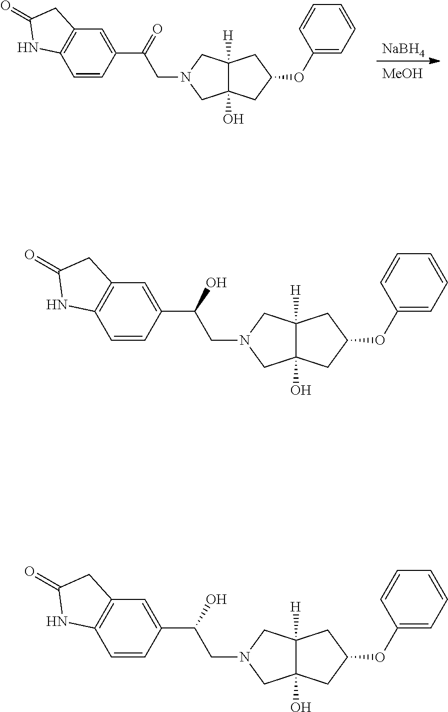 [SMILES: "MeOH" ; O=C1Cc2cc(C(=O)CN3C[C@H]4C[C@H](Oc5ccccc5)C[C@@]4(O)C3)ccc2N1 ; "NaBH4" ; O=C1Cc2cc([C@@H](O)CN3C[C@H]4C[C@H](Oc5ccccc5)C[C@@]4(O)C3)ccc2N1 ; O=C1Cc2cc([C@H](O)CN3C[C@H]4C[C@H](Oc5ccccc5)C[C@@]4(O)C3)ccc2N1]

Using the same method as step 1 of Example 11B, starting from 5-(2-((3aS,5S,6aR)-3a-hydroxy-5-phenoxyhexahydrocyclopenta[c]pyrrol-2(1H)-yl)acetyl)indolin-2-one (250 mg, 0.64 mmol), provided a mixture of Examples 2A and 2B (30 mg).

LCMS: Rt 0.39 min; MS m/z 395.1 [M+H]⁺; Method E.

### Step 3: Chiral Separation

The two diastereomers were separated using the chiral HPLC method below:

Column: Chiralpak IA (10 mm×250 mm, 5 μm), Flow: 15 mL/min

Mobile phase: Hexane (A), 0.1% DEA in IPA:MeOH 1:1 (B), Isocratic: 45:55 (A:B)

Example 2A (chiral HPLC Rt 14.85 min): 10 mg.

LCMS: Rt 0.45 min; MS m/z 395.1 [M+H]⁺; Method E.

¹H NMR (400 MHz, Methanol-d₄) δ 7.28 (s, 1H), 7.26-7.18 (m, 3H), 6.90-6.81 (m, 4H), 4.82-4.75 (m, 1H), 4.74-4.67 (m, 1H), 2.85-2.69 (m, 3H), 2.61 (d, J=9.6 Hz, 1H), 2.54 (dd, J=12.4, 5.2 Hz, 1H), 2.50-2.40 (m, 2H), 2.27 (dd, J=13.2, 5.6 Hz, 1H), 2.18-2.08 (m, 1H), 2.01 (dd, J=13.2, 6.4 Hz, 1H), 1.86-1.77 (m, 1H). 2H under solvent peak.

Example 2B (chiral HPLC Rt 22.07 min): 10 mg.

LCMS: Rt 0.49 min; MS m/z 395.2 [M+H]⁺; Method E.

¹H NMR (400 MHz, Methanol-d₄) δ 7.28 (d, J=2.0 Hz, 1H), 7.25-7.18 (m, 3H), 6.90-6.80 (m, 4H), 4.80-4.75 (m, 1H), 4.74-4.68 (m, 1H), 2.91-2.84 (m, 1H), 2.79 (d, J=9.2 Hz, 1H), 2.74-2.67 (m, 1H), 2.59-2.52 (m, 2H), 2.50-2.41 (m, 2H), 2.24 (dd, J=13.2, 5.2 Hz, 1H), 2.20-2.10 (m, 1H), 1.98 (dd, J=13.2, 6.4 Hz, 1H), 1.88-1.80 (m, 1H). 2H under solvent peak.

### Examples 3A, 3B, 3C and 3D

5-((R)-2-((3aS,4S,5S,6aR)-3a,4-dihydroxy-5-phenoxyhexahydrocyclopenta[c]pyrrol-2(1H)-yl)-1-hydroxyethyl)indolin-2-one
5-((S)-2-((3aS,4S,5S,6aR)-3a,4-dihydroxy-5-phenoxyhexahydrocyclopenta[c]pyrrol-2(1H)-yl)-1-hydroxyethyl)indolin-2-one
5-((R)-2-((3aR,4R,5R,6aS)-3a,4-dihydroxy-5-phenoxyhexahydrocyclopenta[c]pyrrol-2(1H)-yl)-1-hydroxyethyl)indolin-2-one
5-((S)-2-((3aR,4R,5R,6aS)-3a,4-dihydroxy-5-phenoxyhexahydrocyclopenta[c]pyrrol-2(1H-yl)-1-hydroxyethyl)indolin-2-one

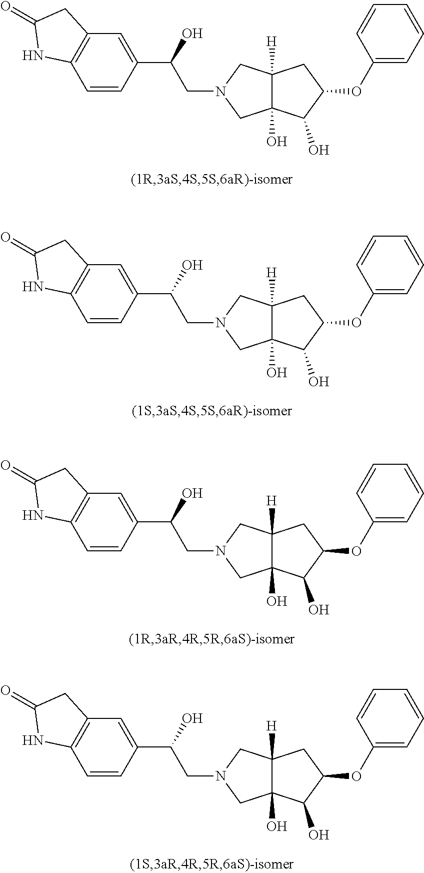 [SMILES: O=C1Cc2cc([C@@H](O)CN3C[C@H]4C[C@H](Oc5ccccc5)[C@H](O)[C@@]4(O)C3)ccc2N1 ; O=C1Cc2cc([C@H](O)CN3C[C@H]4C[C@H](Oc5ccccc5)[C@H](O)[C@@]4(O)C3)ccc2N1 ; O=C1Cc2cc([C@@H](O)CN3C[C@@H]4C[C@@H](Oc5ccccc5)[C@@H](O)[C@]4(O)C3)ccc2N1 ; O=C1Cc2cc([C@H](O)CN3C[C@@H]4C[C@@H](Oc5ccccc5)[C@@H](O)[C@]4(O)C3)ccc2N1]

Using the same methods as Examples 2A/2B, starting from Intermediate 6 and 5-(2-chloroacetyl)indolin-2-one, a mixture of Examples 3A and 3B was obtained. The mixture was separated using the following chiral SFC method: Column: Chiralpak IG (10 mm×250 mm, 5 μm), Flow: 13 mL/min Mobile phase: CO₂ (A), 0.02% NH₃ in IPA (B), Isocratic: 55:45 (A:B)

Example 3A (chiral SFC Rt 7.91 min): 25 mg.

LCMS: Rt 0.13 min; MS m/z 411.1 [M+H]⁺; Method D.

¹H NMR (400 MHz, Methanol-d₄) δ 7.30-7.22 (m, 4H), 6.94-6.85 (m, 4H), 4.75-4.67 (m, 2H), 3.92 (d, J=3.6 Hz, 1H), 2.94 (d, J=9.2 Hz, 1H), 2.79-2.61 (m, 4H), 2.42-2.36 (m, 2H), 2.31-2.23 (m, 1H), 1.67-1.62 (m, 1H). 2H under solvent peak.

Example 3B (chiral SFC Rt 15.41 min): 25 mg.

LCMS: Rt 1.24 min; MS m/z 411.2 [M+H]⁺; Method F.

¹H NMR (400 MHz, Methanol-d₄) δ 7.30-7.22 (m, 4H), 6.94-6.85 (m, 4H), 4.75-4.68 (m, 2H), 3.96 (d, J=3.6 Hz, 1H), 3.00 (d, J=10.0 Hz, 1H), 2.80-2.63 (m, 4H), 2.47 (d, J=9.2 Hz, 1H), 2.52-2.46 (m, 1H), 2.28-2.20 (m, 1H), 1.63-1.59 (m, 1H). 2H under solvent peak.

Using the same methods, starting from Intermediate 5 and 5-(2-chloroacetyl)indolin-2-one, a mixture of Examples 3C and 3D was obtained. The mixture was separated using the following chiral SFC method:

Column: Chiralpak IG (10 mm×250 mm, 5 μm), Flow: 13 mL/min

Mobile phase: CO₂ (A), 0.02% NH₃ in IPA (B), Isocratic: 80:20 (A:B)

Example 3C (chiral SFC Rt 12.08 min): 12 mg.

LCMS: Rt 0.13 min; MS m/z 411.2 [M+H]⁺; Method D.

¹H NMR (400 MHz, Methanol-d₄) δ 7.30-7.22 (m, 4H), 6.94-6.85 (m, 4H), 4.74-4.64 (m, 2H), 3.96 (d, J=3.6 Hz, 1H), 2.99 (d, J=9.2 Hz, 1H), 2.78-2.61 (m, 4H), 2.44-2.36 (m, 2H), 2.27-2.17 (m, 1H), 1.64-1.58 (m, 1H). 2H under solvent peak.

Example 3D (chiral SFC Rt 18.76 min): 12 mg.

LCMS: Rt 0.13 min; MS m/z 411.2 [M+H]⁺; Method D.

¹H NMR (400 MHz, Methanol-d₄) δ 7.30-7.22 (m, 4H), 6.94-6.85 (m, 4H), 4.75-4.69 (m, 2H), 3.92 (d, J=2.8 Hz, 1H), 2.94 (d, J=9.6 Hz, 1H), 2.78-2.61 (m, 4H), 2.40-2.23 (m, 2H), 2.27-2.17 (m, 1H), 1.66-1.62 (m, 1H). 2H under solvent peak.

### Examples 4A, 4B, 4C and 4D

6-((R)-2-((3aS,4S,5S,6aR)-3a,4-dihydroxy-5-phenoxyhexahydrocyclopenta[c]pyrrol-2(1H)-yl)-1-hydroxyethyl)-3,4-dihydroquinolin-2(1H)-one
6-((S)-2-((3aS,4S,5S,6aR)-3a,4-dihydroxy-5-phenoxyhexahydrocyclopenta[c]pyrrol-2(1H)-yl)-1-hydroxyethyl)-3,4-dihydroquinolin-2(1H)-one
6-((R)-2-((3aR,4R,5R,6aS)-3a,4-dihydroxy-5-phenoxyhexahydrocyclopenta[c]pyrrol-2(1H)-yl)-1-hydroxyethyl)-3,4-dihydroquinolin-2(1H)-one
6-((S)-2-((3aR,4R,5R,6aS)-3a,4-dihydroxy-5-phenoxyhexahydrocyclopenta[c]pyrrol-2(1H)-yl)-1-hydroxyethyl)-3,4-dihydroquinolin-2(1H)-one

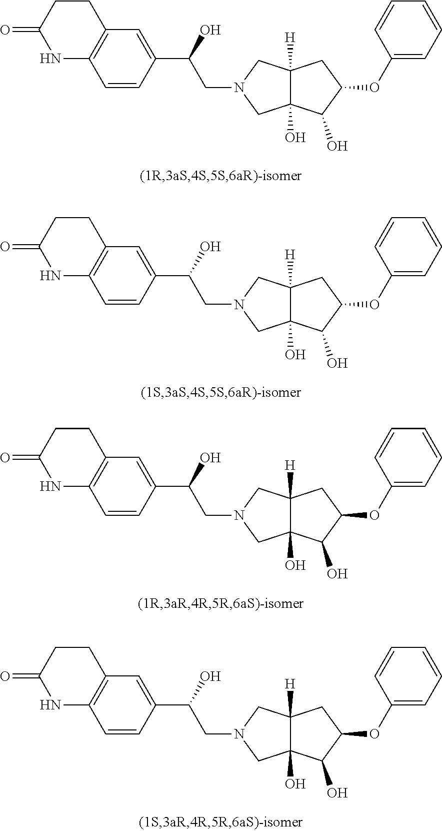 [SMILES: O=C1CCc2cc([C@@H](O)CN3C[C@H]4C[C@H](Oc5ccccc5)[C@H](O)[C@@]4(O)C3)ccc2N1 ; O=C1CCc2cc([C@H](O)CN3C[C@H]4C[C@H](Oc5ccccc5)[C@H](O)[C@@]4(O)C3)ccc2N1 ; O=C1CCc2cc([C@@H](O)CN3C[C@@H]4C[C@@H](Oc5ccccc5)[C@@H](O)[C@]4(O)C3)ccc2N1 ; O=C1CCc2cc([C@H](O)CN3C[C@@H]4C[C@@H](Oc5ccccc5)[C@@H](O)[C@]4(O)C3)ccc2N1]

Using the same methods as Examples 2A/2B, starting from Intermediate 5 and Intermediate 11, a mixture of Examples 4A and 4B was obtained. The mixture was separated using the following chiral HPLC method:

Column: Chiralpak IA (10 mm×250 mm), Flow rate: 9 mL/min

Mobile phase: Hexane (A), EtOH:MeOH 1:1 (B), Isocratic: 60:40 (A:B)

Example 4A (chiral HPLC Rt 14.18 min): 15 mg.

LCMS: Rt 1.24 min; MS m/z 425.4 [M+H]⁺; Method F.

¹H NMR (400 MHz, Methanol-d₄) δ 7.24-7.18 (m, 4H), 6.91-6.82 (m, 4H), 4.70-4.63 (m, 2H), 3.93 (d, J=3.6 Hz, 1H), 2.96 (d, J=9.6 Hz, 1H), 2.89-2.84 (m, 2H), 2.74-2.60 (m, 4H), 2.50-2.34 (m, 4H), 2.25-2.18 (m, 1H), 1.62-1.56 (m, 1H).

Example 4B (chiral HPLC Rt 28.51 min): 15 mg.

LCMS: Rt 1.25 min; MS m/z 425.4 [M+H]⁺; Method F.

¹H NMR (400 MHz, Methanol-d₄) δ 7.24-7.18 (m, 4H), 6.92-6.81 (m, 4H), 4.71-4.64 (m, 2H), 3.90 (d, J=3.2 Hz, 1H), 2.93-2.85 (m, 3H), 2.75-2.59 (m, 4H), 2.44 (t, J=8.4 Hz, 2H), 2.39-2.32 (m, 2H), 2.29-2.21 (m, 1H), 1.64-1.59 (m, 1H).

Using the same methods, starting from Intermediate 6 and Intermediate 11, a mixture of Examples 4C and 4D was obtained. The mixture was separated using the following chiral HPLC method:

Column: C-4, Flow: 20 mL/min

Mobile phase: Hexane (A), 0.1% DEA in EtOH (B), Isocratic: 65:35 (A:B)

Example 4C (chiral HPLC Rt 5.63 min): 30 mg.

LCMS: Rt 0.43 min; MS m/z 425.2 [M+H]⁺; Method D.

¹H NMR (400 MHz, Methanol-d₄) δ 7.26-7.20 (m, 4H), 6.94-6.84 (m, 4H), 4.74-4.66 (m, 2H), 3.93 (d, J=3.6 Hz, 1H), 2.96-2.87 (m, 3H), 2.78-2.62 (m, 4H), 2.49-2.37 (m, 4H), 2.31-2.26 (m, 1H), 1.66-1.61 (m, 1H).

Example 4D (chiral HPLC Rt 6.27 min): 40 mg.

LCMS: Rt 1.24 min; MS m/z 425.4 [M+H]⁺; Method F.

¹H NMR (400 MHz, Methanol-d₄) δ 7.27-7.20 (m, 4H), 6.94-6.84 (m, 4H), 4.74-4.66 (m, 2H), 3.93 (d, J=3.6 Hz, 1H), 2.96-2.87 (m, 3H), 2.78-2.62 (m, 4H), 2.49-2.37 (m, 4H), 2.31-2.24 (m, 1H), 1.67-1.61 (m, 1H).

### Examples 5A, 5B, 5C and 5D

6-((R)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one
6-((R)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one

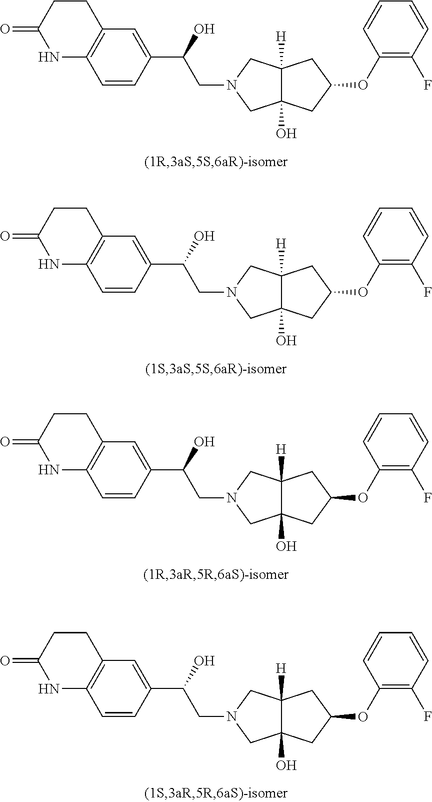 [SMILES: O=C1CCc2cc([C@@H](O)CN3C[C@H]4C[C@H](Oc5ccccc5F)C[C@@]4(O)C3)ccc2N1 ; O=C1CCc2cc([C@H](O)CN3C[C@H]4C[C@H](Oc5ccccc5F)C[C@@]4(O)C3)ccc2N1 ; O=C1CCc2cc([C@@H](O)CN3C[C@@H]4C[C@@H](Oc5ccccc5F)C[C@]4(O)C3)ccc2N1 ; O=C1CCc2cc([C@H](O)CN3C[C@@H]4C[C@@H](Oc5ccccc5F)C[C@]4(O)C3)ccc2N1]

Step 1: A racemic mixture of:
6-(2-((3aS,5S,6aR)-5-(2-fluorophenoxy)-3a-hydroxyhexahydrocyclopenta[c]pyrrol-2(1H)-yl)acetyl)-3,4-dihydroquinolin-2(1H)-one
6-(2-((3aR,5R,6aS)-5-(2-fluorophenoxy)-3a-hydroxyhexahydrocyclopenta[c]pyrrol-2(1H)-yl)acetyl)-3,4-dihydroquinolin-2(1H)-one

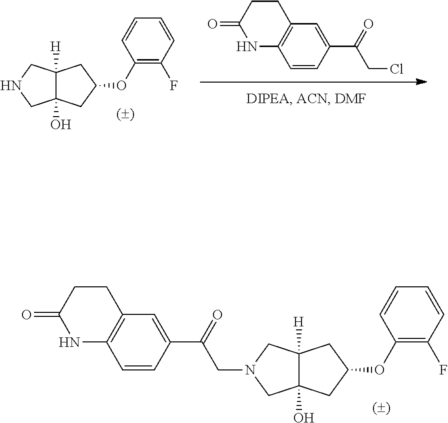

Using the same method as step 1 of Example 1A, starting from Intermediate 3 (75 mg, 0.32 mmol) and Intermediate 11 (106 mg, 0.38 mmol), provided the title intermediates (120 mg).

LCMS: Rt 0.92 min; MS m/z 425.3 [M+H]⁺; Method I.

### Step 2: A mixture of Examples 5A, 5B, 5C and 5D

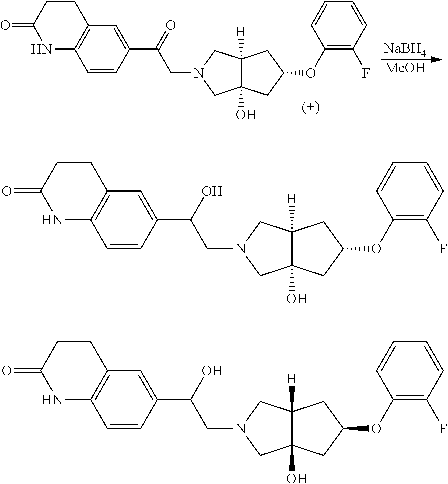

Using the same method as step 1 of Example 1 B, starting with the mixture of intermediates from the previous step (120 mg), provided a mixture of Examples 5A, 5B, 5C and 5D (40 mg).

LCMS: Rt 1.24 min; MS m/z 426.1 [M+H]⁺; Method E.

### Step 3: Chiral separation of Examples 5A, 5B, 5C and 5D

The mixture was first separated using the following chiral SFC method:

Column: Daicel Chiralpak AD (250 mm×30 mm, 10 μm), Flow: 70 g/min

Mobile phase: CO₂ (A), EtOH with 0.1% NH₃·H₂O (B), Isocratic 50:50 (A:B)

This provided two peaks, each containing two of the isomers. Both peaks were further separated using the following chiral SFC method:

Column: Daicel Chiralpak IG (250 mm×50 mm, 10 μm), Flow: 70 g/min

Mobile phase: CO₂ (A), MeOH:ACN (1:1) with 0.1% NH₃·H₂O (B), Isocratic 40:60 (A:B) Example 5A: 6 mg.

Analytical chiral SFC: Rt 1.14 min (Column: Chiralpak IG-3 50×4.6 mm, 3 μm, flow rate 3 mL/min, Mobile phase: 50% MeOH:ACN (1:1) with 0.05% DEA in CO₂).

LCMS: Rt 0.89 min; MS m/z 427.4 [M+H]⁺; Method I.

¹H NMR (400 MHz, CDCl₃) δ 7.66 (s, 1H), 7.23-7.15 (m, 2H), 7.14-6.93 (m, 4H), 6.72 (d, J=7.6 Hz, 1H), 5.01 (s, 1H), 4.82-4.70 (m, 1H), 3.31-3.28 (m, 1H), 3.03-2.93 (m, 3H), 2.86-2.69 (m, 2H), 2.68-2.58 (m, 4H), 2.54-2.45 (m, 2H), 2.39 (d, J=15.4 Hz, 1H), 2.22-2.13 (m, 1H), 1.71-1.60 (m, 1H).

Example 5B: 7 mg.

Analytical chiral SFC: Rt 1.56 min (Column: Chiralpak IG-3 50×4.6 mm, 3 μm, flow rate 3 mL/min, Mobile phase: 50% MeOH:ACN (1:1) with 0.05% DEA in CO₂).

LCMS: Rt 0.89 min; MS m/z 427.4 [M+H]⁺; Method I.

¹H NMR (400 MHz, CDCl₃) δ 7.68 (s, 1H), 7.21 (s, 1H), 7.19-7.15 (m, 1H), 7.13-7.06 (m, 2H), 7.05-6.93 (m, 2H), 6.72 (d, J=8.4 Hz, 1H), 5.02 (s, 1H), 4.75 (d, J=8.4 Hz, 1H), 3.14-3.04 (m, 1H), 3.01-2.93 (m, 3H), 2.88-2.70 (m, 3H), 2.70-2.57 (m, 4H), 2.55-2.46 (m, 1H), 2.38 (d, J=14.4 Hz, 1H), 2.25-2.18 (m, 1H), 1.54-1.43 (m, 1H).

Example 5C: 7 mg.

Analytical chiral SFC: Rt 2.46 min (Column: Chiralpak IG-3 50×4.6 mm, 3 μm, flow rate 3 mL/min, Mobile phase: 50% MeOH:ACN (1:1) with 0.05% DEA in CO₂).

LCMS: Rt 0.90 min; MS m/z 427.4 [M+H]⁺; Method I.

¹H NMR (400 MHz, CDCl₃) δ 7.78 (s, 1H), 7.23-7.15 (m, 2H), 7.14-6.93 (m, 4H), 6.73-6.71 (m, 1H), 5.01 (s, 1H), 4.73 (d, J=9.5 Hz, 1H), 3.12-3.03 (m, 1H), 3.01-2.91 (m, 3H), 2.83 (t, J=11.6 Hz, 1H), 2.78-2.46 (m, 7H), 2.38 (d, J=14.4 Hz, 1H), 2.20 (d, J=13.2 Hz, 1H), 1.69-1.52 (m, 1H).

Example 5D: 8 mg.

Analytical chiral SFC: Rt 5.04 min (Column: Chiralpak IG-3 50×4.6 mm, 3 μm, flow rate 3 mL/min, Mobile phase: 50% MeOH:ACN (1:1) with 0.05% DEA in CO₂).

LCMS: Rt 0.89 min; MS m/z 427.4 [M+H]⁺; Method I.

¹H NMR (400 MHz, CDCl₃) δ 7.76 (s, 1H), 7.25-7.15 (m, 2H), 7.15-7.06 (m, 2H), 7.05-6.93 (m, 2H), 6.73-6.71 (d, J=8.0 Hz, 1H), 5.01 (s, 1H), 4.83-4.69 (m, 1H), 3.39-3.23 (m, 1H), 3.03-2.93 (m, 3H), 2.84-2.72 (m, 2H), 2.68-2.58 (m, 4H), 2.54-2.44 (m, 2H), 2.39 (d, J=14.4 Hz, 1H), 2.22-2.14 (m, 1H), 1.73-1.61 (m, 1H).

### Examples 6A, 6B, 6C and 6D

8-fluoro-6-((R)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one
8-fluoro-6-((S)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1 h)-one
8-fluoro-6-((R)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one
8-fluoro-6-((S)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one

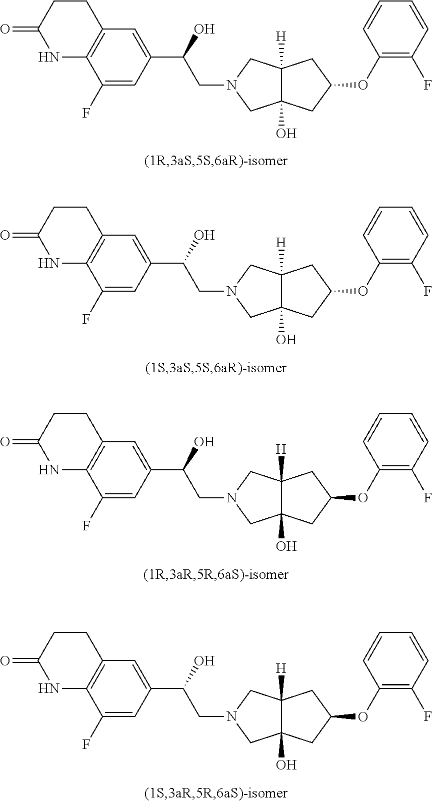 [SMILES: O=C1CCc2cc([C@@H](O)CN3C[C@H]4C[C@H](Oc5ccccc5F)C[C@@]4(O)C3)cc(F)c2N1 ; O=C1CCc2cc([C@H](O)CN3C[C@H]4C[C@H](Oc5ccccc5F)C[C@@]4(O)C3)cc(F)c2N1 ; O=C1CCc2cc([C@@H](O)CN3C[C@@H]4C[C@@H](Oc5ccccc5F)C[C@]4(O)C3)cc(F)c2N1 ; O=C1CCc2cc([C@H](O)CN3C[C@@H]4C[C@@H](Oc5ccccc5F)C[C@]4(O)C3)cc(F)c2N1]

Using the same methods as Examples 5A/5B/5C/5D, starting from Intermediate 3 and Intermediate 20, provided a mixture of Examples 6A/6B/6C/6D. The mixture was separated using the following chiral SFC method:

Column: Daicel Chiralpak AD (250 mm×30 mm, 10 μm), Flow Rate: 70 g/min

Mobile phase: CO₂ (A), EtOH with 0.1% NH₃·H₂O (B), Isocratic 50:50 (A:B)

Example 6A: 16 mg.

Analytical chiral SFC: Rt 0.88 min (Column: Chiralpak AD-3 50×4.6 mm, 3 μm, flow rate 3 mL/min, Mobile phase: 40% EtOH with 0.05% DEA in CO₂).

LCMS: Rt 0.89 min; MS m/z 445.3 [M+H]⁺; Method I.

¹H NMR (400 MHz, CDC₃) δ 7.55 (s, 1H), 7.13-6.95 (m, 6H), 5.01 (br s, 1H), 4.66-4.62 (m, 1H), 3.20-3.19 (m, 1H), 3.05-2.85 (m, 4H), 2.68-2.58 (m, 4H), 2.57-2.46 (m, 3H), 2.42-2.36 (m, 2H), 2.08-2.04 (m, 1H), 1.58-1.54 (m, 1H).

Example 6B: 16 mg.

Analytical chiral SFC: Rt 1.02 min (Column: Chiralpak AD-3 50×4.6 mm, 3 μm, flow rate 3 mL/min, Mobile phase: 40% EtOH with 0.05% DEA in CO₂).

LCMS: Rt 0.89 min; MS m/z 445.3 [M+H]⁺; Method I.

¹H NMR (400 MHz, CDCl₃) δ 7.59 (s, 1H), 7.14-6.93 (m, 6H), 5.01 (br s, 1H), 4.63-4.59 (m, 1H), 3.04-2.78 (m, 5H), 2.75-2.58 (m, 6H), 2.54-2.47 (m, 2H), 2.40-2.36 (m, 1H), 2.12-2.07 (m, 1H), 1.55-1.49 (m, 1H).

Example 6C: 16 mg.

Analytical chiral SFC: Rt 1.54 min (Column: Chiralpak AD-3 50×4.6 mm, 3 μm, flow rate 3 mL/min, Mobile phase: 40% EtOH with 0.05% DEA in CO₂).

LCMS: Rt 0.90 min; MS m/z 445.3 [M+H]⁺; Method I.

¹H NMR (400 MHz, CDCl₃) δ 7.58 (s, 1H), 7.14-7.06 (m, 2H), 7.05-7.00 (m, 2H), 7.00-6.93 (m, 2H), 5.01 (br s, 1H), 4.65-4.58 (m, 1H), 3.80 (br s, 1H), 3.04-2.90 (m, 4H), 2.88-2.81 (m, 1H), 2.75-2.59 (m, 6H), 2.55-2.46 (m, 2H), 2.42-2.35 (m, 1H), 2.14-2.06 (m, 1H), 1.56-1.49 (m, 1H).

Example 6D: 15 mg.

Analytical chiral SFC: Rt 1.81 min (Column: Chiralpak AD-3 50×4.6 mm, 3 μm, flow rate 3 mL/min, Mobile phase: 40% EtOH with 0.05% DEA in CO₂).

LCMS: Rt 0.89 min; MS m/z 445.3 [M+H]⁺; Method I.

¹H NMR (400 MHz, CDCl₃) δ 7.66 (s, 1H), 7.14-7.00 (m, 4H), 6.99-6.92 (m, 2H), 5.01 (br s, 1H), 4.67-4.60 (m, 1H), 3.79 (br s, 1H), 3.20 (d, J=8.8 Hz, 1H), 3.07-2.83 (m, 4H), 2.68-2.46 (m, 7H), 2.42-2.34 (m, 2H), 2.10-2.02 (m, 1H), 1.61-1.51 (m, 1H).

These examples were made as pairs of diastereomers using the same methods as Examples 5A/5B/5C/5D, starting with the intermediates shown, and were separated using the conditions shown.

| Examples | |
|---|---|
| Intermediates | |
| Chiral SFC conditions | |
| Analytical data | Structures and names |
| Examples 7A and 7B Made from Intermediate 2 and 7-(2- chloroacetyl)- 1,3,4,5- tetrahydro-2H- benzo[b]azepin- 2-one (CAS# 154195-54-7) | 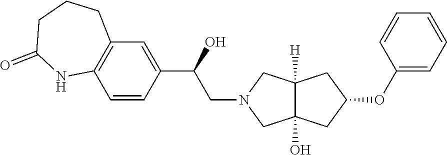 [SMILES: O=C1CCCc2cc([C@@H](O)CN3C[C@H]4C[C@H](Oc5ccccc5)C[C@@]4(O)C3)ccc2N1] (1R,3aS,5S,6aR)-isomer |
| | 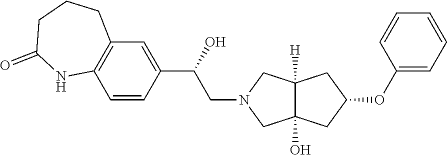 [SMILES: O=C1CCCc2cc([C@H](O)CN3C[C@H]4C[C@H](Oc5ccccc5)C[C@@]4(O)C3)ccc2N1] |
| | (1S,3aS,5S,6aR)-isomer |
| | 7-((R)-1-hydroxy-2-((3aS,5S,6aR)-3a-hydroxy-5- |
| | phenoxyhexahydrocyclopenta[c]pyrrol-2(1 H)-yl)ethyl)-1,3,4,5- |
| | tetrahydro-2H-benzo[b]azepin-2-one |
| | 7-((S)-1-hydroxy-2-((3aS,5S,6aR)-3a-hydroxy-5- |
| | phenoxyhexahydrocyclopenta[c]pyrrol-2(1 H)-yl)ethyl)-1,3,4,5- |
| | tetrahydro-2H-benzo[b]azepin-2-one |

Chiral SFC (separation): Column: Chiralpak AD-H (250 mm × 21 mm, 5 μm), Flow Rate:
80 g/min, Mobile phase: 35-55% MeOH:IPA (1:1) with 10 mM NH₃ in CO₂
Chiral SFC (analytical): Column: Chiralpak AD-3 (100 × 3 mm, 3 μm), Flow Rate: 2.5
mL/min, Mobile phase: 5-55% MeOH:IPA (1:1) with 0.1% NH₃ in CO₂
Example 7A: Analytical chiral SFC: Rt 3.04 min.
LCMS: Rt 1.05 min; MS m/z 423.7 [M + H]⁺; Method B.
¹H NMR (400 MHz, Methanol-d₄) 0 7.31-7.18 (m, 4H), 6.99 (d, J = 8.6 Hz, 1H), 6.91-
6.82 (m, 3H), 4.83-4.72 (m, 2H), 2.99-2.87 (m, 1H), 2.87-2.67 (m, 4H), 2.66-2.55 (m,
2H), 2.54-2.41 (m, 2H), 2.32-2.10 (m, 6H), 2.06-1.96 (m, 1H), 1.90-1.77 (m, 1H).
Example 7B: Analytical chiral SFC: Rt 3.20 min.
LCMS: Rt 1.04 min; MS m/z 423.4 [M + H]⁺; Method B.
¹H NMR (400 MHz, Methanol-d₄) 0 7.33-7.17 (m, 4H), 6.99 (d, J = 8.6 Hz, 1H), 6.92-
6.80 (m, 3H), 4.82-4.71 (m, 2H), 2.90-2.71 (m, 5H), 2.65 (d, J = 9.4 Hz, 1H), 2.57 (dd,
1.2 Hz, 1H), 1.82 (dt, J = 12.5, 5.0 Hz, 1H).
J = 12.4, 4.8 Hz, 1H), 2.53-2.41 (m, 2H), 2.32-2.10 (m, 6H), 2.03 (ddd, J = 13.4, 6.5,
1.2 Hz, 1H), 1.82 (dt, J = 12.5, 5.0 Hz, 1H).

| | |
|---|---|
| Examples 8A and 8B | 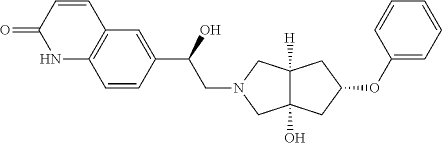 [SMILES: O=c1ccc2cc([C@@H](O)CN3C[C@H]4C[C@H](Oc5ccccc5)C[C@@]4(O)C3)ccc2[nH]1] |
| | (1R,3aS,5S,6aR)-isomer |
| Made from Intermediates 2 and 12 | 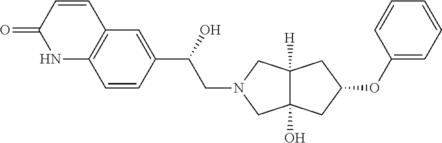 [SMILES: O=c1ccc2cc([C@H](O)CN3C[C@H]4C[C@H](Oc5ccccc5)C[C@@]4(O)C3)ccc2[nH]1] |
| | (1S,3aS,5S,6aR)-isomer |
| | 6-((R)-1-hydroxy-2- |
| | ((3aS,5S,6aR)-3a-hydroxy-5-phenoxyhexahydrocyclopenta[c]pyrrol- |
| | 2(1 H)-yl)ethyl)quinolin-2(1H)-one |
| | 6-((S)-1-hydroxy-2-((3aS,5S,6aR)-3a-hydroxy-5- |
| | phenoxyhexahydrocyclopenta[c]pyrrol-2(1 H)-yl)ethyl)quinolin-2(1H)-one |

Chiral SFC (separation): Column: Phenomenex Lux-cellulose-4 (250 mm × 21 mm),
Flow Rate: 80 g/min, Mobile phase: 40% MeOH with 10 mM NH₄OH in CO₂
Chiral SFC (analytical): Column: Lux Cellulose-4 (100 × 3 mm, 3 μm), Flow Rate: 2.5
mL/min, Mobile phase: 40% MeOH with 0.1% NH₃ in CO₂
Example 8A: Analytical chiral SFC: Rt 2.78 min.
LCMS: Rt 0.95 min; MS m/z 407.2 [M + H]⁺; Method B.
¹H NMR (400 MHz, Methanol-d₄) δ 7.94 (dd, J = 9.6, 0.7 Hz, 1H), 7.69 (d, J = 1.9 Hz, 1H),
7.61 (dd, J = 8.6, 1.9 Hz, 1H), 7.34 (d, J = 8.5 Hz, 1H), 7.25-7.13 (m, 2H), 6.87 (tt, J =
7.4, 1.1 Hz, 1H), 6.83-6.73 (m, 2H), 6.57 (d, J = 9.5 Hz, 1H), 4.74 (p, J = 5.7 Hz, 1H),
2.91 (t, J = 8.4 Hz, 1H), 2.87-2.74 (m, 2H), 2.68 (dd, J = 12.5, 5.3 Hz, 1H), 2.64-2.51
(m, 2H), 2.46 (tt, J = 8.4, 4.2 Hz, 1H), 2.29-2.08 (m, 2H), 1.98 (dd, J = 13.3, 6.1 Hz, 1H),
1.83 (dt, J = 12.9, 5.1 Hz, 1H). 1H under solvent peak.
Example 8B: Analytical chiral SFC: Rt 3.60 min.
LCMS: Rt 0.92 min; MS m/z 407.5 [M + H]⁺; Method B.
¹H NMR (400 MHz, Methanol-d₄) δ 7.94 (d, J = 9.5 Hz, 1H), 7.70 (d, J = 1.9 Hz, 1H), 7.61
(dd, J = 8.5, 1.9 Hz, 1H), 7.35 (d, J = 8.5 Hz, 1H), 7.28-7.11 (m, 2H), 6.88 (tt, J = 7.4,
1.1 Hz, 1H), 6.85-6.74 (m, 2H), 6.58 (d, J = 9.4 Hz, 1H), 4.76 (p, J = 5.5 Hz, 1H), 3.10-
2.39 (m, 7H), 2.30 (dd, J = 13.4, 5.4 Hz, 1H), 2.15 (tdd, J = 8.9, 6.8, 5.5 Hz, 1H), 2.02 (dd,
J = 13.4, 6.1 Hz, 1H), 1.83 (dt, J = 13.2, 5.2 Hz, 1H). 1H under solvent peak.

| | |
|---|---|
| Examples 9A and 9B Made from Intermediates 2 and 13 | 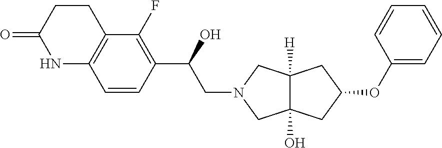 [SMILES: O=C1CCc2c(ccc([C@@H](O)CN3C[C@H]4C[C@H](Oc5ccccc5)C[C@@]4(O)C3)c2F)N1] |
| | (1R,3aS,5S,6aR)-isomer |
| | 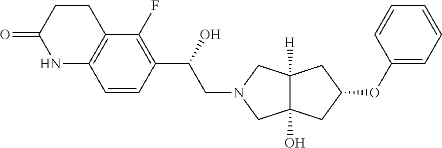 [SMILES: O=C1CCc2c(ccc([C@H](O)CN3C[C@H]4C[C@H](Oc5ccccc5)C[C@@]4(O)C3)c2F)N1] |
| | (1S,3aS,5S,6aR)-isomer |
| | 5-fluoro-6-((R)-1-hydroxy-2-((3aS,5S,6aR)-3a-hydroxy-5- |
| | phenoxyhexahydrocyclopenta[c]pyrrol-2(1 H)-yl)ethyl)-3,4- |
| | dihydroquinolin-2(1H)-one |
| | 5-fluoro-6-((S)-1-hydroxy-2-((3aS,5S,6aR)-3a-hydroxy-5- |
| | phenoxyhexahydrocyclopenta[c]pyrrol-2(1 H)-yl)ethyl)-3,4- |
| | dihydroquinolin-2(1H)-one |

Chiral SFC (separation): Column: Daicel Chiralpak AD (250 mm × 30 mm, 10 μm), Flow
Rate: 80 g/min, Mobile phase: 70% IPA with 0.1% NH₃·H₂O in CO₂
Chiral SFC (analytical): Column: Chiralpak AD-3 (50 × 4.6 mm, 3 μm), Flow Rate: 3
mL/min, Mobile phase: 60% MeOH:ACN (1:1) with 0.05% DEA in CO₂
Example 9A: Analytical chiral SFC: Rt 1.46 min.
LCMS: Rt 0.90 min; MS m/z 427.3 [M + H]⁺; Method I.
¹H NMR (400 MHz, CDCl₃) δ 8.21 (br s, 1H), 7.39-7.27 (m, 3H), 7.06-6.83 (m, 3H), 6.59
(d, J = 8.0 Hz, 1H), 5.06-4.88 (m, 2H), 3.17 (d, J = 9.2 Hz, 1H), 3.03-2.87 (m, 3H), 2.74-
2.32 (m, 10H), 2.15-2.05 (m, 1H), 1.66-1.56 (m, 1H).
Example 9B: Analytical chiral SFC: Rt 2.22 min.
LCMS: Rt 0.89 min; MS m/z 427.3 [M + H]⁺; Method I.
¹H NMR (400 MHz, CDCl₃) δ 8.13 (br s, 1H), 7.40-7.28 (m, 3H), 7.04-6.85 (m, 3H), 6.59
(d, J = 8.0 Hz, 1H), 5.11-4.88 (m, 2H), 3.06-2.90 (m, 3H), 2.87-2.46 (m, 10H), 2.36-
2.32 (m, 1H), 2.18-2.10 (m, 1H), 1.62-1.54 (m, 1H).

| | |
|---|---|
| Examples 10A and 10B | 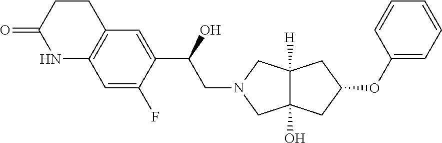 [SMILES: O=C1CCc2cc([C@@H](O)CN3C[C@H]4C[C@H](Oc5ccccc5)C[C@@]4(O)C3)c(F)cc2N1] |
| | (1R,3aS,5S,6aR)-isomer |
| Made from Intermediates 2 and 17 | 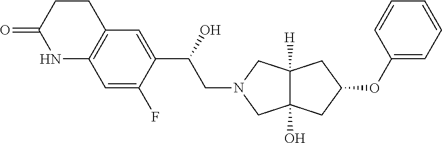 [SMILES: O=C1CCc2cc([C@H](O)CN3C[C@H]4C[C@H](Oc5ccccc5)C[C@@]4(O)C3)c(F)cc2N1] |
| | (1S,3aS,5S,6aR)-isomer |
| | 7-fluoro-6-((R)-1-hydroxy-2-((3aS,5S,6aR)-3a-hydroxy-5- |
| | phenoxyhexahydrocyclopenta[c]pyrrol-2(1 H)-yl)ethyl)-3,4- |
| | dihydroquinolin-2(1H)-one |
| | 7-fluoro-6-((S)-1-hydroxy-2-((3aS,5S,6aR)-3a-hydroxy-5- |
| | phenoxyhexahydrocyclopenta[c]pyrrol-2(1 H)-yl)ethyl)-3,4- |
| | dihydroquinolin-2(1H)-one |

Chiral SFC (separation): Column: Daicel Chiralpak AD (250 mm × 30 mm, 10 μm), Flow
Rate: 70 g/min, Mobile phase: 45% EtOH with 0.1% NH₃•H₂O in CO₂
Chiral SFC (analytical): Column: Chiralpak AD-3 (50 × 4.6 mm, 3 μm), Flow Rate: 3
mL/min, Mobile phase: 40% EtOH with 0.05% DEA in CO₂
Example 10A: Analytical chiral SFC: Rt 0.94 min.
LCMS: Rt 0.90 min; MS m/z 427.4 [M + H]⁺; Method I.
¹H NMR (400 MHz, CDCl₃) δ 7.62-7.53 (m, 1H), 7.38-7.29 (m, 3H), 7.03-6.96 (m, 1H),
6.94-6.89 (m, 2H), 6.45-6.43 (m, 1H), 5.07-4.87 (m, 2H), 3.01-2.81 (m, 4H), 2.78-
2.48 (m, 9H), 2.35-2.33 (m, 1H), 2.16-2.14 (m, 1H), 1.62-1.55 (m, 2H).
Example 10B: Analytical chiral SFC: Rt 1.38 min.
LCMS: Rt 0.90 min; MS m/z 427.4 [M + H]⁺; Method I.
¹H NMR (400 MHz, CDCl₃) δ 7.82 (br s, 1H), 7.37-7.28 (m, 3H), 7.00-6.98 (m, 1H), 6.92-
6.88 (m, 2H), 6.47-6.44 (m, 1H), 5.07-4.85 (m, 2H), 3.18-3.15 (m, 1H), 2.99-2.90
(m, 3H), 2.68-2.42 (m, 9H), 2.33-2.11 (m, 1H), 2.10-1.63 (m, 1H), 1.62-1.60 (m, 2H).

| | |
|---|---|
| Examples 11A and 11B | 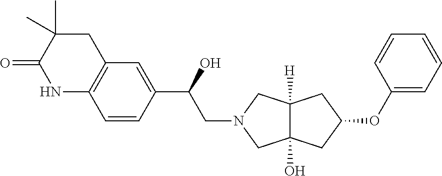 [SMILES: CC1(C)Cc2cc([C@@H](O)CN3C[C@H]4C[C@H](Oc5ccccc5)C[C@@]4(O)C3)ccc2NC1=O] |
| | (1R,3aS,5S,6aR)-isomer |
| Made from Intermediates 2 and 19 | 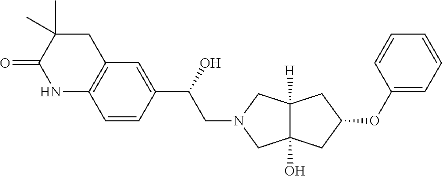 [SMILES: CC1(C)Cc2cc([C@H](O)CN3C[C@H]4C[C@H](Oc5ccccc5)C[C@@]4(O)C3)ccc2NC1=O] |
| | (1S,3aS,5S,6aR)-isomer |
| | 6-((R)-1-hydroxy-2-((3aS,5S,6aR)-3a-hydroxy-5- |
| | phenoxyhexahydrocyclopenta[c]pyrrol-2(1 H)-yl)ethyl)-3,3-dimethyl-3,4- |
| | dihydroquinolin-2(1H)-one |
| | 6-((S)-1-hydroxy-2-((3aS,5S,6aR)-3a-hydroxy-5- |
| | phenoxyhexahydrocyclopenta[c]pyrrol-2(1 H)-yl)ethyl)-3,3-dimethyl-3,4- |
| | dihydroquinolin-2(1H)-one |

Chiral SFC (separation): Column: Daicel Chiralpak AD (250 mm × 30 mm, 10 μm), Flow
Rate: 80 g/min, Mobile phase: 50% EtOH with 0.1% NH₃•H₂O in CO₂
Chiral SFC (analytical): Column: Chiralpak AD-3 (50 × 4.6 mm, 3 μm), Flow Rate: 3
mL/min, Mobile phase: 50% EtOH with 0.05% DEA in CO₂
Example 11A: Analytical chiral SFC: Rt 1.04 min.
LCMS: Rt 0.94 min; MS m/z 437.5 [M + H]⁺; Method I.
¹H NMR (400 MHz, CDCl₃) δ 7.37-7.28 (m, 3H), 7.21-7.14 (m, 2H), 7.01-6.88 (m, 3H),
6.69-6.67 (m, 1H), 4.97 (br s, 1H), 4.74-4.61 (m, 1H), 3.22-3.19 (m, 1H), 3.01-2.87
(m, 1H), 2.82-2.32 (m, 10H), 2.17-2.07 (m, 1H), 1.59-1.47 (m, 2H), 1.21 (s, 6H).
Example 11B: Analytical chiral SFC: Rt 1.47 min.
LCMS: Rt 0.94 min; MS m/z 437.5 [M + H]⁺; Method I.
¹H NMR (400 MHz, CDCl₃) δ 7.40-7.29 (m, 3H), 7.22-7.12 (m, 2H), 7.03-6.89 (m, 3H),
6.69-6.67 (m, 1H), 4.98 (br s, 1H), 4.69-4.66 (m, 1H), 3.03-2.47 (m, 11H), 2.37-2.33
(m, 1H), 2.23-2.12 (m, 1H), 1.59-1.46 (m, 2H), 1.21 (s, 6H).

| | |
|---|---|
| Examples 12A and 12B | 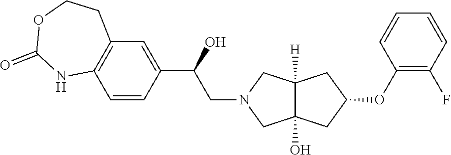 [SMILES: O=C1Nc2ccc([C@@H](O)CN3C[C@H]4C[C@H](Oc5ccccc5F)C[C@@]4(O)C3)cc2CCO1] |
| | (1R,3aS,5S,6aR)-isomer |
| Made from Intermediates 4 and 14 | 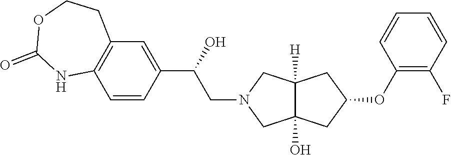 [SMILES: O=C1Nc2ccc([C@H](O)CN3C[C@H]4C[C@H](Oc5ccccc5F)C[C@@]4(O)C3)cc2CCO1] |
| | (1S,3aS,5S,6aR)-isomer |
| | 7-((R)-2-((3aS,5S,6aR)-5-(2-fluorophenoxy)-3a- |
| | hydroxyhexahydrocyclopenta[c]pyrrol-2(1 H)-yl)-1-hydroxyethyl)-4,5- |
| | dihydrobenzo[d][1,3]oxazepin-2(1H)-one |
| | 7-((S)-2-((3aS,5S,6aR)-5-(2-fluorophenoxy)-3a- |
| | hydroxyhexahydrocyclopenta[c]pyrrol-2(1 H)-yl)-1-hydroxyethyl)-4,5- |
| | dihydrobenzo[d][1,3]oxazepin-2(1H)-one |

Chiral SFC (separation): Column: Daicel Chiralpak IG (250 mm × 30 mm, 10 μm), Flow
Rate: 70 g/min, Mobile phase: 55% IPA with 0.1% NH₃•H₂O in CO₂
Chiral SFC (analytical): Column: Chiralpak IG-3 (50 × 4.6 mm, 3 μm), Flow Rate: 3
mL/min, Mobile phase: 50% IPA:ACN (1:1) with 0.05% DEA in CO₂
Example 12A: Analytical chiral SFC: Rt 1.03 min.
LCMS: Rt 0.89 min; MS m/z 443.4 [M + H]⁺; Method I.
¹H NMR (400 MHz, CDCl₃) δ 7.18-6.95 (m, 7H), 6.77-6.75 (m, 1H), 5.02 (br s, 1H), 4.89-
4.58 (m, 1H), 4.54-4.46 (m, 2H), 3.23-3.19 (m, 2H), 3.09-2.34 (m, 10H), 2.28-2.08
(m, 1H). 2H under solvent peak.
Example 12B: Analytical chiral SFC: Rt 1.34 min.
LCMS: Rt 0.88 min; MS m/z 443.5 [M + H]⁺; Method I.
¹H NMR (400 MHz, CDCl₃) δ 7.19-6.93 (m, 7H), 6.77 (d, J = 8.2 Hz, 1H), 5.01 (br s, 1H),
4.80-4.59 (m, 1H), 4.56-4.47 (m, 2H), 3.35-3.13 (m, 3H), 3.06-2.87 (m, 2H), 2.79-
2.35 (m, 7H), 2.23-2.04 (m, 1H). 2H under solvent peak.

| | |
|---|---|
| Examples 13A and 13B | 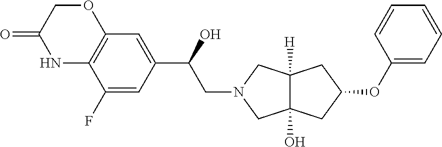 [SMILES: O=C1COc2cc([C@@H](O)CN3C[C@H]4C[C@H](Oc5ccccc5)C[C@@]4(O)C3)cc(F)c2N1] |
| | (1R,3aS,5S,6aR)-isomer |
| Made from Intermediates 2 and 21 | 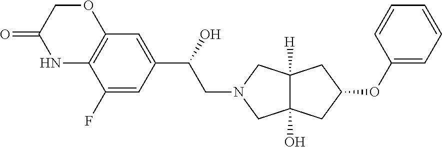 [SMILES: O=C1COc2cc([C@H](O)CN3C[C@H]4C[C@H](Oc5ccccc5)C[C@@]4(O)C3)cc(F)c2N1] |
| | (1S,3aS,5S,6aR)-isomer |
| | 5-fluoro-7-((R)-1-hydroxy-2-((3aS,5S,6aR)-3a-hydroxy-5- |
| | phenoxyhexahydrocyclopenta[c]pyrrol-2(1 H)-yl)ethyl)-2H- |
| | benzo[b][1,4]oxazin-3(4H)-one |
| | 5-fluoro-7-((S)-1-hydroxy-2-((3aS,5S,6aR)-3a-hydroxy-5- |
| | phenoxyhexahydrocyclopenta[c]pyrrol-2(1 H)-yl) ethyl)-2H- |
| | benzo[b][1,4]oxazin-3(4H)-one |

Chiral SFC (separation): Column: Daicel Chiralpak OJ (250 mm × 30 mm, 10 μm), Flow
Rate: 70 g/min, Mobile phase: 50% EtOH with 0.1% NH₃•H₂O in CO₂
Chiral SFC (analytical): Column: Chiralpak OJ-3 (50 × 4.6 mm, 3 μm), Flow Rate: 3
mL/min, Mobile phase: 5-40% EtOH with 0.05% DEA in CO₂
Example 13A: Analytical chiral SFC: Rt 1.99 min.
LCMS: Rt 0.91 min; MS m/z 429.4 [M + H]⁺; Method I.
¹H NMR (400 MHz, CDCl₃) δ 7.67 (br s, 1H), 7.31-7.29 (m, 2H), 6.99-6.92 (m, 1H), 6.92-
6.81 (m, 4H), 4.97 (br s, 1H), 4.64 (s, 3H), 2.85-2.76 (m, 1H), 2.75-2.71 (m, 1H), 2.78-
2.48 (m, 7H), 2.35-2.18 (m, 1H), 2.17-2.14 (m, 1H), 1.61-4.60 (m, 2H).
Example 13B: Analytical chiral SFC: Rt 2.22 min.
LCMS: Rt 0.91 min; MS m/z 429.4 [M + H]⁺; Method I.
1H NMR (400 MHz, CDCl₃) δ 7.58 (br s, 1H), 7.23-7.21 (m, 2H), 6.94-6.89 (m, 1H), 6.86-
6.76 (m, 3H), 6.74 (s, 1H), 4.89 (br s, 1H), 4.64-4.88 (m, 1H), 4.56 (s, 2H), 3.29-3.08
(m, 1H), 3.00-2.94 (m, 1H), 2.82-2.36 (m, 7H), 2.28-2.09 (m, 1H), 2.14-2.03 (m, 1H),
1.58-1.57 (m, 2H).

| | |
|---|---|
| Examples 14A and 14B | 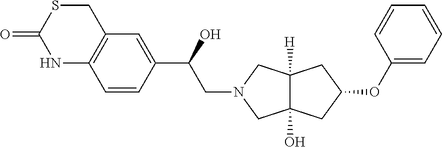 [SMILES: O=C1Nc2ccc([C@@H](O)CN3C[C@H]4C[C@H](Oc5ccccc5)C[C@@]4(O)C3)cc2CS1] |
| | (1R,3aS,5S,6aR)-isomer |
| Made from Intermediates 2 and 15 | 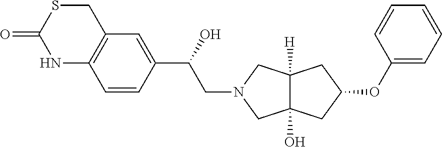 [SMILES: O=C1Nc2ccc([C@H](O)CN3C[C@H]4C[C@H](Oc5ccccc5)C[C@@]4(O)C3)cc2CS1] |
| | (1S,3aS,5S,6aR)-isomer |
| | 6-((R)-1-hydroxy-2-((3aS,5S,6aR)-3a-hydroxy-5- phenoxyhexahydrocyclopenta[c]pyrrol-2(1 H)-yl)ethyl)-1,4-dihydro-2H- benzo[d][1,3]thiazin-2-one 6-((S)-1-hydroxy-2-((3aS,5S,6aR)-3a-hydroxy-5- phenoxyhexahydrocyclopenta[c]pyrrol-2(1 H)-yl)ethyl)-1,4-dihydro-2H- benzo[d][1,3]thiazin-2-one |

Chiral SFC (separation): Column: Daicel Chiralpak AD (250 mm × 30 mm, 10 μm), Flow
Rate: 70 g/min, Mobile phase: 50% EtOH:ACN (1:1) with 0.1% NH₃•H₂O in CO₂
Chiral SFC (analytical): Column: Chiralpak AD-3 (50 × 4.6 mm, 3 μm), Flow Rate: 3
mL/min, Mobile phase: 50% EtOH with 0.05% DEA in CO₂
Example 14A: Analytical chiral SFC: Rt 1.96 min.
LCMS: Rt 0.90 min; MS m/z 427.2 [M + H]⁺; Method I.
¹H NMR (400 MHz, CDCl₃) δ 8.23 (br s, 1H), 7.34-7.28 (m, 2H), 7.27-7.22 (m, 2H), 7.02-
6.96 (m, 1H), 6.94-6.88 (m, 2H), 6.86-6.80 (m, 1H), 4.97 (br s, 1H), 4.72-4.65 (m,
1H), 4.10 (s, 2H), 3.80 (br s, 1H), 3.19 (d, J = 9.2 Hz, 1H), 2.97-2.88 (m, 1H), 2.75-2.62
(m, 2H), 2.60-2.47 (m, 4H), 2.41-2.33 (m, 2H), 2.14-2.05 (m, 1H), 1.62-1.59 (m, 1H).
Example 14B: Analytical chiral SFC: Rt 2.35 min.
LCMS: Rt 0.91 min; MS m/z 427.2 [M + H]⁺; Method I.
¹H NMR (400 MHz, CDCl₃) δ 8.43 (s, 1H), 7.36-7.29 (m, 2H), 7.28-7.23 (m, 2H), 7.04-
6.98 (m, 1H), 6.95-6.90 (m, 2H), 6.86 (d, J = 8.0 Hz, 1H), 4.99 (br s, 1H), 4.75-4.64 (m,
1H), 4.11 (s, 2H), 2.99-2.96 (m, 1H), 2.88-2.86 (m, 1H), 2.82-2.58 (m, 5H), 2.57-2.50
(m, 2H), 2.38-2.35 (m, 1H), 2.20-2.14 (m, 1H), 1.64-1.57 (m, 1H).

| | |
|---|---|
| Examples 15A and 15B | 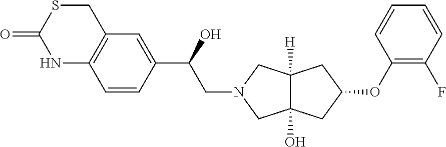 [SMILES: O=C1Nc2ccc([C@@H](O)CN3C[C@H]4C[C@H](Oc5ccccc5F)C[C@@]4(O)C3)cc2CS1] |
| | (1R,3aS,5S,6aR)-isomer |
| Made from Intermediates 4 and 15 | 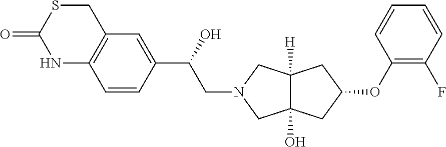 [SMILES: O=C1Nc2ccc([C@H](O)CN3C[C@H]4C[C@H](Oc5ccccc5F)C[C@@]4(O)C3)cc2CS1] |
| | (1S,3aS,5S,6aR)-isomer |
| | 6-((R)-2-((3aS,5S,6aR)-5-(2-fluorophenoxy)-3a- |
| | hydroxyhexahydrocyclopenta[c]pyrrol-2(1 H)-yl)-1-hydroxyethyl)-1,4- |
| | dihydro-2H-benzo[d][1,3]thiazin-2-one |
| | 6-((S)-2-((3aS,5S,6aR)-5-(2-fluorophenoxy)-3a- |
| | hydroxyhexahydrocyclopenta[c]pyrrol-2(1 H)-yl)-1-hydroxyethyl)-1,4- |
| | dihydro-2H-benzo[d][1,3]thiazin-2-one |

Chiral SFC (separation): Column: Daicel Chiralpak AD (250 mm × 50 mm, 10 μm), Flow
Rate: 70 g/min, Mobile phase: 60% MeOH with 0.1% NH₃•H₂O in CO₂
Chiral SFC (analytical): Column: Chiralpak AD-3 (50 × 4.6 mm, 3 μm), Flow Rate: 3
mL/min, Mobile phase: 50% MeOH:ACN (1:1) with 0.05% DEA in CO₂
Example 15A: Analytical chiral SFC: Rt 1.73 min.
LCMS: Rt 0.91 min; MS m/z 445.4 [M + H]⁺; Method I.
¹H NMR (400 MHz, CDCl₃) δ 8.23 (br s, 1H), 7.27-7.22 (m, 2H), 7.14-7.00 (m, 3H), 7.00-
6.91 (m, 1H), 6.84 (d, J = 8.8 Hz, 1H), 5.01 (br s, 1H), 4.72-4.69 (m, 1H), 4.09 (s, 2H),
3.25-3.22 (m, 1H), 2.96-2.92 (m, 1H), 2.74-2.29 (m, 8H), 2.11-2.07 (m, 1H), 1.61-
1.56 (m, 1H).
Example 15B: Analytical chiral SFC: Rt 2.34 min.
LCMS: Rt 0.91 min; MS m/z 445.4 [M + H]⁺; Method I.
¹H NMR (400 MHz, CDCl₃) δ 8.24 (br s, 1H), 7.26-7.20 (m, 2H), 7.14-7.00 (m, 3H), 7.00-
6.92 (m, 1H), 6.83 (d, J = 8.0 Hz, 1H), 5.02 (br s, 1H), 4.72-4.61 (m, 1H), 4.09 (s, 2H),
3.08-2.81 (m, 3H), 2.77-2.71 (m, 1H), 2.67-2.57 (m, 3H), 2.55-2.47 (m, 2H), 2.44-
2.34 (m, 1H), 2.16-2.07 (m, 1H), 1.57-1.48 (m, 1H).

| | |
|---|---|
| Examples 16A and 16B | 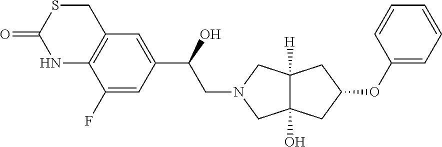 [SMILES: O=C1Nc2c(F)cc([C@@H](O)CN3C[C@H]4C[C@H](Oc5ccccc5)C[C@@]4(O)C3)cc2CS1] |
| | (1R,3aS,5S,6aR)-isomer |
| Made from Intermediates 2 and 16 | 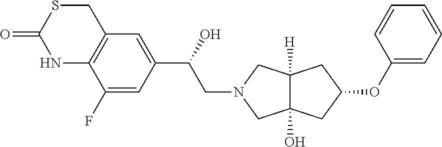 [SMILES: O=C1Nc2c(F)cc([C@H](O)CN3C[C@H]4C[C@H](Oc5ccccc5)C[C@@]4(O)C3)cc2CS1] |
| | (1S,3aS,5S,6aR)-isomer |
| | 8-fluoro-6-((R)-1-hydroxy-2-((3aS,5S,6aR)-3a-hydroxy-5- |
| | phenoxyhexahydrocyclopenta[c]pyrrol-2(1 H)-yl)ethyl)-1,4-dihydro-2H- |
| | benzo[d][1,3]thiazin-2-one |
| | 8-fluoro-6-((S)-1-hydroxy-2-((3aS,5S,6aR)-3a-hydroxy-5- |
| | phenoxyhexahydrocyclopenta[c]pyrrol-2(1 H)-yl)ethyl)-1,4-dihydro-2H- |
| | benzo[d][1,3]thiazin-2-one |

Chiral SFC (separation): Column: Daicel Chiralpak IG (250 mm × 30 mm, 10 μm), Flow
Rate: 70 g/min, Mobile phase: 50% IPA:ACN (1:1) with 0.1% NH_{3•}H₂O in CO₂
Chiral SFC (analytical): Column: Chiralpak IG-3 (50 × 4.6 mm, 3 μm), Flow Rate: 3
mL/min, Mobile phase: 50% IPA:ACN (1:1) with 0.05% DEA in CO₂
Example 16A: Analytical chiral SFC: Rt 1.07 min.
LCMS: Rt 0.74 min; MS m/z 445.2 [M + H]⁺; Method J.
¹H NMR (400 MHz, CDCl₃) δ 7.83-7.66 (m, 1H), 7.35-7.27 (m, 2H), 7.12 (d, J = 11.2
Hz, 1H), 7.07-6.96 (m, 2H), 6.94-6.89 (m, 2H), 4.98 (br s, 1H), 4.69-4.61 (m, 1H), 4.12
(s, 2H), 2.93 (d, J = 9.2 Hz, 1H), 2.83 (d, J = 8.8 Hz, 1H), 2.75-2.48 (m, 7H), 2.36 (d, J =
14.4 Hz, 1H), 2.16-2.09 (m, 1H), 1.58-1.50 (m, 1H).
Example 16B: Analytical chiral SFC: Rt 1.51 min.
LCMS: Rt 0.74 min; MS m/z 445.1 [M + H]⁺; Method J.
¹H NMR (400 MHz, CDCl₃) δ 7.77 (br s, 1H), 7.35-7.28 (m, 2H), 7.12 (d, J = 10.8 Hz,
1H), 7.05-6.95 (m, 2H), 6.91 (d, J = 7.6 Hz, 2H), 4.97 (br s, 1H), 4.69-4.62 (m, 1H),
4.12 (s, 2H), 3.17 (d, J = 9.2 Hz, 1H), 2.98-2.85 (m, 1H), 2.71-2.46 (m, 6H), 2.43-2.30
(m, 2H), 2.09 (m, 1H), 1.65-1.52 (m, 1H).

| | |
|---|---|
| Examples 17A and 17B | 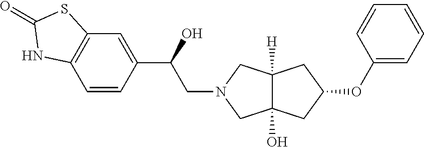 [SMILES: O=c1[nH]c2ccc([C@@H](O)CN3C[C@H]4C[C@H](Oc5ccccc5)C[C@@]4(O)C3)cc2s1] |
| | (1R,3aS,5S,6aR)-isomer |
| Made from Intermediates 2 and 18 | 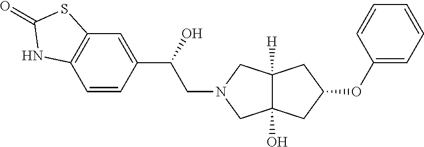 [SMILES: O=c1[nH]c2ccc([C@H](O)CN3C[C@H]4C[C@H](Oc5ccccc5)C[C@@]4(O)C3)cc2s1] |
| | (1S,3aS,5S,6aR)-isomer |
| | 6-((R)-1-hydroxy-2-((3aS,5S,6aR)-3a-hydroxy-5- |
| | phenoxyhexahydrocyclopenta[c]pyrrol-2(1 H)-yl)ethyl)benzo[d]thiazol- |
| | 2(3H)-one |
| | 6-((S)-1-hydroxy-2-((3aS,5S,6aR)-3a-hydroxy-5- |
| | phenoxyhexahydrocyclopenta[c]pyrrol-2(1 H)-yl)ethyl)benzo[d]thiazol- |
| | 2(3H)-one |

Chiral SFC (separation): Column: Daicel Chiralpak AD (250 mm × 30 mm, 10 μm), Flow
Rate: 80 g/min, Mobile phase: 60% EtOH with 0.1% NH₃•H₂O in CO₂
Chiral SFC (analytical): Column: Chiralpak AD-3 (50 × 4.6 mm, 3 μm), Flow Rate: 3
mL/min, Mobile phase: 40% EtOH with 0.05% DEA in CO₂
Example 17A: Analytical chiral SFC: Rt 1.82 min.
LCMS: Rt 0.81 min; MS m/z 413.4 [M + H]⁺; Method I.
¹H NMR (400 MHz, CDCl₃) δ 7.47 (s, 1H), 7.34-7.22 (m, 3H), 7.09-7.04 (m, 1H), 7.01-
6.95 (m, 1H), 6.91 (d, J = 7.6 Hz, 2H), 4.97 (br s, 1H), 4.76-4.69 (m, 1H), 3.21-3.15 (m,
1H), 2.98-2.89 (m, 1H), 2.73-2.30 (m, 8H), 2.13-2.06 (m, 1H), 1.65-1.54 (m, 1H).
Example 17B: Analytical chiral SFC: Rt 3.36 min.
LCMS: Rt 0.81 min; MS m/z 413.4 [M + H]⁺; Method I.
¹H NMR (400 MHz, CDCl₃) δ 7.47 (d, J = 1.2 Hz, 1H), 7.35-7.22 (m, 3H), 7.10-7.04 (m,
1H), 7.01-6.95 (m, 1H), 6.91 (d, J = 7.6 Hz, 2H), 4.98 (br s, 1H), 4.74-4.67 (m, 1H),
2.96-2.48 (m, 9H), 2.39-2.32 (m, 1H), 2.18-2.09 (m, 1H), 1.62-1.53 (m, 1H).

| | |
|---|---|
| Examples 18A and 18B | 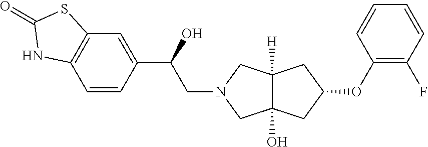 [SMILES: O=c1[nH]c2ccc([C@@H](O)CN3C[C@H]4C[C@H](Oc5ccccc5F)C[C@@]4(O)C3)cc2s1] |
| | (1R,3aS,5S,6aR)-isomer |
| Made from Intermediates 4 and 18 | 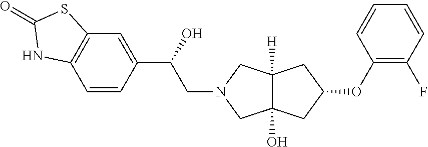 [SMILES: O=c1[nH]c2ccc([C@H](O)CN3C[C@H]4C[C@H](Oc5ccccc5F)C[C@@]4(O)C3)cc2s1] |
| | (1S,3aS,5S,6aR)-isomer |
| | 6-((R)-2-((3aS,5S,6aR)-5-(2-fluorophenoxy)-3a- |
| | hydroxyhexahydrocyclopenta[c]pyrrol-2(1 H)-yl)-1- |
| | hydroxyethyl)benzo[d]thiazol-2(3H)-one |
| | 6-((S)-2-((3aS,5S,6aR)-5-(2-fluorophenoxy)-3a- |
| | hydroxyhexahydrocyclopenta[c]pyrrol-2(1 H)-yl)-1- |
| | hydroxyethyl)benzo[d]thiazol-2(3H)-one |

Chiral SFC (separation): Column: Daicel Chiralpak AD (250 mm × 30 mm, 10 μm), Flow
Rate: 70 g/min, Mobile phase: 70% MeOH:ACN (1:1) with 0.1% NH₃. in CO₂
Chiral SFC (analytical): Column: Chiralpak AD-3 (50 × 4.6 mm, 3 μm), Flow Rate: 3
mL/min, Mobile phase: 60% MeOH:ACN (1:1) with 0.05% DEA in CO₂
Example 18A: Analytical chiral SFC: Rt 0.74 min.
LCMS: Rt 0.80 min; MS m/z 431.4 [M + H]⁺; Method I.
¹H NMR (400 MHz, Methanol-d₄) δ 7.50 (s, 1H), 7.30-7.28 (m, 1H), 7.10-7.01 (m, 3H),
6.98-6.84 (m, 2H), 4.82-4.70 (m, 2H), 2.92-2.68 (m, 3H), 2.64-2.41 (m, 4H), 2.28-
2.15 (m, 2H), 2.09-2.00 (m, 1H), 1.85-1.80 (m, 1H).
Example 18B: Analytical chiral SFC: Rt 1.90 min.
LCMS: Rt 0.83 min; MS m/z 431.2 [M + H]⁺; Method I.
¹H NMR (400 MHz, Methanol-d₄) δ 7.50 (d, J = 1.3 Hz, 1H), 7.30-7.28 (m, 1H), 7.12-
6.99 (m, 3H), 6.98-6.85 (m, 2H), 4.81-4.71 (m, 2H), 2.85-2.68 (m, 3H), 2.63-2.40 (m,
4H), 2.31-2.12 (m, 2H), 2.06-2.00 (m, 1H), 1.82-1.80 (m, 1H).

### Example 19

A mixture of:
(S)-3,8-difluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one
(S)-3,8-difluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one
(R)-3,8-difluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one
(R)-3,8-difluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one

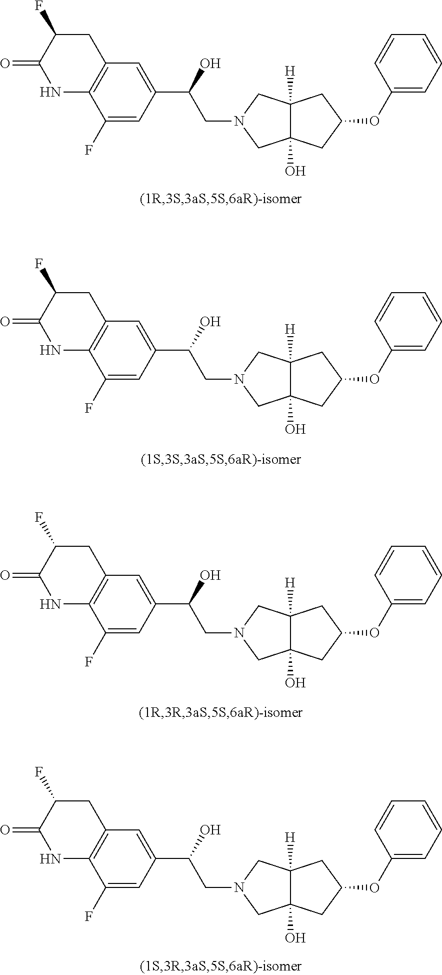 [SMILES: O=C1Nc2c(F)cc([C@@H](O)CN3C[C@H]4C[C@H](Oc5ccccc5)C[C@@]4(O)C3)cc2C[C@@H]1F ; O=C1Nc2c(F)cc([C@H](O)CN3C[C@H]4C[C@H](Oc5ccccc5)C[C@@]4(O)C3)cc2C[C@@H]1F ; O=C1Nc2c(F)cc([C@@H](O)CN3C[C@H]4C[C@H](Oc5ccccc5)C[C@@]4(O)C3)cc2C[C@H]1F ; O=C1Nc2c(F)cc([C@H](O)CN3C[C@H]4C[C@H](Oc5ccccc5)C[C@@]4(O)C3)cc2C[C@H]1F]

Using the same methods as Examples 5A/5B/5C/5D, starting from Intermediate 2 and Intermediate 22, provided Example 19 as a mixture of four diastereomers.

LCMS: Rt 0.90 min; MS m/z 445.4 [M+H]⁺; Method I.

¹H NMR (400 MHz, CDC₃) δ 7.58 (br s, 1H), 7.35-7.31 (m, 2H), 7.14-6.99 (m, 3H), 6.93 (d, J=8.0 Hz, 2H), 5.28-5.08 (m, 1H), 4.99 (br s, 1H), 4.70-4.63 (m, 1H), 3.40-3.34 (m, 2H), 3.22-2.84 (m, 2H), 2.77-2.51 (m, 6H), 2.49-2.31 (m, 2H), 2.18-2.08 (m, 1H).

### Examples 20A and 20B

3,8-difluoro-6-((R)-1-hydroxy-2-((3aS,5S,6aR)-3a-hydroxy-5-phenoxyhexahydrocyclopenta[c]pyrrol-2(1H)-yl)ethyl)quinolin-2(1H)-one
3,8-difluoro-6-((S)-1-hydroxy-2-((3aS,5S,6aR)-3a-hydroxy-5-phenoxyhexahydrocyclopenta[c]pyrrol-2(1H)-yl)ethyl)quinolin-2(1H)-one

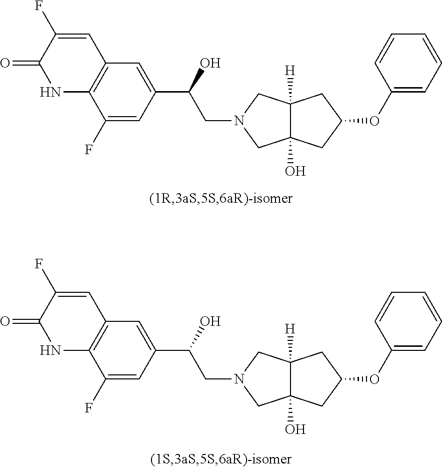 [SMILES: O=c1[nH]c2c(F)cc([C@@H](O)CN3C[C@H]4C[C@H](Oc5ccccc5)C[C@@]4(O)C3)cc2cc1F ; O=c1[nH]c2c(F)cc([C@H](O)CN3C[C@H]4C[C@H](Oc5ccccc5)C[C@@]4(O)C3)cc2cc1F]

### Step 1: 3,8-difluoro-6-(2-((3aS,5S,6aR)-3a-hydroxy-5-phenoxyhexahydrocyclopenta[c]pyrrol-2(1H)-yl)acetyl)quinolin-2(1H)-one

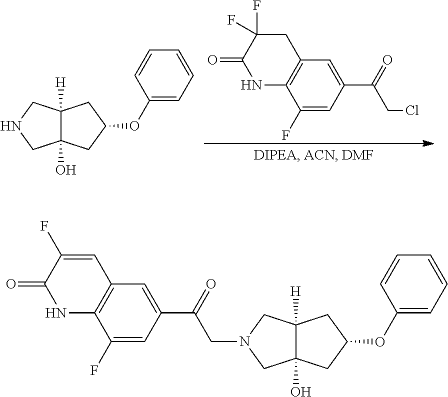

Using the same method as step 1 of Example 1A, starting from Intermediate 2 (260 mg, 1.19 mmol) and Intermediate 23 (300 mg, 1.08 mmol), provided the title intermediate (500 mg) which was used without further purification.

LCMS: Rt 0.74 min; MS m/z 441.2 [M+H]⁺; Method J.

Step 2: A mixture of:
3,8-difluoro-6-((R)-1-hydroxy-2-((3aS,5S,6aR)-3a-hydroxy-5-phenoxyhexahydrocyclopenta[c]pyrrol-2(1H)-yl)ethyl)quinolin-2(1H)-one
3,8-difluoro-6-((S)-1-hydroxy-2-((3aS,5S,6aR)-3a-hydroxy-5-phenoxyhexahydrocyclopenta[c]pyrrol-2(1H)-yl)ethyl)quinolin-2(1H)-one

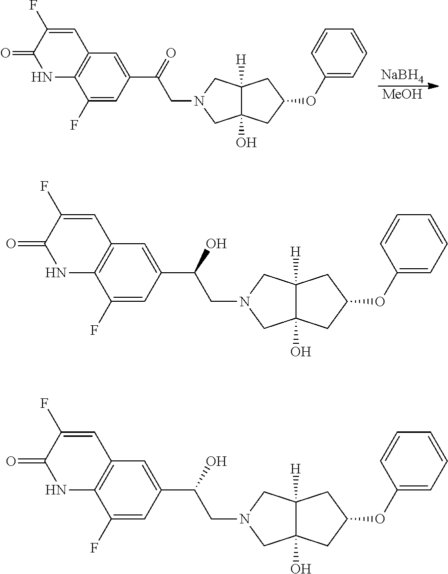

Using the same method as step 1 of Example 1 B, starting from 3,8-difluoro-6-(2-((3aS,5S,6aR)-3a-hydroxy-5-phenoxyhexahydrocyclopenta[c]pyrrol-2(1H)-yl)acetyl)quinolin-2(1H)-one (500 mg), provided a mixture of Examples 20A and 20B (100 mg).

LCMS: Rt 0.85 min; MS m/z 443.4 [M+H]⁺; Method I.

### Step 3: Chiral Separation

The two diastereomers were separated using the chiral SFC method below:

Column: Daicel Chiralpak IG (250 mm×30 mm, 10 μm), Flow: 70 g/min

Mobile phase: 50% IPA:ACN (1:1) with 0.1% NH₃·H₂O in Supercritical CO₂

Example 20A: 21 mg.

Analytical chiral SFC: Rt 1.05 min (Column: Chiralpak IG-3 (50×4.6 mm, 3 μm), Flow Rate: 3 mL/min, Mobile phase: 50% IPA:ACN (1:1) with 0.05% DEA in CO₂).

LCMS: Rt 0.84 min; MS m/z 443.3 [M+H]⁺; Method I.

¹H NMR (400 MHz, CDC₃) 5 9.46 (br s, 1H), 7.51-7.45 (m, 1H), 7.38-7.30 (m, 4H), 7.03-6.99 (m, 1H), 6.96-6.90 (m, 2H), 5.00 (br s, 1H), 4.79-4.71 (m, 1H), 3.93 (br s, 1H), 2.96-2.93 (m, 1H), 2.87-2.84 (m, 1H), 2.78-2.52 (m, 7H), 2.40-2.36 (m, 1H), 2.18-2.11 (m, 1H), 1.58-1.55 (m, 1H).

Example 20B: 20 mg.

Analytical chiral SFC: Rt 1.51 min (Column: Chiralpak IG-3 (50×4.6 mm, 3 μm), Flow Rate: 3 mL/min, Mobile phase: 50% IPA:ACN (1:1) with 0.05% DEA in CO₂).

LCMS: Rt 0.84 min; MS m/z 443.4 [M+H]⁺; Method I.

¹H NMR (400 MHz, CDC₃) 5 9.33 (br s, 1H), 7.48-7.45 (m, 1H), 7.37-7.28 (m, 4H), 7.01-6.97 (m, 1H), 6.96-6.86 (m, 2H), 4.98 (br s, 1H), 4.77-4.74 (m, 1H), 3.90 (br s, 1H), 3.20-3.17 (m, 1H), 2.98-2.87 (m, 1H), 2.72-2.47 (m, 6H), 2.44-2.34 (m, 2H), 2.11-2.06 (m, 1H), 1.61-1.60 (m, 1H).

### Example 21

A mixture of:
(3aS,5S,6aR)-5-(2,4-difluorophenoxy)-2-((R)-2-hydroxy-2-(1H-indazol-5-yl)ethyl)hexahydrocyclopenta[c]pyrrol-3a(1H)-ol
(3aS,5S,6aR)-5-(2,4-difluorophenoxy)-2-((S)-2-hydroxy-2-(1H-indazol-5-yl)ethyl)hexahydrocyclopenta[c]pyrrol-3a(1H)-ol
(3aR,5R,6aS)-5-(2,4-difluorophenoxy)-2-((R)-2-hydroxy-2-(1H-indazol-5-yl)ethyl)hexahydrocyclopenta[c]pyrrol-3a(1H)-ol
(3aR,5R,6aS)-5-(2,4-difluorophenoxy)-2-((R)-2-hydroxy-2-(1H-indazol-5-yl) ethyl) hexahydrocyclopenta[c]pyrrol-3a(1H)-ol

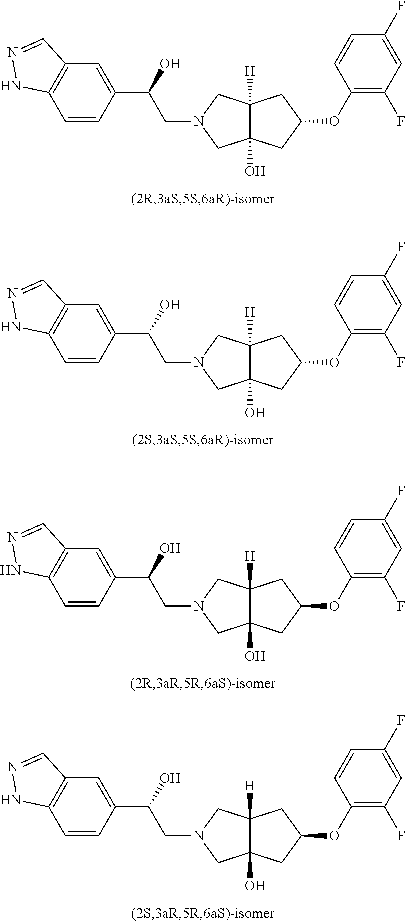 [SMILES: O[C@@H](CN1C[C@H]2C[C@H](Oc3ccc(F)cc3F)C[C@@]2(O)C1)c1ccc2[nH]ncc2c1 ; O[C@H](CN1C[C@H]2C[C@H](Oc3ccc(F)cc3F)C[C@@]2(O)C1)c1ccc2[nH]ncc2c1 ; O[C@@H](CN1C[C@@H]2C[C@@H](Oc3ccc(F)cc3F)C[C@]2(O)C1)c1ccc2[nH]ncc2c1 ; O[C@H](CN1C[C@@H]2C[C@@H](Oc3ccc(F)cc3F)C[C@]2(O)C1)c1ccc2[nH]ncc2c1]

Step 1: A mixture of:
2-((3aS,5S,6aR)-5-(2,4-difluorophenoxy)-3a-hydroxyhexahydrocyclopenta[c]pyrrol-2(1H)-yl)-1-(1-(tetrahydro-2H-pyran-2-yl)-1H-indazol-5-yl)ethan-1-one
2-((3aR,5R,6aS)-5-(2,4-difluorophenoxy)-3a-hydroxyhexahydrocyclopenta[c]pyrrol-2(1H)-yl)-1-(1-(tetrahydro-2H-pyran-2-yl)-1H-indazol-5-yl)ethan-1-one

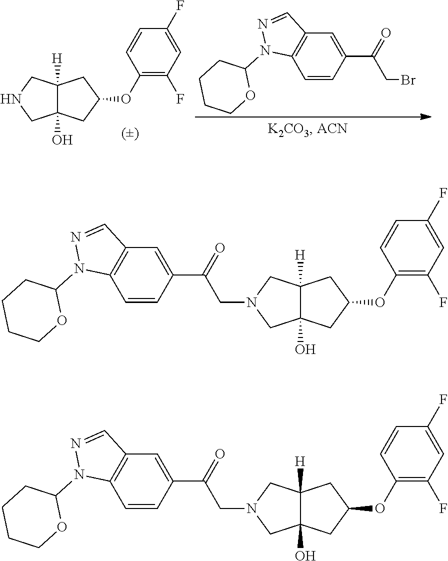

To a solution of Intermediate 10 (74 mg, 0.29 mmol) in ACN (2 mL) was added K₂CO₃ (120 mg, 0.87 mmol) and Intermediate 24 (76 mg, 0.23 mmol). This was stirred at RT for 3 h, then filtered, and the filtrate was concentrated to provide the title intermediates (112 mg) which were used without further purification.

LCMS: Rt 1.03 min; MS m/z 498.4 [M+H]⁺; Method H.

Step 2: A mixture of:
(3aS,5S,6aR)-5-(2,4-difluorophenoxy)-2-((2R)-2-hydroxy-2-(1-(tetrahydro-2H-pyran-2-yl)-1H-indazol-5-yl)ethyl)hexahydrocyclopenta[c]pyrrol-3a(1H)-ol
(3aS,5S,6aR)-5-(2,4-difluorophenoxy)-2-((2S)-2-hydroxy-2-(1-(tetrahydro-2H-pyran-2-yl)-1H-indazol-5-yl)ethyl)hexahydrocyclopenta[c]pyrrol-3a(1H)-ol
(3aR,5R,6aS)-5-(2,4-difluorophenoxy)-2-((2R)-2-hydroxy-2-(1-(tetrahydro-2H-pyran-2-yl)-1H-indazol-5-yl)ethyl)hexahydrocyclopenta[c]pyrrol-3a(1H)-ol
(3aR,5R,6aS)-5-(2,4-difluorophenoxy)-2-((2S)-2-hydroxy-2-(1-(tetrahydro-2H-pyran-2-yl)-1H-indazol-5-yl)ethyl)hexahydrocyclopenta[c]pyrrol-3a(1H)-ol

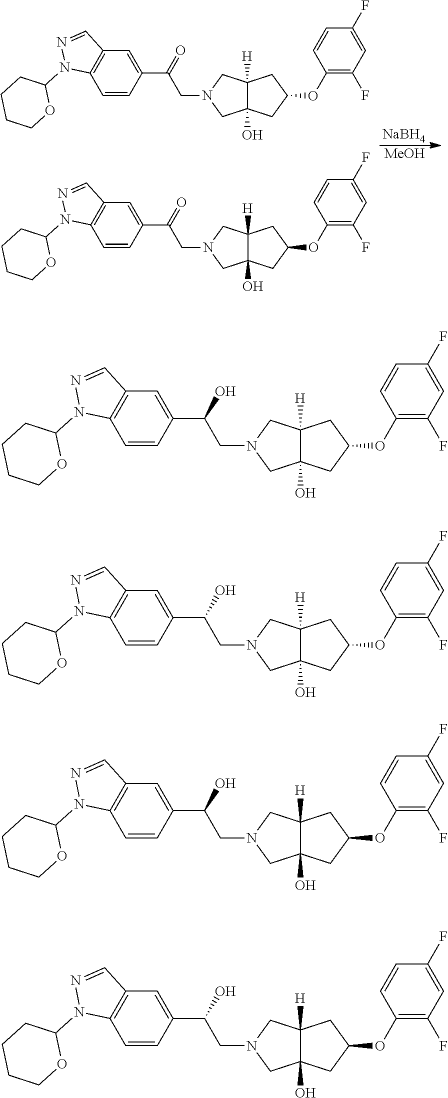

Using the same method as step 1 of Example 1 B, starting from the mixture of intermediates from the previous step (70 mg, 0.14 mmol), provided the title intermediates as a mixture (70 mg).

LCMS: Rt 1.00 min; MS m/z 500.4 [M+H]⁺; Method H.

Step 3: A mixture of:
(3aS,5S,6aR)-5-(2,4-difluorophenoxy)-2-((R)-2-hydroxy-2-(1H-indazol-5-yl)ethyl)hexahydrocyclopenta[c]pyrrol-3a(1H)-ol
(3aS,5S,6aR)-5-(2,4-difluorophenoxy)-2-((S)-2-hydroxy-2-(1H-indazol-5-yl)ethyl)hexahydrocyclopenta[c]pyrrol-3a(1H)-ol
(3aR,5R,6aS)-5-(2,4-difluorophenoxy)-2-((R)-2-hydroxy-2-(1H-indazol-5-yl)ethyl)hexahydrocyclopenta[c]pyrrol-3a(1H)-ol
(3aR,5R,6aS)-5-(2,4-difluorophenoxy)-2-((R)-2-hydroxy-2-(1H-indazol-5-yl)ethyl)hexahydrocyclopenta[c]pyrrol-3a(1H)-ol

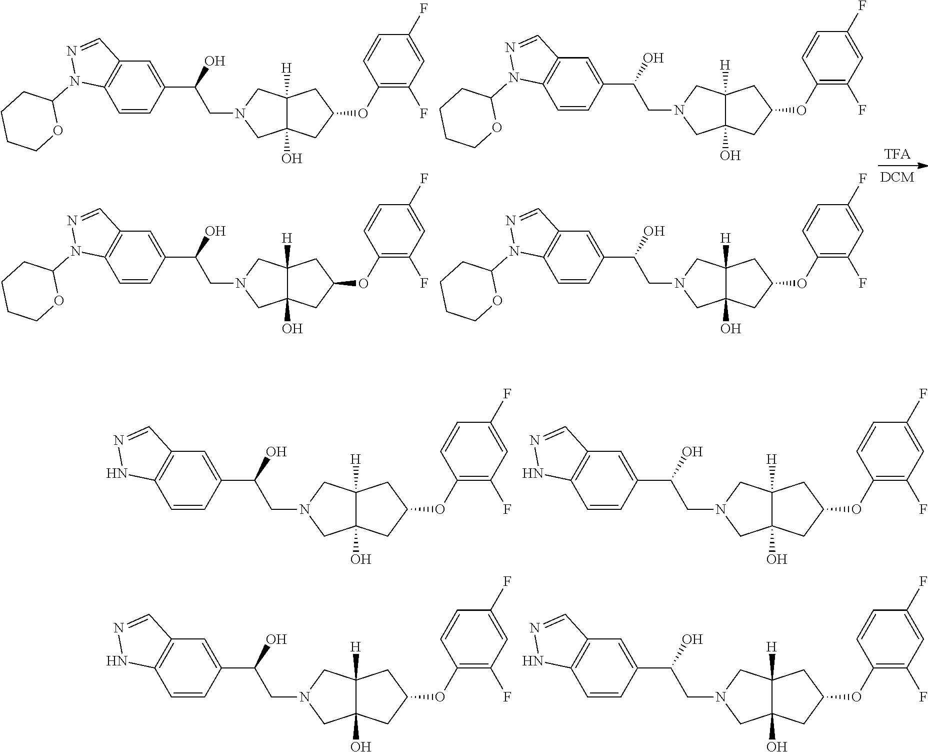

To a solution of the mixture of intermediates from the previous step (70 mg, 0.14 mmol) in DCM (1 mL) was added TFA (1 mL). This was stirred at RT for 2 h, then concentrated and purified by preparative HPLC (Waters Xbridge 5 μm, 30×50 mm, flow rate 75 mL/min, mobile phase A: water with 10 mM NH₄OH, B: acetonitrile with 10 mM NH₄OH, Gradient 25-50% B) to provide Example 21 as a mixture of four diastereomers (34 mg).

LCMS: Rt 1.08 min; MS m/z 416.0 [M+H]⁺; Method B.

¹H NMR (400 MHz, Methanol-d₄) δ 8.00 (t, J=0.8 Hz, 1H), 7.80-7.74 (m, 1H), 7.55-7.41 (m, 2H), 6.98-6.88 (m, 2H), 6.82-6.73 (m, 1H), 4.90-4.85 (m, 1H), 4.75-4.67 (m, 1H), 2.91-2.75 (m, 3H), 2.69-2.41 (m, 4H), 2.27-2.11 (m, 2H), 2.08-1.98 (m, 1H), 1.85-1.72 (m, 1H).

### Examples 22A, 22B, 22C and 22D

6-((R)-2-((3aS,5S,6aR)-5-(4-fluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aS,5S,6aR)-5-(4-fluorophenoxy)-3a-hydroxyhexahydrocyclopenta[c]pyrrol-2(1H)-yl)-1-hydroxyethyl)-3,4-dihydroquinolin-2(1H)-one
6-((R)-2-((3aR,5R,6aS)-5-(4-fluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aR,5R,6aS)-5-(4-fluorophenoxy)-3a-hydroxyhexahydrocyclopenta[c]pyrrol-2(1H)-yl)-1-hydroxyethyl)-3,4-dihydroquinolin-2(1H)-one

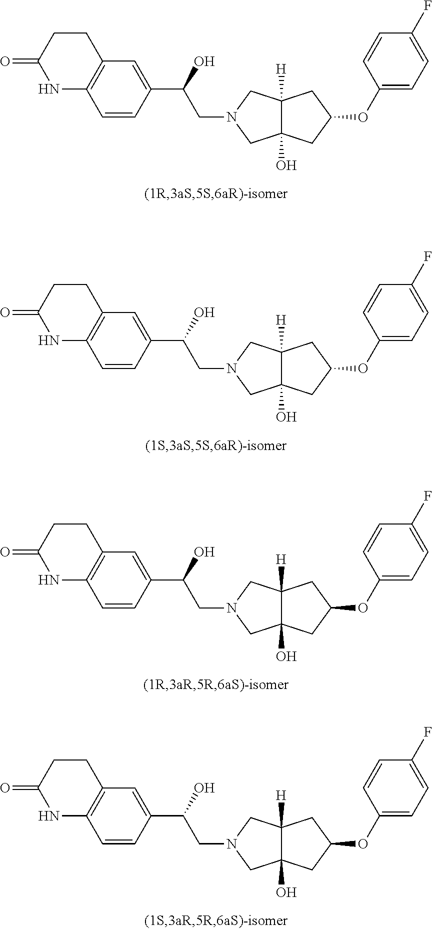 [SMILES: O=C1CCc2cc([C@@H](O)CN3C[C@H]4C[C@H](Oc5ccc(F)cc5)C[C@@]4(O)C3)ccc2N1 ; O=C1CCc2cc([C@H](O)CN3C[C@H]4C[C@H](Oc5ccc(F)cc5)C[C@@]4(O)C3)ccc2N1 ; O=C1CCc2cc([C@@H](O)CN3C[C@@H]4C[C@@H](Oc5ccc(F)cc5)C[C@]4(O)C3)ccc2N1 ; O=C1CCc2cc([C@H](O)CN3C[C@@H]4C[C@@H](Oc5ccc(F)cc5)C[C@]4(O)C3)ccc2N1]

Step 1: A racemic mixture of:
6-(2-((3aS,5S,6aR)-5-(4-fluorophenoxy)-3a-hydroxyhexahydrocyclopenta[c]pyrrol-2(1H)-yl)acetyl)-3,4-dihydroquinolin-2(1H)-one
6-(2-((3aR,5R,6aS)-5-(4-fluorophenoxy)-3a-hydroxyhexahydrocyclopenta[c]pyrrol-2(1H)-yl)acetyl)-3,4-dihydroquinolin-2(1H)-one

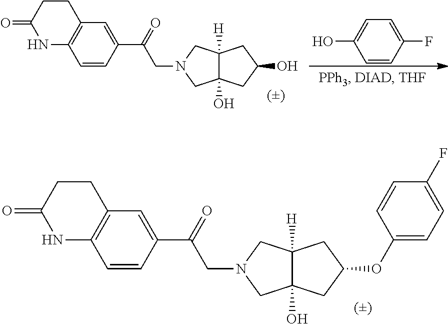 [SMILES: O=C1CCc2cc(C(=O)CN3C[C@H]4C[C@@H](O)C[C@@]4(O)C3)ccc2N1 ; Oc1ccc(F)cc1 ; O=C1CCc2cc(C(=O)CN3C[C@H]4C[C@H](Oc5ccc(F)cc5)C[C@@]4(O)C3)ccc2N1]

To a solution of PPh₃ (179 mg, 0.68 mmol) in THF (0.5 mL) under nitrogen at 0° C. was added DIAD (138 mg, 0.68 mmol), followed by a solution of Intermediate 40 (150 mg, 0.45 mmol) and 4-fluorophenol (76 mg, 0.68 mmol) in THF (1.0 mL). This was stirred at RT for 30 min, then diluted with water (5 mL), extracted with EtOAc (3×5 mL), washed with sat. brine (5 mL), dried with Na₂SO₄, filtered and concentrated. The crude material was purified by FCC (0-15% MeOH:DCM) to provide the title intermediate (140 mg).

LCMS: Rt 0.57 min; MS m/z 425.0 [M+H]⁺; Method J.

Step 2: A mixture of Examples 22A, 22B, 22C, and 22D

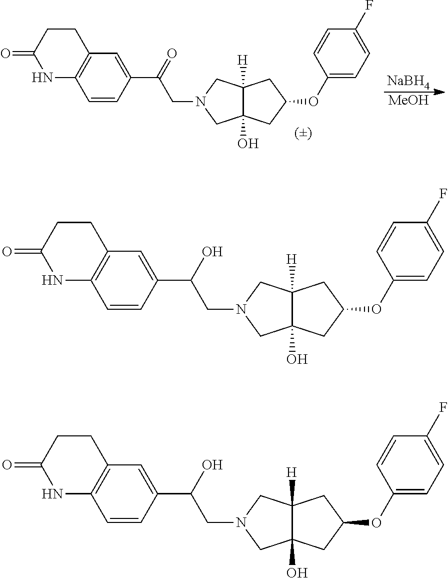

Using the same method as step 1 of Example 1 B, starting from the mixture of intermediates from the previous step (120 mg, 0.14 mmol), provided a mixture of Examples 22A, 22B, 22C and 22D (85 mg).

LCMS: Rt 0.87 min; MS m/z 427.3 [M+H]⁺; Method I.

### Step 3: Chiral separation of Examples 22A, 22B, 22C and 22D

The mixture was separated and the single isomers analyzed using the following chiral SFC methods:

Separation: Column: Daicel Chiralpak AD (250 mm×30 mm, 10 μm), Flow Rate: 80 g/min,

Mobile phase: 60% MeOH with 0.1% NH₃·H₂O in CO₂

Analytical: Column: Chiralpak AD-3 (50×4.6 mm, 3 μm), Flow Rate: 3 mL/min, Mobile

phase: 50% MeOH:ACN (1:1) with 0.05% DEA in CO₂

Example 22A (analytical chiral SFC Rt 0.74 min): 22 mg.

LCMS: Rt 0.87 min; MS m/z 427.3 [M+H]⁺; Method I.

¹H NMR (400 MHz, CDC₃) δ 7.88 (s, 1H), 7.23-7.14 (m, 2H), 7.03-6.95 (m, 2H), 6.89-6.82 (m, 2H), 6.73 (d, J=8.0 Hz, 1H), 4.88 (br s, 1H), 4.69-4.62 (m, 1H), 3.18 (d, J=9.2 Hz, 1H), 3.01-2.89 (m, 3H), 2.73-2.30 (m, 10H), 2.12-2.07 (m, 1H), 1.64-1.53 (m, 1H).

Example 22B (analytical chiral SFC Rt 1.01 min): 20 mg.

LCMS: Rt 0.87 min; MS m/z 427.3 [M+H]⁺; Method I.

¹H NMR (400 MHz, CDC₃) δ 7.99 (br s, 1H), 7.23-7.13 (m, 2H), 7.04-6.95 (m, 2H), 6.89-6.81 (m, 2H), 6.73 (d, J=8.0 Hz, 1H), 4.88 (br s, 1H), 4.73-4.58 (m, 1H), 3.01-2.90 (m, 3H), 2.85-2.73 (m, 2H), 2.70-2.42 (m, 8H), 2.35-2.31 (m, 1H), 2.16-2.11 (m, 1H), 1.60-1.53 (m, 1H).

Example 22C (analytical chiral SFC Rt 2.07 min): 20 mg.

LCMS: Rt 0.87 min; MS m/z 427.3 [M+H]⁺; Method I.

¹H NMR (400 MHz, CDCl₃) δ 8.02 (s, 1H), 7.23-7.14 (m, 2H), 7.03-6.95 (m, 2H), 6.89-6.82 (m, 2H), 6.74 (d, J=8.0 Hz, 1H), 4.88 (br s, 1H), 4.70-4.61 (m, 1H), 3.18 (d, J=9.2 Hz, 1H), 3.02-2.89 (m, 3H), 2.74-2.30 (m, 10H), 2.13-2.07 (m, 1H), 1.63-1.57 (m, 1H).

Example 22D (analytical chiral SFC Rt 2.73 min).

This compound was further purified by the following preparative HPLC method, providing 16 mg.

Column: Phenomenex Gemini NX-C18 (75 mm×30 mm), 3.0 μm

Mobile phase: 10 mM NH₄HCO₃ in water (A), Acetonitrile (B), Gradient 18-48% B over 8 min

LCMS: Rt 0.89 min; MS m/z 427.4 [M+H]⁺; Method I.

¹H NMR (400 MHz, CDCl₃) δ 8.10 (br s, 1H), 7.23-7.10 (m, 2H), 7.06-6.92 (m, 2H), 6.91-6.80 (m, 2H), 6.74 (d, J=8.0 Hz, 1H), 4.88 (br s, 1H), 4.71-4.57 (m, 1H), 3.02-2.90 (m, 3H), 2.86-2.55 (m, 8H), 2.52-2.45 (m, 2H), 2.35-2.31 (m, 1H), 2.16-2.10 (m, 1H), 1.60-1.52 (m, 1H).

### Examples 23A, 23B, 23C and 23D

6-((R)-2-((3aS,5S,6aR)-5-(3-fluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aS,5S,6aR)-5-(3-fluorophenoxy)-3a-hydroxyhexahydrocyclopenta[c]pyrrol-2(1H)-yl)-1-hydroxyethyl)-3,4-dihydroquinolin-2(1H)-one
6-((R)-2-((3aR,5R,6aS)-5-(3-fluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aR,5R,6aS)-5-(3-fluorophenoxy)-3a-hydroxyhexahydrocyclopenta[c]pyrrol-2(1H)-yl)-1-hydroxyethyl)-3,4-dihydroquinolin-2(1H)-one

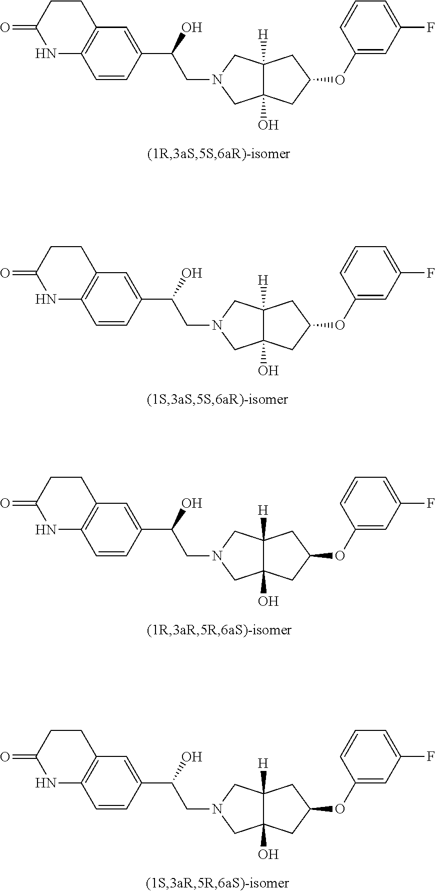 [SMILES: O=C1CCc2cc([C@@H](O)CN3C[C@H]4C[C@H](Oc5cccc(F)c5)C[C@@]4(O)C3)ccc2N1 ; O=C1CCc2cc([C@H](O)CN3C[C@H]4C[C@H](Oc5cccc(F)c5)C[C@@]4(O)C3)ccc2N1 ; O=C1CCc2cc([C@@H](O)CN3C[C@@H]4C[C@@H](Oc5cccc(F)c5)C[C@]4(O)C3)ccc2N1 ; O=C1CCc2cc([C@H](O)CN3C[C@@H]4C[C@@H](Oc5cccc(F)c5)C[C@]4(O)C3)ccc2N1]

Using the same methods as Examples 22A/22B/22C/22D, but using 3-fluorophenol instead of 4-fluorophenol in step 1, provided a mixture of Examples 23A/23B/23C/23D. The mixture was separated using the following chiral SFC method:

Column: Daicel Chiralpak AD (250 mm×30 mm, 10 μm), Flow Rate: 70 g/min, Mobile phase: 40% EtOH with 0.1% NH₃·H₂O in CO₂

This method separated Examples 23A and 23B from the other two isomers, which eluted together. The remaining two isomers were separated using the following chiral SFC method:

Column: Daicel Chiralpak IG (250 mm×30 mm, 10 μm), Flow Rate: 70 g/min, Mobile phase: 50% MeOH:ACN (1:1) with 0.1% NH₃·H₂O in CO₂

Analysis of the separated isomers was performed using the following analytical chiral SFC method:

Column: Chiralpak AD-3 (50×4.6 mm, 3 μm), Flow Rate: 3 mL/min, Mobile phase: 40% MeOH:ACN (1:1) with 0.05% DEA in CO₂

Example 23A (analytical chiral SFC Rt 1.26 min): 11 mg.

LCMS: Rt 0.87 min; MS m/z 427.2 [M+H]⁺; Method I.

¹H NMR (400 MHz, CDCl₃) δ 7.62 (s, 1H), 7.26-7.14 (m, 3H), 6.73-6.59 (m, 4H), 4.93 (br s, 1H), 4.72-4.65 (m, 1H), 3.23-3.18 (m, 1H), 3.03-2.91 (m, 3H), 2.78-2.41 (m, 9H), 2.33-2.15 (m, 1H), 2.19-2.10 (m, 1H), 1.70-1.64 (m, 1H).

Example 23B (analytical chiral SFC Rt 1.47 min): 11 mg.

LCMS: Rt 0.87 min; MS m/z 427.3 [M+H]⁺; Method I.

¹H NMR (400 MHz, CDC₃) δ 7.47-7.37 (m, 1H), 7.26-7.15 (m, 3H), 6.74-6.60 (m, 4H), 4.94 (br s, 1H), 4.69-4.61 (m, 1H), 3.03-2.72 (m, 5H), 2.71-2.46 (m, 8H), 2.39-2.30 (m, 1H), 2.20-2.12 (m, 1H), 1.65-1.59 (m, 1H).

Example 23C (analytical chiral SFC Rt 2.97 min): 13 mg.

LCMS: Rt 0.89 min; MS m/z 427.5 [M+H]⁺; Method I.

¹H NMR (400 MHz, CDCl₃) δ 7.55-7.45 (m, 1H), 7.26-7.13 (m, 3H), 6.74-6.58 (m, 4H), 4.94 (br s, 1H), 4.78-4.68 (m, 1H), 3.08-2.47 (m, 13H), 2.39-2.30 (m, 1H), 2.27-2.19 (m, 1H), 1.68-1.62 (m, 1H).

Example 23D (analytical chiral SFC Rt 3.38 min): 13 mg.

LCMS: Rt 0.89 min; MS m/z 427.5 [M+H]⁺; Method I.

¹H NMR (400 MHz, CDCl₃) δ 7.76-7.52 (m, 1H), 7.26-7.15 (m, 3H), 6.76-6.60 (m, 4H), 4.93 (br s, 1H), 4.72-4.65 (m, 1H), 3.20 (d, J=9.2 Hz, 1H), 3.01-2.92 (m, 3H), 2.77-2.29 (m, 10H), 2.17-2.10 (m, 1H), 1.69-1.60 (m, 1H).

### Examples 24A, 24B, 24C and 24D

6-((R)-2-((3aS,5S,6aR)-5-(2,3-difluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aS,5S,6aR)-5-(2,3-difluorophenoxy)-3a-hydroxyhexahydrocyclopenta[c]pyrrol-2(1H)-yl)-1-hydroxyethyl)-3,4-dihydroquinolin-2(1H)-one
6-((R)-2-((3aR,5R,6aS)-5-(2,3-difluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aR,5R,6aS)-5-(2,3-difluorophenoxy)-3a-hydroxyhexahydrocyclopenta[c]pyrrol-2(1H)-yl)-1-hydroxyethyl)-3,4-dihydroquinolin-2(1H)-one PGP-23C

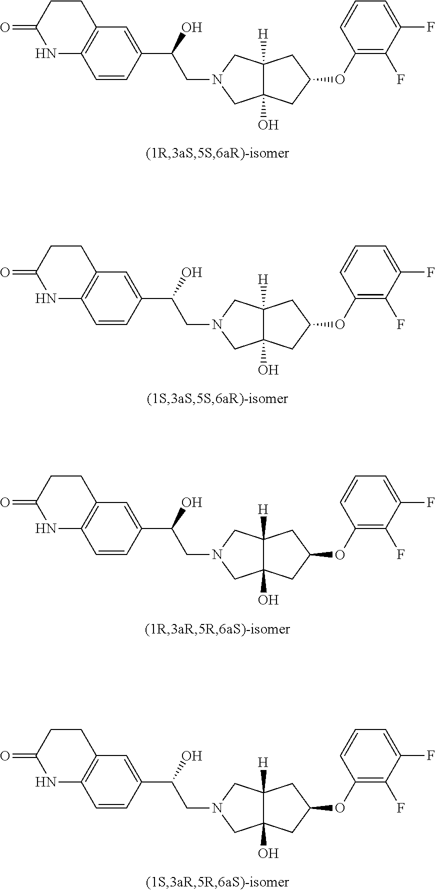 [SMILES: O=C1CCc2cc([C@@H](O)CN3C[C@H]4C[C@H](Oc5cccc(F)c5F)C[C@@]4(O)C3)ccc2N1 ; O=C1CCc2cc([C@H](O)CN3C[C@H]4C[C@H](Oc5cccc(F)c5F)C[C@@]4(O)C3)ccc2N1 ; O=C1CCc2cc([C@@H](O)CN3C[C@@H]4C[C@@H](Oc5cccc(F)c5F)C[C@]4(O)C3)ccc2N1 ; O=C1CCc2cc([C@H](O)CN3C[C@@H]4C[C@@H](Oc5cccc(F)c5F)C[C@]4(O)C3)ccc2N1]

Using the same methods as Examples 22A/22B/22C/22D, but using 2,3-difluorophenol instead of 4-fluorophenol in step 1, provided a mixture of Examples 24A/24B/24C/24D. The mixture was separated using the following chiral SFC method:

Column: Daicel Chiralpak AD (250 mm×30 mm, 10 μm), Flow Rate: 80 g/min, Mobile

phase: 60% MeOH with 0.1% NH₃·H₂O in CO₂

This method separated Examples 24C and 24D from the other two isomers, which eluted together. The remaining two isomers were separated using the following chiral SFC method:

Column: Daicel Chiralpak IG (250 mm×30 mm, 10 μm), Flow Rate: 70 g/min, Mobile phase: 60% MeOH:ACN (1:1) with 0.1% NH₃·H₂O in CO₂

Analysis of the separated isomers was performed using the following analytical chiral SFC method:

Column: Chiralpak IG-3 (50×4.6 mm, 3 μm), Flow Rate: 3 mL/min, Mobile phase: 50% EtOH with 0.05% DEA in CO₂

Example 24A (analytical chiral SFC Rt 1.05 min): 13 mg.

LCMS: Rt 0.91 min; MS m/z 445.4 [M+H]⁺; Method I.

¹H NMR (400 MHz, CDC₃) δ 7.81-7.62 (m, 1H), 7.23-7.14 (m, 2H), 7.06-6.97 (m, 1H), 6.89-6.69 (m, 3H), 5.02 (br s, 1H), 4.69-4.62 (m, 1H), 3.23 (d, J=8.6 Hz, 1H), 3.02-2.88 (m, 3H), 2.81-2.60 (m, 5H), 2.58-2.36 (m, 5H), 2.14-2.06 (m, 1H), 1.65-1.56 (m, 1H).

Example 24B (analytical chiral SFC Rt 1.18 min): 15 mg.

LCMS: Rt 0.91 min; MS m/z 445.5 [M+H]⁺; Method I.

¹H NMR (400 MHz, CDC₃) δ 7.59 (br s, 1H), 7.22-7.14 (m, 2H), 7.05-6.95 (m, 1H), 6.88-6.76 (m, 2H), 6.71 (d, J=8.0 Hz, 1H), 5.02 (br s, 1H), 4.68-4.63 (m, 1H), 3.03-2.95 (m, 3H), 2.91-2.36 (m, 12H), 2.20-2.12 (m, 1H), 1.63-1.53 (m, 1H).

Example 24C (analytical chiral SFC Rt 3.05 min): 17 mg.

LCMS: Rt 0.91 min; MS m/z 445.4 [M+H]⁺; Method I.

¹H NMR (400 MHz, CDC₃) δ 7.92 (s, 1H), 7.22-7.13 (m, 2H), 7.06-6.95 (m, 1H), 6.87-6.76 (m, 2H), 6.74-6.71 (m, 1H), 5.01 (br s, 1H), 4.68-4.63 (m, 1H), 3.88-3.59 (br s, 1H), 3.22 (d, J=9.2 Hz, 1H), 3.03-2.88 (m, 3H), 2.84-2.59 (m, 5H), 2.57-2.35 (m, 5H), 2.14-2.06 (m, 1H), 1.63-1.55 (m, 1H).

Example 24D (analytical chiral SFC Rt 1.73 min): 13 mg.

LCMS: Rt 0.91 min; MS m/z 445.4 [M+H]⁺; Method I.

¹H NMR (400 MHz, CDCl₃) δ 7.88 (s, 1H), 7.23-7.14 (m, 2H), 7.05-6.96 (m, 1H), 6.88-6.70 (m, 3H), 5.01 (br s, 1H), 4.69-4.62 (m, 1H), 3.88-3.59 (br s, 1H), 3.22 (d, J=9.2 Hz, 1H), 3.02-2.87 (m, 3H), 2.83-2.58 (m, 5H), 2.57-2.35 (m, 5H), 2.13-2.06 (m, 1H), 1.63-1.56 (m, 1H).

### Examples 25A, 25B, 25C and 25D

6-((R)-2-((3aS,5S,6aR)-5-(2,4-difluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aS,5S,6aR)-5-(2,4-difluorophenoxy)-3a-hydroxyhexahydrocyclopenta[c]pyrrol-2(1H)-yl)-1-hydroxyethyl)-3,4-dihydroquinolin-2(1H)-one
6-((R)-2-((3aR,5R,6aS)-5-(2,4-difluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aR,5R,6aS)-5-(2,4-difluorophenoxy)-3a-hydroxyhexahydrocyclopenta[c]pyrrol-2(1H)-yl)-1-hydroxyethyl)-3,4-dihydroquinolin-2(1H)-one

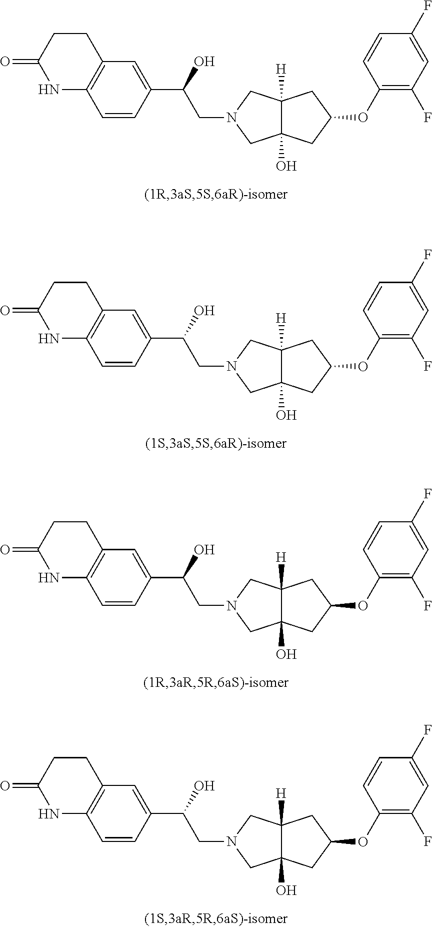 [SMILES: O=C1CCc2cc([C@@H](O)CN3C[C@H]4C[C@H](Oc5ccc(F)cc5F)C[C@@]4(O)C3)ccc2N1 ; O=C1CCc2cc([C@H](O)CN3C[C@H]4C[C@H](Oc5ccc(F)cc5F)C[C@@]4(O)C3)ccc2N1 ; O=C1CCc2cc([C@@H](O)CN3C[C@@H]4C[C@@H](Oc5ccc(F)cc5F)C[C@]4(O)C3)ccc2N1 ; O=C1CCc2cc([C@H](O)CN3C[C@@H]4C[C@@H](Oc5ccc(F)cc5F)C[C@]4(O)C3)ccc2N1]

Using the same methods as Examples 22A/22B/22C/22D, but using 2,4-difluorophenol instead of 4-fluorophenol in step 1, provided a mixture of Examples 25A/25B/25C/25D. The mixture was separated using the following chiral SFC method: Column: Daicel Chiralpak AD (250 mm×30 mm, 10 μm), Flow Rate: 70 g/min, Mobile phase: 50% EtOH with 0.1% NH₃·H₂O in CO₂

This method separated the four isomers into two peaks, each containing two isomers.

The first peak was separated using the following chiral SFC method to provide Examples 25A and 25B: Column: Daicel Chiralpak IG (250 mm×30 mm, 10 μm), Flow Rate: 70 g/min, Mobile phase: 50% MeOH:ACN (1:1) with 0.1% NH₃·H₂O in CO₂

The second peak was separated using the following chiral SFC method to provide Examples 25C and 25D: Column: Daicel Chiralpak IG (250 mm×30 mm, 10 μm), Flow Rate: 70 g/min, Mobile phase: 50% EtOH:ACN (1:1) with 0.1% NH₃·H₂O in CO₂

Analysis of the separated isomers was performed using the following analytical chiral SFC method:

Column: Chiralpak AD-3 (50×4.6 mm, 3 μm), Flow Rate: 3 mL/min, Mobile phase: 40% EtOH with 0.05% DEA in CO₂

Example 25A (analytical chiral SFC Rt 1.03 min): 12 mg.

LCMS: Rt 0.90 min; MS m/z 445.3 [M+H]⁺; Method I.

¹H NMR (400 MHz, CDC₃) δ 7.95 (br s, 1H), 7.23-7.13 (m, 2H), 7.03-6.94 (m, 1H), 6.92-6.78 (m, 2H), 6.73 (d, J=8.0 Hz, 1H), 4.92 (br s, 1H), 4.66-4.57 (m, 1H), 3.01-2.71 (m, 6H), 2.67-2.57 (m, 5H), 2.51-2.43 (m, 2H), 2.41-2.33 (m, 1H), 2.14-2.07 (m, 1H), 1.55-1.47 (m, 1H).

Example 25B (analytical chiral SFC Rt 0.90 min): 14 mg.

LCMS: Rt 0.90 min; MS m/z 445.3 [M+H]⁺; Method I.

¹H NMR (400 MHz, CDC₃) 5 8.21-8.08 (m, 1H), 7.22-7.13 (m, 2H), 7.02-6.94 (m, 1H), 6.92-6.79 (m, 2H), 6.74 (d, J=8.0 Hz, 1H), 4.91 (br s, 1H), 4.70-4.60 (m, 1H), 3.22-3.15 (m, 1H), 3.01-2.85 (m, 3H), 2.74-2.30 (m, 10H), 2.10-2.02 (m, 1H), 1.59-1.50 (m, 1H).

Example 25C (analytical chiral SFC Rt 1.45 min): 13 mg.

LCMS: Rt 0.90 min; MS m/z 445.3 [M+H]⁺; Method I.

¹H NMR (400 MHz, CDC₃) 5 8.08 (br s, 1H), 7.22-7.13 (m, 2H), 7.02-6.94 (m, 1H), 6.92-6.79 (m, 2H), 6.74 (d, J=8.0 Hz, 1H), 4.92 (br s, 1H), 4.68-4.59 (m, 1H), 3.00-2.73 (m, 6H), 2.67-2.58 (m, 5H), 2.52-2.43 (m, 2H), 2.39-2.34 (m, 1H), 2.13-2.07 (m, 1H), 1.55-1.48 (m, 1H).

Example 25D (analytical chiral SFC Rt 1.30 min): 17 mg.

LCMS: Rt 0.90 min; MS m/z 445.3 [M+H]⁺; Method I.

¹H NMR (400 MHz, CDC₃) δ 7.90 (s, 1H), 7.23-7.11 (m, 2H), 7.03-6.94 (m, 1H), 6.93-6.78 (m, 2H), 6.73 (d, J=8.0 Hz, 1H), 4.92 (br s, 1H), 4.72-4.59 (m, 1H), 3.22-3.16 (m, 1H), 3.00-2.88 (m, 3H), 2.74-2.29 (m, 10H), 2.10-2.03 (m, 1H), 1.58-1.52 (m, 1H).

### Examples 26A, 26B, 26C and 26D

6-((R)-2-((3aS,5S,6aR)-5-(2,5-difluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aS,5S,6aR)-5-(2,5-difluorophenoxy)-3a-hydroxyhexahydrocyclopenta[c]pyrrol-2(1H)-yl)-1-hydroxyethyl)-3,4-dihydroquinolin-2(1H)-one
6-((R)-2-((3aR,5R,6aS)-5-(2,5-difluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aR,5R,6aS)-5-(2,5-difluorophenoxy)-3a-hydroxyhexahydrocyclopenta[c]pyrrol-2(1H)-yl)-1-hydroxyethyl)-3,4-dihydroquinolin-2(1H)-one

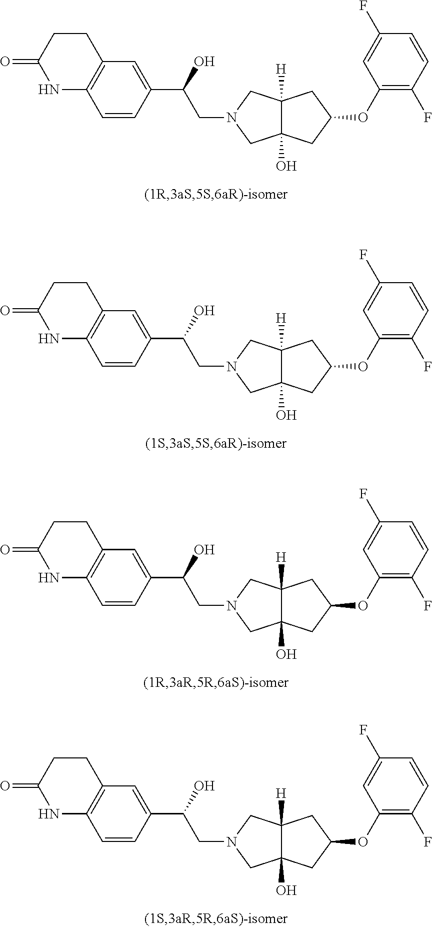 [SMILES: O=C1CCc2cc([C@@H](O)CN3C[C@H]4C[C@H](Oc5cc(F)ccc5F)C[C@@]4(O)C3)ccc2N1 ; O=C1CCc2cc([C@H](O)CN3C[C@H]4C[C@H](Oc5cc(F)ccc5F)C[C@@]4(O)C3)ccc2N1 ; O=C1CCc2cc([C@@H](O)CN3C[C@@H]4C[C@@H](Oc5cc(F)ccc5F)C[C@]4(O)C3)ccc2N1 ; O=C1CCc2cc([C@H](O)CN3C[C@@H]4C[C@@H](Oc5cc(F)ccc5F)C[C@]4(O)C3)ccc2N1]

Using the same methods as Examples 22A/22B/22C/22D, but using 2,5-difluorophenol instead of 4-fluorophenol in step 1, provided a mixture of Examples 26A/26B/26C/26D. The mixture was separated using the following chiral SFC method:

Column: Daicel Chiralpak AD (250 mm×30 mm, 10 μm), Flow Rate: 80 g/min, Mobile phase: 50% EtOH with 0.1% NH₃·H₂O in CO₂

This method separated the four isomers into two peaks, each containing two isomers.

The first peak was separated using the following chiral SFC method to provide Examples 26A and 26B: Column: Daicel Chiralpak IG (250 mm×30 mm, 10 μm), Flow Rate: 70 g/min, Mobile phase: 50% EtOH with 0.1% NH₃·H₂O in CO₂

The second peak was separated using the following chiral SFC method to provide Examples 26C and 26D: Column: Daicel Chiralpak IG (250 mm×30 mm, 10 μm), Flow Rate: 80 g/min, Mobile phase: 70% EtOH with 0.1% NH₃·H₂O in CO₂

Analysis of the separated isomers was performed using the following analytical chiral SFC method:

Column: Chiralpak AD-3 (50×4.6 mm, 3 μm), Flow Rate: 3 mL/min, Mobile phase: 40% EtOH with 0.05% DEA in CO₂

Example 26A (analytical chiral SFC Rt 0.94 min).

This compound was further purified by the following preparative HPLC method, providing 12 mg.

Column: Waters Xbridge (150 mm×25 mm), 5 μm

Mobile phase: 10 mM NH₄HCO₃ in water (A), Acetonitrile (B), Gradient 27-57% B over 10 min

LCMS: Rt 0.90 min; MS m/z 445.3 [M+H]⁺; Method I.

¹H NMR (400 MHz, CDC₃) δ 7.71 (s, 1H), 7.23-7.14 (m, 2H), 7.10-6.99 (m, 1H), 6.78-6.70 (m, 2H), 6.69-6.60 (m, 1H), 4.97 (br s, 1H), 4.69-4.62 (m, 1H), 3.70 (br s, 1H), 3.21 (d, J=8.8 Hz, 1H), 3.01-2.88 (m, 3H), 2.79-2.59 (m, 5H), 2.57-2.46 (m, 3H), 2.42-2.34 (m, 2H), 2.13-2.05 (m, 1H), 1.63-1.59 (m, 1H).

Example 26B (analytical chiral SFC Rt 1.05 min): 14 mg.

LCMS: Rt 0.89 min; MS m/z 445.3 [M+H]⁺; Method I.

¹H NMR (400 MHz, CDC₃) δ 7.75 (s, 1H), 7.23-7.14 (m, 2H), 7.09-7.00 (m, 1H), 6.79-6.69 (m, 2H), 6.69-6.61 (m, 1H), 4.97 (br s, 1H), 4.68-4.60 (m, 1H), 3.02-2.91 (m, 3H), 2.90-2.71 (m, 3H), 2.67-2.57 (m, 5H), 2.54-2.47 (m, 2H), 2.40-2.36 (m, 1H), 2.17-2.08 (m, 1H), 1.60-1.55 (m, 1H).

Example 26C (analytical chiral SFC Rt 1.46 min): 13 mg.

LCMS: Rt 0.90 min; MS m/z 445.3 [M+H]⁺; Method I.

¹H NMR (400 MHz, CDC₃) δ 7.84 (s, 1H), 7.25-7.15 (m, 2H), 7.12-7.00 (m, 1H), 6.82-6.71 (m, 2H), 6.70-6.62 (m, 1H), 4.99 (br s, 1H), 4.73-4.58 (m, 1H), 3.04-2.93 (m, 3H), 2.91-2.74 (m, 3H), 2.69-2.58 (m, 5H), 2.56-2.49 (m, 2H), 2.42-2.35 (m, 1H), 2.19-2.10 (m, 1H), 1.62-1.56 (m, 1H).

Example 26D (analytical chiral SFC Rt 1.62 min): 13 mg.

LCMS: Rt 0.90 min; MS m/z 445.3 [M+H]⁺; Method I.

¹H NMR (400 MHz, CDCl₃) δ 7.79 (s, 1H), 7.23-7.14 (m, 2H), 7.09-7.00 (m, 1H), 6.79-6.68 (m, 2H), 6.68-6.61 (m, 1H), 4.97 (br s, 1H), 4.71-4.59 (m, 1H), 3.21 (d, J=9.2 Hz, 1H), 3.02-2.88 (m, 3H), 2.74-2.33 (m, 10H), 2.14-2.07 (m, 1H), 1.63-1.58 (m, 1H).

### Examples 27A, 27B, 27C and 27D

6-((R)-2-((3aS,5S,6aR)-5-(2,6-difluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aS,5S,6aR)-5-(2,6-difluorophenoxy)-3a-hydroxyhexahydrocyclopenta[c]pyrrol-2(1H)-yl)-1-hydroxyethyl)-3,4-dihydroquinolin-2(1H)-one
6-((R)-2-((3aR,5R,6aS)-5-(2,6-difluorophenoxy)-3a-hydroxyhexahydrocyclopenta[c]pyrrol-2(1H)-yl)-1-hydroxyethyl)-3,4-dihydroquinolin-2(1H)-one
6-((S)-2-((3aR,5R,6aS)-5-(2,6-difluorophenoxy)-3a-hydroxyhexahydrocyclopenta[c]pyrrol-2(1H)-yl)-1-hydroxyethyl)-3,4-dihydroquinolin-2(1H)-one

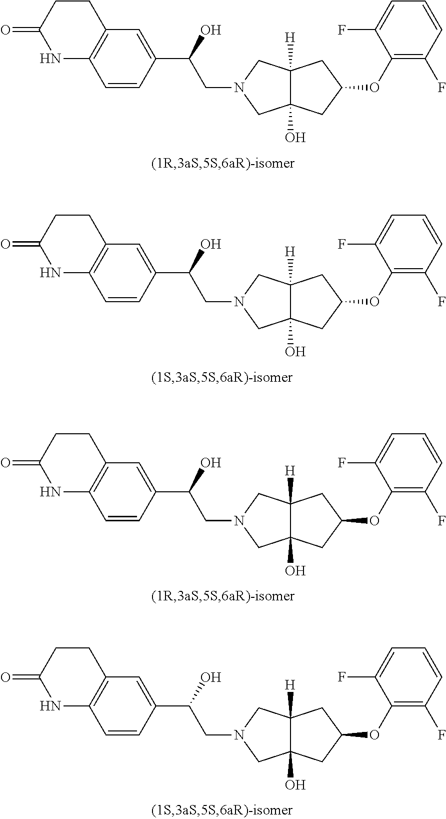 [SMILES: O=C1CCc2cc([C@@H](O)CN3C[C@H]4C[C@H](Oc5c(F)cccc5F)C[C@@]4(O)C3)ccc2N1 ; O=C1CCc2cc([C@@H](O)CN3C[C@H]4C[C@H](Oc5c(F)cccc5F)C[C@@]4(O)C3)ccc2N1 ; O=C1CCc2cc([C@@H](O)CN3C[C@@H]4C[C@@H](Oc5c(F)cccc5F)C[C@]4(O)C3)ccc2N1 ; O=C1CCc2cc([C@H](O)CN3C[C@@H]4C[C@@H](Oc5c(F)cccc5F)C[C@]4(O)C3)ccc2N1]

Using the same methods as Examples 22A/22B/22C/22D, but using 2,6-difluorophenol instead of 4-fluorophenol in step 1, provided a mixture of Examples 27A/27B/27C/27D. The mixture was separated using the following chiral SFC method:

Column: Daicel Chiralpak AD (250 mm×30 mm, 10 μm), Flow Rate: 80 g/min, Mobile phase: 50% EtOH with 0.1% NH₃·H₂O in CO₂

This method separated the four isomers into two peaks, each containing two isomers.

The first peak was separated using the following chiral SFC method to provide Examples 27A and 27B: Column: Daicel Chiralpak IG (250 mm×30 mm, 10 μm), Flow Rate: 70 g/min, Mobile phase: 60% MeOH with 0.1% NH₃·H₂O in CO₂

The second peak was separated using the following chiral SFC method to provide Examples 27C and 27D: Column: Daicel Chiralpak IG (250 mm×30 mm, 10 μm), Flow Rate: 80 g/min, Mobile phase: 60% EtOH:ACN (1:1) with 0.1% NH₃·H₂O in CO₂

Analysis of the separated isomers was performed using the following analytical chiral SFC method:

Column: Chiralpak IG-3 (50×4.6 mm, 3 μm), Flow Rate: 3 mL/min, Mobile phase: 60% EtOH with 0.05% DEA in CO₂

Example 27A (analytical chiral SFC Rt 0.74 min): 15 mg.

LCMS: Rt 0.90 min; MS m/z 445.5 [M+H]⁺; Method I.

¹H NMR (400 MHz, CDC₃) δ 7.88 (s, 1H), 7.23-7.12 (m, 2H), 7.05-6.89 (m, 3H), 6.73 (d, J=8.0 Hz, 1H), 5.03 (br s, 1H), 4.67-4.64 (m, 1H), 3.23-3.21 (m, 1H), 3.05-2.82 (m, 4H), 2.74-2.58 (m, 5H), 2.55-2.35 (m, 4H), 2.10-2.05 (m, 1H), 1.55-1.52 (m, 1H).

Example 27B (analytical chiral SFC Rt 0.89 min): 10 mg.

LCMS: Rt 0.90 min; MS m/z 445.4 [M+H]⁺; Method I.

¹H NMR (400 MHz, CDC₃) δ 7.87-7.74 (m, 1H), 7.23-7.12 (m, 2H), 7.06-6.91 (m, 3H), 6.73-6.71 (m, 1H), 5.04 (br s, 1H), 4.64-4.61 (m, 1H), 3.06-2.89 (m, 4H), 2.86-2.80 (m, 1H), 2.79-2.59 (m, 6H), 2.51-2.39 (m, 3H), 2.09-2.06 (m, 1H), 1.51-1.45 (m, 1H).

Example 27C (analytical chiral SFC Rt 1.09 min): 15 mg.

LCMS: Rt 0.90 min; MS m/z 445.5 [M+H]⁺; Method I.

¹H NMR (400 MHz, CDC₃) 5 8.03 (br s, 1H), 7.22-7.13 (m, 2H), 7.06-6.88 (m, 3H), 6.74-6.72 (m, 1H), 5.03 (br s, 1H), 4.64-4.61 (m, 1H), 3.08-2.89 (m, 4H), 2.85-2.77 (m, 1H), 2.79-2.59 (m, 6H), 2.52-2.38 (m, 3H), 2.09-2.05 (m, 1H), 1.51-1.44 (m, 1H).

Example 27D (analytical chiral SFC Rt 1.99 min): 15 mg.

LCMS: Rt 0.90 min; MS m/z 445.4 [M+H]⁺; Method I.

¹H NMR (400 MHz, CDC₃) δ 7.75 (s, 1H), 7.16-7.01 (m, 2H), 6.98-6.79 (m, 3H), 6.64 (d, J=8.2 Hz, 1H), 4.95 (br s, 1H), 4.58-4.55 (m, 1H), 4.04-3.23 (m, 1H), 3.15-3.13 (m, 1H), 2.96-2.75 (m, 4H), 2.66-2.26 (m, 9H), 1.97-1.93 (m, 1H), 1.46-1.38 (m, 1H).

### Examples 28A and 28B

6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one
6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one

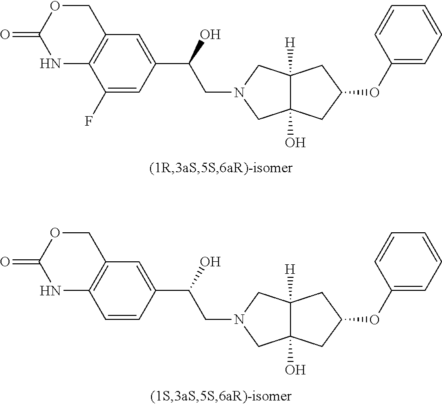 [SMILES: O=C1Nc2c(F)cc([C@@H](O)CN3C[C@H]4C[C@H](Oc5ccccc5)C[C@@]4(O)C3)cc2CO1 ; O=C1Nc2ccc([C@H](O)CN3C[C@H]4C[C@H](Oc5ccccc5)C[C@@]4(O)C3)cc2CO1]

Step 1: A mixture of:
6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one
6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one

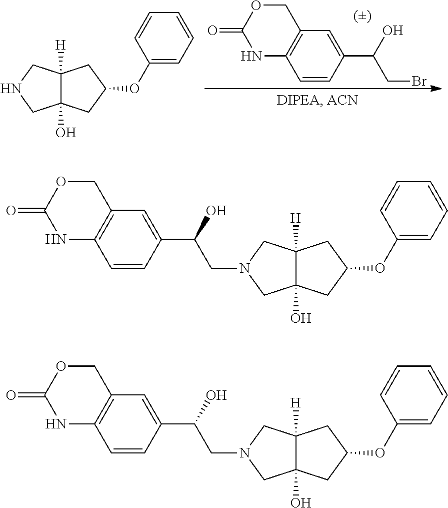

To a solution of Intermediate 2 (80 mg, 0.36 mmol) and Intermediate 25 (190 mg, 0.70 mmol) in ACN (2 mL) was added DIPEA (170 mg, 0.23 mL, 1.31 mmol) and this was stirred at 40° C. for 16 h. The reaction was filtered and the filtrate was purified by preparative HPLC (column: Waters Xbridge (150×25 mm×5 μm); mobile phase: Water with 0.05% NH₄HCO₃ v/v (A); ACN (B); 5-50% B over 10 min; Flow rate: 25 mL/min) to provide a mixture of Examples 28A and 28B and two undesired regioisomers.

LCMS: Rt 0.88 min; MS m/z 411.4 [M+H]⁺; Method I.

Step 2: Chiral separation of Examples 28A and 28B

The mixture was separated using the following chiral SFC method:

Column: Daicel Chiralpak AD (250 mm×30 mm, 10 μm), Flow Rate: 70 g/min, Mobile phase: 60% EtOH with 0.1% NH₃·H₂O in CO₂

This method gave, in order, an undesired regioisomer, then Example 28A, then a mixture of Example 28B and another undesired regioisomer.

The remaining mixture was separated using the following chiral SFC method: Column: Daicel Chiralpak OJ-H (250 mm×30 mm, 5 μm), Flow Rate: 65 g/min, Mobile phase: 35% MeOH with 0.1% NH₃·H₂O in CO₂

Analysis of the separated isomers was performed using the following analytical chiral SFC method:

Column: Chiralpak AD-3 (50×4.6 mm, 3 μm), Flow Rate: 3 mL/min, Mobile phase: 50% EtOH with 0.05% DEA in CO₂

Example 28A (analytical chiral SFC Rt 1.30 min): 15 mg.

LCMS: Rt 0.87 min; MS m/z 411.4 [M+H]⁺; Method I.

¹H NMR (400 MHz, CDCl₃) δ 7.86-7.81 (m, 1H), 7.33-7.28 (m, 2H), 7.24 (s, 1H), 7.18 (s, 1H), 7.01-6.96 (m, 1H), 6.92-6.85 (m, 2H), 6.80-6.78 (m, 1H), 5.32 (s, 2H), 4.96 (br s, 1H), 4.70-4.66 (m, 1H), 3.19-3.16 (m, 1H), 2.95-2.90 (m, 1H), 2.67-2.46 (m, 6H), 2.41-2.33 (m, 2H), 2.12-2.07 (m, 1H), 1.61-1.59 (m, 2H).

Example 28B (analytical chiral SFC Rt 1.91 min): 15 mg.

LCMS: Rt 0.88 min; MS m/z 411.2 [M+H]⁺; Method I.

¹H NMR (400 MHz, Methanol-d₄) δ 7.35-7.33 (m, 1H), 7.27-7.22 (m, 3H), 6.92-6.88 (m, 4H), 5.34-5.26 (m, 2H), 4.86 (br s, 2H), 3.09-2.90 (m, 7H), 2.75-2.64 (m, 1H), 2.43-2.37 (m, 1H), 2.23-2.15 (m, 1H), 1.93-1.87 (m, 1H).

### Examples 29A and 29B

8-fluoro-6-((R)-2-((3aS,4S,5S,6aR)-5-(2-fluorophenoxy)-3a,4-dihydroxyhexahydrocyclopenta[c]pyrrol-2(1H)-yl)-1-hydroxyethyl)-3,4-dihydroquinolin-2(1H)-one
8-fluoro-6-((S)-2-((3aS,4S,5S,6aR)-5-(2-fluorophenoxy)-3a,4-dihydroxyhexahydrocyclopenta[c]pyrrol-2(1H)-yl)-1-hydroxyethyl)-3,4-dihydroquinolin-2(1H)-one

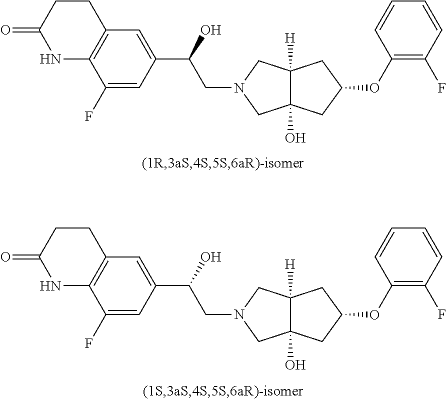 [SMILES: O=C1CCc2cc([C@@H](O)CN3C[C@H]4C[C@H](Oc5ccccc5F)C[C@@]4(O)C3)cc(F)c2N1 ; O=C1CCc2cc([C@H](O)CN3C[C@H]4C[C@H](Oc5ccccc5F)C[C@@]4(O)C3)cc(F)c2N1]

Using the same method as Examples 28A/28B, starting from Intermediates 8 and 26, a mixture of Examples 29A and 29B and two undesired regioisomers was obtained. The mixture was separated using the following chiral SFC method:

Column: Daicel Chiralpak IG (250 mm×30 mm, 10 μm), Flow Rate: 80 g/min

Mobile Phase: 60% EtOH with 0.1% NH₃·H₂O in CO₂

This method gave, in order, an undesired regioisomer, then a mixture of Example 29A and another undesired regioisomer, then Example 29B.

The remaining mixture was separated using the following chiral SFC method:

Column: Daicel Chiralcel OJ (250 mm×30 mm, 10 μm), Flow Rate: 70 g/min

Mobile Phase: 40% MeOH with 0.1% NH₃·H₂O in CO₂

Analysis of the separated isomers was performed using the following analytical chiral SFC method:

Column: Chiralpak IG-3 (50×4.6 mm, 3 μm), Flow Rate: 3 mL/min, Mobile phase: 50% EtOH with 0.05% DEA in CO₂

Example 29A (analytical chiral SFC Rt 2.29 min): 18 mg.

LCMS: Rt 0.84 min; MS m/z 461.4 [M+H]⁺; Method I.

¹H NMR (400 MHz, CDC₃) δ 7.60 (s, 1H), 7.15-6.96 (m, 6H), 4.86-4.81 (m, 1H), 4.67-4.59 (m, 1H), 4.03-4.02 (m, 1H), 3.05-2.96 (m, 4H), 2.86-2.84 (m, 1H), 2.76-2.70 (m, 1H), 2.68-2.60 (m, 4H), 2.59-2.56 (m, 1H), 2.54-2.48 (m, 1H), 2.43-2.33 (m, 1H), 1.54-1.48 (m, 1H).

Example 29B (analytical chiral SFC Rt 3.40 min): 20 mg.

LCMS: Rt 0.85 min; MS m/z 461.4 [M+H]⁺; Method I.

¹H NMR (400 MHz, CDC₃) δ 7.60 (s, 1H), 7.17-6.92 (m, 6H), 4.84-4.81 (m, 1H), 4.66-4.62 (m, 1H), 3.99-3.98 (m, 1H), 3.69-3.45 (m, 1H), 3.26-3.24 (m, 1H), 3.05-2.86 (m, 4H), 2.70-2.60 (m, 4H), 2.58-2.50 (m, 2H), 2.43-2.30 (m, 2H), 1.58-1.49 (m, 2H).

### Examples 30A and 30B

9-fluoro-7-((R)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one
9-fluoro-7-((S)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one

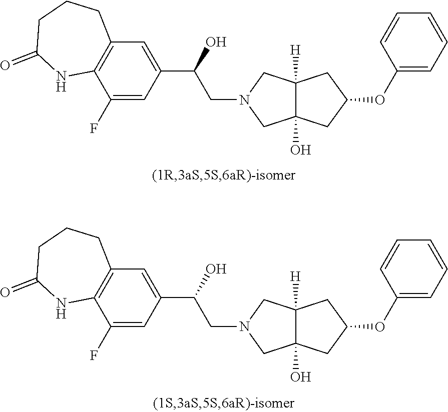 [SMILES: O=C1CCCc2cc([C@@H](O)CN3C[C@H]4C[C@H](Oc5ccccc5)C[C@@]4(O)C3)cc(F)c2N1 ; O=C1CCCc2cc([C@H](O)CN3C[C@H]4C[C@H](Oc5ccccc5)C[C@@]4(O)C3)cc(F)c2N1]

Using the same method as Examples 28A/28B, starting from Intermediates 2 and 27, a mixture of Examples 30A and 30B was obtained. The mixture was separated using the following chiral SFC method:

Column: Daicel Chiralpak AD (250 mm×30 mm, 10 μm), Flow Rate: 70 g/min

Mobile Phase: 40% IPA:ACN (1:1) with 0.1% NH₃·H₂O in CO₂

Analysis of the separated isomers was performed using the following analytical chiral SFC method:

Column: Chiralpak AD-3 (50×4.6 mm, 3 μm), Flow Rate: 3 mL/min, Mobile phase: 40% IPA:ACN (1:1) with 0.05% DEA in CO₂

Example 30A (analytical chiral SFC Rt 0.74 min): 13 mg.

LCMS: Rt 0.91 min; MS m/z 441.4 [M+H]⁺; Method I.

¹H NMR (400 MHz, CDC₃) δ 7.34-7.28 (m, 2H), 7.14 (br s, 1H), 7.10-6.96 (m, 3H), 6.91 (d, J=7.8 Hz, 2H), 4.97 (br s, 1H), 4.67-4.65 (m, 1H), 3.18 (d, J=9.2 Hz, 1H), 2.97-2.89 (m, 1H), 2.84 (t, J=7.2 Hz, 2H), 2.70-2.63 (m, 1H), 2.61-2.47 (m, 4H), 2.43-2.33 (m, 4H), 2.27-2.25 (m, 2H), 2.10-2.00 (m, 1H), 1.64-1.56 (m, 1H).

Example 30B (analytical chiral SFC Rt 0.93 min): 10 mg.

LCMS: Rt 0.91 min; MS m/z 441.4 [M+H]⁺; Method I.

¹H NMR (400 MHz, CDC₃) δ 7.33-7.29 (m, 2H), 7.07-6.97 (m, 4H), 6.94-6.88 (m, 2H), 4.98 (br s, 1H), 4.66-4.63 (m, 1H), 3.84 (br s, 1H), 2.93-2.85 (m, 1H), 2.83-2.81 (m, 3H), 2.77-2.49 (m, 7H), 2.43-2.33 (m, 3H), 2.30-2.21 (m, 2H), 2.14-2.10 (m, 1H), 1.57 (br s, 1H).

### Examples 31A and 31 B

8-fluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one
8-fluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one

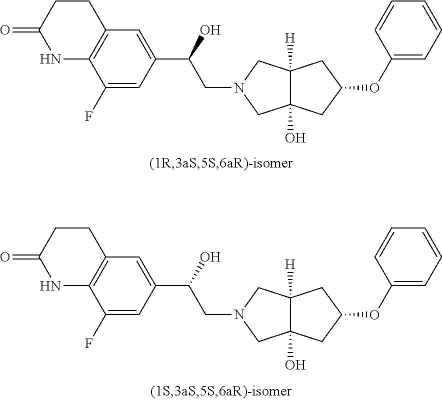 [SMILES: O=C1CCc2cc([C@@H](O)CN3C[C@H]4C[C@H](Oc5ccccc5)C[C@@]4(O)C3)cc(F)c2N1 ; O=C1CCc2cc([C@H](O)CN3C[C@H]4C[C@H](Oc5ccccc5)C[C@@]4(O)C3)cc(F)c2N1]

Step 1: A mixture of:
6-((R)-1-((tert-butyldimethylsilyl)oxy)-2-((3aS,5S,6aR)-3a-hydroxy-5-phenoxyhexahydrocyclopenta[c]pyrrol-2(1H)-yl)ethyl)-8-fluoro-3,4-dihydroquinolin-2(1H)-one
6-((S)-1-((tert-butyldimethylsilyl)oxy)-2-((3aS,5S,6aR)-3a-hydroxy-5-phenoxyhexahydrocyclopenta[c]pyrrol-2(1H)-yl)ethyl)-8-fluoro-3,4-dihydroquinolin-2(1H)-one

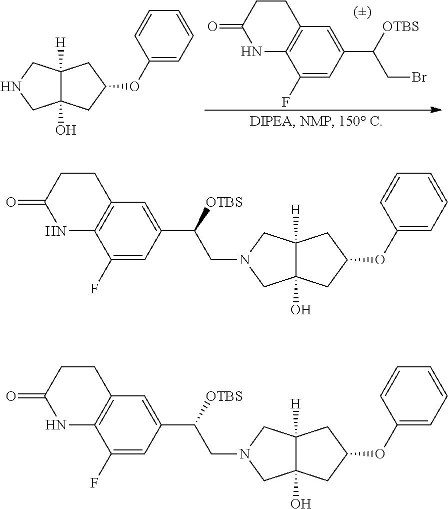

In a microwave vial, to a solution of Intermediate 2 (100 mg, 0.456 mmol) and Intermediate 37 (200 mg, 0.547 mmol) in NMP (2 mL) was added DIPEA (177 mg, 0.226 mL, 1.37 mmol). The vial was sealed and reacted under microwave irradiation in a Biotage Smith Synthesizer at 150° C. for 2 h. The reaction was diluted with water (5 mL), extracted with EtOAc (3×10 mL), washed with sat. brine (5 mL), dried with Na₂SO₄, filtered and concentrated. The crude material was purified by FCC (0-20% MeOH:DCM) to provide the title intermediates (200 mg) as a yellow oil.

LCMS: Rt 0.81 min; MS m/z 541.0 [M+H]⁺; Method J.

Step 2: A mixture of Examples 31A and 31 B

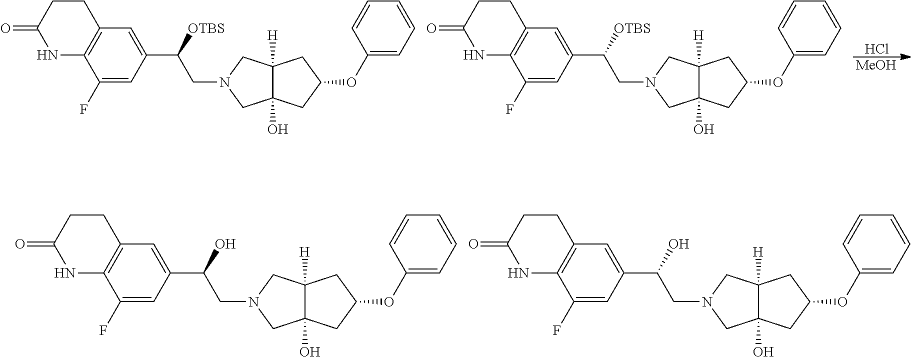

To a solution of the intermediates from the previous step (200 mg, 0.37 mmol) in MeOH (7.4 mL) was added conc. HCl (7.4 mL) slowly, and this was stirred at RT for 1 h. The reaction was concentrated and purified by preparative HPLC (column: Phenomenex Gemini NX-C18 (75×30 mm×3 μm); mobile phase: Water with 10 mM NH₄HCO₃ (A); Acetonitrile (B); 20-50% B over 8 min) to provide the title compounds (90 mg) as a white solid.

LCMS: Rt 0.90 min; MS m/z 427.4 [M+H]⁺; Method I.

Step 3: Chiral separation of Examples 31A and 31 B

The mixture was separated using the following chiral SFC method:

Column: Daicel Chiralpak OJ (250 mm×50 mm, 10 μm), Flow Rate: 55 g/min

Mobile Phase: 25% MeOH (0.1% NH₃·H₂O) in Supercritical CO₂

Example 31A: 42 mg.

Analytical chiral SFC: Rt 1.79 min (Column: Chiralcel OJ-3 50×4.6 mm, 3 μm, flow rate 3 mL/min, Mobile phase: 5-40% MeOH with 0.05% DEA in CO₂).

LCMS: Rt 0.90 min; MS m/z 427.3 [M+H]⁺; Method I.

¹H NMR (400 MHz, CDC₃) δ 7.56 (s, 1H), 7.35-7.28 (m, 2H), 7.06-6.96 (m, 3H), 6.94-6.88 (m, 2H), 4.97 (br s, 1H), 4.66-4.57 (m, 1H), 3.81 (br s, 1H), 3.05-2.90 (m, 3H), 2.83 (d, J=8.4 Hz, 1H), 2.75-2.58 (m, 7H), 2.56-2.47 (m, 2H), 2.37-2.34 (m, 1H), 2.17-2.10 (m, 1H), 1.59-1.54 (m, 1H).

Example 31 B: 35 mg.

Analytical chiral SFC: Rt 1.92 min (Column: Chiralcel OJ-3 50×4.6 mm, 3 μm, flow rate 3 mL/min, Mobile phase: 5-40% MeOH with 0.05% DEA in CO₂).

LCMS: Rt 0.90 min; MS m/z 427.3 [M+H]⁺; Method I.

¹H NMR (400 MHz, CDCl₃) δ 7.57 (s, 1H), 7.34-7.28 (m, 2H), 7.08-6.96 (m, 3H), 6.94-6.88 (m, 2H), 4.97 (br s, 1H), 4.69-4.58 (m, 1H), 3.18 (d, J=8.8 Hz, 1H), 3.03-2.89 (m, 3H), 2.72-2.44 (m, 8H), 2.40-2.33 (m, 2H), 2.12-2.06 (m, 1H), 1.63-1.57 (m, 1H).

### Examples 32A and 32B

9-fluoro-7-((R)-1-hydroxy-2-((3aS,5S,6aR)-3a-hydroxy-5-phenoxyhexahydrocyclopenta[c]pyrrol-2(1H)-yl)ethyl)-1,5-dihydrobenzo[e][1,4]oxazepin-2(3H)-one
9-fluoro-7-((S)-1-hydroxy-2-((3aS,5S,6aR)-3a-hydroxy-5-phenoxyhexahydrocyclopenta[c]pyrrol-2(1H)-yl)ethyl)-1,5-dihydrobenzo[e][1,4]oxazepin-2(3H)-one

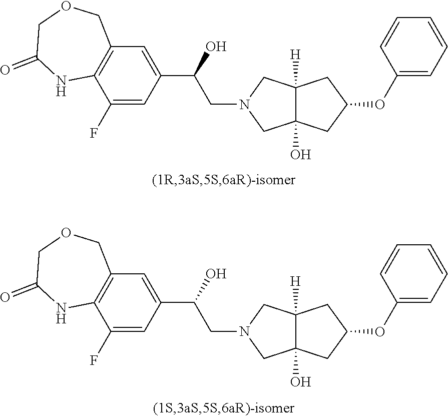 [SMILES: O=C1COCc2cc([C@@H](O)CN3C[C@H]4C[C@H](Oc5ccccc5)C[C@@]4(O)C3)cc(F)c2N1 ; O=C1COCc2cc([C@H](O)CN3C[C@H]4C[C@H](Oc5ccccc5)C[C@@]4(O)C3)cc(F)c2N1]

Step 1: A mixture of:
7-((R)-1-((tert-butyldimethylsilyl)oxy)-2-((3aS,5S,6aR)-3a-hydroxy-5-phenoxyhexahydrocyclopenta[c]pyrrol-2(1H)-yl)ethyl)-9-fluoro-1,5-dihydrobenzo[e][1,4]oxazepin-2(3H)-one
7-((S)-1-((tert-butyldimethylsilyl)oxy)-2-((3aS,5S,6aR)-3a-hydroxy-5-phenoxyhexahydrocyclopenta[c]pyrrol-2(1H)-yl)ethyl)-9-fluoro-1,5-dihydrobenzo[e][1,4]oxazepin-2(3H)-one

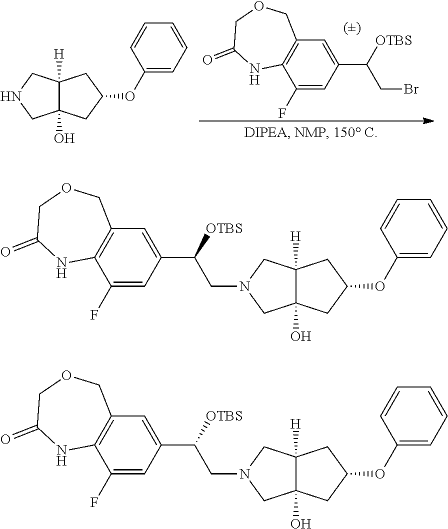

Using the same method as step 1 of Examples 31A/31B, starting from Intermediate 2 (300 mg, 1.37 mmol) and Intermediate 29 (700 mg, 1.67 mmol), provided a mixture of the title intermediates (400 mg) as a yellow oil.

LCMS: Rt 1.17 min; MS m/z 557.6 [M+H]⁺; Method I.

Step 2: A mixture of Examples 32A and 32B

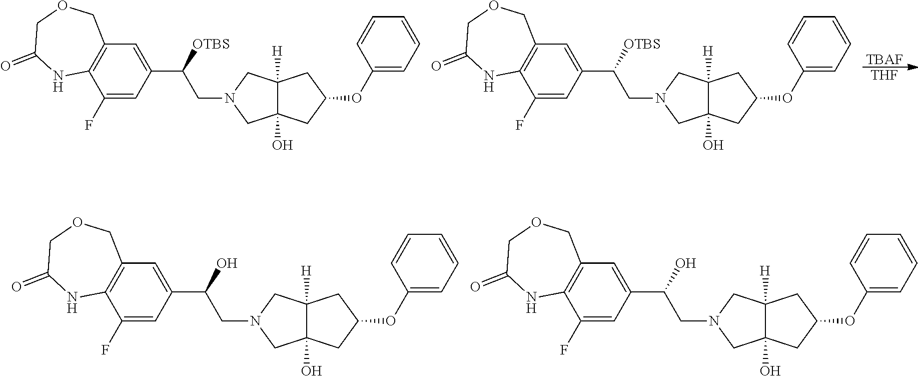

To a solution of the intermediates from the previous step (200 mg, 0.36 mmol) in THF (4.5 mL) was added TBAF (1 M in THF, 0.36 mL, 0.36 mmol) and this was stirred at RT for 2 h.

The reaction was diluted with water (3 mL), extracted with EtOAc (3×5 mL), dried with Na₂SO₄, filtered and concentrated. The crude material was purified by reverse phase FCC (column: C18; mobile phase: Water with 0.05% TFA v/v (A); ACN (B); gradient 5-95% B), then purified further by preparative TLC (15:1 DCM:MeOH with 1% NH₃·H₂O, Rf=0.6). The band containing product was taken up in 15:1 DCM:MeOH (15 mL) for 30 minutes, then filtered and concentrated to provide the title intermediates (60 mg) as a white solid.

LCMS: Rt 0.88 min; MS m/z 443.3 [M+H]⁺; Method I.

Step 3: Chiral separation of Examples 32A and 32B

The mixture was separated using the following chiral SFC method:

Column: Daicel Chiralpak AD (250 mm×30 mm, 10 μm), Flow Rate: 70 g/min

Mobile Phase: 70% EtOH (0.1% NH₃·H₂O) in Supercritical CO₂

Example 32A: 10 mg.

Analytical chiral SFC: Rt 1.69 min (Column: Chiralcel AD-3 50×4.6 mm, 3 μm, flow rate 3 mL/min, Mobile phase: 60% EtOH with 0.05% DEA in CO₂).

LCMS: Rt 0.89 min; MS m/z 444.3 [M+H]⁺; Method I.

¹H NMR (400 MHz, CDCl₃) δ 7.78-7.76 (m, 1H), 7.33-7.28 (m, 2H), 7.15-7.11 (m, 1H), 7.02-6.97 (m, 4H), 4.97 (br s, 1H), 4.77 (s, 2H), 4.70-4.64 (m, 1H), 4.62 (s, 2H), 3.21-3.17 (m, 1H), 2.99-2.93 (m, 1H), 2.68-2.34 (m, 9H), 2.14-2.09 (m, 1H), 1.67-1.58 (in, 1H).

Example 32 Suu: 18 mg.

Analytical chiral SFC: Rt 2.23 m (Column: Chiralcel AD-3 50×4.6 mm, 3 μm, flow rate 3 mL/min, Mobile phase: 60% EtOH with 0.05% IDEA in CO₂).

LCMS: Rt 0.87 min; MS m/z 444.3 [M+H]⁺; Method 1.

¹H NMR (400 MHz, CDCl₃) δ 7.78-7.76 (m, 1H), 7.33-7.28 (m, 2H), 7.15-7.11 (m, 1H), 7.02-6.97 (m, 4H), 4.97 (br s, 1H), 4.77 (s, 2H), 4.70-4.64 (m, 1H), 4.62 (s, 2H), 3.21-3.17 (m, 1H), 2.99-2.93 (m, 1H), 2.69-2.58 (m, 8H), 2.55-2.33 (m, 1H), 2.14-2.09 (m, 1H), 1.67-1.58 (m, 1H).

These examples were made as pairs of diastereomers using the same methods as Examples 31A/31 B, starting with the intermediates shown, and were separated using the conditions shown.

| | |
|---|---|
| Examples | |
| Intermediates | Structures and names |

Chiral SFC conditions
Analytical data

| | |
|---|---|
| Examples 33A and 33B | 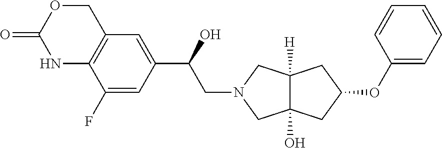 [SMILES: O=C1Nc2c(F)cc([C@@H](O)CN3C[C@H]4C[C@H](Oc5ccccc5)C[C@@]4(O)C3)cc2CO1] |
| | (1R,3aS,5S,6aR)-isomer |
| Made from Intermediates 2 and 31 | 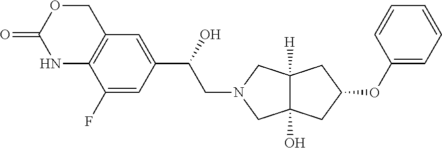 [SMILES: O=C1Nc2c(F)cc([C@H](O)CN3C[C@H]4C[C@H](Oc5ccccc5)C[C@@]4(O)C3)cc2CO1] |
| | (1S,3aS,5S,6aR)-isomer |
| | 8-fluoro-6-((R)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |
| | 8-fluoro-6-((S)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |

Chiral SFC (separation): Column: Daicel Chiralpak IG (250 mm × 30 mm, 10 μm), Flow
Rate: 70 g/min, Mobile phase: 60% IPA:ACN (1:1) with 0.1% NH₃•H₂O in CO₂
Chiral SFC (analytical): Column: Chiralpak IG-3 (50 × 4.6 mm, 3 μm), Flow Rate: 3 mL/min,
Mobile phase: 50% IPA:ACN (1:1) with 0.05% DEA in CO₂
Example 33A: Analytical chiral SFC: Rt 0.93 min.
LCMS: Rt 0.89 min; MS m/z 429.3 [M + H]⁺; Method I.
¹H NMR (400 MHz, CDCl₃) δ 7.43 (br s, 1H), 7.34-7.28 (m, 2H), 7.12 (d, J = 10.8 Hz,
1H), 7.02-6.95 (m, 2H), 6.94-6.88 (m, 2H), 5.33 (s, 2H), 4.97 (br s, 1H), 4.66-4.62
(m, 1H), 2.95-2.78 (m, 2H), 2.75-2.46 (m, 7H), 2.33-2.20 (m, 1H), 2.13-2.10 (m,
1H), 1.57-1.54 (m, 1H).
Example 33B: Analytical chiral SFC: Rt 1.29 min.
LCMS: Rt 0.89 min; MS m/z 429.4 [M + H]⁺; Method I.
¹H NMR (400 MHz, CDCl₃) δ 7.36-7.28 (m, 3H), 7.15-7.10 (m, 1H), 7.03-6.88 (m,
4H), 5.34 (s, 2H), 4.97 (br s, 1H), 4.67-4.64 (m, 1H), 3.99-3.69 (m, 1H), 3.21-2.87
(m, 2H), 2.74-2.48 (m, 6H), 2.42-2.33 (m, 2H), 2.10-2.09 (m, 1H), 1.61-1.54 (m,
1H).

| | |
|---|---|
| Examples 34A and 34B | 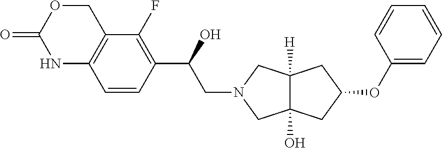 [SMILES: O=C1Nc2ccc([C@@H](O)CN3C[C@H]4C[C@H](Oc5ccccc5)C[C@@]4(O)C3)c(F)c2CO1] |
| | (1R,3aS,5S,6aR)-isomer |
| Made from Intermediates 2 and 30 | 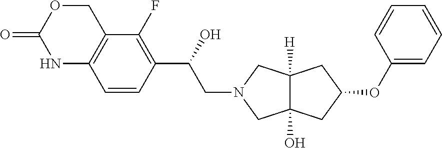 [SMILES: O=C1Nc2ccc([C@H](O)CN3C[C@H]4C[C@H](Oc5ccccc5)C[C@@]4(O)C3)c(F)c2CO1] |
| | (1S,3aS,5S,6aR)-isomer |
| | 5-fluoro-6-((R)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |
| | 5-fluoro-6-((S)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |

Chiral SFC (separation): Column: Daicel Chiralpak IG (250 mm × 30 mm, 10 μm), Flow
Rate: 70 g/min, Mobile phase: 55% IPA:ACN (1:1) with 0.1% NH₃•H₂O in CO₂
Chiral SFC (analytical): Column: Chiralpak IG-3 (50 × 4.6 mm, 3 μm), Flow Rate: 3 mL/min,
Mobile phase: 60% IPA:ACN (1:1) with 0.05% DEA in CO₂
Example 34A: Analytical chiral SFC: Rt 0.45 min.
LCMS: Rt 0.89 min; MS m/z 429.2 [M + H]⁺; Method I.
¹H NMR (400 MHz, CDCl₃) δ 7.98 (br s, 1H), 7.48-7.44 (m, 1H), 7.31 (t, J = 8.0 Hz, 2H),
6.99 (t, J = 7.2 Hz, 1H), 6.91 (d, J = 8.0 Hz, 2H), 6.64-6.62 (m, 1H), 5.41 (s, 2H), 5.05-
4.88 (m, 2H), 2.92 (d, J = 9.2 Hz, 1H), 2.73-2.70 (m, 1H), 2.77-2.62 (m, 4H), 2.61-
2.47 (m, 3H), 2.34-2.32 (m, 1H), 2.13-2 .10 (m, 1H), 1.57-1.52 (m, 1H).
Example 34B: Analytical chiral SFC: Rt 0.61 min.
LCMS: Rt 0.89 min; MS m/z 429.3 [M + H]⁺; Method I.
¹H NMR (400 MHz, CDCl₃) δ 8.05 (br s, 1H), 7.47-7.43 (m, 1H), 7.31-7.28 (m, 2H),
6.98-6.96 (m, 1H), 6.91 (d, J = 8.0 Hz, 2H), 6.64 (d, J = 8.4 Hz, 1H), 5.41 (s, 2H), 4.96
(br s, 2H), 4.39-3.32 (m, 1H), 3.16 (d, J = 9.2 Hz, 1H), 3.00-2.86 (m, 1H), 2.73-2.31
(m, 8H), 2.11-2.10 (m, 1H), 1.59-1.58 (m, 1H).

| | |
|---|---|
| Examples 35A and 35B | 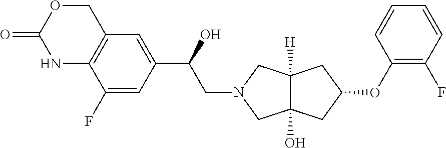 [SMILES: O=C1Nc2c(F)cc([C@@H](O)CN3C[C@H]4C[C@H](Oc5ccccc5F)C[C@@]4(O)C3)cc2CO1] |
| | (1R,3aS,5S,6aR)-isomer |
| Made from Intermediates 4 and 31 | 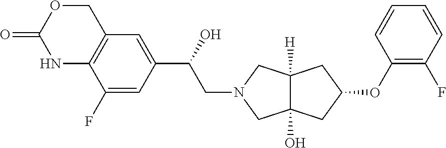 [SMILES: O=C1Nc2c(F)cc([C@H](O)CN3C[C@H]4C[C@H](Oc5ccccc5F)C[C@@]4(O)C3)cc2CO1] |
| | (1S,3aS,5S,6aR)-isomer |
| | 8-fluoro-6-((R)-2-((3aS,5S,6aR)-5-(2-fluorophenoxy)-3a- |
| | hydroxyhexahydrocyclopenta[c]pyrrol-2(1H)-yl)-1-hydroxyethyl)-1,4- |
| | dihydro-2H-benzo[d][1,3]oxazin-2-one |
| | 8-fluoro-6-((S)-2-((3aS,5S,6aR)-5-(2-fluorophenoxy)-3a- |
| | hydroxyhexahydrocyclopenta[c]pyrrol-2(1H)-yl)-1-hydroxyethyl)-1,4- |
| | dihydro-2H-benzo[d][1,3]oxazin-2-one |

Chiral SFC (separation): Column: Daicel Chiralpak IG (250 mm × 30 mm, 10 μm), Flow
Rate: 80 g/min, Mobile phase: 50% IPA:ACN (1:1) with 0.1% NH₃•H₂O in CO₂
Chiral SFC (analytical): Column: Chiralpak IG-3 (50 × 4.6 mm, 3 μm), Flow Rate: 3 mL/min,
Mobile phase: 60% IPA:ACN (1:1) with 0.05% DEA in CO₂
Example 35A: Analytical chiral SFC: Rt 0.51 min.
LCMS: Rt 0.90 min; MS m/z 447.3 [M + H]⁺; Method I.
¹H NMR (400 MHz, CDCl₃) δ 7.16-7.06 (m, 4H), 7.05-7.00 (m, 1H), 7.00-6.94 (m,
2H), 5.34 (s, 2H), 5.02 (br s, 1H), 4.65-4.62 (m, 1H), 3.01-2.80 (m, 3H), 2.73-2.59
(m, 4H), 2.53-5.49 (m, 2H), 2.41-2.36 (m, 1H), 2.11-2.07 (m, 1H), 1.54-1.52 (m,
2H).
Example 35B: Analytical chiral SFC: Rt 0.68 min.
LCMS: Rt 0.90 min; MS m/z 447.3 [M + H]⁺; Method I.
¹H NMR (400 MHz, CDCl₃) δ 7.15-7.06 (m, 4H), 7.05-7.00 (m, 1H), 6.99-6.93 (m,
2H), 5.34 (s, 2H), 5.01 (br s, 1H), 4.68-4.65 (m, 1H), 3.21-3.19 (m, 1H), 3.01-2.82
(m, 2H), 2.66-2.46 (m, 5H), 2.43-2.34 (m, 2H), 2.07-2.03 (m, 1H), 1.60-1.54 (m,
2H).

| | |
|---|---|
| Examples 36A and 36B | 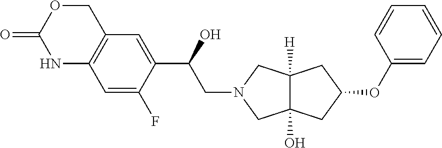 [SMILES: O=C1Nc2cc(F)c([C@@H](O)CN3C[C@H]4C[C@H](Oc5ccccc5)C[C@@]4(O)C3)cc2CO1] |
| | (1R,3aS,5S,6aR)-isomer |
| Made from Intermediates 2 and 32 | 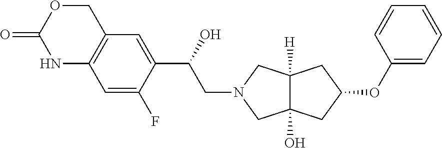 [SMILES: O=C1Nc2cc(F)c([C@H](O)CN3C[C@H]4C[C@H](Oc5ccccc5)C[C@@]4(O)C3)cc2CO1] |
| | (1S,3aS,5S,6aR)-isomer |
| | 7-fluoro-6-((R)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |
| | 7-fluoro-6-((S)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |

Chiral SFC (separation): Column: Daicel Chiralpak IG (250 mm × 30 mm, 10 μm), Flow
Rate: 80 g/min, Mobile phase: 60% EtOH in CO₂
Chiral SFC (analytical): Column: Chiralpak IG-3 (50 × 4.6 mm, 3 μm), Flow Rate: 3 mL/min,
Mobile phase: 60% EtOH with 0.05% DEA in CO₂
Example 36A: Analytical chiral SFC: Rt 0.65 min.
LCMS: Rt 0.89 min; MS m/z 429.4 [M + H]⁺; Method I.
¹H NMR (400 MHz, CDCl₃) δ 7.80-7.60 (m, 1H), 7.37-7.28 (m, 3H), 7.01-6.97 (m,
1H), 6.91 (d, J = 7.6 Hz, 2H), 6.53 (d, J = 10.0 Hz, 1H), 5.33-5.26 (m, 2H), 5.05-4.92
(m, 2H), 3.00-2.81 (m, 2H), 2.75-2.48 (m, 7H), 2.37-2.33 (m, 1H), 2.22-2.10 (m,
1H), 1.64-1.55 (m, 2H).
Example 36B: Analytical chiral SFC: Rt 1.15 min.
LCMS: Rt 0.89 min; MS m/z 429.4 [M + H]⁺; Method I.
¹H NMR (400 MHz, CDCl₃) δ 7.83-7.63 (m, 1H), 7.37-7.28 (m, 3H), 7.02-6.96 (m,
1H), 6.93-6.88 (m, 2H), 6.53 (d, J = 10.0 Hz, 1H), 5.29 (d, J = 3.2 Hz, 2H), 5.05-4.92
(m, 2H), 3.17 (d, J = 9.6 Hz, 1H), 3.01-2.90 (m, 1H), 2.69-2.32 (m, 8H), 2.13-2.05
(m, 1H), 1.64-1.55 (m, 2H).

| | |
|---|---|
| Examples 37A and 37B | 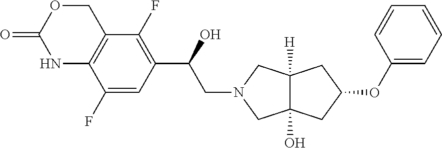 [SMILES: O=C1Nc2c(F)cc([C@@H](O)CN3C[C@H]4C[C@H](Oc5ccccc5)C[C@@]4(O)C3)c(F)c2CO1] |
| | (1R,3aS,5S,6aR)-isomer |
| Made from Intermediates 2 and 33 | 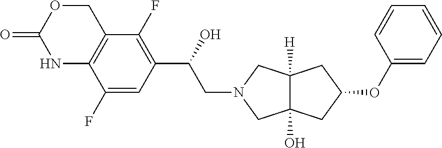 [SMILES: O=C1Nc2c(F)cc([C@H](O)CN3C[C@H]4C[C@H](Oc5ccccc5)C[C@@]4(O)C3)c(F)c2CO1] |
| | (1S,3aS,5S,6aR)-isomer |
| | 5,8-difluoro-6-((R)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |
| | 5,8-difluoro-6-((S)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |

Chiral SFC (separation): Column: Daicel Chiralpak AS (250 mm × 30 mm, 10 μm),
Flow Rate: 70 g/min, Mobile phase: 40% EtOH with 0.1% NH₃•H₂O in CO₂
Chiral SFC (analytical): Column: Chiralpak IG-3 (50 × 4.6 mm, 3 μm), Flow Rate: 3 mL/min,
Mobile phase: 50% IPA:ACN (1:1) with 0.05% DEA in CO₂
Example 37A: Analytical chiral SFC: Rt 0.69 min.
LCMS: Rt 0.90 min; MS m/z 447.3 [M + H]⁺; Method I.
¹H NMR (400 MHz, CDCl₃) δ 7.37-7.30 (m, 3H), 7.03-6.98 (m, 1H), 6.92 (d, J = 8.0 Hz,
2H), 5.43 (s, 2H), 4.99 (br s, 2H), 2.99-2.82 (m, 2H), 2.76-2.50 (m, 7H), 2.38-2.35
(m, 1H), 2.17-2.12 (m, 1H), 1.63-1.54 (m, 2H).
Example 37B: Analytical chiral SFC: Rt 1.25 min.
LCMS: Rt 0.90 min; MS m/z 447.4 [M + H]⁺; Method I.
¹H NMR (400 MHz, CDCl₃) δ 7.43 (br s, 1H), 7.37-7.29 (m, 3H), 7.02-6.98 (m, 1H),
6.92 (d, J = 8.0 Hz, 2H), 5.43 (s, 2H), 4.98 (d, J = 3.2 Hz, 2H), 3.19 (d, J = 9.2 Hz, 1H),
3.00-2.93 (m, 1H), 2.71-2.44 (m, 7H), 2.39-2.35 (m, 1H), 2.13-2.08 (m, 1H), 1.65-
1.58 (m, 1H).

| | |
|---|---|
| Examples 38A and 38B | 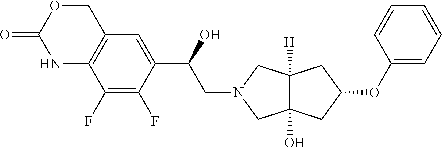 [SMILES: O=C1Nc2c(cc([C@@H](O)CN3C[C@H]4C[C@H](Oc5ccccc5)C[C@@]4(O)C3)c(F)c2F)CO1] |
| | (1R,3aS,5S,6aR)-isomer |
| Made from Intermediates 2 and 34 | 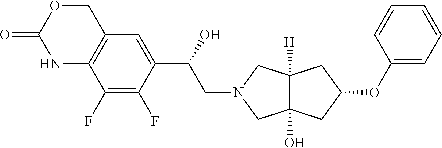 [SMILES: O=C1Nc2c(cc([C@H](O)CN3C[C@H]4C[C@H](Oc5ccccc5)C[C@@]4(O)C3)c(F)c2F)CO1] |
| | (1S,3aS,5S,6aR)-isomer |
| | 7,8-difluoro-6-((R)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |
| | 7,8-difluoro-6-((S)-1-hydroxy-2-((3aS,5S,6aR)-3a-hydroxy-5- |
| | phenoxyhexahydrocyclopenta[c]pyrrol-2(1H)-yl)ethyl)-1,4-dihydro-2H- |
| | benzo[d][1,3]oxazin-2-one |

Chiral SFC (separation): Column: Daicel Chiralpak IG (250 mm × 30 mm, 10 μm), Flow
Rate: 70 g/min, Mobile phase: 50% IPA:ACN (1:1) with 0.1% NH₃•H₂O in CO₂
Chiral SFC (analytical): Column: Chiralpak IG-3 (50 × 4.6 mm, 3 μm), Flow Rate: 3 mL/min,
Mobile phase: 50% IPA:ACN (1:1) with 0.05% DEA in CO₂
Example 38A: Analytical chiral SFC: Rt 0.57 min.
LCMS: Rt 0.91 min; MS m/z 447.3 [M + H]⁺; Method I.
¹H NMR (400 MHz, CDCl₃) δ 7.49 (br s, 1H), 7.34-7.28 (m, 2H), 7.16-7.14 (m, 1H),
7.01-6.97 (m, 1H), 6.91 (d, J = 8.0 Hz, 2H), 5.37-5.25 (m, 2H), 5.03-4.94 (m, 2H),
2.91 (d, J = 9.2 Hz, 1H), 2.82 (d, J = 8.4 Hz, 1H), 2.75-2.57 (m, 6H), 2.56-2.47 (m,
1H), 2.35 (m, 1H), 2.16-2.09 (m, 1H), 1.63-1.50 (m, 2H).
Example 38B: Analytical chiral SFC: Rt 0.79 min.
LCMS: Rt 0.90 min; MS m/z 447.4 [M + H]⁺; Method I.
¹H NMR (400 MHz, CDCl₃) δ 7.57 (br s, 1H), 7.36-7.29 (m, 2H), 7.17-7.15 (m, 1H),
7.02-6.98 (m, 1H), 6.92 (d, J = 8.0 Hz, 2H), 5.38-5.26 (m, 2H), 5.06-4.95 (m, 2H),
3.17 (d, J = 9.2 Hz, 1H), 3.03-2.94 (m, 1H), 2.79-2.45 (m, 7H), 2.39-2.35 (m, 1H),
2.15-2.07 (m, 1H), 1.67-1.44 (m, 2H).

| | |
|---|---|
| Examples 39A and 39B | 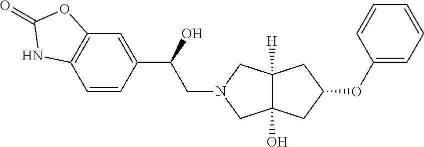 [SMILES: O=c1[nH]c2ccc([C@@H](O)CN3C[C@H]4C[C@H](Oc5ccccc5)C[C@@]4(O)C3)cc2o1] |
| | (1R,3aS,5S,6aR)-isomer |
| Made from Intermediates 2 and 35 | 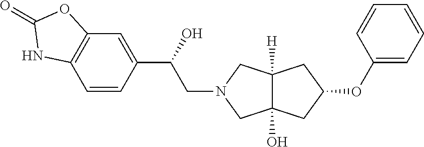 [SMILES: O=c1[nH]c2ccc([C@H](O)CN3C[C@H]4C[C@H](Oc5ccccc5)C[C@@]4(O)C3)cc2o1] |
| | (1S,3aS,5S,6aR)-isomer |
| | 6-((R)-1-hydroxy-2-((3aS,5S,6aR)-3a-hydroxy-5- |
| | phenoxyhexahydrocyclopenta[c]pyrrol-2(1H)-yl)ethyl)benzo[d]oxazol- |
| | 2(3H)-one |
| | 6-((S)-1-hydroxy-2-((3aS,5S,6aR)-3a-hydroxy-5- |
| | phenoxyhexahydrocyclopenta[c]pyrrol-2(1H)-yl)ethyl)benzo[d]oxazol- |
| | 2(3H)-one |

Chiral SFC (separation): Column: Daicel Chiralpak IG (250 mm × 30 mm, 10 μm), Flow
Rate: 70 g/min, Mobile phase: 50% IPA:ACN (1:1) with 0.1% NH₃•H₂O in CO₂
Chiral SFC (analytical): Column: Chiralpak IG-3 (50 × 4.6 mm, 3 μm), Flow Rate: 3 mL/min,
Mobile phase: 40% IPA:ACN (1:1) with 0.05% DEA in CO₂
Example 39A: Analytical chiral SFC: Rt 1.38 min.
LCMS: Rt 0.74 min; MS m/z 397.1 [M + H]⁺; Method J.
¹H NMR (400 MHz, CDCl₃) δ 7.35-7.28 (m, 3H), 7.17-7.15 (m, 1H), 7.05-6.96 (m,
2H), 6.91 (d, J = 7.6 Hz, 2H), 5.03-4.92 (m, 1H), 4.79-4.68 (m, 1H), 3.18 (d, J = 9.2 Hz,
1H), 2.98-2.88 (m, 1H), 2.74-2.30 (m, 8H), 2.12-2.09 (m, 1H).
Example 39B: Analytical chiral SFC: Rt 1.85 min.
LCMS: Rt 0.74 min; MS m/z 397.1 [M + H]⁺; Method J.
¹H NMR (400 MHz, CDCl₃) δ 7.37-7.30 (m, 3H), 7.18-7.16 (m, 1H), 7.05-6.97 (m,
2H), 6.93-6.91 (m, 2H), 5.08-4.90 (m, 1H), 4.80-4.66 (m, 1H), 3.28-2.04 (m, 11H).

| | |
|---|---|
| Examples 40A and 40B | 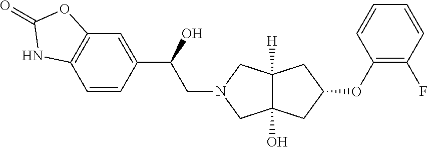 [SMILES: O=c1[nH]c2ccc([C@@H](O)CN3C[C@H]4C[C@H](Oc5ccccc5F)C[C@@]4(O)C3)cc2o1] |
| | (1R,3aS,5S,6aR)-isomer |
| Made from Intermediates 4 and 35 | 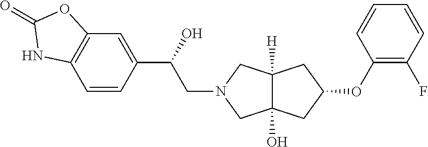 [SMILES: O=c1[nH]c2ccc([C@H](O)CN3C[C@H]4C[C@H](Oc5ccccc5F)C[C@@]4(O)C3)cc2o1] |
| | (1S,3aS,5S,6aR)-isomer |
| | 6-((R)-2-((3aS,5S,6aR)-5-(2-fluorophenoxy)-3a- |
| | hydroxyhexahydrocyclopenta[c]pyrrol-2(1H)-yl)-1- |
| | hydroxyethyl)benzo[d]oxazol-2(3H)-one |
| | 6-((S)-2-((3aS,5S,6aR)-5-(2-fluorophenoxy)-3a- |
| | hydroxyhexahydrocyclopenta[c]pyrrol-2(1H)-yl)-1- |
| | hydroxyethyl)benzo[d]oxazol-2(3H)-one |

Chiral SFC (separation): Column: Daicel Chiralpak IG (250 mm × 30 mm, 10 μm), Flow
Rate: 70 g/min, Mobile phase: 50% IPA:ACN (1:1) with 0.1% NH₃•H₂O in CO₂
Chiral SFC (analytical): Column: Chiralpak IG-3 (50 × 4.6 mm, 3 μm), Flow Rate: 3 mL/min,
Mobile phase: 40% IPA:ACN (1:1) with 0.05% DEA in CO₂
Example 40A: Analytical chiral SFC: Rt 1.07 min.
LCMS: Rt 0.76 min; MS m/z 415.1 [M + H]⁺; Method J.
¹H NMR (400 MHz, CDCl₃) δ 7.22 (s, 1H), 7.11-6.83 (m, 6H), 4.93 (br s, 1H), 4.65 (d, J =
7.2 Hz, 1H), 3.19-3.08 (m, 1H), 2.92-2.77 (m, 2H), 2.65-2.26 (m, 7H), 2.05-1.93
(m, 1H).
Example 40B: Analytical chiral SFC: Rt 1.38 min.
LCMS: Rt 0.76 min; MS m/z 415.1 [M + H]⁺; Method J.
¹H NMR (400 MHz, CDCl₃) δ 7.21 (s, 1H), 7.11-6.83 (m, 6H), 4.94 (br s, 1H), 4.64-
4.61 (m, 1H), 3.05-2.19 (m, 10H), 2.09-1.98 (m, 1H).

| | |
|---|---|
| Examples 41A and 41B | 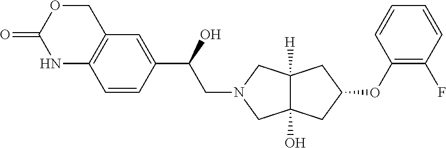 [SMILES: O=C1Nc2ccc([C@@H](O)CN3C[C@H]4C[C@H](Oc5ccccc5F)C[C@@]4(O)C3)cc2CO1] |
| | (1R,3aS,5S,6aR)-isomer |
| Made from Intermediates 4 and 36 | 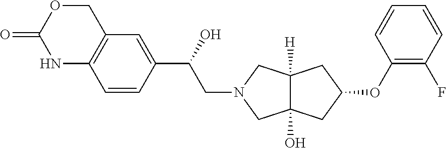 [SMILES: O=C1Nc2ccc([C@H](O)CN3C[C@H]4C[C@H](Oc5ccccc5F)C[C@@]4(O)C3)cc2CO1] |
| | (1S,3aS,5S,6aR)-isomer |
| | 6-((R)-2-((3aS,5S,6aR)-5-(2-fluorophenoxy)-3a- |
| | hydroxyhexahydrocyclopenta[c]pyrrol-2(1H)-yl)-1-hydroxyethyl)-1,4- |
| | dihydro-2H-benzo[d][1,3]oxazin-2-one |
| | 6-((S)-2-((3aS,5S,6aR)-5-(2-fluorophenoxy)-3a- |
| | hydroxyhexahydrocyclopenta[c]pyrrol-2(1H)-yl)-1-hydroxyethyl)-1,4- |
| | dihydro-2H-benzo[d][1,3]oxazin-2-one |

Chiral SFC (separation): Column: Daicel Chiralpak AD (250 mm × 30 mm, 10 μm),
Flow Rate: 80 g/min, Mobile phase: 70% MeOH:ACN (1:1) with 0.1% NH₃•H₂O in CO₂
Chiral SFC (analytical): Column: Chiralpak AD-3 (50 × 4.6 mm, 3 μm), Flow Rate: 3 mL/min,
Mobile phase: 60% MeOH:ACN (1:1) with 0.05% DEA in CO₂
Example 41A: Analytical chiral SFC: Rt 0.86 min.
LCMS: Rt 0.89 min; MS m/z 429.3 [M + H]⁺; Method I.
¹H NMR (400 MHz, CDCl₃) δ 7.95 (s, 1H), 7.27-7.23 (m, 1H), 7.18 (s, 1H), 7.13-6.93
(m, 4H), 6.81-6.78 (m, 1H),
s, 1H), 3.22-5.32 (s, 2H), 5.01 (br s, 1H), 4.72-4.64 (m, 1H), 3.79 (br
2.11-3.20 (m, 1H),
2.02 (m, 2.99-2.85 (m, 2H), 2.69-2.46 (m, 5H), 2.43-2.34 (m, 2H),
1H), 1.60-1.53 (m, 1H).
Example 41B: Analytical chiral SFC: Rt 1.75 min.
LCMS: Rt 0.89 min; MS m/z 429.3 [M + H]⁺; Method I.
¹H NMR (400 MHz, CDCl₃) δ 7.47 (s, 1H), 7.27-7.23 (m, 1H), 7.18 (s, 1H), 7.14-6.93
(m, 4H), 6.78-6.76 (m, 1H), 5.32 (s, 2H), 5.02 (br s, 1H), 4.69-4.61 (m, 1H), 3.01-2.89
(m, 2H), 2.88-2.82 (m, 1H), 2.76-2.69 (m, 1H), 2.67-2.58 (m, 3H), 2.55-2.46 (m,
2H), 2.42-2.35 (m, 1H), 2.15-2.06 (m, 1H), 1.57-1.46 (m, 2H).

| | |
|---|---|
| Examples 42A and 42B | 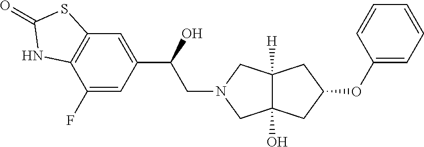 [SMILES: O=c1[nH]c2c(F)cc([C@@H](O)CN3C[C@H]4C[C@H](Oc5ccccc5)C[C@@]4(O)C3)cc2s1] |
| | (1R,3aS,5S,6aR)-isomer |
| Made from Intermediates 2 and 28 | 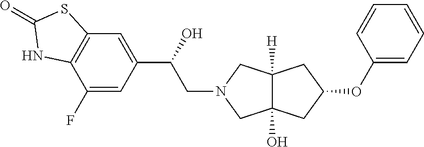 [SMILES: O=c1[nH]c2c(F)cc([C@H](O)CN3C[C@H]4C[C@H](Oc5ccccc5)C[C@@]4(O)C3)cc2s1] |
| | (1S,3aS,5S,6aR)-isomer |
| | 4-fluoro-6-((R)-1-hydroxy-2-((3aS,5S,6aR)-3a-hydroxy-5- |
| | phenoxyhexahydrocyclopenta[c]pyrrol-2(1H)-yl)ethyl)benzo[d]thiazol- |
| | 2(3H)-one |
| | 4-fluoro-6-((S)-1-hydroxy-2-((3aS,5S,6aR)-3a-hydroxy-5- |
| | phenoxyhexahydrocyclopenta[c]pyrrol-2(1H)-yl)ethyl)benzo[d]thiazol- |
| | 2(3H)-one |

Chiral SFC (separation): Column: Daicel Chiralpak IG (250 mm × 30 mm, 10 μm), Flow
Rate: 70 g/min, Mobile phase: 50% IPA:ACN (1:1) with 0.1% NH₃•H₂O in CO₂
Chiral SFC (analytical): Column: Chiralpak IG-3 (50 × 4.6 mm, 3 μm), Flow Rate: 3 mL/min,
Mobile phase: 50% IPA:ACN (1:1) with 0.05% DEA in CO₂
Example 42A: Analytical chiral SFC: Rt 0.62 min.
LCMS: Rt 0.74 min; MS m/z 431.0 [M + H]⁺; Method J.
¹H NMR (400 MHz, CDCl₃) δ 7.35-7.25 (m, 2H), 7.23 (s, 1H), 7.13-7.05 (m, 1H), 7.03-
6.95 (m, 1H), 6.94-6.87 (m, 2H), 4.97 (s, 1H), 4.73-4.66 (m, 1H), 2.93 (d, J = 9.2 Hz,
1H), 2.83 (d, J = 9.2 Hz, 1H), 2.79-2.47 (m, 6H), 2.40-2.31 (m, 1H), 2.17-2.09 (m,
1H), 1.61-1.52 (m, 1H).
Example 42B: Analytical chiral SFC: Rt 0.89 min.
LCMS: Rt 0.74 min; MS m/z 431.1 [M + H]⁺; Method J.
¹H NMR (400 MHz, CDCl₃) δ 7.35-7.26 (m, 2H), 7.24 (s, 1H), 7.11-7.05 (m, 1H), 7.02-
6.95 (m, 1H), 6.94-6.88 (m, 2H), 4.97 (s, 1H), 4.75-4.67 (m, 1H), 3.21-3.14 (m, 1H),
2.96-2.88 (m, 1H), 2.69-2.33 (m, 7H), 2.12-2.05 (m, 1H), 1.64-1.55 (m, 1H).

| | |
|---|---|
| Examples 43A and 43B | 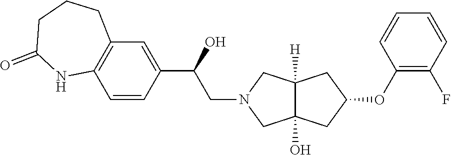 [SMILES: O=C1CCCc2cc([C@@H](O)CN3C[C@H]4C[C@H](Oc5ccccc5F)C[C@@]4(O)C3)ccc2N1] |
| | (1R,3aS,5S,6aR)-isomer |
| Made from Intermediates 4 and 38 | 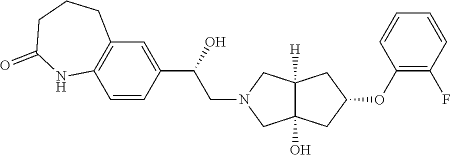 [SMILES: O=C1CCCc2cc([C@H](O)CN3C[C@H]4C[C@H](Oc5ccccc5F)C[C@@]4(O)C3)ccc2N1] |
| | (1S,3aS,5S,6aR)-isomer |
| | 7-((R)-2-((3aS,5S,6aR)-5-(2-fluorophenoxy)-3a- |
| | hydroxyhexahydrocyclopenta[c]pyrrol-2(1H)-yl)-1-hydroxyethyl)- |
| | 1,3,4,5-tetrahydro-2H-benzo[b]azepin-2-one |
| | 7-((S)-2-((3aS,5S,6aR)-5-(2-fluorophenoxy)-3a- |
| | hydroxyhexahydrocyclopenta[c]pyrrol-2(1H)-yl)-1-hydroxyethyl)- |
| | 1,3,4,5-tetrahydro-2H-benzo[b]azepin-2-one |

Chiral SFC (separation): Column: Daicel Chiralpak IG (250 mm × 30 mm, 10 μm), Flow
Rate: 80 g/min, Mobile phase: 60% EtOH with 0.1% NH₃•H₂O in CO₂
Chiral SFC (analytical): Column: Chiralpak IG-3 (50 × 4.6 mm, 3 μm), Flow Rate: 3 mL/min,
Mobile phase: 60% EtOH with 0.05% DEA in CO₂
Example 43A: Analytical chiral SFC: Rt 1.01 min.
LCMS: Rt 0.91 min; MS m/z 441.3 [M + H]⁺; Method I.
¹H NMR (400 MHz, CDCl₃) δ 7.28-7.23 (m, 2H), 7.20 (br s, 1H), 7.15-7.08 (m, 2H),
7.07-7.00 (m, 1H), 7.00-6.93 (m, 2H), 5.04 (br s, 1H), 4.87-4.68 (m, 1H), 3.09-2.93
(m, 2H), 2.89-2.57 (m, 8H), 2.56-2.50 (m, 1H), 2.42-2.35 (m, 3H), 2.29-2.21 (m,
3H), 1.67-1.59 (m, 1H).
Example 43B: Analytical chiral SFC: Rt 1.44 min.
LCMS: Rt 0.91 min; MS m/z 441.3 [M + H]⁺; Method I.
¹H NMR (400 MHz, CDCl₃) δ 7.28-7.23 (m, 2H), 7.15-7.02 (m, 3H), 7.01-6.93 (m,
2H), 5.03 (br s, 1H), 4.76-4.67 (m, 1H), 3.27-3.24 (m, 1H), 2.97-2.92 (m, 1H), 2.86-
2.79 (m, 2H), 2.77-2.62 (m, 2H), 2.61-2.47 (m, 3H), 2.44-2.34 (m, 4H), 2.29-2.22
(m, 2H), 2.14-2.07 (m, 1H), 1.63-1.56 (m, 1H).

### Examples 44A and 44B

(3aS,4S,5S,6aR)-5-(2-fluorophenoxy)-2-((R)-2-hydroxy-2-(1H-pyrrolo[2,3-b]pyridin-5-yl)ethyl) hexahydrocyclopenta[c]pyrrole-3a,4(1H)-diol
(3aS,4S,5S,6aR)-5-(2-fluorophenoxy)-2-((S)-2-hydroxy-2-(1H-pyrrolo[2,3-b]pyridin-5-yl)ethyl) hexahydrocyclopenta[c]pyrrole-3a,4(1H)-diol

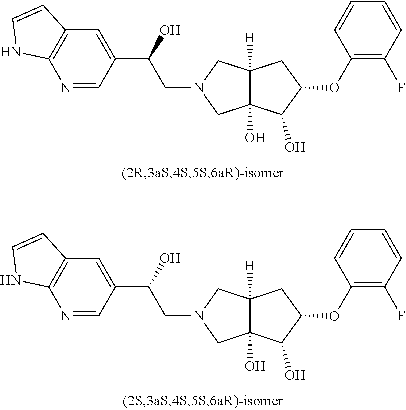 [SMILES: O[C@@H](CN1C[C@H]2C[C@H](Oc3ccccc3F)[C@H](O)[C@@]2(O)C1)c1cnc2[nH]ccc2c1 ; O[C@H](CN1C[C@H]2C[C@H](Oc3ccccc3F)[C@H](O)[C@@]2(O)C1)c1cnc2[nH]ccc2c1]

Step 1: A mixture of:
(3aS,4S,5S,6aR)-5-(2-fluorophenoxy)-2-((S)-2-hydroxy-2-(1-tosyl-1H-pyrrolo[2,3-c]pyridin-5-yl)ethyl) hexahydrocyclopenta[c]pyrrole-3a,4(1H)-diol
(3aS,4S,5S,6aR)-5-(2-fluorophenoxy)-2-((R)-2-hydroxy-2-(1-tosyl-1H-pyrrolo[2,3-c]pyridin-5-yl)ethyl)hexahydrocyclopenta[c]pyrrole-3a,4(1H-diol

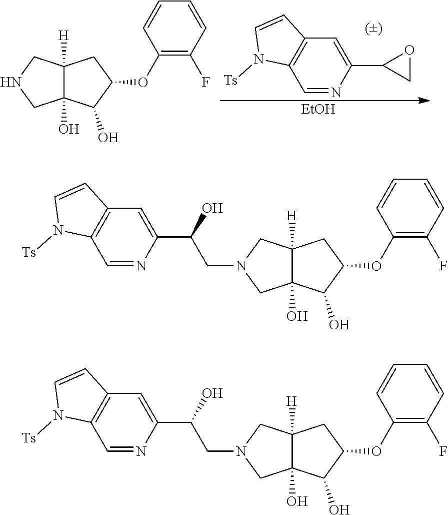

A solution of Intermediate 8 (200 mg, 0.79 mmol) and Intermediate 39 (248 mg, 0.79 mmol) in EtOH (10 mL) was stirred at 90° C. for 4 h. The reaction was concentrated and purified by FCC (5% MeOH:DCM) to provide the title intermediates (200 mg).

LCMS: Rt 0.45 min; MS m/z 568.3 [M+H]⁺; Method D.

Step 2: A mixture of Examples 44A and 44B

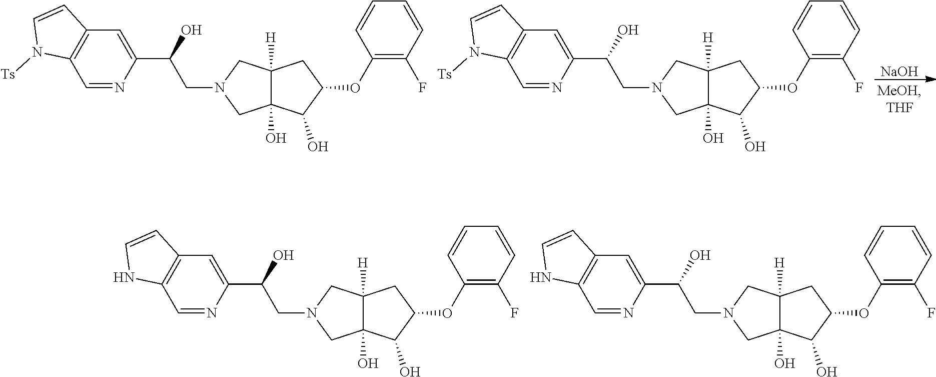

To a solution of the intermediates from the previous step (200 mg, 0.35 mmol) in THF (5 mL) and MeOH (1 mL) was added 1 N aq. NaOH (1.05 mL, 1.05 mmol) and this was stirred at 60° C. for 6 h. The reaction mixture was concentrated, neutralized with 1N HCl, and basified with saturated aqueous NaHCO₃, then extracted with DCM, dried with Na₂SO₄, filtered and concentrated. The crude material was purified by the following preparative HPLC method to provide the title intermediates (90 mg).

Column: Kinetex Evo (150 mm×21.2 mm), 5.0 μm, Flow: 18.0 mL/min

Mobile phase: 0.02% NH₄OH in water (A), Acetonitrile (B)

LCMS: Rt 0.11 min; MS m/z 414.3 [M+H]⁺; Method D.

Step 3: Chiral separation of Examples 44A and 44B

The mixture was separated using the following chiral HPLC method:

Column: Chiralpak IC (10 mm×250 mm, 5 micron), Flow: 8 mL/min

Mobile phase: Hexane (A), EtOH:MeOH 1:1 (B), Isocratic: 65:35 (A:B)

Example 44A (chiral HPLC Rt 6.42 min): 35 mg.

LCMS: Rt 0.12 min; MS m/z 414.0 [M+H]⁺; Method D.

¹H NMR (400 MHz, Methanol-d₄) δ 8.21 (d, J=2.0 Hz, 1H), 8.03 (d, J=2.0 Hz, 1H), 7.36 (d, J=3.2 Hz, 1H), 7.08-6.90 (m, 4H), 6.46 (d, J=3.6 Hz, 1H), 4.68-4.65 (m, 1H), 3.93 (d, J=3.6 Hz, 1H), 3.01 (d, J=9.6 Hz, 1H), 2.88-2.82 (m, 1H), 2.74-2.63 (m, 3H), 2.44-2.37 (m, 2H), 2.27-2.20 (m, 1H), 1.55-1.50 (m, 1H). 1H under solvent peak.

Example 44B (chiral HPLC Rt 7.75 min): 35 mg.

LCMS: Rt 0.12 min; MS m/z 414.2 [M+H]⁺; Method D.

¹H NMR (400 MHz, Methanol-d₄) δ 8.21 (d, J=2.0 Hz, 1H), 8.03 (d, J=2.0 Hz, 1H), 7.36 (d, J=3.2 Hz, 1H), 7.08-6.90 (m, 4H), 6.46 (d, J=3.6 Hz, 1H), 4.68-4.65 (m, 1H), 3.93 (d, J=3.6 Hz, 1H), 3.01 (d, J=9.6 Hz, 1H), 2.88-2.82 (m, 1H), 2.74-2.63 (m, 3H), 2.44-2.37 (m, 2H), 2.27-2.20 (m, 1H), 1.55-1.50 (m, 1H). 1H under solvent peak.

Biological Assays and Data

The activity of a compound according to the present disclosure can be assessed by the following in vitro & in vivo methods.

### Example 1: NR2B Rat Cortical Neuron Calcium Influx Assay Protocol

Embryonic day 18 timed pregnant Sprague Dawley rats were euthanized according to Institutional Animal Care and Use Committee (IACUC) protocol. After cutting medially through the skin and exposing the uterus and embryos, fetuses were removed and placed in cold Hibernate medium. Each embryo's brain was extracted and cerebral cortices were isolated by removing the midbrain and meninges. The dissected cortices were then dissociated into the neurons using papain dissociation system (Worthington Biochemical Corporation) according the manufacturer's protocol.

Dissociated neurons were counted and plated into 384-well poly-D-lysine coated plates (Corning® BioCoat™) at a density of 20,000 cells/well in 30 μL of Neurobasal/B27 complete medium. Neurons were cultured at 37° C. for 2 days. On the assay day, medium was removed and cells were incubated with 20 μL/well of calcium dye (Calcium 6 Assay Kit, Molecular Devices) suspended in HBSS with 1.8 mM Ca²⁺ (Ca-HBSS) according to the manufacturer's instruction.

Compounds of interest from 10 mM stock were serially diluted into 3× of desired concentrations in 1.8 mM Ca-HBSS, and 10 μL were added to the wells. Compound and the neurons were incubated at 37° C. for 2 hours in the dark.

On FDSS7000EX (Hamamatsu Photonics), a fluorescence measuring instrument, 10 μL of 4× ligand solution containing glutamate and glycine made in 1.8 mM Ca²⁺-HBSS were added to each well. The fluorescent signals were collected before and after the addition of ligands for a total of 2 minutes. The data were converted to a ratio of the peak fluorescence to the fluorescence at the beginning of the measurement.

Each data point was measured in duplicates. Dose response curves were used to identify IC₅₀ and maximal inhibition values. IC₅₀ represents the concentration in μM of compound at which there is a half-maximal compound effect. Maximal inhibition of a compound is expressed as a percent of the highest inhibition of activity over a no compound control.

TABLE 1
NR2B rat cortical neuron calcium influx assay,
MDCK-MDR1 ER and rat hepatocyte clearance data

| | | MDCK-MDR1 | Hepatocyte Clearance, |
|---|---|---|---|
| Example | IC50 (uM) | ER | CL (hep), rat |
| 1A | 0.0006 | 3.02 | 29.6 |
| 1B | 0.0016 | 2.30 | 49.7 |
| 2A | 0.1 | 3.30 | NT* |
| 2B | 0.063 | 3.54 | NT |
| 3A | 5.49 | 7.49 | NT |
| 3B | >10 | 6.36 | NT |
| 3C | 0.4 | NT | NT |
| 3D | 0.075 | NT | NT |
| 4A | 0.013 | 6.99 | NT |
| 4B | 0.0036 | 7.74 | 15.0 |
| 4C | 3.05 | 6.75 | NT |
| 4D | 0.25 | 6.87 | NT |
| 5A | 0.038 | 1.05 | NT |
| 5B | 0.13 | NT | NT |
| 5C | 0.00059 | 2.07 | 76.0 |
| 5D | 0.00061 | 1.93 | 117.4 |
| 6A | 0.52 | 1.01 | NT |
| 6B | 1.06 | NT | NT |
| 6C | 0.0038 | 1.07 | 87.0 |
| 6D | 0.0024 | 1.29 | NT |
| 7A | 0.014 | 2.73 | NT |
| 7B | 0.0019 | 2.07 | 39.1 |
| 8A | 0.0008 | 3.68 | 4.9 |
| 8B | 0.00057 | 3.46 | 4.0 |
| 9A | 0.0027 | 1.06 | NT |
| 9B | 0.0041 | 1.40 | NT |
| 10A | 0.00041 | 1.11 | 83.0 |
| 10B | 0.00056 | 2.49 | NT |
| 11A | 0.0019 | 1.74 | NT |
| 11B | 0.0026 | NT | NT |
| 12A | 0.0003 | 2.81 | NT |
| 12B | 0.00025 | NT | NT |
| 13A | 0.086 | 1.22 | NT |
| 13B | 0.057 | 1.11 | NT |
| 14A | 0.000011 | 1.51 | NT |
| 14B | 0.00018 | 2.06 | NT |
| 15A | 0.0000015 | 1.60 | NT |
| 15B | 0.0000019 | 1.92 | NT |
| 16A | 0.000051 | 0.66 | NT |
| 16B | 0.00008 | 0.75 | NT |
| 17A | 0.000013 | 1.11 | 145.0 |
| 17B | 0.000049 | 2.06 | 91.0 |
| 18A | <0.0000021 | 1.61 | NT |
| 18B | 0.000012 | NT | NT |
| 19 | 0.0087 | NT | NT |
| 20A | 0.002 | NT | NT |
| 20B | 0.00033 | 3.32 | NT |
| 21 | 0.011 | 1.94 | 57.5 |
| 22A | 0.023 | NT | NT |
| 22B | 0.42 | NT | NT |
| 22C | 0.0014 | 1.43 | NT |
| 22D | 0.0016 | NT | NT |
| 23A | 0.033 | NT | NT |
| 23B | 0.28 | NT | NT |
| 23C | 0.0014 | 3.75 | NT |
| 23D | 0.0013 | 3.28 | NT |
| 24A | 0.016 | NT | NT |
| 24B | 0.057 | NT | NT |
| 24C | 0.000021 | 1.46 | NT |
| 24D | 0.000056 | 2.64 | NT |
| 25A | >1.1 | NT | NT |
| 25B | 0.014 | NT | NT |
| 25C | 0.00024 | 2.74 | NT |
| 25D | 0.00022 | NT | NT |
| 26A | 0.25 | NT | NT |
| 26B | 0.47 | NT | NT |
| 26C | 0.0013 | 3.27 | NT |
| 26D | 0.0019 | 2.52 | |
| 27A | >1.1 | NT | NT |
| 27B | 0.29 | NT | NT |
| 27C | 0.0017 | 1.52 | NT |
| 27D | 0.0014 | 2.05 | NT |
| 28A | 0.00083 | 3.32 | NT |
| 28B | 0.0017 | 3.18 | NT |
| 29A | 0.13 | 3.23 | NT |
| 29B | 0.011 | 4.24 | NT |
| 30A | 0.28 | 1.18 | NT |
| 30B | 0.017 | NT | NT |
| 31A | 0.01 | 1.63 | NT |
| 31B | 0.0048 | NT | NT |
| 32A | 0.96 | 0.83 | NT |
| 32B | 0.6 | NT | NT |
| 33A | 0.0049 | 1.87 | 35.0 |
| 33B | 0.0036 | 0.91 | 71.0 |
| 34A | 0.0027 | 3.81 | NT |
| 34B | 0.0011 | 3.47 | NT |
| 35A | 0.0012 | 1.00 | NT |
| 35B | 0.0013 | 2.04 | NT |
| 36A | 0.00049 | 4.04 | NT |
| 36B | 0.0015 | 2.44 | NT |
| 37A | 0.033 | 1.15 | NT |
| 37B | 0.0065 | NT | NT |
| 38A | 0.0051 | 1.58 | NT |
| 38B | 0.0078 | NT | NT |
| 39A | 0.026 | NT | NT |
| 39B | 0.086 | NT | NT |
| 40A | 0.0069 | NT | NT |
| 40B | 0.028 | NT | NT |
| 41A | 0.0003 | 1.86 | NT |
| 41B | 0.00023 | 2.44 | NT |
| 42A | 0.00019 | 0.93 | 40.0 |
| 42B | 0.00004 | 0.90 | 91.0 |
| 43A | 0.000034 | 2.16 | NT |
| 43B | 0.0012 | NT | NT |
| 44A | 0.17 | 6.09 | NT |
| 44B | 1.19 | 7.34 | NT |

*NT = not tested

### Example 2. Microsome and Hepatocyte Assay Protocols

Microsome Incubations: The experiments were performed in 96-well format with shaking incubation at 37° C. on an automated platform. Test compounds, at a concentration of 10 mM in DMSO, were diluted 1:5000 into a 100 mM potassium phosphate, pH 7.4 (KPi) solution containing cofactor (2 mM NADPH, 4 mM MgCl₂) to a concentration of 2 μM. The reaction was initiated by adding equal volume to rat or human liver microsomal protein (1 mg/mL) suspended in 100 mM KPi buffer. At specific reaction time points (0, 5, 15, and 30 minutes), reaction aliquots were removed and reactions were terminated by the addition of three volumes of acetonitrile containing the analytical internal standard (0.4 μM glyburide). The samples were then centrifuged at 4000×g at 4° C. for 10 minutes, and the supernatants were analyzed by LC/MS/MS for quantification of the remaining test compound. The percentage of test compound remaining, relative to time zero minute incubation, was used to estimate the in vitro elimination-rate constant (k_{mic}), which was subsequently used to calculate the in vitro metabolic clearance rates.

Hepatocyte Incubations: The experiments were performed in 96-well format with shaking incubation at 37° C. on an automated platform. Test compounds, at a concentration of 10 mM in DMSO, were diluted 1:5000 into a Leibovitz's L15 medium (L-15) solution to a concentration of 2 μM. The reaction was initiated by adding equal volume to suspended rat or human hepatocytes at 2 million cells/mL in L-15 media solution. At specific reaction time points (0, 10, 20, 40, 60, and 80 minutes), reaction aliquots were removed and reactions were terminated by the addition of three volumes of acetonitrile containing the analytical internal standard (0.4 μM glyburide). The samples were then centrifuged at 4000×g at 4° C. for 10 minutes, and the supernatants were analyzed by LC/MS/MS for quantification of the remaining test compound. The percentage of test compound remaining, relative to time zero minute incubation, was used to estimate the in vitro elimination-rate constant (k_{mic}), which was subsequently used to calculate the in vitro metabolic clearance rates.

LC/MS/MS Analysis: Samples were analyzed on a high performance liquid chromatography (HPLC)-tandem mass spectrometry (LC/MS/MS) system consisting of Shimadzu 30 series autosampler and HPLC pump coupled to an AB Sciex AP16500. Compound specific parameters (precursor ion, product ion, declustering potential, and collision energy for single reaction monitoring) were obtained by automatic tuning using the Multiquant software V3.0. Samples were loaded onto an ACE 3 C18, 2.1 mm×30 mm, 3 μm column by means of the Shimadzu 30 series autosampler. The components were eluted with a gradient of 0.1% formic acid in water (mobile phase A) and 0.1% formic acid in acetonitrile (mobile phase B) at a flow of 700 μL/min using the following gradient: 0 min 2% B; 0.25 min 2% B; 1.00 min 98% B; 1.55 min 98% B; 1.95 min 2% B; 2.00 min 2% B. The analyte concentration was calculated from the chromatographic peak area ratio of analyte to internal standard (glyburide, m/z 494→169), using Multiquant software V3.0 (Sciex, Framingham, MA).

### Example 3. hERG Qpatch Assay Protocol

This assay was performed by the method described in Skepper et al., J. Med. Chem. 2020, 63, 7773-7816:

hERG expressing cell lines were produced in-house at Novartis using CHO-K1 T-Rex inducible plasmid system (Invitrogen) as described previously (Cao et al., Assay Drug Dev. Technol. 2010, 8, 766-780). Cell lines were maintained in Ham's F12 nutrient mixture containing 10% FBS, blasticidin (10 mg/mL; InvivoGen), hygromycin B (200 mg/mL; InvivoGen), Zeocin (200 mg/mL, Invitrogen), and neomycin (200 mg/mL, Invitrogen) using SelecT automated cell culture system (TAP Biosystems, Cambridge, U.K.). hERG and hCav1.2 channels expression was induced with tetracycline (0.25-1 μg/mL, Invitrogen) at least 24 h prior to the experiment.

hERG currents were recorded using the Qpatch automated patch clamp systems (Sophion Bioscience Inc., North Brunswick, NJ) in the whole (single) cell configuration. hERG expressing CHO-K1 cells were harvested with Detachin (Genlantis) and stored in the modified serum-free SFM-2 media (Life Technologies) at room temperature. The extracellular solution contained (in mM) NaCl (145), KCl (4), MgCl₂ (1), CaCl₂ (2), and HEPES (10), pH 7.4, with NaOH. The intracellular solution contained KCl (135), MgCl₂ (1.75), CaCl₂ (5.4), EGTA (10), K2-ATP (4), and HEPES (10), pH 7.2, with KOH. After whole cell configuration was achieved, the cell was held at −90 mV, and a 0.1 s pulse to −50 mV was delivered to measure the leaking current, which was subtracted from the tail current online. Then the cell was depolarized to +20 mV for 4 s (prepulse), followed by a 4 s test pulse to −50 mV to reveal the hERG tail current. To monitor changes in the current amplitude, this voltage protocol was repeatedly applied every 20 s. Test compounds were first diluted in DMSO for six dose-response experiments and then dissolved in the extracellular solution using Freedom EVO liquid handling robotic system (Tecan, Männedorf, Switzerland). The final DMSO concentration in samples was 0.3% v/v. Amitriptyline (Sigma) was tested as a positive control. Data were analyzed using in-house developed MatLab-based program (MathWorks, Natick, MA

### Example 4. Experimental Measurement of Efflux with MDCK-MDR1 Protocol

Cell Culture. MDCK-MDR1 cells were cultured at 37° C. under a 5% CO₂ atmosphere, at 95% relative humidity in DMEM containing 10% FBS, penicillin-streptomycin (100 μg/mL), and 2 mM Ala-Gln. Cells were passaged every 3-4 days. For assay purposes, cells were seeded at a density of approximately 265,000 cells/cm² of a 96-well Transwell plate (Corning Life Sciences, Acton, MA) and cultured in the same media noted above for a period of 4 days.

Assay. The determination of the apparent permeability (Pₐₚₚ) was performed in both the A→B (apical to basal) and B→A (basal to apical) directions where each compound was assayed in triplicate. The zwitterion bestatin, a poorly permeably compound, was used as marker of monolayer integrity. To initiate the assay, media was aspirated, and the cells and basal chambers were washed three times with Hank's Balanced Salt Solution (HBSS) containing 10 mM HEPES (pH 7.4). Compound test solutions were prepared in triplicate in HBSS containing 10 mM HEPES (pH 7.4) and 0.02% bovine serum albumin (BSA) to a final concentration of 10 μM and centrifuged for 2 min at 4000×g, then applied to the donor compartment at time zero. Additionally, at time zero, a 37° C. solution without test articles (HBSS+10 mM HEPES (pH 7.4) plus 0.02% BSA) was added to the receiver chamber of the Transwell plate. A time zero sample of the donor solution was also sampled for further analysis. The assay was conducted for a period of 120 min at 37° C. without shaking. At the time of assay termination, samples were taken from each donor compartment, and each acceptor compartment of the Transwell plate. To each of the 0 and 120 min samples was added an internal standard solution containing glyburide in water:acetonitrile, 50:50 (v:v). Concentration curves were prepared using a Labcyte Echo in the same matrix noted above. Samples and concentration curve samples were centrifuged for 10 min at 4000×g and subsequently analyzed by mass spectroscopy.

Mass Spectroscopy. Assay samples were loaded onto a RapidFire C4 cartridge by means of a RapidFire autosampler (Agilent, Santa Clara, CA). Chromatography was performed at a flow rate of 1.25 mL/min, loading with 0.1% formic acid in water and eluting in 0.1% formic acid in methanol. Mass spectroscopy was performed using an AB Sciex AP15500 (Sciex, Frammingham, MA) equipped with a turbo ion spray source. The analyte concentration was calculated from the chromatographic peak area ratio of analyte to internal standard (glibenclamide, m/z 494→169), using Multiquant software V3.0 (Sciex, Framingham, MA).

Calculations. Pₐₚₚ values were determined as

Papp=VAS[D0]×A120tPapp=VAS[D0]×A120t

Percent recovery values were determined as:

% Recovery=100×(A120+D120D0)% Recovery=100×A120+D120D0

where V_{A} is the volume of the acceptor (mL), S is the surface area of the membrane, D₀ is the donor solution concentration at t=0, D₁₂₀ is the donor solution concentration at t=120, A₁₂₀ is the acceptor solution concentration at t=120, and t=time (seconds).

Hepatocytes is used to determine the in vitro intrinsic clearance of a compound. The use of species-specific cryopreserved hepatocytes can be used to enable an understanding of interspecies differences. Hepatocyte clearance [CL(hep.)], for instance in rat, is one of the important markers for assessing rat oral bioavailability. Compounds profiled in this assay are tabulated in Table 1.

The suitability of a compound for oral dosing and/or for use as a CNS therapeutic is usually conducted by MDCK-MDR1 permeability assay to investigate its drug efflux potential mediated by P-glycoprotein (P-gp). MDCK-MDR1 permeability has been used as a predictor of blood brain barrier permeability in terms of efflux ratio (ER). Selected compounds profiled in this assay are tabulated in Table 1.

TABLE 2
hERG Qpatch data.

| Example | hERG QPatch IC50 (uM) |
|---|---|
| 1A | 10.7 |
| 1B | 7.6 |
| 4B | 9.2 |
| 5C | 6.0 |
| 5D | 5.9 |
| 6C | 13.3 |
| 7B | 15.2 |
| 8A | 2.8 |
| 10A | 2.1 |
| 14B | 1.3 |
| 16A | 0.4 |
| 16B | 0.2 |
| 17A | 0.2 |
| 18B | 1.2 |
| 21 | 3.6 |
| 24C | 1.2 |
| 28A | 2.4 |
| 29B | 12.5 |
| 31A | 7.3 |
| 31B | 3.9 |
| 33A | 8.2 |
| 33B | 4.9 |
| 36A | 5.5 |
| 42A | 2.2 |
| 42B | 0.8 |
| 44B | 15.7 |

TABLE 3
Comparison of in vitro ADME and hERG Qpatch data between matched pairs
containing the hydroxy core (present disclosure) vs. des-hydroxy cores
(comparative compounds).

| Structure | | | Example | |
|---|---|---|---|---|
| Rat Microsome | Human Microsome | Rat Hepatocyte | Human Hepatocyte | hERG Qpatch |
| CLᵢₙₜ | CLᵢₙₜ | CLᵢₙₜ | CLᵢₙₜ | IC₅₀ (μM) |
| 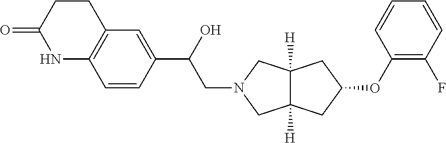 [SMILES: O=C1CCc2cc(C(O)CN3C[C@H]4C[C@H](Oc5ccccc5F)C[C@H]4C3)ccc2N1] | | | Mixture of two isomers at benzylic position | |
| 281 | 38 | 91 | 24 | 0.5 |
| 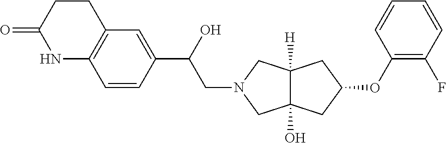 [SMILES: O=C1CCc2cc(C(O)CN3C[C@H]4C[C@H](Oc5ccccc5F)C[C@@]4(O)C3)ccc2N1] | | | Example 5C | |
| 114 | 46 | 76 | 8 | 6.0 |
| 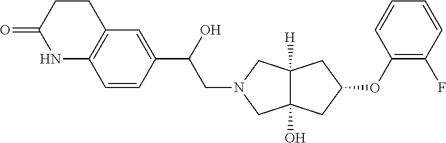 [SMILES: O=C1CCc2cc(C(O)CN3C[C@H]4C[C@H](Oc5ccccc5F)C[C@@]4(O)C3)ccc2N1] | | | Example 5D | |
| 303 | 69 | 117 | 8 | 5.9 |

As illustrated by Table 3, compounds from the present disclosure have improved properties compared to comparative compounds lacking the core hydroxy group. Furthermore, as seen in Tables 1 and 2, preferred compounds from the present disclosure generally have overall balanced and desirable profiles suitable for oral administration as a CNS therapeutics. These include lower clearance in hepatocytes, which is believed to be associated with a more desirable pharmacokinetic profile; good MDCK-MRD1 efflux ratio (ER) which is an indicator for blood brain barrier penetration, and furthermore, the compounds of the present disclosure have less activity in the hERG Qpatch assay, which is believed to be associated with an improved cardiosafety profile.

1. A compound of Formula (I): 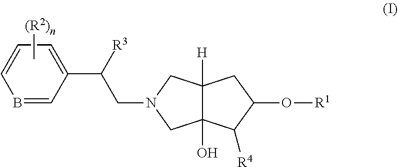
or a pharmaceutically acceptable salt thereof, wherein:
R¹ is a C₃₋₆ cycloalkyl, C₃₋₇ heterocyclyl, phenyl, naphthyl, or heteroaryl, each of which is optionally substituted with one or more R⁵;
R² is OH, CN, halogen, OR⁶, SH, SR⁶, C₁₋₆ alkyl, haloC₁₋₆ alkyl, NH₂, NHR⁶, hydroxyC₁₋₆ alkyl, N(R⁶)(R⁶′), NHS(O)₂R⁶, or NHCOR⁶, wherein R² is not OH when in the para position; or two R² groups, together with ring carbon atoms to which the two R² groups are attached, combine to form a five- to seven-membered heterocyclic ring or a five- or six-membered heteroaryl ring;
R³ is H, O, or OH;
R⁴ is H or OH;
R⁵ is halogen, OH, C₁₋₆ alkyl, OR⁶, CN, NH₂, NHR⁶, N(R⁶)(R⁶′), SH, SR⁶, SOR⁶, SO₂R⁶, SO₂NHR⁶, SO₂N(R⁶)(R⁶′), CONH₂, CONHR⁶, or CON(R⁶)(R⁶′);
each R⁶ and R⁶′ is independently selected from the group consisting of H, O—C₁₋₆ alkyl, C₁₋₆ alkyl, and haloC₁₋₆ alkyl;
B is N or CRx;
each Rx is independently H, C₁₋₃ alkyl, or halogen; and
each n is independently 0, 1, 2, 3, or 4.

2. A compound according to claim 1 of Formula II: 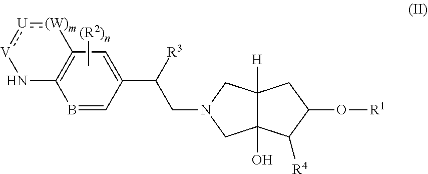
or a pharmaceutically acceptable salt thereof, wherein:
wherein R¹ is a C₃₋₈ cycloalkyl, C₃₋₇ heterocyclyl, phenyl, naphthyl, or heteroaryl, each of which is optionally substituted with one or more R⁵;
R² is OH, CN, halogen, OR⁶, SH, SR⁶, C₁₋₆ alkyl, haloC₁₋₆ alkyl, NH₂, NHR⁶, hydroxyC₁₋₆ alkyl, N(R⁶)(R⁶′), NHS(O)₂R⁶, or NHCOR⁶;
R³ is H, O, or OH;
R⁴ is H or OH;
R⁵ is halogen, OH, C₁₋₆ alkyl, OR⁶, CN, NH₂, NHR⁶, N(R⁶)(R⁶′), SH, SR⁶, SOR⁶, SO₂R⁶, SO₂NHR⁶, SO₂N(R⁶)(R⁶′), CONH₂, CONHR⁶, or CON(R⁶)(R⁶′);
each R⁶ and R⁶′ is independently selected from the group consisting of H, O—C₁₋₆ alkyl, C₁₋₆ alkyl, and haloC₁₋₆ alkyl;
B is N or CRx;
V is carbonyl, CH, or N;
U is O, S, CRx, or CRxRx;
each Rx is independently H, C₁₋₃ alkyl, or halogen;
each W is independently O, CH, or CH₂; [symbol]
is an optional double bond;
m is 0, 1, or 2; and
each n is independently 0, 1, 2, 3, or 4.

3. A compound according to claim 2 of Formula (III): 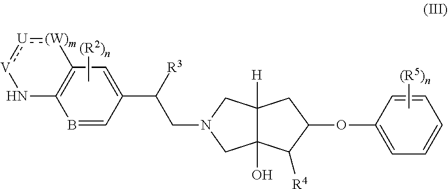
or a pharmaceutically acceptable salt thereof, wherein:
R² is OH, CN, halogen, OR⁶, SH, SR⁶, C₁₋₆ alkyl, haloC₁₋₆ alkyl, NH₂, NHR⁶, hydroxyC₁₋₆ alkyl, N(R⁶)(R⁶′), NHS(O)₂R⁶, or NHCOR⁶;
R³ is H, O, or OH;
R⁴ is H or OH;
R⁵ is halogen, OH, C₁₋₆ alkyl, OR⁶, CN, NH₂, NHR⁶, N(R⁶)(R⁶′), SH, SR⁶, SOR⁶, SO₂R⁶, SO₂NHR⁶, SO₂N(R⁶)(R⁶′), CONH₂, CONHR⁶, or CON(R⁶)(R⁶′);
each R⁶ and R⁶′ is independently selected from the group consisting of H, O—C₁₋₆ alkyl, C₁₋₆ alkyl, and haloC₁₋₆ alkyl;
B is N or CRx;
V is carbonyl, CH, or N;
U is O, S, CRx, or CRxRx;
each Rx is independently H, C₁₋₃ alkyl, or halogen;
each W is independently O, CH, or CH₂; [symbol]
is an optional double bond;
m is 0, 1, or 2; and
each n is independently 0, 1, 2, 3, or 4.

4. A compound according to claim 2 of Formula (IV): 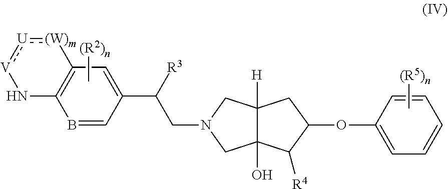
or a pharmaceutically acceptable salt thereof, wherein:
R² is halogen;
R³ is H or OH;
R⁴ is H or OH;
R⁵ is halogen;
B is N or CH;
V is carbonyl, CH, or N;
U is O, S, CRx, or CRxRx;
each Rx is independently H, C₁₋₃alkyl, or halogen;
each W is independently O, CH, or CH₂; [symbol]
is an optional double bond;
m is 0, 1, or 2; and
each n is independently 0, 1, 2, 3, or 4.

5. The compound according to claim 4 of a formula selected from the group consisting of Formula IVa, IVb, IVc, IVd, IVe, and IVf: 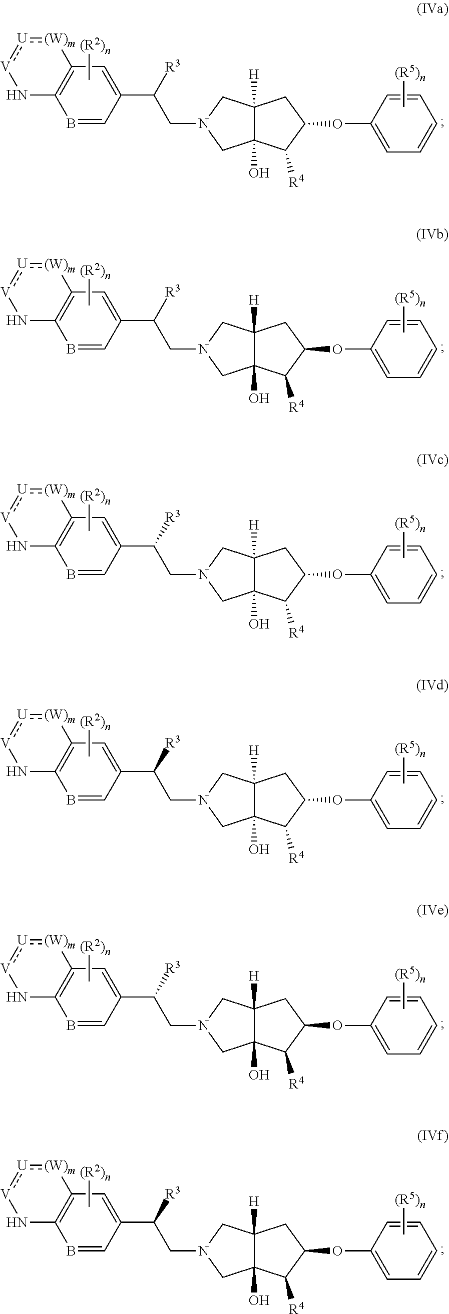
or a pharmaceutically acceptable salt thereof.

6-10. (canceled)

11. The compound according to claim 4 of Formula V: 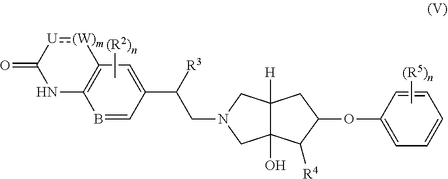
or a pharmaceutically acceptable salt thereof.

12. The compound according to claim 4 of a formula selected from the group consisting of Formula Va, Vb, Vc, Vd, Ve, and Vf: 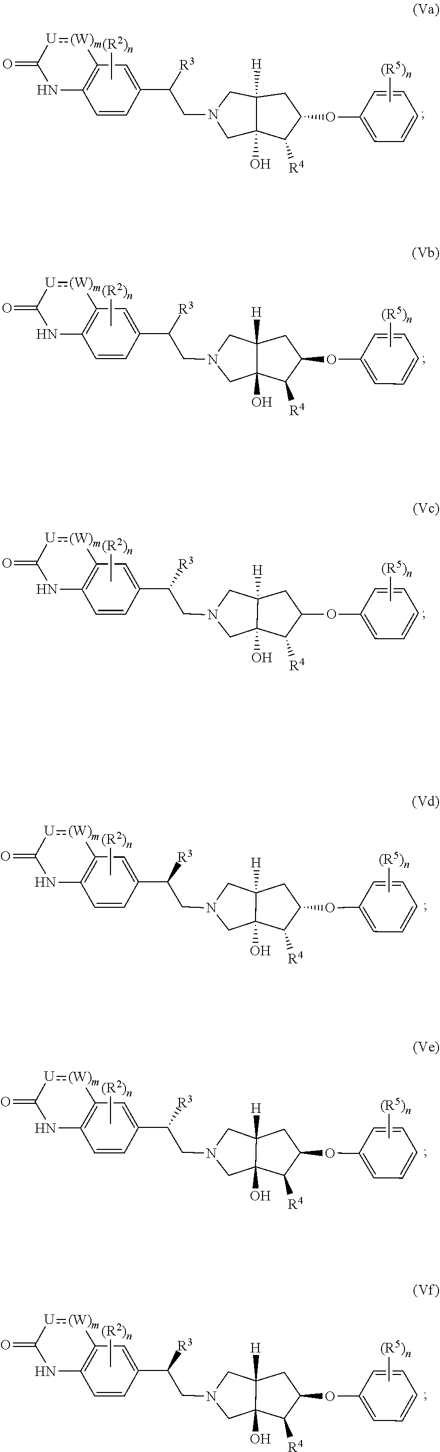
or a pharmaceutically acceptable salt thereof.

13-17. (canceled)

18. The compound according to claim 11, wherein U is CRxRx, and W is CH₂, or a pharmaceutically acceptable salt thereof.

19. The compound according to claim 18, wherein m is 1 or 2, or a pharmaceutically acceptable salt thereof.

20. (canceled)

21. The compound according to claim 11, wherein U is CRx, W is CH, and m is 1, or a pharmaceutically acceptable salt thereof.

22. The compound according to claim 11, wherein U is CRxRx, W is O, and m is 1, or a pharmaceutically acceptable salt thereof.

23. The compound according to claim 11, wherein U is CRxRx, one W is O, one W is CH₂, and m is 2, or a pharmaceutically acceptable salt thereof.

24. The compound according to claim 11, wherein U is CRxRx, and m is O, or a pharmaceutically acceptable salt thereof.

25. The compound according to claim 11, wherein U is O, and W is CH₂, or a pharmaceutically acceptable salt thereof.

26. The compound according to claim 25, wherein m is 1 or 2, or a pharmaceutically acceptable salt thereof.

27. (canceled)

28. The compound according to claim 11, wherein U is O, and m is 0, or a pharmaceutically acceptable salt thereof.

29. The compound according to claim 11, wherein U is S, W is CH₂, and m is 1, or a pharmaceutically acceptable salt thereof.

30. The compound according to claim 11, wherein U is S, and m is 0, or a pharmaceutically acceptable salt thereof.

31. The compound according to claim 4 of Formula VI: 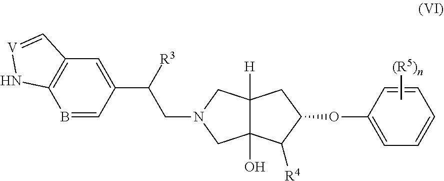
or a pharmaceutically acceptable salt thereof, wherein:
R³ is H or OH;
R⁴ is H or OH;
R⁵ is halogen;
V is CH or N;
B is N or CH; and
each n is independently 0, 1, 2, 3, or 4.

32. The compound according to claim 4 of a formula selected from the group consisting of Formula VIa, VIb, VIc, VId, VIe, and VIf: 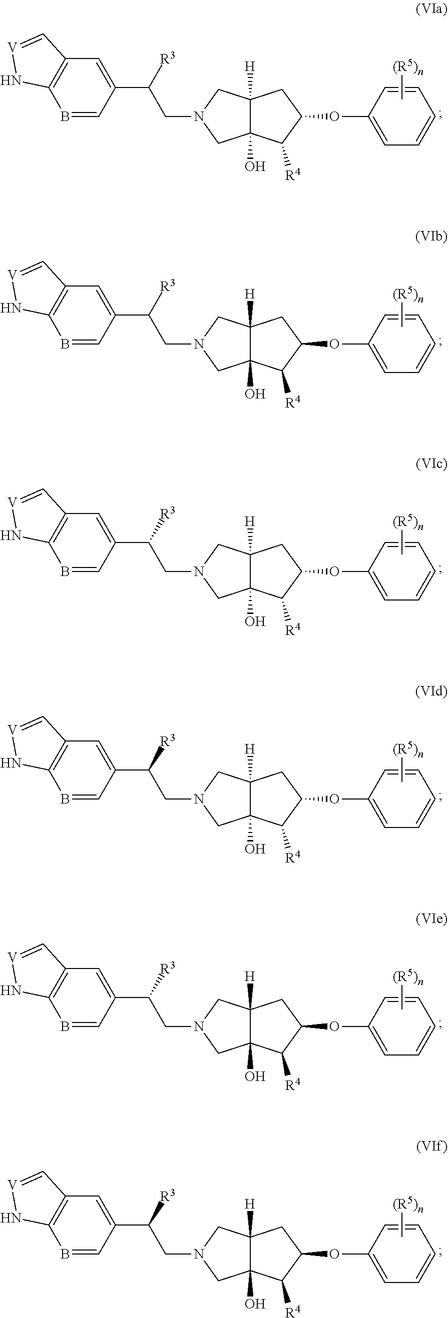
or a pharmaceutically acceptable salt thereof.

33-37. (canceled)

38. The compound of Formula (I) according to claim 1 wherein R² or R⁵ is F.

39. The compound of Formula (I) according to claim 1 wherein R³ is H.

40. The compound of Formula (I) according to claim 1 wherein R³ is OH.

41. The compound of Formula (I) according to claim 1 wherein R⁴ is H.

42. The compound of Formula (I) according to claim 1 wherein R⁴ is OH.

43. The compound of Formula (I) according to claim 1 wherein R² is CN, halogen, OR⁶, SH, SR⁶, C₁₋₆ alkyl, haloC₁₋₆ alkyl, or hydroxyC₁₋₆ alkyl.

44-45. (canceled)

46. The compound of Formula (I) according to claim 1 wherein R⁵ is halogen, OH, C₁₋₆ alkyl, OR⁶, CN, SH, or SR⁶.

47-48. (canceled)

49. A compound which is:
6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
5-((R)-1-hydroxy-2-((3aS,5S,6aR)-3a-hydroxy-5-phenoxyhexahydrocyclopenta[c]pyrrol-2(1H)-yl)ethyl)indolin-2-one;
5-((S)-1-hydroxy-2-((3aS,5S,6aR)-3a-hydroxy-5-phenoxyhexahydrocyclopenta[c]pyrrol-2(1H)-yl)ethyl)indolin-2-one;
5-((R)-2-((3aS,4S,5S,6aR)-3a,4-dihydroxy-5-phenoxyhexahydrocyclopenta[c]pyrrol-2(1H)-yl)-1-hydroxyethyl)indolin-2-one;
5-((S)-2-((3aS,4S,5S,6aR)-3a,4-dihydroxy-5-phenoxyhexahydrocyclopenta[c]pyrrol-2(1H)-yl)-1-hydroxyethyl)indolin-2-one;
5-((R)-2-((3aR,4R,5R,6aS)-3a,4-dihydroxy-5-phenoxyhexahydrocyclopenta[c]pyrrol-2(1H)-yl)-1-hydroxyethyl)indolin-2-one;
5-((S)-2-((3aR,4R,5R,6aS)-3a,4-dihydroxy-5-phenoxyhexahydrocyclopenta[c]pyrrol-2(1H)-yl)-1-hydroxyethyl)indolin-2-one;
6-((R)-2-((3aS,4S,5S,6aR)-3a,4-dihydroxy-5-phenoxyhexahydrocyclopenta[c]pyrrol-2(1H)-yl)-1-hydroxyethyl)-3,4-dihydroquinolin-2(1H)-one;
6-((S)-2-((3aS,4S,5S,6aR)-3a,4-dihydroxy-5-phenoxyhexahydrocyclopenta[c]pyrrol-2(1H)-yl)-1-hydroxyethyl)-3,4-dihydroquinolin-2(1H)-one;
6-((R)-2-((3aR,4R,5R,6aS)-3a,4-dihydroxy-5-phenoxyhexahydrocyclopenta[c]pyrrol-2(1H)-yl)-1-hydroxyethyl)-3,4-dihydroquinolin-2(1H)-one;
6-((S)-2-((3aR,4R,5R,6aS)-3a,4-dihydroxy-5-phenoxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one;
8-fluoro-6-((R)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one;
8-fluoro-6-((S)-2-((3aS,5S,6aR)-5-(2-fluorophenoxy)-3a-hydroxyhexahydrocyclopenta[c]pyrrol-2(1H)-yl)-1-hydroxyethyl)-3,4-dihydroquinolin-2(1H)-one;
8-fluoro-6-((R)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one;
8-fluoro-6-((S)-2-((3aR,5R,6aS)-5-(2-fluorophenoxy)-3a-hydroxyhexahydrocyclopenta[c]pyrrol-2(1H)-yl)-1-hydroxyethyl)-3,4-dihydroquinolin-2(1H)-one;
7-((R)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one;
7-((S)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one;
6-((R)-1-hydroxy-2-((3aS,5S,6aR)-3a-hydroxy-5-phenoxyhexahydrocyclopenta[c]pyrrol-2(1H)-yl)ethyl)quinolin-2(1H)-one;
6-((S)-1-hydroxy-2-((3aS,5S,6aR)-3a-hydroxy-5-phenoxyhexahydrocyclopenta[c]pyrrol-2(1H)-yl)ethyl)quinolin-2(1H)-one;
5-fluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
5-fluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
7-fluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
7-fluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
6-((R)-1-hydroxy-2-((3aS,5S,6aR)-3a-hydroxy-5-phenoxyhexahydrocyclopenta[c]pyrrol-2(1H)-yl)ethyl)-3,3-dimethyl-3,4-dihydroquinolin-2(1H)-one;
6-((S)-1-hydroxy-2-((3aS,5S,6aR)-3a-hydroxy-5-phenoxyhexahydrocyclopenta[c]pyrrol-2(1H)-yl)ethyl)-3,3-dimethyl-3,4-dihydroquinolin-2(1H)-one;
7-((R)-2-((3aS,5S,6aR)-5-(2-fluorophenoxy)-3a-hydroxyhexahydrocyclopenta[c]pyrrol-2(1H)-yl)-1-hydroxyethyl)-4,5-dihydrobenzo[d][1,3]oxazepin-2(1H)-one;
7-((S)-2-((3aS,5S,6aR)-5-(2-fluorophenoxy)-3a-hydroxyhexahydrocyclopenta[c]pyrrol-2(1H)-yl)-1-hydroxyethyl)-4,5-dihydrobenzo[d][1,3]oxazepin-2(1H)-one;
5-fluoro-7-((R)-1-hydroxy-2-((3aS,5S,6aR)-3a-hydroxy-5-phenoxyhexahydrocyclopenta[c]pyrrol-2(1H)-yl)ethyl)-2H-benzo[b][1,4]-oxazin-3(4H)-one;
5-fluoro-7-((S)-1-hydroxy-2-((3aS,5S,6aR)-3a-hydroxy-5-phenoxyhexahydrocyclopenta[c]pyrrol-2(1H)-yl)ethyl)-2H-benzo[b][1,4]-oxazin-3(4H)-one;
6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]thiazin-2-one;
6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]thiazin-2-one;
6-((R)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]thiazin-2-one;
6-((S)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]thiazin-2-one;
8-fluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]-thiazin-2-one;
8-fluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]-thiazin-2-one;
6-((R)-1-hydroxy-2-((3aS,5S,6aR)-3a-hydroxy-5-phenoxyhexahydrocyclopenta[c]pyrrol-2(1H)-yl)ethyl)benzo[d]thiazol-2(3H)-one;
6-((S)-1-hydroxy-2-((3aS,5S,6aR)-3a-hydroxy-5-phenoxyhexahydrocyclopenta[c]pyrrol-2(1H)-yl)ethyl)benzo[d]thiazol-2(3H)-one;
6-((R)-2-((3aS,5S,6aR)-5-(2-fluorophenoxy)-3a-hydroxyhexahydrocyclopenta[c]pyrrol-2(1H)-yl)-1-hydroxyethyl)benzo[d]thiazol-2(3H)-one;
6-((S)-2-((3aS,5S,6aR)-5-(2-fluorophenoxy)-3a-hydroxyhexahydrocyclopenta[c]pyrrol-2(1H)-yl)-1-hydroxyethyl)benzo[d]thiazol-2(3H)-one; or
a mixture of two or more compounds that are:
(S)-3,8-difluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
(S)-3,8-difluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
(R)-3,8-difluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
(R)-3,8-difluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
3,8-difluoro-6-((R)-1-hydroxy-2-((3aS,5S,6aR)-3a-hydroxy-5-phenoxyhexahydrocyclopenta[c]pyrrol-2(1H)-yl)ethyl)quinolin-2(1H)-one;
3,8-difluoro-6-((S)-1-hydroxy-2-((3aS,5S,6aR)-3a-hydroxy-5-phenoxyhexahydrocyclopenta[c]pyrrol-2(1H)-yl)ethyl)quinolin-2(1H)-one;
(3aS,5S,6aR)-5-(2,4-difluorophenoxy)-2-((R)-2-hydroxy-2-(1H-indazol-5-yl)ethyl)hexahydrocyclopenta[c]pyrrol-3a(1H)-ol;
(3aS,5S,6aR)-5-(2,4-difluorophenoxy)-2-((S)-2-hydroxy-2-(1H-indazol-5-yl)ethyl)hexahydrocyclopenta[c]pyrrol-3a(1H)-ol;
(3aR,5R,6aS)-5-(2,4-difluorophenoxy)-2-((R)-2-hydroxy-2-(1H-indazol-5-yl)ethyl)hexahydrocyclopenta[c]pyrrol-3a(1H)-ol;
(3aR,5R,6aS)-5-(2,4-difluorophenoxy)-2-((S)-2-hydroxy-2-(1H-indazol-5-yl)ethyl)hexahydrocyclopenta[c]pyrrol-3a(1H)-ol;
6-((R)-2-((3aS,5S,6aR)-5-(4-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(4-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(4-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(4-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(3-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(3-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(3-fluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(3-fluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2,3-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2,3-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2,3-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2,3-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2,4-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2,4-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2,4-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2,4-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2,5-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2,5-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2,5-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2,5-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aS,5S,6aR)-5-(2,6-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aS,5S,6aR)-5-(2,6-difluorophenoxy)-3a-hydroxyhexahydrocyclopenta[c]pyrrol-2(1H)-yl)-1-hydroxyethyl)-3,4-dihydroquinolin-2(1H)-one;
6-((R)-2-((3aR,5R,6aS)-5-(2,6-difluorophenoxy)-3a-hydroxyhexahydrocyclopenta[c]pyrrol-2(1H)-yl)-1-hydroxyethyl)-3,4-dihydroquinolin-2(1H)-one;
6-((S)-2-((3aR,5R,6aS)-5-(2,6-difluorophenoxy)-3a-hydroxyhexahydrocyclopenta[c]pyrrol-2(1H)-yl)-1-hydroxyethyl)-3,4-dihydroquinolin-2(1H)-one;
6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
8-fluoro-6-((R)-2-((3aS,4S,5S,6aR)-5-(2-fluorophenoxy)-3a,4-dihydroxyhexahydrocyclopenta[c]pyrrol-2(1H)-yl)-1-hydroxyethyl)-3,4-dihydroquinolin-2(1H)-one;
8-fluoro-6-((S)-2-((3aS,4S,5S,6aR)-5-(2-fluorophenoxy)-3a,4-dihydroxyhexahydrocyclopenta[c]pyrrol-2(1H)-yl)-1-hydroxyethyl)-3,4-dihydroquinolin-2(1H)-one;
9-fluoro-7-((R)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one;
9-fluoro-7-((S)-1-hydroxy-2-((3aS,5S,6aR)-3a-hydroxy-5-phenoxyhexahydrocyclopenta[c]pyrrol-2(1H)-yl)ethyl)-1,3,4,5-tetrahydro-2H-benzo[b]azepin-2-one;
8-fluoro-6-((R)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
8-fluoro-6-((S)-1-hydroxy-2-((3aS,5S,6aR)-3a-hydroxy-5-phenoxyhexahydrocyclopenta[c]pyrrol-2(1H)-yl)ethyl)-3,4-dihydroquinolin-2(1H)-one;
9-fluoro-7-((R)-1-hydroxy-2-((3aS,5S,6aR)-3a-hydroxy-5-phenoxyhexahydrocyclopenta[c]pyrrol-2(1H)-yl)ethyl)-1,5-dihydrobenzo[e][1,4]oxazepin-2(3H)-one;
9-fluoro-7-((S)-1-hydroxy-2-((3aS,5S,6aR)-3a-hydroxy-5-phenoxyhexahydrocyclopenta[c]pyrrol-2(1H)-yl)ethyl)-1,5-dihydrobenzo[e][1,4]oxazepin-2(3H)-one;
8-fluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
8-fluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
5-fluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
5-fluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
8-fluoro-6-((R)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]oxazin-2-one;
8-fluoro-6-((S)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]oxazin-2-one;
7-fluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
7-fluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
5,8-difluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
5,8-difluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
7,8-difluoro-6-((R)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
7,8-difluoro-6-((S)-1-hydroxy-2-((3aS,5S,6aR)-3a-hydroxy-5-phenoxyhexahydrocyclopenta[c]pyrrol-2(1H)-yl)ethyl)-1,4-dihydro-2H-benzo[d][1,3]oxazin-2-one;
6-((R)-1-hydroxy-2-((3aS,5S,6aR)-3a-hydroxy-5-phenoxyhexahydrocyclopenta[c]pyrrol-2(1H)-yl)ethyl)benzo[d]oxazol-2(3H)-one;
6-((S)-1-hydroxy-2-((3aS,5S,6aR)-3a-hydroxy-5-phenoxyhexahydrocyclopenta[c]pyrrol-2(1H)-yl)ethyl)benzo[d]oxazol-2(3H)-one;
6-((R)-2-((3aS,5S,6aR)-5-(2-fluorophenoxy)-3a-hydroxyhexahydrocyclopenta[c]pyrrol-2(1H)-yl)-1-hydroxyethyl)benzo[d]oxazol-2(3H)-one;
6-((S)-2-((3aS,5S,6aR)-5-(2-fluorophenoxy)-3a-hydroxyhexahydrocyclopenta[c]pyrrol-2(1H)-yl)-1-hydroxyethyl)benzo[d]oxazol-2(3H)-one;
6-((R)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]oxazin-2-one;
6-((S)-2-((3aS,5S,6aR)-5-(2-fluorophenoxy)-3a-hydroxyhexahydrocyclopenta[c]pyrrol-2(1H)-yl)-1-hydroxyethyl)-1,4-dihydro-2H-benzo[d][1,3]oxazin-2-one;
4-fluoro-6-((R)-1-hydroxy-2-((3aS,5S,6aR)-3a-hydroxy-5-phenoxyhexahydrocyclopenta[c]pyrrol-2(1H)-yl)ethyl)benzo[d]thiazol-2(3H)-one;
4-fluoro-6-((S)-1-hydroxy-2-((3aS,5S,6aR)-3a-hydroxy-5-phenoxyhexahydrocyclopenta[c]pyrrol-2(1H)-yl)ethyl)benzo[d]thiazol-2(3H)-one;
7-((R)-2-((3aS,5S,6aR)-5-(2-fluorophenoxy)-3a-hydroxyhexahydrocyclopenta[c]pyrrol-2(1H)-yl)-1-hydroxyethyl)-1,3,4,5-tetrahydro-2H-benzo[b]azepin-2-one;
7-((S)-2-((3aS,5S,6aR)-5-(2-fluorophenoxy)-3a-hydroxyhexahydrocyclopenta[c]pyrrol-2(1H)-yl)-1-hydroxyethyl)-1,3,4,5-tetrahydro-2H-benzo[b]azepin-2-one;
(3aS,4S,5S,6aR)-5-(2-fluorophenoxy)-2-((R)-2-hydroxy-2-(1H-pyrrolo[2,3-b]pyridin-5-yl)ethyl)hexahydrocyclopenta[c]pyrrole-3a,4(1H)-diol; and/or
(3aS,4S,5S,6aR)-5-(2-fluorophenoxy)-2-((S)-2-hydroxy-2-(1H-pyrrolo[2,3-b]pyridin-5-yl)ethyl)hexahydrocyclopenta[c]pyrrole-3a,4(1H)-diol, or a pharmaceutically acceptable salt thereof.

50. A pharmaceutical composition comprising a compound according to claim 1 or a pharmaceutically acceptable salt thereof.

51. A method for the treatment of Parkinson's disease, Huntington's disease, Rett syndrome, amyotrophic lateral sclerosis, multiple sclerosis, seizure disorders, autism, autism spectrum disorders, Fragile X syndrome, tuberous sclerosis, Down's syndrome, pain, migraine, tinnitus, bipolar disorder, obsessive-compulsive disorder, anxiety disorder, post-traumatic stress disorder (PTSD), cocaine use disorder, major depressive disorder, refractory or treatment resistant depression, or suicidality comprising administering a therapeutically effective amount of a compound according to claim 1 or a pharmaceutically acceptable salt thereof to a patient in need of treatment thereof.
